(19) United States
(12) Patent Application Publication
(10) Pub. No.: US 2009/0156535 A1
(43) Pub. Date: Jun. 18, 2009

(54) MicroRNAs for Modulating Herpes Virus Gene Expression

(75) Inventors: Jiri Vanicek, St-Sulpice (CH); Eain Murphy, Blawenburg, NJ (US); Harlan Robins, Seattle, WA (US); Arnold J. Levine, Carversville, PA (US); Thomas Shenk, Princeton, NJ (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: The Trustees of Princeton University, Princeton, NJ (US); The Institute For Advanced Study - Louis Bamberger and Mrs. Felix Fuld Foundation, Princeton, NJ (US)

(21) Appl. No.: 12/240,336

(22) Filed: Sep. 29, 2008

Related U.S. Application Data
(60) Provisional application No. 60/995,531, filed on Sep. 27, 2007.

(51) Int. Cl. A61K 31/711; G06F 19/00; C07H 21/02; C12N 15/63

(52) U.S. Cl. 514/44; 702/19; 536/24.5; 435/320.1

(57) ABSTRACT

An algorithm for identification of microRNA (miRNA) targets within viral and cellular RNA is disclosed. Also disclosed are essential herpes virus genes whose transcripts contain one or more targets of miRNAs encoded by herpes viruses or by host cells as predicted by the algorithm, and the use of such targets, miRNAs and their derivatives for modulating viral replication and latency.

This claims benefit of U.S. Provisional Application No. 60/995,531, which included specification, claims, drawings, abstract and three (3) appendices, filed Sep. 27, 2007, the entire contents of which are incorporated by reference herein.

Pursuant to 35 U.S.C. §202(c), it is acknowledged that the United States government may have certain rights in the invention described herein, which was made in part with funds from the National Institutes of Health under Grant No: CA85786.

### FIELD OF THE INVENTION

This invention relates to the fields of molecular biology and control of gene expression, particularly viral gene expression within a virus-infected cell. In particular, the invention is related to the identification of essential herpes virus genes whose transcripts are targeted by microRNAs (miRNAs) of both viral and cellular origin, and the use of such miRNAs and their derivatives for modulating viral replication and latency.

### BACKGROUND OF THE INVENTION

Various publications, including patents, published applications, technical articles and scholarly articles are cited throughout the specification. Each of these cited publications is incorporated by reference herein, in its entirety.

Mature microRNAs (miRNAs) are ˜22-nucleotide noncoding RNAs that regulate gene expression. They are produced by excision of a 60- to 80-nucleotide stem-loop precursor from a primary transcript by the ribonuclease Drosha; transported to the cytoplasm by exportin 5; and further processed by the ribonuclease Dicer, which excises a duplex that is unwound to produce the miRNA. The miRNA enters an RNA-induced silencing complex (RISC) containing multiple proteins. Within the complex, miRNAs regulate gene expression by forming imperfectly base-paired duplexes with target mRNAs, most often within the 3′ non-coding region of the message. Generally, miRNAs inhibit translation of target mRNAs, although in some cases they might also reduce the half life and therefore the level of targeted mRNAs. Perfectly base-paired miRNAs, often termed siRNAs, appear to sponsor cleavage of target mRNAs.

The human genome encodes several hundred miRNAs (reviewed in Jackson and Standart, Sci STKE 2007:re1, 2007). An individual miRNA can control multiple target mRNAs and an individual mRNA can be targeted by multiple miRNAs, and the action of a single miRNA can produce multiple functional consequences that lead to a coordinated physiological response. For example, the D. melanogaster miRNA that is encoded by bantam induces tissue growth by both stimulating cell proliferation and inhibiting apoptosis. Viruses also encode miRNAs, suggesting that, like their host cells, they employ these RNAs for gene regulation (reviewed in Sullivan and Ganem, 2005, Mol. Cell 20, 3-7). Multiple members of the human herpesvirus family have been shown to encode miRNAs, including Epstein-Barr virus (EBV, Pfeffer et al., 2004, Science 304, 734-736), Kaposi's sarcoma-associated herpesvirus (KSHV, Cai et al., 2005, Proc Natl Acad Sci USA 102, 5570-5575; Pfeffer et al., 2005, Nat Methods 2, 269-276; Samols et al., 2005, J Virol 79, 9301-9305), human cytomegalovirus (HCMV, Dunn et al., 2005, Cell Microbiol 7, 1684-1695; Grey et al., 2005, J Virol 79, 12095-12099; Pfeffer et al., 2005, supra), and herpes simplex virus (HSV, Pfeffer et al., 2005, supra; Cui et al., 2006, J Virol 80, 5499-5508; Gupta et al., 2007, Nature 442, 82-85).

Because of their role in regulating gene expression at the post-transcriptional level, miRNAs are being widely investigated as therapeutic agents for numerous disease states, including the control of infectious agents and proliferative disorders. Several algorithms have been developed for predicting microRNA targets; for the most part, these have been used for prediction of targets in Drosophila, C. elegans, and humans. One such algorithm is Miranda (Enright et al., 2003, Genome Biology, 5, R1.1-R1.14), which predicts targets by computing an approximate free energy of binding between the microRNA and the 3′UTR as well as a score based on various empirically determined rules derived from microRNA-target pairs known from experiments. Another algorithm (Robins et al., 2005, Proc. Natl. Acad. Sci. USA 102, 4006-4009), uses the RNA structure of the 3′UTR and essentially searches for potential binding sites only in the single stranded regions of the 3′UTR. Other algorithms utilize conservation among species in their parameters (e.g., Lewis et al, 2005, Cell 120, 15-20; Robins & Press, 2005, Proc. Natl. Acad. Sci. USA 102, 15557-15562); these algorithms search for potential binding sites only in the conserved part of the 3′UTR.

In spite of the interest in exploiting miRNA for therapeutic use, the targets of miRNAs remain largely unknown. This is in part because, as outlined above, current computational methods employ structural or energetic parameters based on the molecular basis of miRNA-target interaction, which is not yet completely understood. Accordingly there is a need for improved predictive techniques and for the resultant identification of molecular targets for miRNAs.

### SUMMARY OF THE INVENTION

One aspect of the present invention features a method of identifying miRNA hybridization targets in a population of mRNA molecules, wherein the population of mRNA molecules corresponds to mRNAs encoded by one or more selected genomes. The method comprises the steps of:

a) providing one or more databases comprising selected miRNA sequences and sequences representing 3′ untranslated regions (3′UTRs) of the population of mRNA molecules;

b) determining one or more seed oligomers for each of the selected miRNA molecules;

c) computing the probability (p) of finding an oligomer complementary to a seed oligomer at any position of a random background sequence generated using a kth order Markov model based on the sequence composition of the 3′ UTRs;

d) counting the number (c) of occurrences of an oligomer in each 3′UTR that is complementary to a seed oligomer, thereby creating a collection of miRNA-3′UTR pairs;

e) providing a score for each miRNA-3′UTR pair, wherein the score is determined by a single hypothesis p-value PV_{SH} of a binomial distribution, computed by

$${PV}_{SH} ( l , c , p ) = \frac{B ( p , c , l - c + 1 )}{B ( c , l - c + 1 )} ;$$

wherein l is the length of the 3′ UTR, B(x,a,b) is the incomplete beta function and B(a,b) is the usual beta function, defined by

$$B ( x , a , b ) = {\int }_{0}^{x} {{u}^{a - 1} ( 1 - u )}^{b - 1} d u , \\ B ( a , b ) = B ( 1 , a , b ) ;$$

f) ranking the miRNA-3′UTR pairs according to their score PV_{SH}, wherein the highest rank corresponds to the smallest PV_{SH};

g) evaluating the statistical significance of the t highest-ranking microRNA-target pairs, wherein t is an integer number between 1 and the total number of pairs tested, by generating N random genomes analogous to the selected genome, wherein each random genome comprises the same number of 3′UTRs as the selected genome, and each corresponding 3′UTR is of the same length and is based on the same kth Markov model as the corresponding 3′UTR in the selected genome.

h) repeating steps c) through f) for each of the N random genomes;

i) evaluating the statistical significance of the t highest-ranking miRNA-3′UTR pairs from step f) for the selected genome by (1) counting the number Nₜ of the randomly generated genomes in which the tth pair exhibits PV_{SH} smaller than the tth pair in the selected genome and (2) computing the p-value PV_{MH}(t) corrected for Multiple Hypothesis Testing from the formula

$${PV}_{MH} ( t ) = \frac{{N}_{t}}{N} ;$$

wherein PV_{MH}(t) is the probability of finding higher scores for the t highest-ranking miRNA-3′UTR pairs in the random genome as compared with the selected genome; and

j) identifying the miRNA hybridization targets by assessing each PV_{MH}(t), wherein a smaller PV_{MH}(t), correlates with a higher probability that the predicted targets are miRNA hybridization targets.

The seed oligomers can be heptamers or hexamers, and are typically determined from positions 2-8 from the 5′ end of the miRNA sequences. The 3′UTRs may be determined experimentally or computationally. In various embodiments, the miRNA sequences are human or viral and the one or more selected genomes is a virus genome. In particular, the one or more selected genomes are from herpes viruses.

Another aspect of the invention features a system for identifying miRNA hybridization targets. The system comprises: an input interface for inputting mRNA sequences, a database of mRNA sequences or a link for connecting to a remote data input interface, data or a database of mRNA sequences; an input interface for inputting miRNA sequences, a database of miRNA sequences or a link for connecting to a remote data input interface, data or a database of miRNA sequences; a processor with instructions for comparing mRNA sequences to miRNA sequences to identify miRNA hybridization targets according to the method of claim 1. In certain embodiments, the system comprises a link for connecting to a database of mRNA sequences. Supplementally or alternatively, the system may comprise an input interface for inputting miRNA sequences.

Another aspect of the invention features a computer program comprised in a computer readable medium for implementation on a computer system for identifying miRNA hybridization targets. The computer program comprises instructions for performing the steps of the method recited above.

Another aspect of the invention features a complex comprising an mRNA hybridization target to which is hybridized a miRNA, or chemically modified miRNA or siRNA derivative thereof, wherein the hybridization of the miRNA or derivative thereof to the mRNA hybridization target is predicted by a method comprising the steps set forth hereinabove. In one embodiment, the mRNA hybridization targets are viral 3′ untranslated regions (3′UTRs). In particular, the viral 3′UTRs are from herpes simplex virus 1 or 2 (HSV), Epstein-Barr virus (EBV), human cytomegalovirus (HCMV), Kaposi's sarcoma-related herpesvirus (KSHV) or varicella zoster virus (VZV). In specific embodiments, the viral 3′UTRs are set forth in Table 9 and elsewhere herein, and are:

a) HSV 3′UTRs RL1 (ICP 34.5), RL2 (ICP0), UL1, UL2, UL5, UL9, UL11, UL13, UL14, UL16, UL20, UL24, UL34, UL35, UL37, UL39, UL42, UL47, UL49A, UL51, UL52, US1 (US 1.5, ICP22), US8, US8A, US9, US11, or US12 (ICP47);

b) EBV 3′UTRs BALF2, BALF3, BALF5, BARF0, BaRF1, BARF1, BBLF4, BDLF 3.5, BDLF4, BFRF2, BGLF1, BGLF2, BGLF3, BGLF 3.5, BHLF1, BHRF1, BLLF3, BMRF1, BNRF1, BOLF1, BRLF1, BSLF2/BMLF1, BVLF1, BXLF1, BXRF1, BZLF1, BZLF2, LF3, LMP-1, LMP-2A, or LMP-2B;

c) HCMV 3′UTRs IE1 (UL123), IE2 (UL122), RL1, RL10, UL3, UL16, UL17, UL20, UL26, UL29, UL31, UL32, UL33, UL34, UL37, UL38, UL40, UL43, UL44, UL45, UL50, UL51, UL52, UL54, UL57, UL60, UL61, UL67, UL69, UL78, UL79, UL80, UL86, UL87, UL91, UL92, UL95, UL97, UL98, UL10, UL103, UL105, UL107, UL112-113, UL117, UL120, UL137, UL141a, UL151, UL151a, UL153, US7, US10, US12, US14, US24, US26, US27, US28, New ORF1, or New ORF3;

d) KSHV 3′UTRs ORF6, ORF7, ORF8, ORF9, ORF16, ORF18, ORF21, ORF25, ORF26, ORF28, ORF32, ORF40, ORF47, ORF49, ORF 50 (Rta), ORF56, ORF57, ORF58, ORF59, ORF63, ORF72, ORF73 (LANA), ORF74, ORF75, ORFK4, ORFK8 (Zta), ORFK13, and ORFK14; or

e) VZV 3′UTRs ORF16, ORF47, ORF52, ORF55, ORF59, ORF61, or ORF62.

In specific embodiments, the miRNAs are from HSV, EBV, HCMV, KSHV or humans. In particular, the miRNAs comprise those set forth in Table 9 herein. Sequences complementary thereto, as appropriate, are also encompassed. More particularly, the miRNAs comprise those set forth in any of Tables 1, 2, 3, 4, 5, 6, 7 or 8 herein.

In various embodiments, the complex comprises the miRNA-target pairs set forth in Table 1 and Table 2 herein. In other embodiments, the complex comprises the miRNA-target pairs set forth in Tables 3C, 4C, 5C, 6C and 7 herein. In particular, the mRNA hybridization targets are 3′UTRs of immediate early (IE) genes set forth in Table 8 herein, wherein the pairs are: ebv-miR-BART15 targeting EBV 3′UTRs of BZLF1 or BRLF1; ebv-miR-BHRF1-3 targeting EBV 3′UTRs of BZLF1 or BRLF1; hcmv-miR-UL112-1 targeting HCMV 3′UTR of IE (UL123); or kshv-miR-K12-6-3p targeting KSHV 3′UTRs of Zta (ORFK8) or Rta (ORF 50). More particularly, the mRNA hybridization targets are 3′UTRs of HCMV E genes and the pairs are hcmv-miR-UL112-1 targeting IE1 (UL123); or any one of human-encoded miRNAs hsa-miR-200b, hsa-miR-200c and hsa-miR-429, targeting IE2 (UL122), as described in detail in Examples 2 and 3.

Another aspect of the invention features a siRNA or a chemically modified analog of a miRNA, which hybridizes with one or more mRNA targets selected from the viral 3′UTRs set forth above. The siRNA or chemically modified miRNA, comprises a seed sequence of any of the miRNAs set forth in Table 9, and may comprise a seed sequence of a miRNA selected from the representative miRNA sequences of Table 9, namely SEQ ID NOS: 216-428. In particular embodiments, the siRNA or chemically modified miRNA contains a seed sequence that comprises, as at least a portion thereof, one of the hexamer or heptamer sequences set forth in Tables 3A, 4A, 5A or 6A, or its complement. In other embodiments, the siRNA or chemically modified analog of miRNA is based on any of the miRNAs set forth in Table 9, and more particularly as set forth in Tables 1, 2, 3, 4, 5, 6, 7 or 8.

Another aspect of the invention features a vector comprising a polynucleotide which, when expressed in a mammalian cell, produces a transcript that is processed within the cell to form a miRNA or a siRNA derivative thereof, which is capable of binding to a viral 3′UTR selected from any of those viral 3′UTRs set forth hereinabove. In particular, the vector comprises a polynucleotide which, when expressed in a mammalian cell, produces a transcript that is processed within the cell to form a miRNA or an siRNA derivative of a miRNA comprising one or more of the miRNAs set forth in Table 9 herein. In particular embodiments, the miRNA or siRNA derivative is selected from those listed respectively in Tables 1, 2, 3, 4, 5, 6, 7 or 8.

Another aspect of the invention features a pharmaceutical composition for treatment of herpes virus infection caused by HSV, EBV, HCMV, KSHV or VSV, comprising a pharmaceutical carrier and miRNA which is capable of binding to a viral 3′UTR selected from any of those viral 3′UTRs set forth hereinabove. In particular, the miRNA is one or more of the miRNAs set forth in Table 9 herein. In particular embodiments, the miRNA is selected from those listed respectively in Tables 1, 2, 3, 4, 5, 6, 7 or 8. In certain embodiments, the miRNA comprises at least one chemical modification. In other embodiments, the miRNA is replaced with a siRNA that hybridizes with the herpes virus sequence with which the miRNA hybridizes in situ. In yet other embodiments, the miRNA is provided as a vector with a polynucleotide that, when transcribed and processed in a mammalian cell, produces the one or more miRNAs. In these embodiments, the polynucleotide may be customized to produce a siRNA that hybridizes with the herpes virus sequence with which the miRNA hybridizes in situ. The pharmaceutical composition can comprise more than one miRNA or derivative, and further may comprise one or more other antiviral agents.

Another aspect of the invention features a kit or article of manufacture comprising the above-described pharmaceutical composition and instructions for administering the composition to treat a herpes virus infection. Optionally, the kit or article may contain one or more other antiviral agents and instructions for their use in conjunction with the pharmaceutical composition.

Another aspect of the invention features a method of treating a herpes virus infection in a patient. The method comprises administering to the patient a pharmaceutical composition comprising a miRNA or derivative thereof as described above, for a time and in an amount effective to treat the herpes virus infection in the patient.

Another aspect of the invention features a method of modulating herpes virus replication in a cell. The method comprises exposing the cell to one or more miRNAs, or chemically modified or siRNA derivatives thereof, under conditions permitting the miRNA to interact with a hybridization target thereof on a viral transcript within the cell, whereupon the interaction modulates the herpes virus replication in the cell. Again, the miRNAs are selected from Table 9, or more particularly from any one of Tables 1, 2, 3, 4, 5, 6, 7 and 8.

Other features and advantages of the invention will be understood by reference to the drawings, detailed description and examples that follow.

### BRIEF DESCRIPTION OF THE DRAWINGS

FIG. 1. miR-UL112-1 is predicted to bind to the IE1 3′UTR. The predicted miR-UL112-1 binding site within the HCMV major IE locus. At the top of the diagram, the spliced mRNAs that encode IE1 and IE2 are depicted with the non-coding exon 1 (Ex1) shown as an open box and the coding exons (Ex2-5) depicted as grey boxes. IE1 and IE2 share Ex2 and Ex3. The PolyA sites and the location of the miR-UL112-1 binding site in the 3′UTR (grey pinhead) are shown. At the bottom of the diagram, the IE1 3′UTR sequence is expanded and the putative miRNA/mRNA base pairing is depicted. The grey box denotes nucleotides within the miRNA seed sequence.

FIG. 2. miR-UL112-1 inhibits expression from a reporter mRNA containing the IE1 3′UTR. Reporter assay for miR-UL112-1 function. 293T cells were co-transfected with firefly luciferase expression plasmids containing either the wild-type (light grey) or mutant IE1 3′UTR (dark grey) as well as a Renilla luciferase internal control. Cells were additionally co-transfected with the indicated amounts of a miR-UL112-1 expressing plasmid, and transfection mixtures were balanced with the expression plasmid lacking an insert. Firefly luciferase units were normalized to Renilla luciferase. The luciferase units are shown relative to the amount of luciferase from the reporter construct in the absence of miRNA expression plasmids. Asterisks denote p-values<0.05 as determined by the Student's T-test.

FIG. 3. Viruses that lack miR-UL112-1 or its binding site synthesize more IE1 protein. (A) MRC5 fibroblasts were mock-infected (M) or infected with BFXwt (WT), BFXsub112-1⁻ (112-1⁻), BFXsub112-1r (112-1r) or BFXdlE1cis⁻ (IE1cis⁻). Cells were ³⁵S-labeled for 1 h before harvesting at the indicated times after infection. Lysates were prepared and analyzed by western blot for IE1, the late virus-coded pp28 or tubulin (top panel) or immunoprecipitation followed by electrophoresis for ³⁵S-labeled IE1 (bottom panel). The experiment shown is a representative of 6 independent immunoprecipitations. (B, top panel) Quantification of ³⁵S-labeled IE1 relative to tubulin. IE1 protein levels were quantified by phosphorimager analysis of immunoprecipated complexes from two independent experiments, each of which was analyzed by three independent immunoprecipitations, such as that displayed at the bottom of panel A. The levels of IE1 protein were normalized to tubulin levels from the Western blot in panel A. The mutant and revertant viruses are normalized to WT levels for each time point. P-values were determined by the Student's T-test. (B, middle panel) Quantification of IE1 RNA relative to UL37 RNA by qRT-PCR. Mutant and repaired viruses are normalized to WT levels for each time point. (C, bottom panel) ratio IE1 protein (from top panel) to IE1 RNA (from middle panel).

FIG. 4. hsa-miR-200b, hsa-miR-200c and hsa-miR-429 are predicted to bind to the IE1 3′UTR. The predicted hsa-miR-200b binding site within the HCMV IE2 3′UTR locus is shown as a representative miRNA:mRNA interaction. At the top of the diagram, the spliced mRNAs that encode IE1 and IE2 are shown. The PolyA sites and the location of the hsa-miR-200b binding site in the IE2 3′UTR (grey pinhead) are shown. At the bottom of the diagram, the IE2 3′UTR sequence is expanded and the putative miRNA/mRNA base pairing is depicted. The grey box denotes nucleotides within the miRNA seed sequence.

FIG. 5. Retrovirus transduced 4T07 cells overexpress hsa-miR-200b and hsa-miR-200c. Murine cells were transduced with two different retroviruses which over express both hsa-miR-200b and hsa-miR-200c (4T07:C1C2). The expression levels of the miRNAs were assayed by qRT-PCR using TaqMan probe sets specific to the two miRNAs. The amount of miRAN expression was normalized to the levels of the endogenous small nucleolar RNA RNU44. Relative amounts of the miRNA expression are shown.

FIG. 6. Luciferase reporter mRNA containing the IE2 3′UTR is inhibited in cells over-expressing hsa-miR-200b, hsa-miR-200c and hsa-miR-429. A mouse mammary tumor cell line, was transduced with either lentiviruses containing scrambled DNA (4T07) or lentiviruses which over express the hsa-miR-200b, hsa-miR-200c and hsa-miR-429 miRNAs (4T07/C1C2). These cells were co-transfected with firefly luciferase expression plasmids containing either a non-specific 3′UTR (Empty vector), the wild type 3′UTR of IE2 (IE2 3′UTR), the IE2 3′UTR with four nucleotides within the seed sequence mutated to four cysteines (Mutant IE2 3′UTR) or a 3′UTR which contains a sequence complementary to the hsa-miR-200b sequence (miR-200b pos control). Cells were additionally co-transfected with a Renilla luciferase plasmid to control for transfection efficiencies and luciferase assays. Firefly luciferase units were normalized to Renilla luciferase. The luciferase units for each plasmid are shown relative to the amount of luciferase activity in the absence of the overexpressed miRNAs.

### DETAILED DESCRIPTION OF ILLUSTRATIVE EMBODIMENTS

Various terms relating to the methods and other aspects of the present invention are used throughout the specification and claims. Such terms are to be given their ordinary meaning in the art unless otherwise indicated. Other specifically defined terms are to be construed in a manner consistent with any particular definitions provided throughout the specification. It is to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

The term “complementary” (or “complementarity”) refers to the specific base pairing of nucleotide bases in nucleic acids. The term “perfect complementarity” as used herein refers to complete (100%) complementarity within a contiguous region of double stranded nucleic acid, such as between a hexamer or heptamer seed sequence in a miRNA and its complementary sequence in a target polynucleotide, as described in greater detail herein.

“Encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system. “Exogenous” refers to any material introduced from or produced outside an organism, cell, tissue or system.

The term “expression” as used herein is defined as the transcription and/or translation of a particular nucleotide sequence driven by its promoter.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

“Homologous, homology” or “identical, identity” as used herein, refer to comparisons among amino acid and nucleic acid sequences. When referring to nucleic acid molecules, “homology,” “identity,” or “percent identical” refers to the percent of the nucleotides of the subject nucleic acid sequence that have been matched to identical nucleotides by a sequence analysis program. Homology can be readily calculated by known methods. Nucleic acid sequences and amino acid sequences can be compared using computer programs that align the similar sequences of the nucleic or amino acids and thus define the differences. In preferred methodologies, the BLAST programs (NCBI) and parameters used therein are employed, and the DNAstar system (Madison, Wis.) is used to align sequence fragments of genomic DNA sequences. However, equivalent alignments assessments can be obtained through the use of any standard alignment software.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Unless it is particularly specified otherwise herein, the proteins, virion complexes, antibodies and other biological molecules forming the subject matter of the present invention are isolated, or can be isolated.

The term, “miRNA” or “microRNA” is used herein in accordance with its ordinary meaning in the art. miRNAs are single-stranded RNA molecules of about 20-24 nucleotides, although shorter or longer miRNAs, e.g., between 18 and 26 nucleotides in length, have been reported. miRNAs are encoded by genes that are transcribed from DNA but not translated into protein (non-coding RNA), although some miRNAs are coded by sequences that overlap protein-coding genes. miRNAs are processed from primary transcripts known as pri-miRNA to short stem-loop structures called pre-miRNA and finally to functional miRNA. Typically, a portion of the precursor miRNA is cleaved to produce the final miRNA molecule. The stem-loop structures may range from, for example, about 50 to about 80 nucleotides, or about 60 nucleotides to about 70 nucleotides (including the miRNA residues, those pairing to the miRNA, and any intervening segments). Mature miRNA molecules are partially complementary to one or more messenger RNA (mRNA) molecules, and they function to regulate gene expression, as described in greater detail herein. Thus, in various aspects of the present invention, the miRNAs can be processed from a portion of an miRNA transcript (i.e., a precursor miRNA) that, in some embodiments, can fold into a stable hairpin (i.e., a duplex) or a stem-loop structure.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning and amplification technology, and the like, and by synthetic means. An “oligonucleotide” as used herein refers to a short polynucleotide, typically less than 100 bases in length.

The term “siRNA” (also “short interfering RNA” or “small interfering RNA”) is given its ordinary meaning, and refers to small strands of RNA (21-23 nucleotides) that interfere with the translation of messenger RNA in a sequence-specific manner. SiRNA binds to the complementary portion of the target messenger RNA and is believed to tag it for degradation. This function is distinguished from that of miRNA, which is believed to repress translation of mRNA but not to specify its degradation.

The term “therapeutic” as used herein means a treatment and/or prophylaxis. A therapeutic effect is obtained by suppression, remission, or eradication of a disease state, particularly a disease state associated with a herpes virus infection.

The term “treatment” as used within the context of the present invention is meant to include therapeutic treatment as well as prophylactic, or suppressive measures for the disease or disorder. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease. This includes for instance, prevention of CMV propagation to uninfected cells of an organism. The phrase “diminishing CMV infection” is sometimes used herein to refer to a treatment method that involves reducing the level of infection in a patient infected with CMV, as determined by means familiar to the clinician.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a replicon, such as plasmids, phagemids, cosmids, baculoviruses, bacmids, bacterial artificial chromosomes (BACs), yeast artificial chromosomes (YACs), as well as other bacterial, yeast and viral vectors, to which another nucleic acid segment may be operably inserted so as to bring about the replication or expression of the segment. “Expression vector” refers to a vector comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The inventors have developed an improved algorithm for the prediction of mRNAs that are targeted by known miRNAs. The algorithm can be used to predict miRNA targets in any organism, but is expected to be particularly useful in predicting targets in viral mRNA. In an exemplary embodiment described in detail in the examples, the algorithm was employed to identify the targets of cell-coded and virus-coded miRNAs in mRNAs encoded by herpes viruses. Certain of these predictions have been validated experimentally. These naturally occurring miRNAs target mRNAs encoding essential herpes virus proteins. Consequently, they can be used and developed to inhibit acute replication and pathogenesis of the herpes viruses and prevent the re-emergence of herpes viruses from latency.

Algorithm for prediction of miRNA targets: The miRNA-target-predicting algorithm described herein is superior to currently available methodology in that it allows prediction of viral targets of both human and viral microRNAs without detailed knowledge of the molecular basis of microRNA-target interaction, the mechanism of which is not well understood. The inventors' algorithm compensates the incomplete experimental understanding of target selection with a bioinformatics approach that scores each potential miRNA target site with a probability that it would appear by chance in a random sequence with similar composition. Multiple miRNAs and multiple potential 3′UTR targets are tested. The algorithm evaluates the statistical significance of the scores of the most likely targets by a Monte Carlo simulation in which p-values are corrected for Multiple Hypothesis Testing. While the algorithm is general and can be used to predict miRNA targets in any organism, the algorithm is expected to be particularly predictive in viruses, due to the small size of their genomes. Further, based on both computational results of the algorithm and the experimental confirmation described below, the algorithm will be extremely useful for understanding and identifying opportunities for manipulating regulation of immediate early genes and genes involved in DNA replication, regulation of the lytic and latent infection in herpesviruses, and interaction with the immune system of the host.

The algorithm of the invention is based on the assumption that the target 3′UTR sequence, particularly but not exclusively in viruses, coevolved with the sequence of the miRNA. The method makes use of the experimental fact that the miRNA binding requires a perfect complementarity of a “seed” oligomer sequence near the 5′ end of the miRNA to an oligomer sequence in the 3′UTR. As a result of coevolution, the number of actual seed oligomers present in the 3′ UTR of a targeted gene will be higher than the number expected based on a random background sequence. The algorithm orders miRNA-3′ UTR pairs according to the increasing probability (p-value) that the observed number of seed sites is smaller than that which would occur in the random sequence (the most likely targets have the smallest p-value). This part of the algorithm is described in steps 1-6 below. Due to Multiple Hypothesis Testing, these p-values are considered only as scores for ranking the potential targets. The statistical significance of the highest ranking potential targets is evaluated rigorously in the end by a Monte-Carlo simulation in which p-values corrected for Multiple Hypothesis Testing are computed (described in steps 7-10 below). This latter method is needed because the discrete nature of the data does not allow the standard methods for analyzing Multiple Hypothesis Testing problems. That is, most genes have 0 binding sites for a given microRNA, and therefore most single hypothesis p-values are 1, whereas in the continuous case, the p-values close to 1 have a uniform distribution.

The typical steps in the algorithm are set forth below.
Step 1. Determine the seed sequences of the microRNAs of interest. In a preferred practice, heptamers (sequences consisting of 7 nucleotides) at positions 2-8 from the 5′ end of the microRNAs are considered. (More generally, n-mers are considered, but most often n=6 or 7.)
Step 2. Determine the 3′UTRs of the genes of interest. The first choice is to use experimentally determined 3′UTR sequences. If these are not known, the second choice is to determine the 3′ UTRs computationally by the experimentally determined positions of polyadenylation sites. If even these are not known, the third choice is to find the first polyadenylation site motif in the sequence downstream of the stop codon of each gene computationally.
Step 3. Compute the probability p of finding an oligomer complementary to a given seed oligomer at any given position of a random background sequence based on the kth order Markov model [which considers composition of the 3′ UTR up to (k+1)-mers]. By “global” is meant that the composition of 3′UTRs of all genes are taken together to form the Markov model. In the present case, k=2 is preferred. To be more specific, assume that the combined length of all 3′UTR is lₜₒₜₐₗ and that one is interested in determining the probability p of finding an n-mer X₁X₂ . . . Xₙ in a hypothetical 3′ UTR based on the k-th order Markov model. Let c(X₁X₂.Xⱼ) denote the count of j-mer X₁X₂ . . . Xⱼ for 0≦j≦k+1. Frequency of X₁X₂ . . . Xⱼ is f(X₁ . . . Xⱼ)=C(X₁ . . . Xⱼ)/lₜₒₜₐₗ. Denoting p (Xⱼ₊₁|X₁ . . . Xⱼ) the conditional probability of (J+1)-st nucleotide being Xⱼ₊₁ if it is preceded by a j-mer X₁ . . . Xⱼ, we compute p as

$$p = p ( {X}_{n} \vert {X}_{n - k} … {X}_{n - 1} ) … p ( {X}_{k + 1} \vert {X}_{1} … {X}_{k} ) f ( {X}_{1} … {X}_{k} ) = \frac{f ( {X}_{n - k} … {X}_{n} ) … f ( {X}_{1} … {X}_{k + 1} )}{f ( {X}_{n - k} … {X}_{n - 1} ) … f ( {X}_{2} … {X}_{k} )} .$$

Step 4. Count the number c of occurrences of an oligomer complementary to each seed oligomer in each 3′UTR.
Step 5. Give each microRNA-3′UTR pair a score, given by the single hypothesis p-value PV_{SH} of a binomial distribution, computed by

$${PV}_{SH} ( l , c , p ) = \frac{B ( p , c , l - c + 1 )}{B ( c , l - c + l )} .$$

Here l is the length of the 3′ UTR, B(x,a,b) is the incomplete beta function and B(a,b) is the usual beta function,

$$B ( x , a , b ) = {\int }_{0}^{x} {{u}^{a - 1} ( 1 - u )}^{b - 1} d u , \\ B ( a , b ) = B ( 1 , a , b ) .$$

Step 6. Rank the microRNA-3′UTR pairs according to their score PV_{SH} (the 1^{st} pair is the one with the smallest PV_{SH}).
Step 7. Evaluate the statistical significance of the top microRNA-target pairs by the following procedure: First generate N random genomes analogous to the actual genome of interest. This means that each genome will have exactly the same number of 3′UTR as the genome of interest, each corresponding 3′UTR will be of the same length and will be based on the same kth Markov model as the 3′UTR in the actual genome.
Step 8. Repeat the analysis in steps 3) to 6) for each of the N random genomes.
Step 9. Now evaluate the statistical significance of the top t microRNA-target pairs in the results from step 6) for the actual genome by counting the number Nₜ of the randomly generated genomes in which the tth top pair has PV_{SH} smaller than the tth pair in the actual genome. For each t, compute the p-value PV_{MH}(t) corrected for Multiple Hypothesis Testing by

$${PV}_{MH} ( t ) = \frac{{N}_{t}}{n} .$$

Step 10. PV_{MH}(t) is the probability of finding better scores for the top t potential microRNA-3′UTR pairs in a random genome with similar properties as the actual genome. The smaller PV_{MH}(t), the higher the chance that the predicted targets are real targets.

Optionally, certain variations and extensions of the algorithm may be incorporated. For instance, if information on conservation among various strains of a specific virus is available, it is advantageous to consider this conservation. In this instance, the count c in step 4) denotes only the count of the conserved n-mers complementary to a given seed n-mer among several strains, and 1 in step 5) denotes the total count of all conserved n-mers instead of the total length of the 3′UTR.

As another non-limiting example, if it is preferred to increase sensitivity and decrease specificity, seed hexamers instead of heptamers can be used. If this alternative is selected, hexamers complementary to positions 2-7 as well as 3-8 in the microRNAs are recommended. Positions 3-8, as well as the standard 2-7 should be considered because it is often experimentally determined that the extent of microRNA seed sequence varies by one nucleotide. Additionally, the experimental error in determining the precise extent of a mature miRNA is typically one nucleotide.

As yet another illustration, if it is suspected that the overall sequence composition in a viral genome is not homogeneous, then a local Markov model should be used, i.e., a separate Markov model should be created for each 3′UTR. In such a case, lₜₒₜₐₗ in step 3) is replaced by the length of the given 3′UTR l and the various counts denote counts in the given 3′UTR rather than in a combination of all 3′UTRs. The benefit of the “global” model is that it provides enough statistics to consider higher order Markov models. The advantage of the “local” model is that it captures inhomogeneity of the genome such as the so-called isochores in genomes of higher animals (such an inhomogeneity however should not play a major role in the very small genomes of viruses). For herpesviruses, the statistics should be sufficient to consider up to about the 4^{th} order global Markov model and up to the 1^{st} order local Markov model.

The methods outlined above differ in several important aspects from previously used algorithms for predicting miRNA targets. As mentioned earlier, the other algorithms utilize such parameters as free energy of binding and certain empirically determined rules derived from known miRNA-target pairs (Enright et al., 2003, supra), RNA structure of the 3′ UTR (Robins et al., 2005, supra), and conservation among species (Lewis et al., 2005, supra; Robins & Press, 2005, supra).

In contrast, the algorithm of the present invention does not use the free energy of binding or the RNA structure, and can rarely use conservation because (1) miRNAs are not conserved among different viral species, and (2) with the exception of human CMV, sufficient information on conservation among strains of a given species typically is not available. Instead, the algorithm described herein uses a computation of a p-value score, which is based solely on a rigorous evaluation of the statistical significance of the seed binding and does not rely on any empirical information other than the requirement of seed binding (which is the only requirement common to all experimentally known microRNA-target pairs). Similar to the algorithm of Robins and Press based on conservation among species, the presently described algorithm also use a Markov model as a model of a random 3′UTR. But while the Robins and Press algorithm estimates the overall probability that a given gene as a target of any subset of all human microRNAs, the algorithm of this invention computes the p-value for each gene and microRNA separately. Most importantly, the algorithm of the present invention uses a different method for scoring (single hypothesis p-value computed exactly) and analysis of statistical significance of the results (multiple hypothesis p-value computed numerically without any approximation) while the Robins and Press algorithm uses an approximate Poisson odds ratio method. Other less central, but significant differences are (1) the Robins and Press algorithm uses hexamer seeds while the present algorithm preferentially uses heptamer seeds to increase specificity, and (2) the Robins and Press algorithm uses a local Markov model, whereas the present algorithm preferentially uses a global Markov model, particularly for the preferred target population of viral genomes, which are fairly small and do not have isochores.

Predicted viral mRNA targets of viral and cellular miRNAs: The above-described methods were used to predict herpes virus targets of both viral and human miRNAs. Among the most frequently predicted targets were the following important groups of genes: (1) immediate early genes (IE genes); (2) genes involved in DNA replication (DNA rep.); and (3) viral inhibitors of apoptosis (vIAP) and other immune evasion genes.

The algorithm predicts that the following cellular or viral miRNAs will target at least one 3′UTR within a particular virus.
(1) Herpes simplex virus types 1 and 2 (HSV1 HSV2): hsv1-miR-H1, hsv1-miR-LAT;
(2) Epstein-Barr virus (EBV): ebv-miR-BART1-3p, ebv-miR-BART1-5p, ebv-miR-BART2, ebv-miR-BART3-3p, ebv-miR-BART3-5p, ebv-miR-BART4, ebv-miR-BART5, ebv-miR-BART6-3p, ebv-miR-BART6-5p, ebv-miR-BART7, ebv-miR-BART8-3p, ebv-miR-BART8-5p, ebv-miR-BART9, ebv-miR-BART10, ebv-miR-BART11-3p, ebv-miR-BART11-5p, ebv-miR-BART12, ebv-miR-BART13, ebv-miR-BART14-3p, ebv-miR-BART14-5p, ebv-miR-BART15, ebv-miR-BART16, ebv-miR-BART17-3p, ebv-miR-BART17-5p, ebv-miR-BART18, ebv-miR-BART19, ebv-miR-BART20-3p, ebv-miR-BART20-5p, ebv-miR-BHRF1-1, ebv-miR-BHRF1-2*, and ebv-miR-BHRF1-3;
(3) Human cytomegalovirus (HCMV): hcmv-miR-UL22-1, hcmv-miR-UL22A-1*, hcmv-miR-UL31-1, hcmv-miR-UL36-1, hcmv-miR-UL36-1-N, hcmv-miR-UL53-1, hcmv-miR-UL54-1, hcmv-miR-UL70-3p, hcmv-miR-UL70-5p, hcmv-miR-UL102-1, hcmv-miR-UL102-2, hcmv-miR-UL111a-1, hcmv-miR-UL112-1, hcmv-miR-UL148D-1, hcmv-miR-US4, hcmv-miR-US5-1, hcmv-miR-US5-2, hcmv-miR-US5-2-N, hcmv-miR-US25-1, hcmv-miR-US25-2-5p, hcmv-miR-US25-2-3p, hcmv-miR-US29-1, and hcmv-miR-US33-1;
(4) Kaposi's sarcoma sarcoma-associated herpesvirus (KSHV or HHV-8): kshv-miR-K12-1, kshv-miR-K12-2, kshv-miR-K12-3, kshv-miR-K12-3*, kshv-miR-K12-4-5p, kshv-miR-K12-4-3p, kshv-miR-K12-5, kshv-miR-K12-6-5p, kshv-miR-K12-6-3p, kshv-miR-K12-7, kshv-miR-K12-8, kshv-miR-K12-9*, kshv-miR-K12-9, kshv-miR-K12-10a, kshv-miR-K12-10b, kshv-miR-K12-11, and kshv-miR-K12-12;
(5) Human cellular (Homo sapiens):
Targeting HSV: hsa-miR-138, hsa-miR-205, hsa-miR-326, hsa-miR-381, hsa-miR-425, hsa-miR-492, and hsa-miR-522;
Targeting EBV: hsa-miR-24, hsa-miR-214, hsa-miR-296, hsa-miR-328, hsa-miR-346, and hsa-miR-502;
Targeting HCMV: hsa-miR-15a, hsa-miR-15b, hsa-miR-16, hsa-miR-103, hsa-miR-107, hsa-miR-126, hsa-miR-142-5p, hsa-miR-184, hsa-miR-194, hsa-miR-195, hsa-miR-200b, hsa-miR-200c, hsa-miR-202, hsa-miR-326, hsa-miR-330-5p, hsa-miR-367, hsa-miR-424, hsa-miR-429, hsa-miR-450-b-3p, hsa-miR-497, hsa-miR-503, hsa-miR-548d-3p, hsa-miR-548k, hsa-miR-551a, hsa-miR-551b, hsa-miR-552, hsa-miR-592, hsa-miR-598, hsa-miR-652, hsa-miR-769-3-p, and hsa-miR-1226;
Targeting KSHV: hsa-let-7a, hsa-let-7b, hsa-let-7c, hsa-let-7d, hsa-let-7e, hsa-let-7f, hsa-let-7g, hsa-let-7i, hsa-miR-1, hsa-miR-9, hsa-miR-15a, hsa-miR-15b, hsa-miR-16, hsa-miR-17-5p, hsa-miR-18a, hsa-miR-18b, hsa-miR-20a, hsa-miR-20b, hsa-miR-23a, hsa-miR-23b, hsa-miR-30a-5p, hsa-miR-30a-3p, hsa-miR-30b, hsa-miR-30c, hsa-miR-30e-5p, hsa-miR-30e-3p, hsa-miR-93, hsa-miR-98, hsa-miR-105, hsa-miR-106a, hsa-miR-106b, hsa-miR-125a, hsa-miR-125b, hsa-miR-129, hsa-miR-134, hsa-miR-137, hsa-miR-141, hsa-miR-142-3p, hsa-miR-145, hsa-miR-150, hsa-miR-154, hsa-miR-181a, hsa-miR-181b, hsa-miR-181c, hsa-miR-181d, hsa-miR-182*, hsa-miR-194, hsa-miR-195, hsa-miR-196a, hsa-miR-196b, hsa-miR-199a, hsa-miR-199b, hsa-miR-200a, hsa-miR-205, hsa-miR-206, hsa-miR-210, hsa-miR-213, hsa-miR-299-3p, hsa-miR-302a, hsa-miR-302b, hsa-miR-302c, hsa-miR-302d, hsa-miR-324-3p, hsa-miR-326, hsa-miR-329, hsa-miR-337, hsa-miR-338, hsa-miR-340, hsa-miR-346, hsa-miR-372, hsa-miR-373, hsa-miR-424, hsa-miR-448, hsa-miR-450, hsa-miR-453, hsa-miR-455, hsa-miR-490, hsa-miR-491, hsa-miR-492, hsa-miR-497, hsa-miR-518b, hsa-miR-518c, hsa-miR-518d, hsa-miR-519d, hsa-miR-520a, hsa-miR-520b, hsa-miR-520c, hsa-miR-520d, hsa-miR-520g, hsa-miR-520h, hsa-miR-525, and hsa-miR-526b;
Targeting VZV: hsa-miR-99a, hsa-miR-99b, hsa-miR-100, hsa-miR-124a, hsa-miR-132, hsa-miR-141, hsa-miR-150, hsa-miR-197, hsa-miR-200a, hsa-miR-212, hsa-miR-219, hsa-miR-330, hsa-miR-374, hsa-miR-371, hsa-miR-339, hsa-miR-451, hsa-miR-495, and hsa-miR-510.

Within particular viruses, the algorithm predicts miRNA (cellular or viral) targets within the 3′UTRs of the following genes:
(1) Herpes simplex virus types 1 and 2 (HSV1, HSV2): RL1 (ICP 34.5), RL2 (ICP0), UL1, UL2, UL5, UL9, UL11, UL13, UL14, UL16, UL20, UL24, UL34, UL35, UL37, UL39, UL42, UL47, UL49A, UL51, UL52, US1 (US 1.5, ICP22), US8, US8A, US9, US11, and US12 (ICP47);
(2) Epstein-Barr virus (EBV): BALF2, BALF3, BALF5, BARF0, BaRF1, BARF1, BBLF4, BDLF 3.5, BDLF4, BFRF2, BGLF1, BGLF2, BGLF3, BGLF 3.5, BHLF1, BHRF1, BLLF3, BMRF1, BNRF1, BOLF1, BRLF1, BSLF2/BMLF1, BVLF1, BXLF1, BXRF1, BZLF1, BZLF2, LF3, LMP-1, LMP-2A, and LMP-2B;
(3) Human cytomegalovirus (HCMV): IE1 (UL123), IE2 (UL122), RL1, RL10, UL3, UL16, UL17, UL20, UL26, UL29, UL31, UL32, UL33, UL34, UL37, UL38, UL40, UL43, UL44, UL45, UL50, UL51, UL52, UL54, UL57, UL60, UL61, UL67, UL69, UL78, UL79, UL80, UL86, UL87, UL91, UL92, UL95, UL97, UL98, UL100, UL103, UL105, UL107, UL112-113, UL117, UL120, UL137, UL141a, UL151, UL151a, UL153, US7, US10, US12, US14, US24, US26, US27, US28, New ORF1, and New ORF3;
(4) Kaposi's sarcoma sarcoma-associated herpesvirus (KSHV or HHV-8): ORF6, ORF7, ORF8, ORF9, ORF16, ORF18, ORF21, ORF25, ORF26, ORF28, ORF32, ORF40, ORF47, ORF49, ORF 50 (Rta), ORF56, ORF57, ORF58, ORF59, ORF63, ORF72, ORF73 (LANA), ORF74, ORF75, ORFK4, ORFK8 (Zta), ORFK13, and ORFK14;
(5) Varicella zoster virus (VZV): ORF16, ORF47, ORF52, ORF55, ORF59, ORF61, and ORF62.

Representative examples of miRNAs and their predicted targets of particular biological significance are listed below in Tables 1 and 2. Additional lists of miRNAs, 3′UTRs and miRNA-3′UTR pairs are set forth in Example 1.

TABLE 1
Selected viral miRNAs and their viral 3′UTR targets

Herpes simplex virus types 1 and 2 (HSV-1 and HSV-2):
hsv1-miR-LAT targeting ICP0 (=RL2): IE gene; UL9 (=oriBP = DNA origin binding
protein): DNA rep.; UL42 (=DNA polymerase processivity factor): DNA rep.; ICP34.5
(=RL1): immune evasion
Epstein-Barr Virus (EBV):
ebv-miR-BHRF1-3 and ebv-miR-BART15 targeting BZLF1 and BRLF1: IE genes
ebv-miR-BART2 (perfect complementarity) and ebv-miR-BART6-3p targeting BALF5
(=DNA polymerase): DNA rep.
ebv-miR-BART1-3p targeting BHRF1 (=vBCL-2): vIAP
ebv-miR-BART10 targeting BBLF4 (=helicase-primase subunit): DNA rep.
ebv-miR-BHRF1-3 targeting BSLF2/BMLF1 (=Mta): transactivator
ebv-miR-BART17-5p targeting BMRF1 (=DNA polymerase processivity factor): DNA
rep.
ebv-miR-BART6-3p (perfect complementarity) targeting LF3
Human cytomegalovirus (HCMV):
hcmv-miR-UL112-1 targeting IE1 (=UL123): IE gene
hcmv-miR-UL36-1 (almost perfect complementarity) targeting UL37: IE gene and vIAP
hcmv-miR-UL53-1 (perfect complementarity) targeting UL52
hcmv-miR-UL54-1 targeting UL112-113 (organization of DNA replication centers): DNA
rep., UL45 (=ribonucleotide reductase): DNA rep.
hcmv-miR-US25-2-5p targeting UL57 (=SSB = single-stranded DNA binding protein):
DNA rep.
hcmv-miR-UL148D-1 targeting UL26: transactivator of IE promoter, UL98
(=deoxyribonuclease), UL103, UL151a (perfect complementarity)
hcmv-miR-US5-1 and US5-2 (both perfect complementarity) targeting US7
hcmv-miR-US25-2-3p targeting UL32
hcmv-miR-US33-1 (perfect complementarity) targeting US28: chemokine receptor
Kaposi's sarcoma-associated herpesvirus (KSHV or HHV-8):
kshv-miR-K12-6-3p targeting Zta (=ORF K8) and Rta (=ORF 50): IE genes
kshv-miR-K12-8 targeting ORF9 (=DNA polymerase): DNA rep.
kshv-miR-K12-10b targeting LANA (=ORF73 = latency associated nuclear antigen):
latent gene

TABLE 2
Selected human miRNAs and their viral 3′UTR targets

Herpes simplex virus types 1 and 2 (HSV-1 and HSV-2):
hsa-miR-138 targeting ICP0 (=RL2): IE gene
hsa-miR-425 targeting UL47 (=virion protein transactivating): IE gene
hsa-miR-381 targeting ICP22 (US1) and US1.5: IE genes
hsa-miR-522 targeting UL5 (=DNA helicase-primase component): DNA rep.
hsa-miR-326 targeting ICP47 (=US12): IE gene
hsa-miR-205 targeting UL2 (=uracil DNA glycosylase): DNA rep.
hsa-miR-492 targeting UL52 (=DNA helicase-primase component): DNA rep.
Epstein-Barr Virus (EBV):
hsa-miR-24 targeting BHRF1 (=vBCL-2): vIAP
hsa-miR-214 targeting BXLF1 (=thymidine kinase): DNA rep.
hsa-miR-296 targeting BALF5 (=DNA polymerase): DNA rep.
hsa-miR-296 and hsa-miR-328 targeting LMP-2A and LMP-2B: latent genes
hsa-miR-346 and hsa-miR-502 targeting LMP-1: latent gene
Human cytomegalovirus (HCMV):
hsa-miR-200b, 200c, 429 targeting IE2 (=UL122): IE gene
hsa-miR-769-3-p, 450-b-3p targeting IE1 (=UL 123): IE gene
hsa-miR-503 targeting UL44 (=DNA polymerase processivity factor): DNA rep.;
UL37: IE gene and vIAP
hsa-miR-503, 592 targeting UL54 (=DNA polymerase): DNA rep.
hsa-miR-142-5p targeting UL105 (=DNA helicase-primase): DNA rep.; UL97
(=phosphotransferase and ganciclovir kinase); UL33 (=viral glucocorticoid receptor, vGCRs);
US 27 (=viral glucocorticoid receptor, vGCRs)
hsa-miR-103, 107, 202, 15a, 15b, 16, 195, 424, 497 targeting UL38: Viap
hsa-miR-367 targeting UL57: DNA rep.
hsa-miR-1226 targeting UL50: Nuclear egress
hsa-miR-184 targeting UL31 (=dUTPase family)
hsa-miR-16, 15b, 195, 424, 15a, 497 (almost the same as those targeting UL38) targeting UL78
(=GCPR family)
hsa-miR-652 targeting New ORF3
hsa-miR-552 targeting UL91
hsa-miR-548k targeting UL29: temperance in RPE cells
hsa-miR-330-5p, 326 targeting New ORF1
hsa-miR-548d-3p targeting UL107
hsa-miR-598 targeting UL60
hsa-miR-126 targeting UL20 (=T-cell receptor homolog)
hsa-miR-194 targeting UL17 (=7TM membrane glycol-protein)
hsa-miR-551a, 551b targeting UL100
hsa-miR-503 targeting RL1
Kaposi′s sarcoma-associated herpesvirus (KSHV or HHV-8):
hsa-miR-302b*, 105, 150, 210, 142-3p, 302a-d, 372, 373, 520a-e, 526b*, 93, 17-5p, 519d, 20a-b,
106a-b, 199a-b, 520g-h targeting ORF6 (=ssDNA binding protein): DNA rep.
hsa-miR-329, 141, 200a, 324-3p, 213, 182*, 105, 455, 518b-d, 453, hsa-let-7a-g and i, and
hsa-miR-98, targeting LANA (=ORF73 latency associated nuclear antigen): latent gene
hsa-miR-199a-b, 137, 205, 154, 346, 340, 490, 9, 1, 206, 492, 299-3p, 491 targeting ORF56
(=DNA helicase-primase subunit): DNA rep.
hsa-miR-129, 450, 448, 134, 196a-b, 337, 141, 200a, 194, 30a-5p, 30a-3p, 30b-d, 30e-5p, 30e-3p,
195, 15a-b, 16, 424, 497 targeting ORF58 (=DNA polymerase processivity factor):
DNA rep.
hsa-miR-326, 181a-d, 181a, 23a-b, 125a-b, 340, 18a-b, 520a*, 525, 145, 338 targeting ORF21
(=thymidine kinase): DNA rep.
Varicella zoster virus (VZV):
hsa-miR-132, 212, 451, 495 targeting ORF62: IE gene
hsa-miR-510, 150, 124a, 330 targeting ORF61: IE gene
hsa-miR-197 targeting ORF52 (=helicase-primase subunit)
hsa-miR-374 targeting ORF16 (=DNA polymerase processivity subunit)
hsa-miR-371, 219, 339 targeting ORF47 (=tegument serine/threonine protein kinase)
hsa-miR-141, 200a targeting ORF59 (=uracil-DNA glycosylase)
hsa-miR-99a, 99b, 100 targeting ORF55 (=helicase-primase helicase subunit)

The miRNAs identified in accordance with the present invention are natural regulators of viral gene expression. As a consequence, modulating, i.e., inhibiting or augmenting, these miRNA activities can be expected to perturb viral replication, latency and pathogenesis. As discussed in greater detail below, small inhibitory RNAs (siRNAs) that inhibit expression of the virus-coded mRNAs at the same site targeted by the naturally occurring miRNAs, and derivatives of the miRNAs and siRNAs that have been modified to enhance their efficacy, e.g., to extend their half life and/or enhance their entry into cells, are expected to function as efficiently or even more efficiently than the naturally occurring miRNAs in the prevention and treatment of herpes virus disease. Finally, it is likely that artificial miRNAs, siRNAs and their derivatives that target all of the mRNAs or a subset of the mRNAs targeted by the naturally occurring miRNAs, but at a different site within the mRNAs than is targeted by the naturally occurring miRNAs, will also have therapeutic efficacy.

Why is it expected that inhibiting or augmenting these miRNAs will have therapeutic benefit? Because, for a variety of reasons, naturally occurring miRNAs and their derivatives that recognize the same or similar target elements in mRNAs are expected to exhibit therapeutic efficacy that is superior to that of artificial miRNAs and their derivatives that target different sites in the same mRNAs. One rationale for this view is evolutionary: evolution selects for efficient function, and therefore, naturally occurring miRNAs would be expected to be optimized for a specific physiological outcome. Another rationale is based on the observation that a single miRNA can regulate multiple targets. Consequently, it is possible that cell-coded miRNAs controlling the function of a viral gene also control one or more additional viral or cellular genes that contribute to successful virus replication and spread. Individual miRNAs are known to sponsor multiple functional consequences that lead to a coordinated physiological response, so there is precedent for the view that a single naturally occurring miRNA can influence the dynamics of viral replication and pathogenesis by modulation of a set of virus-coded and cell-coded mRNAs.

Regulation of gene expression: Thus, one aspect of the present invention provides methods and compositions for regulating the expression of a gene. The term “regulating” is used interchangeably with the term “modulating” throughout the specification. In particular embodiments, gene expression is regulated within a cell, e.g., a mammalian cell. In more particular embodiments, viral gene expression within a virus-infected cell is regulated. The regulation may take place in cultured cells or in cells present within a living organism. As used herein, the term “regulation of gene expression” and similar phrases inclusively refer to modulation of processes at the transcriptional or post-transcriptional level. In a preferred embodiment, gene expression is regulated at the post-transcriptional level in accordance with the typical function of a miRNA. In a specific embodiment, such regulation is accomplished through interaction between a miRNA or derivative thereof and a target element in the 3′UTR of a mRNA molecule. However, at least in part because many miRNAs have multiple targets, the interaction may also be with a coding portion of an mRNA sequence in some cases, i.e., to a portion of a mRNA which is translated to produce a protein. Thus, it should be understood that the description herein with respect to binding (also referred to as annealing or hybridizing) of miRNAs to UTRs of mRNAs is one embodiment only, and in other embodiments of the present invention, certain miRNAs may bind to coding portions of the mRNA, and/or both the coding portions and the UTR portions of the mRNA.

Typically, miRNA and siRNA function by a mechanism that results in inhibition of the production of the encoded polypeptide; in the case of miRNA, through repression of translation with possible enhanced degradation of non-translated mRNA molecules, and, in the case of siRNA, through cleavage and subsequent degradation of the mRNA. Accordingly, gene expression can be inhibited by increasing the amount and/or stability of specific miRNAs in a cell. The amount of miRNA in a cell may be increased by stimulating expression of an endogenous miRNA-encoding gene or by adding exogenous miRNA. The latter may be accomplished by administering an miRNA in mature form or as a pre-miRNA of a duplex or a stem-loop structure, which is processed by the cell to a mature form. Alternatively or additionally, a cell may be transfected with a sequence encoding a miRNA, e.g., a miRNA-encoding gene. For instance, a vector comprising a miRNA-encoding sequence under the control of regulatory elements (either its own, or heterologous elements) may be transfected into a cell using techniques known to those of ordinary skill in the art and described in greater detail below, and the sequence may be expressed by the cell (in addition to any normal miRNA), thereby resulting in amounts of the miRNA within the cell that are higher than would be observed in the absence of such transfection.

Likewise, gene expression may also be increased in a cell by reducing the function of a specific miRNA in the cell. This may be accomplished by inhibiting expression of the miRNA-encoding gene, or by interfering with miRNA activity; e.g., by administering an antisense oligonucleotide that competes with the miRNA's natural substrate for binding to the miRNA (i.e., the miRNA preferentially binds to the antisense oligonucleotide instead of its target on the cellular mRNA).

In preferred embodiments, the methods and biological interactions identified in accordance with the present invention have many utilities in modulation of the herpes virus lifecycle in cells, and ultimately in treatment of herpes virus disease. Described below are four specific examples of such embodiments.

First, viral replication may be prevented by stimulating the expression of naturally occurring miRNAs (those that are predicted to suppress genes involved in essential virus functions, such as DNA replication) or by augmenting expression by delivery of analogous artificial miRNAs into the cell.

Second, reactivation of the virus may be prevented by stimulating the expression of naturally occurring miRNAs (those that are predicted to suppress viral genes needed to exit latency and resume replication, such as the major immediate early genes) or by delivery of analogous artificial miRNAs into the cell.

Alternatively, in instances in which the first approach of preventing virus replication is successful, it may be advantageous to use a combination therapy of the first approach together with enhancing reactivation by suppressing miRNAs that inhibit immediate early genes. This way the virus would be forced out of latency and at the same time would be prevented from replicating and spreading. The advantage of this approach over the second approach listed above, for instance, would be the possibility of a full cure of the herpes virus disease. That is, this combined approach could prevent the chronic disease as opposed to preventing only the acute disease as addressed by the above-stated second approach. Another advantage of the combined approach is that by forcing the virus out of latency, the virus would become visible and therefore susceptible to the immune system of the host.

Another approach involves improving the efficacy of current antiviral compounds. Specific miRNAs could be combined with small molecule drugs to interfere with viral replication or emergence from latency by multiple and potentially synergistic mechanisms.

Design and production of miRNA, variants and chemically modified derivatives: The naturally occurring miRNAs identified in accordance with the present invention are believed to require perfect complementarity of a “seed” oligomer sequence near the 5′ end of the miRNA, typically within the first 7, 8 or 9 nucleotides, to its target oligomer sequence in the mRNA. The degree of complementarity of the remaining miRNA is believed to govern the mechanism by which the miRNA regulates its target mRNA. That is, once incorporated into a cytoplasmic RISC, the miRNA will specify cleavage if the mRNA has sufficient complementarity to the miRNA, or it will repress productive translation if the mRNA does not have sufficient complementarity to be cleaved but does have a threshold level of complementarity to the miRNA (reviewed by Bartel, D., 2004, Cell, 116, 281-297). Accordingly, a person of skill in the art will appreciate that, outside the “seed” sequence, the sequence of a naturally occurring miRNA can be altered to increase or decrease the level of complementarity between the miRNA and a target sequence, while still maintaining, or even improving on, the ability of the miRNA to repress translation. Indeed, the present invention contemplates such modifications, particularly directed to increasing overall complementarity. In one embodiment, the naturally occurring miRNA sequence can be modified to achieve full complementarity with its target sequence, thereby creating a siRNA that would be expected to specify cleavage of the mRNA at the target sequence.

Furthermore, in embodiments of the invention in which gene expression is regulated by introducing mature miRNA into a cell, such miRNA can be modified in accordance with known methods, for instance to improve stability of the molecules, to improve binding/annealing to a target, or to introduce other pharmaceutically desirable attributes, as discussed for siRNAs in, for example, Fougerolles et al., 2007 (Nature Reviews Drug Discovery 6, 443-453). Methods of chemically modifying oligonucleotides, particularly as used for RNA interference, to achieve such ends are well known in the art. For instance, numerous such methods are set forth in U.S. Publication No. 2006/0211642 to McSwiggen et al., directed in part to chemically modified siRNA molecules that retain their RNAi activity.

By way of a further non-limiting representative example, the miRNA molecules may be designed to resist degradation by modifying it to include phosphorothioate, or other linkages, methylphosphonate, sulfone, sulfate, ketyl, phosphorodithioate, phosphoramidate, phosphate esters, and the like. Modifications designed to increase in vivo stability include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine. In addition, chemically synthesizing nucleic acid molecules with modifications (base, sugar and/or phosphate) can prevent their degradation by serum ribonucleases, which can increase their potency.

The miRNAs may also be provided as conjugates and/or complexes of miRNAs or their variants or derivatives. Such conjugates and/or complexes can be used to facilitate delivery of miRNA molecules into a biological system, such as a cell. The conjugates and complexes can impart therapeutic activity by transferring therapeutic compounds across cellular membranes, altering the pharmacokinetics, and/or modulating the localization of nucleic acid molecules of the invention. Such conjugates are known in the art, and include, but are not limited to, small molecules, lipids, cholesterol, phospholipids, nucleosides, nucleotides, nucleic acids, antibodies, toxins, negatively charged polymers and other polymers, for example, proteins, peptides, hormones, carbohydrates, polyethylene glycols, or polyamines.

In other embodiments, miRNA can be provided as an miRNA-encoding gene or polynucleotide and produced in situ by expression of the polynucleotide operably linked into to a vector comprising a promoter/regulatory sequence (either the miRNA gene's homologous sequences, or heterologous elements) such that the vector is capable of directing transcription of the miRNA in a manner enabling its processing in situ. The vector comprises a nucleic acid sequence encoding at least one miRNA molecule as described herein. It can encode one or both strands of a miRNA duplex, or a single self-complementary strand that self hybridizes into a miRNA duplex.

The miRNA encoding polynucleotide can be cloned into a number of types of vectors, including RNA vectors or DNA plasmids or viral vectors. Viral vectors can be constructed based on, but not limited to, adeno-associated virus, retrovirus/lentivirus, adenovirus, or alphavirus. The recombinant vectors capable of expressing the miRNA molecules can be delivered as described below, and persist in target cells. Alternatively, viral vectors can be used that provide for transient expression of nucleic acid molecules.

Those of skill in the art of molecular biology generally know how to use regulatory elements to control gene expression. If homologous regulatory elements are not utilized, it is understood that heterologous elements can be constitutive, tissue-specific, inducible, and/or useful under the appropriate conditions to direct high level expression of the introduced DNA segment.

A promoter sequence exemplified in the experimental examples is the immediate early cytomegalovirus (CMV) promoter sequence. This promoter sequence is a strong constitutive promoter capable of driving high levels of expression of any polynucleotide sequence operatively linked to it. Another exemplified promoter sequence is the U6 promoter. Promoters derived from genes encoding U6 small nuclear (snRNA), transfer RNA (tRNA) and adenovirus VA RNA are useful in generating high concentrations of desired RNA molecules such as miRNA in cells.

Other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter and Rous sarcoma virus promoter. Suitable human gene promoters include, but are not limited to, the actin promoter, the myosin promoter, the hemoglobin promoter, and the muscle creatine promoter. Examples of inducible promoters include, but are not limited, to a metallothionine promoter, a glucocorticoid promoter, a progesterone promoter, and a tetracycline promoter.

To assess the expression of the miRNA, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like. Suitable reporter genes may include genes encoding luciferase, beta-galactosidase, chloramphenicol acetyl transferase, secreted alkaline phosphatase, or green fluorescent protein, among others.

Delivery to host cells and tissues: As mentioned above, the miRNA molecules identified in accordance with the invention can be used to regulate expression of target genes within cultured cells and tissues, or ex vivo in cells or tissues that have been removed from a subject and, optionally, will be returned to the same subject or a different subject. Alternatively, the miRNA molecules are used to regulate gene expression in situ, in cells or tissues within a living subject.

In certain embodiments of the invention involving delivery of miRNA to cultured cells, the cultured cells are mammalian cells, more particularly human cells. In specific embodiments, the cells are cell lines typically used to study or screen for agents that affect viral infection, replication and other aspects of a viral life cycle, especially of herpes viruses. Nonlimiting examples of suitable cultured cell types include: fibroblasts, such as human embryonic lung fibroblasts or human foreskin fibroblasts; endothelial cells, such as human umbilical vein endothelial cells or other vascular endothelial cells; and epithelial cells, such as retinal pigmented epithelial cells or kidney epithelial cells, various neuronal cell types, and various stem cell types, including CD34+ hematopoietic stem cells.

In other embodiments, miRNA molecules are used in ex vivo applications; e.g., they are introduced into tissue or cells that are transplanted into a subject for therapeutic effect. The cells and/or tissue can be derived from a subject that later receives the explant, or can be derived from another subject prior to transplantation. For instance, in one non-limiting example, bone marrow cells to be transplanted from a donor to a recipient could be treated with therapeutic miRNAs (introduced either as an RNA molecule, a modified RNA molecule or by expression from a vector) which interfere with replication of HCMV. Such a treatment would protect the recipient from reactivation of latent virus and efficient replication of active virus within the transplanted cells.

Methods of delivering oligonucleotides or polynucleotides, such as miRNAs or miRNA-encoding genes, to cells are well known in the art, e.g., as described by Sambrook et al., 2001, supra or Ausubel et al., 2007, supra. For instance, physical methods for introducing a polynucleotide into a host cell include calcium phosphate precipitation, lipofection, particle bombardment, microinjection, electroporation, and the like.

Biological methods for introducing a polynucleotide of interest into a host cell include the use of DNA and RNA vectors as described above. Viral vectors, and especially retroviral vectors, have become a widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell or otherwise expose a cell to the miRNA of the present invention, in order to confirm the presence of the recombinant nucleotide sequence in the host cell, a variety of assays may be performed. Such assays include, for example, molecular biological assays well known to those of skill in the art, such as DNA and RNA blotting, RT-PCR and PCR; or through the use of selectable markers or reporter genes.

In other embodiments, miRNAs or variants/derivatives thereof as described herein are used as therapeutic agents to regulate expression of one or more target genes in a subject. In particular embodiments, the target genes are viral genes, particularly herpes virus genes, and more particularly genes involved in herpes virus replication or latency. In general, such methods involve introducing the miRNA molecules into the subject under conditions suitable to modulate (e.g., inhibit) the expression of the one or more target genes in the subject, to achieve a therapeutic effect, e.g., reduction or elimination of viral infection. One or more miRNAs may be administered, targeting expression of one or more genes. The miRNAs may be administered with other therapeutic agents, as described in greater detail below.

Administration of the miRNA therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses.

The miRNA molecules of the invention can be formulated for and administered by infusion or injection (intravenously, intraarterially, intramuscularly, intracutaneously, subcutaneously, intrathecally, intraduodenally, intraperitoneally, and the like). The miRNA molecules of the invention can also be administered intranasally, vaginally, rectally, orally, topically, buccally, transmucosally, or transdermally.

Compositions and kits: The miRNAs, miRNA-encoding polynucleotides and vectors, and miRNA derivatives and variants described herein can be formulated into compositions for use in cultured cells, in ex vivo cell or tissue explants, or in vivo for delivery of therapeutic agents. Such compositions comprise one or more of the miRNA molecules listed above, and a biologically or pharmaceutically acceptable carrier or medium. The term “biologically acceptable medium” refers to a carrier, diluent, excipient and/or salt that is compatible with the other components of the composition and is not deleterious to the cells or tissues to which the composition is introduced. A “pharmaceutically acceptable medium” is a carrier, diluent, excipient, and/or salt that is compatible with the other ingredients of the formulation, and not deleterious to the recipient thereof. Compositions formulated for pharmaceutical use are referred to herein as “pharmaceutical compositions.”

Pharmaceutical compositions containing miRNA therapeutic agents can be prepared by procedures known in the art using well known and readily available ingredients. They can be formulated as solutions appropriate for parenteral administration, for instance by intramuscular, subcutaneous or intravenous routes. They can also take the form of an aqueous or anhydrous solution or dispersion, or alternatively the form of an emulsion or suspension. Suitable components of pharmaceutical compositions, and methods of making such compositions are described in Remington's Pharmaceutical Sciences, a standard reference text in this field.

The pharmaceutical compositions may incorporate additional substances to function as stabilizing agents, preservatives, buffers, wetting agents, emulsifying agents, dispersing agents, and monosaccharides, polysaccharides, and salts for varying the osmotic balance. They may further include one or more antioxidants. Exemplary reducing agents include mercaptopropionyl glycine, N-acetylcysteine, P-mercaptoethylamine, glutathione, ascorbic acid and its salts, sulfite, or sodium metabisulfite, or similar species. In addition, antioxidants can include natural antioxidants such as vitamin E, C, leutein, xanthine, beta carotene and minerals such as zinc and selenium.

As mentioned above, all compositions contemplated herein, including the pharmaceutical compositions, may contain a plurality of different miRNA, which may be present in modified or unmodified form, or as a miRNA-encoding polynucleotide. Moreover, the pharmaceutical compositions can contain one or more additional active ingredients to achieve a desired therapeutic effect. In one embodiment, the additional active ingredient is an antiviral agent or combination of antiviral agents, which may target herpesviruses, or other viruses, or combinations thereof in accordance with their pharmaceutical indications. Nonlimiting examples of such agents include: abacavir, aciclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, famciclovir, fomivirsen, fosamprenavir, foscamet, fosfonet, ganciclovir, gardasil, ibacitabine, idoxuridine, imiquimod, indinavir, various interferons, lamivudine, lopinavir, loviride, maraviroc, moroxydine, nelfinavir, nevirapine, oseltamivir, penciclovir, peramivir, pleconaril, podophyllotoxin, ribavirin, rimantadine, ritonavir, saquinavir, stavudine, tenofovir, tipranavir, trifluridine, trizivir, tromantadine, truvada, valaciclovir, valganciclovir, vicriviroc, vidarabine, viramidine, zalcitabine, zanamivir and zidovudine.

Another aspect of the invention features articles of manufacture, sometimes referred to as “kits,” to facilitate practice of various aspects the invention. The kits typically comprise one or more miRNAs, or derivatives or variants thereof, or miRNA-encoding polynucleotides, together with one or more other drugs or reagents, biologically or pharmaceutically acceptable media or components thereof, and instructions for using the components to practice one or more of the methods described herein. The components typically are packaged together or separately for convenience and ease of use. The kits may comprise any one or more of the miRNAs, vectors, delivery vehicles, media, additional active ingredients or supplemental components described herein.

The following examples are provided to describe the invention in more detail. They are intended to illustrate, not to limit, the invention.

### Example 1

### Use of Algorithm to Predict Herpes Virus Targets of Viral and Human Cellular miRNAs

The algorithm described herein was used to predict miRNA targets within the 3′UTRs of herpes virus mRNAs. The miRNAs that were evaluated included all database-accessible miRNAs from herpes simplex virus (HSV), Epstein-Barr virus (EBV), human cytomegalovirus (HCMV), Kaposi's sarcoma-associated herpesvirus (KSHV or HHV-8) and Homo sapiens (humans).

The 3′UTRs that were queried by the algorithm included 3′ UTRs from herpes viruses, which have been either (1) experimentally determined, (2) determined computationally by experimentally determined positions of the polyadenylation sites, or (3) determined computationally based on the first polyadenylation sites in the sequences downstream from the stop codons of the genes.

Materials and Methods:

Viral genome sequences were obtained at http://www.ncbi.nlm.nih.gov. The RefSeq accession numbers as follow: (i) HSV-1, NC001806.1; (ii) EBV, NC_{—}007605.1; (iii) HCMV clinical isolates: Toledo-BAC, AC146905; FIX-BAC, AC146907; PH-BAC, AC146904; TR-BAC, 146906; and HCMV laboratory strains: AD169-BAC, AC146999; Towne-BAC, AC146851; (iv) KSHV sequence NC_{—}003409.1. Accessed databases or other miRNA-containing information included the miRBase at the following url: microrna.sanger.ac.uk/sequences/index.shtml, as well as sequences from the published literature referred to herein.

For herpesvirus genes for which the 3′UTR was not tabulated, we used a simple computational algorithm to detect them: we detected the polyadenylation (polyA) signal (AATAAA) nearest to the stop codon of the coding sequence and considered the 3′UTR to be the sequence from the stop codon to the polyA signal. In cases where the resulting 3′UTR was longer than 500 nucleotides, we did not analyze the part beyond 500, in order to avoid considering exceedingly long 3′UTRs when a non-standard polyadenylation signal was present. In KSHV it is known that the Zta and Rta genes have 3′UTRs longer than 500 (reference), so in this virus, we performed the analysis with all 3′UTRs extending all the way to the nearest downstream polyA signal, with no restriction on the length.

The most common experimentally observed seed binding sequence in a 3′UTR for a miRNA is either the hexamer sequence from position 2 to 7 (denoted 2-7) or the heptamer 2-8, both counted from the 5′ end of the miRNA. In order to increase specificity of our algorithm, we used the heptamer 2-8 whenever possible. In cases where too much sensitivity was lost (for HSV-1 and KSHV), we used hexamers 2-7 or 3-8 as the seed. The reason to use a seed 3-8 besides 2-7 is that the extents of the same miRNA sequences often differ by one or two nucleotides in different publications.

The random background sequence used in our computations is based on the k-th order Markov model (MM) that considers composition of the 3′UTR up to (k+1)-mers. For example, the second order Markov model considers the nucleotide, dinucleotide, and trinucleotide count in the 3′UTR. Two approaches are used for constructing the background sequence: either each 3′UTR is considered separately or all 3′UTRs are combined. The advantage of the first approach is that it captures local properties of the sequence. The benefit of the second approach is that it provides sufficient statistical power to consider higher order Markov models. In the end we used two combinations for comparison: either the first order Markov model based on local sequence composition, or the third order Markov model based on global sequence composition. Both cases take into account the dinucleotide content in order to capture such features as the under-representation of CpG dinucleotides in eukaryotic sequences.

To be more specific, let us assume that the length of the 3′UTR is l and that we are interested in determining the probability p of finding an n-mer X₁X₂ . . . Xₙ in the given 3′UTR based on the k-th order Markov model. Let c(X₁X₂ . . . Xₖ) denote the count of k-mer X₁X₂ . . . Xₖ. Frequency of X₁X₂ . . . Xₖ is clearly f(X₁ . . . Xₖ)=c(X₁ . . . Xₖ)/l . Denoting by p (Xₖ₊₁|X₁ . . . Xₖ) the conditional probability of the (k+1)-st nucleotide being Xₖ₊₁ if it is preceded by a k-mer X₁ . . . Xₖ, we compute p as

$$p = p ( {X}_{n} \vert {X}_{n - k} … {X}_{n - 1} ) … p ( {X}_{k + 1} \vert {X}_{1} … {X}_{k} ) f ( {X}_{1} … {X}_{k} ) = \frac{f ( {X}_{n - k} … {X}_{n} ) … f ( {X}_{1} … {X}_{k + 1} )}{f ( {X}_{n - k} … {X}_{n - 1} ) … f ( {X}_{2} … {X}_{k} )} .$$

In higher organisms, miRNAs and their targets have often been predicted by using evolutionary conservation among species, given is the prediction that the miRNA binding sites within 3′UTRs will be more conserved than the surrounding sequences. So far there has been very little evidence for conservation in the case of virus miRNAs. The sole exception is the conservation of nine miRNAs between EBV and the rhesus lymphocryptovirus (RLCV), but since there are over 20 known miRNAs in EBV, we did not use conservation in order not to miss any targets.

As for HCMV, conservation with the chimpanzee cytomegalovirus (CCMV) was used to predict several HCMV miRNAs but the corresponding CCMV miRNAs were not experimentally verified. Therefore instead of using conservation among species we employed conservation among six strains of the virus (both laboratory strains and clinical isolates): AD 169, FIX, PH, Toledo, Towne, and TR. We aligned these six genomes and counted only heptamers conserved among all six strains. The only change in the algorithm was that in the formula set forth in the next section for the p-value PV_{SH}, the actual count of the seed heptamer c was replaced by its conserved count and the 3′UTR length l was replaced by the count of all conserved heptamers.

Computation. In order to determine the likelihood that a particular miRNA-3′UTR pair was functional, we computed the corresponding probability PV_{SH}. Let c denote the actual count of seed n-mers in the 3′UTR of length l and p the probability (based on the MM described above) that any given n-mer in the random background sequence is the seed n-mer. Then our p-value PV_{SH} gives the probability of finding at least c seed n-mers in a background sequence of length l which is equal to the p-value of the binomial distribution,

$${PV}_{SH} = {PV}_{bin} ( l - n + 1 , c , p ) = {\sum }_{i = c}^{l - n + 1} ( \begin{matrix}l - n + 1 \\ i\end{matrix} ) {{p}^{i} ( 1 - p )}^{l - n + 1 - i} .$$

In practice, l is of the order of 100 or 1000. For a hexamer seed sequence (n=6), a typical p is 1/4⁶=1/4096 (exactly if all hexamers were equally likely) and therefore a typical c is zero, making the equation above impractical. An alternative exact expression for PV_{SH} which is numerically efficient is

$${PV}_{SH} = {PV}_{bin} ( l - n + 1 , c , p ) = \frac{B ( p , c , l - n - c + 2 )}{B ( c , l - n - c + 2 )}$$

where B(x,a,b) is the incomplete beta function and B(a,b) is the usual beta function,

$$B ( x , a , b ) = {\int }_{0}^{x} {{u}^{a - 1} ( 1 - u )}^{b - 1} d u , \\ B ( a , b ) = B ( 1 , a , b ) .$$

The statistical significance of the top miRNA-target pairs was evaluated by calculating probability PV_{MH}. Because the majority of p-values PV_{SH} is equal to 1, we could not use the standard method of estimating the False Discovery Rate. Instead we used the following Monte Carlo procedure: First we generated N=1000 random genomes analogous to the actual genome of interest. This means that each genome will have exactly the same number of 3′UTRs as the genome of interest and each generated 3′UTR will be of the same length as the corresponding real 3′UTR. Each random 3′UTR is generated using the kth order MM based on the composition of the corresponding 3′UTR in the real genome.

For each of the N randomly generated genomes, we repeated the same analysis of computing PV_{SH} as we did for the real genome: i.e., we computed the score PV_{SH} for each miRNA-3′UTR and sorted them. Next we evaluated the statistical significance of the top t miRNA-target pairs for the actual genome by counting the number Nₜ of the randomly generated genomes in which the tth top microRNA-3′UTR pair has PV_{SH} smaller than the tth pair in the actual genome. For each t, the p-value PV_{MH}(t) corrected for Multiple Hypothesis Testing was computed by

$${PV}_{MH} ( t ) = \frac{{N}_{t}}{N} .$$

PV_{MH}(t) is the probability of finding better scores for the top t potential microRNA-3′UTR pairs in a random genome with similar properties as the actual genome. The smaller PV_{MH}(t), the higher the chance that the predicted targets are real targets.

Results:

Tables 3-6 below set forth predicted miRNAs, UTRs and the best miRNA-UTR pairs predicted by the algorithm. For Tables 3-6, the following annotations are used: MM=Markov model; o.=order; PV-SH=single hypothesis p-value; miRNA name=notation from microRNA database at http://microma.sanger.ac.uk/sequences/; miRNA #=miRNA number used in other tables as a shorthand; hexamer=a hexamer complementary to the seed miRNA sequence; actual=actual oligomer count; predicted=predicted count based on the MM; Log=logarithm with the base 10 length=3′UTR length or the count of conserved oligomers in the 3′ UTR when conservation is taken into account (in HCMV only); PV_MH=p-value corrected for multiple hypothesis testing.

TABLE 3A

HSV-1 miRNAs: Combined effect on all 3′ UTRs using
hexamers complementary to positions 3-8 in miRNA

| | |
|---|---|
| Local 1st o. MM | Global 3rd o. MM |

| | | | | | | | |
|---|---|---|---|---|---|---|---|
| miRNA name | miRNA# | Hexamer | Actual | Predicted | Log (PV_SH) | Predicted | Log (PV_SH) |

| | | | | | | | |
|---|---|---|---|---|---|---|---|
| hsv1-miR-H1 | 1 | TCCTTC | 5 | 5.08 | −0.24 | 4.41 | −0.35 |
| hsv1-miR-LAT | 2 | GGCCGC | 33 | 20.57 | −2.16 | 23.74 | −1.38 |
| Total: | | | 38 | 25.65 | | 28.15 | |

TABLE 3B
Best HSV-1 3′ UTR targets: Combined effect of all microRNAs based on
heptamer complementary to positions 3-8 in miRNA

| | | | Local 1st o. MM | | Global 3rd o. MM | |
|---|---|---|---|---|---|---|
| | | Ac- | | Log | | Log |
| 3′ UTR | Length | tual | Predicted | (PV_SH) | Predicted | (PV_SH) |
| UL35 | 33 | 1 | 0.05 | −1.30 | 0.05 | −1.30 |
| RL1 | 274 | 3 | 0.88 | −1.22 | 0.43 | −2.03 |
| RL1 | 274 | 3 | 0.88 | −1.22 | 0.43 | −2.03 |
| RL2 | 146 | 1 | 0.10 | −1.03 | 0.23 | −0.69 |
| RL2 | 186 | 1 | 0.10 | −1.01 | 0.29 | −0.60 |
| US9 | 82 | 1 | 0.11 | −0.99 | 0.13 | −0.92 |
| UL42 | 53 | 1 | 0.14 | −0.88 | 0.08 | −1.10 |
| US8A | 444 | 2 | 0.65 | −0.86 | 0.69 | −0.82 |
| UL20 | 500 | 2 | 0.76 | −0.75 | 0.78 | −0.74 |
| UL1 | 500 | 2 | 0.83 | −0.70 | 0.78 | −0.74 |
| UL34 | 477 | 2 | 0.83 | −0.69 | 0.74 | −0.77 |
| UL24 | 192 | 1 | 0.23 | −0.69 | 0.30 | −0.59 |
| UL9 | 500 | 2 | 1.03 | −0.56 | 0.78 | −0.74 |
| UL52 | 500 | 1 | 0.35 | −0.53 | 0.78 | −0.27 |
| UL51 | 500 | 1 | 0.38 | −0.50 | 0.78 | −0.27 |
| UL11 | 500 | 1 | 0.38 | −0.50 | 0.78 | −0.27 |
| UL47 | 500 | 2 | 1.17 | −0.49 | 0.78 | −0.74 |
| UL16 | 500 | 1 | 0.44 | −0.45 | 0.78 | −0.27 |
| UL49A | 500 | 1 | 0.51 | −0.40 | 0.78 | −0.27 |
| UL13 | 500 | 1 | 0.57 | −0.37 | 0.78 | −0.27 |
| UL37 | 500 | 1 | 0.58 | −0.35 | 0.78 | −0.27 |
| UL39 | 500 | 1 | 0.66 | −0.32 | 0.78 | −0.27 |
| UL14 | 500 | 1 | 0.68 | −0.31 | 0.78 | −0.27 |
| US11 | 500 | 1 | 0.71 | −0.30 | 0.78 | −0.27 |
| US8 | 500 | 1 | 0.86 | −0.24 | 0.78 | −0.27 |

TABLE 3C
Best HSV-1 miRNA - 3′UTR target pairs based on hexamer complementary to
positions 3-8 in miRNA

| | | | | Local 1st o. MM | | | Global 3rd o. MM | |
|---|---|---|---|---|---|---|---|---|
| 3′ UTR | Length | miRNA # | Actual | Predicted | Log (PV_SH) | PV_MH | Predicted | Log (PV_SH) |
| UL35 | 33 | 1 | 1 | 0.05 | −1.35 | 0.50 | 0.01 | −2.10 |
| RL1 | 274 | 2 | 3 | 0.84 | −1.28 | 0.38 | 0.36 | −2.23 |
| RL1 | 274 | 2 | 3 | 0.84 | −1.28 | 0.31 | 0.36 | −2.23 |
| RL2 | 186 | 2 | 1 | 0.07 | −1.18 | 0.28 | 0.24 | −0.66 |
| RL2 | 146 | 2 | 1 | 0.08 | −1.12 | 0.25 | 0.19 | −0.76 |
| US9 | 82 | 1 | 1 | 0.11 | −0.99 | 0.33 | 0.02 | −1.70 |
| UL20 | 500 | 2 | 2 | 0.55 | −0.98 | 0.33 | 0.66 | −0.85 |
| UL24 | 192 | 1 | 1 | 0.11 | −0.97 | 0.27 | 0.05 | −1.34 |
| UL42 | 53 | 2 | 1 | 0.13 | −0.92 | 0.26 | 0.07 | −1.17 |
| UL34 | 477 | 1 | 1 | 0.14 | −0.89 | 0.25 | 0.12 | −0.96 |
| UL1 | 500 | 2 | 2 | 0.69 | −0.82 | 0.27 | 0.66 | −0.85 |
| UL49A | 500 | 2 | 1 | 0.25 | −0.66 | 0.45 | 0.66 | −0.32 |
| UL52 | 500 | 2 | 1 | 0.27 | −0.63 | 0.41 | 0.66 | −0.32 |
| US8A | 444 | 1 | 1 | 0.28 | −0.62 | 0.40 | 0.11 | −0.99 |
| UL9 | 500 | 2 | 2 | 0.95 | −0.61 | 0.38 | 0.66 | −0.85 |
| UL11 | 500 | 2 | 1 | 0.33 | −0.56 | 0.44 | 0.66 | −0.32 |
| UL51 | 500 | 2 | 1 | 0.34 | −0.55 | 0.42 | 0.66 | −0.32 |
| UL39 | 500 | 2 | 1 | 0.34 | −0.54 | 0.38 | 0.66 | −0.32 |
| UL47 | 500 | 2 | 2 | 1.10 | −0.52 | 0.41 | 0.66 | −0.85 |
| US8A | 444 | 2 | 1 | 0.38 | −0.51 | 0.40 | 0.58 | −0.35 |
| UL16 | 500 | 2 | 1 | 0.38 | −0.50 | 0.37 | 0.66 | −0.32 |
| UL13 | 500 | 2 | 1 | 0.43 | −0.46 | 0.44 | 0.66 | −0.32 |
| UL37 | 500 | 2 | 1 | 0.51 | −0.40 | 0.49 | 0.66 | −0.32 |
| UL14 | 500 | 2 | 1 | 0.54 | −0.38 | 0.48 | 0.66 | −0.32 |
| US11 | 500 | 2 | 1 | 0.63 | −0.33 | 0.48 | 0.66 | −0.32 |

TABLE 4A

EBV miRNAs: Combined effect on all 3′ UTRs using
hexamers complementary to positions 2-8 in miRNA

| | |
|---|---|
| Local 1st o. MM | Global 3rd o. MM |

| | | | | | | | |
|---|---|---|---|---|---|---|---|
| miRNA name | miRNA # | Heptamer | Actual | Predicted | Log (PV_SH) | Predicted | Log (PV_SH) |

| | | | | | | | |
|---|---|---|---|---|---|---|---|
| ebv-miR-BART1-3p | 1 | CGGTGCT | 5 | 1.97 | −1.30 | 1.68 | −1.55 |
| ebv-miR-BART1-5p | 2 | CACTAAG | 2 | 1.39 | −0.39 | 0.66 | −0.85 |
| ebv-miR-BART2 | 3 | AGAAAAT | 2 | 1.14 | −0.50 | 1.38 | −0.40 |
| ebv-miR-BART3-3p | 4 | GTGGTGC | 2 | 3.57 | −0.06 | 4.38 | −0.03 |
| ebv-miR-BART3-5p | 5 | ACTAGGT | 0 | 1.20 | 0.00 | 0.42 | 0.00 |
| ebv-miR-BART4 | 6 | ATCAGGT | 0 | 1.57 | 0.00 | 1.92 | 0.00 |
| ebv-miR-BART5 | 7 | TCACCTT | 6 | 2.00 | −1.78 | 1.86 | −1.92 |
| ebv-miR-BART6-3p | 8 | GATCCCC | 3 | 3.46 | −0.17 | 1.92 | −0.52 |
| ebv-miR-BART6-5p | 9 | GACCAAC | 5 | 2.28 | −1.09 | 2.22 | −1.13 |
| ebv-miR-BART7 | 10 | CTATGAT | 0 | 1.23 | 0.00 | 1.44 | 0.00 |
| ebv-miR-BART8-3p | 11 | ATTGTGA | 1 | 1.66 | −0.09 | 1.50 | −0.11 |
| ebv-miR-BART8-5p | 12 | AAACCGT | 0 | 0.80 | 0.00 | 0.90 | 0.00 |
| ebv-miR-BART9 | 13 | AAGTGTT | 0 | 1.34 | 0.00 | 1.20 | 0.00 |
| ebv-miR-BART10 | 14 | GGTTATG | 3 | 1.40 | −0.78 | 1.62 | −0.66 |
| ebv-miR-BART11-3p | 15 | GTGTGCG | 2 | 2.07 | −0.21 | 1.68 | −0.30 |
| ebv-miR-BART11-5p | 16 | AAACTGT | 0 | 1.47 | 0.00 | 1.74 | 0.00 |
| ebv-miR-BART12 | 17 | CCACAGG | 4 | 4.68 | −0.16 | 4.02 | −0.25 |
| ebv-miR-BART13 | 18 | AAGTTAC | 3 | 0.76 | −1.39 | 0.78 | −1.35 |
| ebv-miR-BART14-3p | 19 | AGCATTT | 2 | 1.45 | −0.37 | 1.92 | −0.24 |
| ebv-miR-BART14-5p | 20 | GTAGGGT | 0 | 1.66 | 0.00 | 0.54 | 0.00 |
| ebv-miR-BART15 | 21 | AAACCAC | 2 | 1.90 | −0.25 | 1.98 | −0.23 |
| ebv-miR-BART16 | 22 | CACTCTA | 1 | 1.48 | −0.11 | 1.02 | −0.19 |
| ebv-miR-BART17-3p | 23 | GCATACA | 1 | 1.42 | −0.12 | 1.07 | −0.18 |
| ebv-miR-BART17-5p | 24 | GTCCTCT | 3 | 2.28 | −0.40 | 2.64 | −0.31 |
| ebv-miR-BART18 | 25 | CGAACTT | 0 | 0.91 | 0.00 | 0.42 | 0.00 |
| ebv-miR-BART1 9 | 26 | ACAAAAC | 0 | 1.49 | 0.00 | 1.79 | 0.00 |
| ebv-miR-BART20-3p | 27 | CCTTCAT | 2 | 1.95 | −0.24 | 1.86 | −0.26 |
| ebv-miR-BART20-5p | 28 | CCTGCTA | 1 | 2.55 | −0.04 | 3.29 | −0.02 |
| ebv-miR-BHRF1-1 | 29 | TCAGGTT | 1 | 1.74 | −0.08 | 1.20 | −0.16 |
| ebv-miR-BHRF1-2 | 30 | AAAAGAT | 1 | 1.14 | −0.17 | 1.62 | −0.10 |
| ebv-miR-BHRF1-2* | 31 | CAGAATT | 2 | 1.35 | −0.41 | 1.98 | −0.23 |
| ebv-miR-BHRF1-3 | 32 | TCCCGTT | 3 | 1.24 | −0.89 | 1.08 | −1.02 |
| Total: | | | 57 | 56.55 | | 53.73 | |

TABLE 4B
Best EBV 3′ UTR targets: Combined effect of all microRNAs based on
heptamer complementary to positions 2-8 in miRNA

| | | | Local 1st o. MM | | Global 3rd o. MM | |
|---|---|---|---|---|---|---|
| 3′ UTR | Length | Actual | Predicted | Log (PV_SH) | Predicted | Log (PV_SH) |
| BZLF1 | 53 | 2 | 0.10 | −2.35 | 0.10 | −2.38 |
| BLLF3 | 24 | 1 | 0.03 | −1.54 | 0.04 | −1.39 |
| BNRF1 | 148 | 2 | 0.33 | −1.36 | 0.27 | −1.53 |
| BZLF2 | 500 | 3 | 0.91 | −1.19 | 0.90 | −1.21 |
| BALF3 | 500 | 3 | 0.93 | −1.17 | 0.90 | −1.21 |
| BHLF1 | 257 | 2 | 0.58 | −0.93 | 0.46 | −1.11 |
| BALF2 | 370 | 2 | 0.68 | −0.83 | 0.67 | −0.84 |
| BALF5 | 500 | 2 | 0.73 | −0.78 | 0.90 | −0.65 |
| BVLF1 | 171 | 1 | 0.19 | −0.77 | 0.31 | −0.58 |
| BARF1 | 500 | 2 | 0.85 | −0.68 | 0.90 | −0.65 |
| BDLF3.5 | 500 | 2 | 0.85 | −0.68 | 0.90 | −0.65 |
| BGLF3 | 500 | 2 | 0.86 | −0.67 | 0.90 | −0.65 |
| BGLF3.5 | 500 | 2 | 0.90 | −0.65 | 0.90 | −0.65 |
| BaRF1 | 500 | 2 | 0.91 | −0.64 | 0.90 | −0.65 |
| BMRF1 | 500 | 2 | 0.99 | −0.59 | 0.90 | −0.65 |
| BRLF1 | 500 | 2 | 1.07 | −0.54 | 0.90 | −0.65 |
| LF3 | 500 | 2 | 1.10 | −0.52 | 0.04 | −1.39 |
| BGLF1 | 500 | 2 | 1.12 | −0.51 | 0.90 | −0.65 |
| LMP-1 | 500 | 2 | 1.26 | −0.45 | 0.90 | −0.65 |
| BOLF1 | 500 | 1 | 0.68 | −0.31 | 0.90 | −0.23 |
| BARF0 | 500 | 1 | 0.69 | −0.30 | 0.90 | −0.23 |
| BFRF2 | 485 | 1 | 0.75 | −0.28 | 0.87 | −0.24 |
| BDLF4 | 500 | 1 | 0.77 | −0.27 | 0.90 | −0.23 |
| BGLF2 | 378 | 1 | 0.80 | −0.26 | 0.68 | −0.31 |
| BXRF1 | 500 | 1 | 0.83 | −0.25 | 0.90 | −0.23 |

TABLE 4C
Best EBV miRNA - 3′UTR target pairs based on hexamer complementary to
positions 2-8 in miRNA

| | | | | Local 1st o. MM | | | Global 3rd o. MM | |
|---|---|---|---|---|---|---|---|---|
| 3′ UTR | Length | miRNA # | Actual | Predicted | Log (PV_SH) | PV_MH | Predicted | Log (PV_SH) |
| BALF3 | 500 | 9 | 2 | 0.07 | −2.68 | 0.22 | 0.04 | −3.17 |
| BNRF1 | 148 | 23 | 1 | 0.01 | −2.25 | 0.27 | 0.01 | −2.28 |
| BZLF1 | 53 | 21 | 1 | 0.01 | −2.24 | 0.17 | 0.00 | −2.46 |
| BZLF1 | 53 | 32 | 1 | 0.01 | −2.07 | 0.23 | 0.00 | −2.71 |
| BALF3 | 500 | 30 | 1 | 0.01 | −2.00 | 0.23 | 0.03 | −1.58 |
| BKRF2 | 500 | 3 | 1 | 0.01 | −2.00 | 0.20 | 0.02 | −1.64 |
| BFRF2 | 485 | 18 | 1 | 0.01 | −1.95 | 0.21 | 0.01 | −1.89 |
| BNRF1 | 148 | 7 | 1 | 0.01 | −1.94 | 0.20 | 0.01 | −2.04 |
| BLLF3 | 24 | 27 | 1 | 0.01 | −1.91 | 0.21 | 0.00 | −2.83 |
| BRLF1 | 500 | 1 | 1 | 0.01 | −1.88 | 0.22 | 0.03 | −1.56 |
| BSLF2/ | 500 | 32 | 1 | 0.02 | −1.80 | 0.28 | 0.02 | −1.74 |
| BMLF1 | | | | | | | | |
| BHLF1 | 257 | 14 | 1 | 0.02 | −1.80 | 0.26 | 0.01 | −1.86 |
| BLRF2 | 500 | 18 | 1 | 0.02 | −1.79 | 0.22 | 0.01 | −1.87 |
| BSLF1 | 500 | 19 | 1 | 0.02 | −1.78 | 0.23 | 0.03 | −1.50 |
| BHRF1 | 500 | 1 | 1 | 0.02 | −1.75 | 0.26 | 0.03 | −1.56 |
| BaRF1 | 500 | 21 | 1 | 0.02 | −1.73 | 0.27 | 0.03 | −1.49 |
| LF1 | 500 | 18 | 1 | 0.02 | −1.70 | 0.30 | 0.01 | −1.87 |
| BDLF3.5 | 500 | 32 | 1 | 0.02 | −1.69 | 0.28 | 0.02 | −1.74 |
| BGRF1/ | 500 | 31 | 1 | 0.03 | −1.60 | 0.42 | 0.03 | −1.48 |
| BDRF1 | | | | | | | | |
| BARF1 | 500 | 7 | 1 | 0.03 | −1.58 | 0.43 | 0.03 | −1.52 |
| BGLF2 | 378 | 1 | 1 | 0.03 | −1.58 | 0.42 | 0.02 | −1.68 |
| BaRF1 | 500 | 29 | 1 | 0.03 | −1.58 | 0.40 | 0.02 | −1.71 |
| BZLF2 | 500 | 31 | 1 | 0.03 | −1.58 | 0.40 | 0.03 | −1.48 |
| BHLF1 | 257 | 22 | 1 | 0.03 | −1.55 | 0.41 | 0.01 | −2.06 |
| LF3 | 500 | 8 | 1 | 0.03 | −1.55 | 0.42 | 0.03 | −1.50 |

TABLE 5A

HCMV miRNAs: Combined effect on all 3′ UTRs using FIX and
conserved hexamer complementary to positions 2-8 in miRNA

| | | | |
|---|---|---|---|
| Local 1st o. MM | Global 3rd o. MM | Local 1st o. MM | Global 3rd o. MM |

| | | | | | | | | | | | | |
|---|---|---|---|---|---|---|---|---|---|---|---|---|
| miRNA | | | | | Log | | Log | | | Log | | Log |
| name | # | Heptamer | Actual | Predicted | (PV_SH) | Predicted | (PV_SH) | Actual | Predicted | (PV_{—SH)} | Predicted | (PV_{—SH)} |

| | | | | | | | | | | | | |
|---|---|---|---|---|---|---|---|---|---|---|---|---|
| hcmv- | 1 | TCCCGTG | 4 | 4.85 | −0.15 | 5.24 | −0.12 | 1 | 2.39 | −0.04 | 2.68 | −0.03 |
| miR- | | | | | | | | | | | | |
| UL22-1 | | | | | | | | | | | | |
| hcmv- | 2 | GCTAGTT | 0 | 2.07 | 0.00 | 1.71 | 0.00 | 0 | 0.97 | 0.00 | 0.92 | 0.00 |
| miR- | | | | | | | | | | | | |
| UL22A- | | | | | | | | | | | | |
| 1 | | | | | | | | | | | | |
| hcmv- | 3 | TCTGGTG | 3 | 3.88 | −0.13 | 7.06 | −0.01 | 2 | 1.93 | −0.24 | 3.34 | −0.07 |
| miR- | | | | | | | | | | | | |
| UL22A- | | | | | | | | | | | | |
| 1 | | | | | | | | | | | | |
| hcmv- | 4 | ACATGCC | 1 | 3.57 | −0.01 | 2.92 | −0.02 | 0 | 1.74 | 0.00 | 1.58 | 0.00 |
| miR- | | | | | | | | | | | | |
| UL31-1 | | | | | | | | | | | | |
| hcmv- | 5 | TTCAACG | 6 | 4.54 | −0.52 | 4.50 | −0.53 | 3 | 2.28 | −0.40 | 2.18 | −0.43 |
| miR- | | | | | | | | | | | | |
| UL36-1 | | | | | | | | | | | | |
| hcmv- | 6 | AGGTGTC | 2 | 3.13 | −0.09 | 2.68 | −0.13 | 2 | 1.40 | −0.39 | 1.71 | −0.29 |
| miR- | | | | | | | | | | | | |
| UL36- | | | | | | | | | | | | |
| 1-N | | | | | | | | | | | | |
| hcmv- | 7 | CTCGCGC | 9 | 13.55 | −0.04 | 8.05 | −0.38 | 6 | 8.26 | −0.08 | 4.02 | −0.66 |
| miR- | | | | | | | | | | | | |
| UL53-1 | | | | | | | | | | | | |
| hcmv- | 8 | GACGCGC | 16 | 15.52 | −0.31 | 12.43 | −0.73 | 12 | 9.37 | −0.63 | 6.37 | −1.52 |
| miR- | | | | | | | | | | | | |
| UL54-1 | | | | | | | | | | | | |
| hcmv- | 9 | CCATCCC | 6 | 3.75 | −0.75 | 4.27 | −0.59 | 1 | 1.91 | −0.07 | 2.15 | −0.05 |
| miR- | | | | | | | | | | | | |
| UL70- | | | | | | | | | | | | |
| 3p | | | | | | | | | | | | |
| hcmv- | 10 | GAGACGC | 6 | 7.30 | −0.13 | 8.89 | −0.06 | 4 | 3.90 | −0.26 | 4.26 | −0.21 |
| miR- | | | | | | | | | | | | |
| UL70- | | | | | | | | | | | | |
| 5p | | | | | | | | | | | | |
| hcmv- | 11 | CATGGCC | 3 | 3.57 | −0.16 | 4.51 | −0.08 | 1 | 1.72 | −0.09 | 2.33 | −0.05 |
| miR- | | | | | | | | | | | | |
| UL102- | | | | | | | | | | | | |
| 1 | | | | | | | | | | | | |
| hcmv- | 12 | CGACGCC | 16 | 12.00 | −0.81 | 15.59 | −0.31 | 9 | 6.80 | −0.61 | 7.77 | −0.43 |
| miR- | | | | | | | | | | | | |
| UL102- | | | | | | | | | | | | |
| 2 | | | | | | | | | | | | |
| hcmv- | 13 | CAACGTC | 11 | 6.00 | −1.37 | 8.39 | −0.65 | 2 | 3.05 | −0.09 | 4.10 | −0.04 |
| miR- | | | | | | | | | | | | |
| UL111 | | | | | | | | | | | | |
| a-1 | | | | | | | | | | | | |
| hcmv- | 14 | CGTCACT | 13 | 5.34 | −2.45 | 4.75 | −2.88 | 6 | 2.80 | −1.19 | 2.45 | −1.41 |
| miR- | | | | | | | | | | | | |
| UL112- | | | | | | | | | | | | |
| 1 | | | | | | | | | | | | |
| hcmv- | 15 | GAGGACG | 23 | 5.98 | −7.02 | 11.34 | −2.81 | 10 | 2.91 | −3.06 | 5.70 | −1.19 |
| miR- | | | | | | | | | | | | |
| UL148 | | | | | | | | | | | | |
| D-1 | | | | | | | | | | | | |
| hcmv- | 16 | CCATGTC | 4 | 3.33 | −0.37 | 4.03 | −0.24 | 2 | 1.61 | −0.32 | 2.24 | −0.18 |
| miR- | | | | | | | | | | | | |
| US4 | | | | | | | | | | | | |
| hcmv- | 17 | GCTTGTC | 4 | 4.56 | −0.18 | 2.93 | −0.47 | 1 | 2.46 | −0.04 | 1.70 | −0.09 |
| miR- | | | | | | | | | | | | |
| USS-1 | | | | | | | | | | | | |
| hcmv- | 18 | TATCATA | 3 | 2.05 | −0.47 | 2.06 | −0.47 | 1 | 0.81 | −0.26 | 1.03 | −0.19 |
| miR- | | | | | | | | | | | | |
| USS-2 | | | | | | | | | | | | |
| hcmv- | 19 | ACCTATC | 5 | 2.02 | −1.26 | 2.31 | −1.07 | 2 | 0.95 | −0.61 | 1.03 | −0.56 |
| miR- | | | | | | | | | | | | |
| USS- | | | | | | | | | | | | |
| 2-N | | | | | | | | | | | | |
| hcmv- | 20 | GAGCGGT | 3 | 4.76 | −0.07 | 5.61 | −0.04 | 1 | 2.39 | −0.04 | 2.80 | −0.03 |
| miR- | | | | | | | | | | | | |
| US25-1 | | | | | | | | | | | | |
| hcmv- | 21 | AGACCGC | 6 | 5.40 | −0.34 | 6.32 | −0.22 | 3 | 2.78 | −0.28 | 2.77 | −0.28 |
| miR- | | | | | | | | | | | | |
| US25- | | | | | | | | | | | | |
| 2-5p | | | | | | | | | | | | |
| hcmv- | 22 | AAGTGGA | 2 | 2.51 | −0.15 | 2.92 | −0.10 | 1 | 1.12 | −0.17 | 1.34 | −0.13 |
| miR- | | | | | | | | | | | | |
| US25- | | | | | | | | | | | | |
| 2-3p | | | | | | | | | | | | |
| hcmv- | 23 | ACATCCA | 8 | 3.09 | −1.86 | 3.78 | −1.41 | 0 | 1.44 | 0.00 | 1.97 | 0.00 |
| miR- | | | | | | | | | | | | |
| US29-1 | | | | | | | | | | | | |
| hcmv- | 24 | GCACAAT | 3 | 3.35 | −0.19 | 2.08 | −0.46 | 2 | 1.52 | −0.35 | 1.10 | −0.52 |
| miR- | | | | | | | | | | | | |
| US33-1 | | | | | | | | | | | | |
| Total: | | | 157 | 126.12 | | 134.37 | | 72 | 66.51 | | 67.54 | |

TABLE 5B
Best HCMV 3′ UTR targets: Combined effect of all microRNAs based on
heptamer complementary to positions 2-8 in miRNA

| Fix strain only | | | | | | | Conserved among 6 strains | | | | | | |
|---|---|---|---|---|---|---|---|---|---|---|---|---|---|
| | | | Local | | Global | | | | | Local | | Global | |
| | | | 1st o. MM | | 3rd o. MM | | | | | 1st o. MM | | 3rd o. MM | |
| | | | | Log | | Log | | | | | Log | | Log |
| 3′ UTR | L | Act | Pred | (PV_SH) | Pred | (PV_SH) | 3′ UTR | L | Act | Pred | (PV_SH) | Pred | (PV_SH) |
| UL61 | 500 | 5 | 1.01 | −2.42 | 1.10 | −2.27 | UL80 | 34 | 1 | 0.02 | −1.63 | 0.08 | −1.12 |
| UL103 | 500 | 5 | 1.18 | −2.14 | 1.10 | −2.27 | UL34 | 14 | 1 | 0.03 | −1.53 | 0.03 | −1.50 |
| UL120 | 500 | 4 | 0.91 | −1.86 | 1.10 | −1.59 | UL98 | 413 | 3 | 0.80 | −1.33 | 0.94 | −1.16 |
| UL16 | 500 | 4 | 0.97 | −1.76 | 1.10 | −1.59 | UL103 | 21 | 1 | 0.05 | −1.32 | 0.05 | −1.32 |
| US7 | 383 | 3 | 0.56 | −1.72 | 0.84 | −1.27 | UL16 | 430 | 3 | 0.82 | −1.30 | 0.97 | −1.12 |
| UL153 | 161 | 2 | 0.24 | −1.62 | 0.36 | −1.30 | UL112- | 67 | 1 | 0.05 | −1.29 | 0.15 | −0.85 |
| UL34 | 14 | 1 | 0.03 | −1.53 | 0.03 | −1.50 | 113 | | | | | | |
| UL137 | 500 | 4 | 1.18 | −1.49 | 1.10 | −1.59 | UL3 | 57 | 1 | 0.09 | −1.06 | 0.13 | −0.92 |
| US26 | 45 | 1 | 0.04 | −1.46 | 0.10 | −1.03 | RL10 | 57 | 1 | 0.10 | −1.02 | 0.13 | −0.92 |
| UL80 | 57 | 1 | 0.04 | −1.40 | 0.13 | −0.92 | UL57 | 426 | 3 | 1.09 | −1.02 | 0.97 | −1.13 |
| UL60 | 500 | 3 | 0.76 | −1.39 | 1.10 | −1.00 | UL31 | 62 | 1 | 0.12 | −0.94 | 0.14 | −0.88 |
| UL141a | 500 | 4 | 1.31 | −1.36 | 1.10 | −1.59 | UL86 | 424 | 3 | 1.21 | −0.91 | 0.96 | −1.13 |
| UL44 | 500 | 5 | 1.99 | −1.29 | 1.10 | −2.27 | UL60 | 402 | 2 | 0.63 | −0.89 | 0.91 | −0.64 |
| US12 | 500 | 3 | 0.85 | −1.26 | 1.10 | −1.00 | UL92 | 394 | 3 | 1.26 | −0.88 | 0.89 | −1.21 |
| UL117 | 500 | 3 | 0.90 | −1.21 | 1.10 | −1.00 | UL52 | 377 | 3 | 1.34 | −0.82 | 0.86 | −1.26 |
| UL98 | 500 | 3 | 0.96 | −1.13 | 1.10 | −1.00 | UL67 | 183 | 1 | 0.20 | −0.73 | 0.41 | −0.47 |
| UL92 | 500 | 4 | 1.58 | −1.12 | 1.10 | −1.59 | UL87 | 182 | 2 | 0.79 | −0.73 | 0.41 | −1.19 |
| UL112- | 111 | 1 | 0.09 | −1.05 | 0.24 | −0.66 | UL43 | 368 | 2 | 0.80 | −0.72 | 0.84 | −0.69 |
| 113 | | | | | | | UL37 | 396 | 2 | 0.81 | −0.71 | 0.90 | −0.64 |
| US10 | 500 | 3 | 1.07 | −1.03 | 1.10 | −1.00 | UL79 | 329 | 2 | 0.81 | −0.71 | 0.75 | −0.76 |
| UL40 | 51 | 1 | 0.12 | −0.96 | 0.11 | −0.98 | UL123 | 92 | 1 | 0.22 | −0.70 | 0.21 | −0.72 |
| UL26 | 97 | 1 | 0.12 | −0.96 | 0.21 | −0.72 | US14 | 455 | 2 | 0.89 | −0.65 | 1.03 | −0.56 |
| UL57 | 500 | 3 | 1.30 | −0.85 | 1.10 | −1.00 | UL69 | 253 | 1 | 0.27 | −0.63 | 0.57 | −0.36 |
| UL86 | 500 | 3 | 1.45 | −0.75 | 1.10 | −1.00 | UL51 | 444 | 2 | 0.99 | −0.59 | 1.01 | −0.57 |
| UL151 | 500 | 3 | 1.49 | −0.73 | 1.10 | −1.00 | UL45 | 442 | 2 | 1.02 | −0.56 | 1.00 | −0.58 |
| US24 | 20 | 1 | 0.21 | −0.72 | 0.05 | −1.36 | UL95 | 379 | 2 | 1.03 | −0.56 | 0.86 | −0.67 |

TABLE 5C
Best HCMV miRNA - 3′UTR target pairs based on hexamer complementary to
positions 2-8 in miRNA

| | | | | Local 1st o. | | | Global 3rd o. | |
|---|---|---|---|---|---|---|---|---|
| | | | | MM | | | MM | |
| 3′ UTR | L | MiRNA # | Act | Pred | Log (PV_SH) | PV_MH | Pred | Log (PV_SH) |

Fix strain only

| | | | | | | | | |
|---|---|---|---|---|---|---|---|---|
| US9 | 500 | 15 | 2 | 0.033 | −3.26 | 0.19 | 0.093 | −2.39 |
| UL141a | 500 | 10 | 2 | 0.059 | −2.77 | 0.23 | 0.073 | −2.60 |
| UL103 | 500 | 18 | 1 | 0.002 | −2.75 | 0.14 | 0.017 | −1.79 |
| UL112- | 111 | 8 | 1 | 0.002 | −2.75 | 0.09 | 0.023 | −1.65 |
| 113 | | | | | | | | |
| UL103 | 500 | 15 | 2 | 0.076 | −2.56 | 0.11 | 0.093 | −2.39 |
| UL34 | 14 | 14 | 1 | 0.004 | −2.41 | 0.13 | 0.001 | −2.96 |
| UL61 | 500 | 7 | 2 | 0.102 | −2.32 | 0.14 | 0.066 | −2.68 |
| UL153 | 161 | 21 | 1 | 0.005 | −2.29 | 0.12 | 0.017 | −1.78 |
| UL123 | 92 | 14 | 1 | 0.006 | −2.21 | 0.14 | 0.007 | −2.14 |
| UL80 | 57 | 10 | 1 | 0.006 | −2.20 | 0.11 | 0.008 | −2.08 |
| UL69 | 323 | 24 | 1 | 0.007 | −2.19 | 0.10 | 0.011 | −1.95 |
| UL57 | 500 | 21 | 2 | 0.128 | −2.13 | 0.11 | 0.052 | −2.89 |
| UL92 | 500 | 15 | 2 | 0.140 | −2.05 | 0.13 | 0.093 | −2.39 |
| UL7 | 314 | 21 | 1 | 0.012 | −1.92 | 0.21 | 0.032 | −1.50 |
| US14 | 500 | 10 | 1 | 0.012 | −1.91 | 0.20 | 0.073 | −1.15 |
| US7 | 383 | 19 | 1 | 0.014 | −1.87 | 0.22 | 0.014 | −1.85 |
| UL67 | 213 | 7 | 1 | 0.015 | −1.82 | 0.25 | 0.028 | −1.56 |
| UL102 | 500 | 24 | 1 | 0.015 | −1.81 | 0.23 | 0.017 | −1.76 |
| UL98 | 500 | 6 | 1 | 0.016 | −1.81 | 0.21 | 0.022 | −1.66 |
| UL61 | 500 | 20 | 1 | 0.016 | −1.80 | 0.20 | 0.046 | −1.35 |
| RL4 | 246 | 1 | 1 | 0.016 | −1.80 | 0.18 | 0.021 | −1.68 |
| UL101 | 500 | 16 | 1 | 0.016 | −1.79 | 0.18 | 0.033 | −1.48 |
| UL153 | 161 | 23 | 1 | 0.016 | −1.79 | 0.17 | 0.010 | −2.00 |
| UL138 | 318 | 5 | 1 | 0.017 | −1.78 | 0.16 | 0.023 | −1.64 |
| UL60 | 500 | 17 | 1 | 0.017 | −1.77 | 0.16 | 0.024 | −1.62 |

Conserved among 6 strains

| | | | | | | | | |
|---|---|---|---|---|---|---|---|---|
| UL103 | 21 | 18 | 1 | 0.000 | −4.11 | 0.04 | 0.001 | −3.14 |
| UL112- | 67 | 8 | 1 | 0.001 | −2.96 | 0.16 | 0.014 | −1.85 |
| 113 | | | | | | | | |
| RL10 | 57 | 17 | 1 | 0.003 | −2.52 | 0.27 | 0.003 | −2.49 |
| UL31 | 62 | 14 | 1 | 0.003 | −2.46 | 0.23 | 0.005 | −2.29 |
| UL80 | 34 | 10 | 1 | 0.004 | −2.42 | 0.19 | 0.005 | −2.31 |
| UL34 | 14 | 14 | 1 | 0.004 | −2.41 | 0.16 | 0.001 | −2.94 |
| UL3 | 57 | 10 | 1 | 0.005 | −2.33 | 0.16 | 0.008 | −2.09 |
| UL69 | 253 | 24 | 1 | 0.005 | −2.29 | 0.14 | 0.009 | −2.03 |
| UL57 | 426 | 21 | 2 | 0.108 | −2.27 | 0.13 | 0.040 | −3.12 |
| UL123 | 92 | 14 | 1 | 0.006 | −2.21 | 0.13 | 0.008 | −2.12 |
| US14 | 455 | 10 | 1 | 0.011 | −1.95 | 0.31 | 0.065 | −1.20 |
| UL101 | 393 | 16 | 1 | 0.012 | −1.91 | 0.32 | 0.030 | −1.54 |
| UL98 | 413 | 6 | 1 | 0.013 | −1.89 | 0.32 | 0.024 | −1.63 |
| UL67 | 183 | 7 | 1 | 0.014 | −1.86 | 0.32 | 0.025 | −1.61 |
| RL4 | 246 | 1 | 1 | 0.016 | −1.80 | 0.38 | 0.022 | −1.66 |
| UL87 | 182 | 12 | 2 | 0.197 | −1.77 | 0.39 | 0.047 | −2.96 |
| US28 | 416 | 24 | 1 | 0.018 | −1.75 | 0.41 | 0.015 | −1.82 |
| UL16 | 430 | 16 | 1 | 0.019 | −1.73 | 0.40 | 0.032 | −1.50 |
| UL16 | 430 | 6 | 1 | 0.021 | −1.68 | 0.48 | 0.025 | −1.61 |
| UL18 | 330 | 22 | 1 | 0.022 | −1.67 | 0.47 | 0.015 | −1.83 |
| UL93 | 406 | 15 | 1 | 0.022 | −1.66 | 0.44 | 0.078 | −1.13 |
| UL60 | 402 | 19 | 1 | 0.024 | −1.63 | 0.48 | 0.014 | −1.86 |
| UL104 | 387 | 11 | 1 | 0.025 | −1.61 | 0.49 | 0.030 | −1.53 |
| UL86 | 424 | 8 | 2 | 0.245 | −1.59 | 0.49 | 0.091 | −2.41 |
| US23 | 429 | 19 | 1 | 0.026 | −1.59 | 0.47 | 0.015 | −1.83 |

TABLE 6A

KSHV miRNAs: Combined effect on all 3′ UTRs using
hexamers complementary to positions 3-8 in miRNA

| | |
|---|---|
| Local 1st o. MM | Global 3rd o. MM |

| | | | | | | | |
|---|---|---|---|---|---|---|---|
| miRNA name | miRNA# | Hexamer | Actual | Predicted | Log (PV_SH) | Predicted | Log (PV_SH) |

| | | | | | | | |
|---|---|---|---|---|---|---|---|
| kshv-miR-K12-1 | 1 | CCTGTA | 25 | 24.65 | −0.30 | 30.56 | −0.06 |
| kshv-miR-K12-2 | 2 | CTACAG | 34 | 23.31 | −1.66 | 27.53 | −0.89 |
| kshv-miR-K12-3 | 3 | GAATGT | 32 | 24.56 | −1.07 | 24.35 | −1.11 |
| kshv-miR-K12-3* | 4 | GACCGC | 34 | 30.66 | −0.53 | 33.83 | −0.29 |
| kshv-miR-K12-4-5p | 5 | GTTTAG | 21 | 19.52 | −0.40 | 19.67 | −0.39 |
| kshv-miR-K12-4-3p | 6 | GTATTC | 21 | 16.22 | −0.84 | 18.26 | −0.54 |
| kshv-miR-K12-5 | 7 | GCATCC | 36 | 31.64 | −0.62 | 31.48 | −0.63 |
| kshv-miR-K12-6-5p | 8 | GCTGCT | 42 | 33.53 | −1.06 | 39.07 | −0.47 |
| kshv-miR-K12-6-3p | 9 | AACCAT | 26 | 27.59 | −0.19 | 21.67 | −0.70 |
| kshv-miR-K12-7 | 10 | TGGGAT | 34 | 31.74 | −0.44 | 33.55 | −0.31 |
| kshv-miR-K12-8 | 11 | CGCGCC | 43 | 30.46 | −1.73 | 47.81 | −0.11 |
| kshv-miR-K12-9* | 12 | AGCTGG | 57 | 34.14 | −3.67 | 45.27 | −1.29 |
| kshv-miR-K12-9 | 13 | ATACCC | 24 | 23.25 | −0.33 | 25.83 | −0.18 |
| kshv-miR-K12-10a | 14 | CAACAC | 42 | 41.04 | −0.34 | 40.75 | −0.35 |
| kshv-miR-K12-10b | 15 | CAACAC | 42 | 41.04 | −0.34 | 40.75 | −0.35 |
| kshv-miR-K12-11 | 16 | AGCATT | 15 | 24.16 | −0.01 | 19.88 | −0.05 |
| kshv-miR-K12-12 | 17 | GGCCTG | 51 | 44.65 | −0.72 | 52.63 | -0.22 |
| Total: | | | 579 | 502.16 | | 552.89 | |

TABLE 6B
Best KSHV 3′ UTR targets: Combined effect of all microRNAs based on
heptamer complementary to positions 3-8 in miRNA

| | | | Local 1st o. MM | | Global 3rd o. MM | |
|---|---|---|---|---|---|---|
| 3′ UTR | Length | Actual | Predicted | Log (PV_SH) | Predicted | Log (PV_SH) |
| ORF_49 | 1123 | 11 | 5.12 | −1.80 | 5.75 | −1.49 |
| ORF_73 | 1041 | 10 | 4.94 | −1.53 | 5.33 | −1.35 |
| ORF_K8 | 1144 | 10 | 4.99 | −1.51 | 5.86 | −1.13 |
| ORF_40 | 858 | 8 | 3.98 | −1.31 | 4.39 | −1.11 |
| ORF_16 | 4069 | 26 | 18.33 | −1.28 | 20.83 | −0.82 |
| ORF_56 | 1640 | 12 | 7.34 | −1.16 | 8.40 | −0.85 |
| ORF_18 | 1544 | 11 | 6.63 | −1.13 | 7.90 | −0.76 |
| ORF_K14 | 6226 | 37 | 29.11 | −1.05 | 31.87 | −0.69 |
| ORF_25 | 1833 | 13 | 8.61 | −1.01 | 9.38 | −0.82 |
| ORF_72 | 26 | 1 | 0.11 | −0.98 | 0.13 | −0.90 |
| ORF_74 | 4756 | 28 | 22.14 | −0.89 | 24.34 | −0.60 |
| ORF_63 | 2452 | 18 | 13.36 | −0.89 | 12.55 | −1.07 |
| ORF_8 | 1337 | 10 | 6.69 | −0.86 | 6.84 | −0.81 |
| ORF_50 | 2084 | 13 | 9.21 | −0.86 | 10.67 | −0.56 |
| ORF_6 | 396 | 4 | 2.02 | −0.84 | 2.03 | −0.83 |
| ORF_7 | 3858 | 24 | 19.13 | −0.80 | 19.75 | −0.71 |
| ORF_28 | 1151 | 8 | 5.23 | −0.80 | 5.89 | −0.62 |
| ORF_K13 | 50 | 1 | 0.18 | −0.79 | 0.26 | −0.65 |
| ORF_75 | 38 | 1 | 0.18 | −0.79 | 0.20 | −0.75 |
| ORF_59 | 1056 | 8 | 5.32 | −0.77 | 5.41 | −0.75 |
| ORF_47 | 2061 | 12 | 9.07 | −0.69 | 10.55 | −0.44 |
| ORF_K4 | 199 | 2 | 0.85 | −0.68 | 1.02 | −0.57 |
| ORF_32 | 1303 | 8 | 5.72 | −0.66 | 6.67 | −0.45 |
| ORF_26 | 890 | 6 | 4.04 | −0.66 | 4.56 | −0.51 |
| ORF_57 | 63 | 1 | 0.27 | −0.63 | 0.32 | −0.56 |

TABLE 6C
Best KSHV miRNA - 3′UTR target pairs based on hexamer complementary to
positions 3-8 in miRNA

| | | | | Local 1st o. MM | | | Global 3rd o. MM | |
|---|---|---|---|---|---|---|---|---|
| | | | | | Log | | | Log |
| 3′ UTR | Length | miRNA # | Actual | Predicted | (PV_SH) | PV_MH | Predicted | (PV_SH) |
| ORF_K8 | 1144 | 9 | 4 | 0.38 | −3.20 | 0.09 | 0.23 | −4.02 |
| ORF_50 | 2084 | 9 | 5 | 0.73 | −3.01 | 0.07 | 0.42 | −4.13 |
| ORF_74 | 4756 | 5 | 4 | 0.59 | −2.50 | 0.19 | 0.87 | −1.93 |
| ORF_32 | 1303 | 3 | 3 | 0.47 | −1.93 | 0.48 | 0.29 | −2.47 |
| ORF_K4 | 199 | 13 | 1 | 0.01 | −1.92 | 0.41 | 0.05 | −1.33 |
| ORF_25 | 1833 | 14 | 3 | 0.47 | −1.91 | 0.34 | 0.69 | −1.48 |
| ORF_25 | 1833 | 15 | 3 | 0.47 | −1.91 | 0.28 | 0.69 | −1.48 |
| ORF_49 | 1123 | 6 | 2 | 0.17 | −1.90 | 0.26 | 0.19 | −1.80 |
| ORF_18 | 1544 | 11 | 3 | 0.53 | −1.79 | 0.30 | 0.68 | −1.49 |
| ORF_16 | 4069 | 4 | 4 | 0.99 | −1.73 | 0.32 | 1.27 | −1.39 |
| ORF_57 | 63 | 6 | 1 | 0.02 | −1.73 | 0.30 | 0.01 | −1.98 |
| ORF_28 | 1151 | 8 | 3 | 0.56 | −1.72 | 0.27 | 0.42 | −2.06 |
| ORF_56 | 1640 | 7 | 3 | 0.58 | −1.68 | 0.27 | 0.48 | −1.89 |
| ORF_K14 | 6226 | 5 | 4 | 1.03 | −1.68 | 0.23 | 1.13 | −1.55 |
| ORF_49 | 1123 | 13 | 2 | 0.23 | −1.66 | 0.23 | 0.27 | −1.52 |
| ORF_16 | 4069 | 8 | 6 | 2.14 | −1.65 | 0.23 | 1.47 | −2.39 |
| ORF_31 | 2634 | 3 | 3 | 0.60 | −1.64 | 0.23 | 0.59 | −1.65 |
| ORF_63 | 2452 | 2 | 3 | 0.64 | −1.57 | 0.24 | 0.63 | −1.59 |
| ORF_72 | 26 | 5 | 1 | 0.03 | −1.55 | 0.25 | 0.01 | −2.33 |
| ORF_K4 | 199 | 10 | 1 | 0.03 | −1.51 | 0.26 | 0.06 | −1.22 |
| ORF_8 | 1337 | 8 | 2 | 0.28 | −1.50 | 0.24 | 0.48 | −1.07 |
| ORF_59 | 1056 | 17 | 3 | 0.68 | −1.50 | 0.23 | 0.51 | −1.81 |
| ORF_67 | 1866 | 13 | 2 | 0.28 | −1.50 | 0.22 | 0.45 | −1.13 |
| ORF_27 | 1705 | 8 | 3 | 0.71 | −1.46 | 0.24 | 0.62 | −1.61 |
| ORF_64 | 2848 | 6 | 2 | 0.29 | −1.46 | 0.23 | 0.48 | −1.07 |

Tables 3-6 show three pieces of information for each virus. First, there is a list (Table 3A-6A) for each miRNA of the total actual and predicted number of binding sites across all 3′UTRs with associated p-values. miRNAs with smaller p-values are more likely to regulate some (unspecified) viral genes. The total number of functional binding sites for miRNAs can be estimated from the difference of the total numbers of actual and predicted seed binding sites (21).

Second, there is a list (Table 3B-6B) of the top 25 3′UTR targets, sorted according to the p-value based on the total actual and predicted binding-site counts across all miRNAs. 3′UTRs with small p-values are likely to be regulated by some combination of viral miRNAs. Third, there is a list (Table 3 C-6C) of the top 25 miRNA-3′UTR pairs. Pairs with small p-values are most likely to be functional pairs. The ranks of the IE genes in Table 8 below are derived from this list.

Predicting targets of HCMV-coded miRNAs within the HCMV genome. To test our hypothesis that herpesvirus miRNAs might inhibit expression of viral genes needed for efficient lytic replication and thereby favor latency, we asked whether viral miRNAs had potential to target viral 3′UTRs. Instead of listing all conserved potential miRNA binding sites or computing scores based on various empirical rules, our algorithm uses a combination of analytical expressions and Monte Carlo simulations to determine exact probabilities that predicted miRNA targets would occur by chance. We use the standard assumption that the 3′UTR sequence has coevolved with the sequence of the miRNA and the experimental observation that miRNA binding requires a perfect complementarity of a “seed” sequence near the 5′ end of the miRNA to a sequence in the 3′UTR. This seed is usually a heptamer at positions 2-8 from the 5′ end of the miRNA. As a result of coevolution, the number of actual seed oligomers present in the 3′UTR of a targeted gene will be higher than the number that would appear by chance in a random sequence with similar composition. The algorithm predicts functional miRNA targets in two steps:

First, for each miRNA-3′UTR pair, our model computes an approximate probability PV_{SH} (p-value for single hypothesis testing) that it would appear by chance in the random sequence; the smaller PV_{SH} is, the more likely the given pair is to be biologically functional. (Probability PV_{SH} is very nearly exact: The only approximation is that we assume independence between consecutive oligomers.) This procedure alone allows testing whether a given miRNA is likely to target a given 3′UTR.

Second, if we are interested in finding functional targets of multiple miRNAs among multiple 3′UTRs, we need to take into account multiple hypothesis testing. The model does this by performing a Monte Carlo simulation in which we compute the probability PV_{MH} (P-value for multiple hypothesis testing) that the top, say 10, miRNA-target pairs in a randomly generated genome with similar properties would have their PV_{SH} lower than the corresponding top 10 miRNA-target pairs in the real genome. We used this approach instead of the now standard False Discovery Rate analysis (FDR) of Benjamini and Hochberg (1995, J R. Statist. Soc. B 57:289-300) because of the discrete nature of our data. In our data, most PV_{SH} values are 1 and so FDR analysis is not applicable since it requires a fairly uniform distribution of PV_{SH} except a small overrepresentation at values close to 0.

Table 7 below shows the 10 most probable miRNA-target pairs of the 4896 total possible miRNA-3′UTR pairs for the HCMV genome. For each pair, the table shows the score PV_{SH} and the statistical significance PV_{MH} of all predictions up to this one. For instance, the 10^{th} miRNA-target prediction, miR-UL112-1 targeting the IE transactivator protein 1 mRNA (IE1, encoded by the UL123 ORF, highlighted), has a score PV_{SH}=10^{-2.21}=0.0062 and PV_{MH}=0.125, meaning only 12.5% of randomly generated genomes have top 10 p-values better or equal to PV_{SH}=10^{-2.21}. For top 25 most probable miRNA-target pairs in HCMV, see Table 5C above. In fact, the data set in that table suggests that the most significant predictions are the top 10 listed in Table 7 since there is a sharp increase in PV_{MH} from the 10^{th} to 11^{th} prediction: PV_{MH} (10)=0.125 and PV_{MH} (11)=0.309. Naturally, PV_{MH} (k) increases towards 1 for larger k. In our analysis, we required that a target be conserved in six sequenced strains of HCMV. If conservation among strains is not taken into account, PV_{MH} suggests that there are many more significant targets (35 with PV_{MH}<0.20, see SI Table 5C). Finally, the PV_{MH} values listed in Table 7 are conservative upper bounds because we considered all published sequences of detected potential miRNAs although several are only slight variations of each other and some others are perhaps not real miRNAs.

TABLE 7
Top 10 predicted miRNA-target pairs in HCMV
when sorted by PV_{SH} score

| 3′ UTR | | | | 1^{st} order local MM | | |
|---|---|---|---|---|---|---|
| HCMV ORF | Length* | hcmv-miR | Act.^{†} | Exp.^{‡} | Log₁₀ PV_{SH} | PV_{MH} |
| UL103 | 21 | US5-2 | 1 | 0.000 | −4.11 | 0.036 |
| UL112-113 | 67 | UL54-1 | 1 | 0.001 | −2.96 | 0.155 |
| RL10 | 57 | US5-1 | 1 | 0.003 | −2.52 | 0.273 |
| UL31 | 62 | UL112-1 | 1 | 0.003 | −2.46 | 0.229 |
| UL80 | 34 | UL70-5p | 1 | 0.004 | −2.42 | 0.187 |
| UL34 | 14 | UL112-1 | 1 | 0.004 | −2.41 | 0.155 |
| UL3 | 57 | UL70-5p | 1 | 0.005 | −2.33 | 0.155 |
| UL69 | 253 | US33-1 | 1 | 0.005 | −2.29 | 0.144 |
| UL57 | 426 | US25-2- | 2 | 0.108 | −2.27 | 0.127 |
| | | 5p | | | | |
| UL123(IE1) | 92 | UL112-1 | 1 | 0.006 | −2.21 | 0.125 |

The table shows the top 10 of 4896 possible miRNA-3′UTR pairs for the HCMV genome. The statistical significance of the top targets is measured by the multiple hypothesis p-value PV_{MH}. The random background used is the 1^{st} order local MM. IE1 (UL123) is highlighted.
*Length denotes the total number of all conserved heptamers in the 3′UTR.
^{†}Act. denotes the actual count (in the 3′UTR) of conserved heptamers complementary to the miRNA seed.
^{‡}Exp. denotes the count expected in the random sequence.

Predictions of targets for miRNAs coded by other herpesviruses. As described above, the algorithm was applied to an analysis of three additional human herpesviruses. HSV-1, EBV, and KSHV each proved to encode miRNAs predicted to inhibit the expression of viral proteins, including IE proteins. Table 8 displays the rank of the IE-targeting miRNAs among all possible miRNA-3′UTR pairs (the total number is equal to the number of 3′UTRs times the number of miRNAs). The rank is again based on the p-value PV_{SH} computed according to the local first order MM or the global third order MM. ICP0 in HSV-1, BZLF1 and BRLF1 in EBV, and Zta and Rta in KSHV are among the virus-specific targets most likely to be targeted virus-coded miRNAs (top 0.5-2% of virus-specific targets). The BZLF1/BRLF1 3′UTR of EBV is predicted to be targeted by two miRNAs.

TABLE 8
Whole genome ranks for predicted miRNA-IE target pairs in four herpesviruses.

| Virus | 3′ UTR* | Length | miRNA | Seed | Count | Rank A^{†} | Percentile | Rank B |
|---|---|---|---|---|---|---|---|---|
| HSV-1 | ICP0 | 186 | hsv1-miR-LAT | 3-8 | 1 | 4 of 154 | 97.40 | 12 of 154 |
| EBV | BZLF1, BRLF1 | 53 | ebv-miR-BART15 | 2-8 | 1 | 3 of 2720 | 99.89 | 4 of 2720 |
| EBV | BZLF1, BRLF1 | 53 | ebv-miR-BHRF1-3 | 2-8 | 1 | 4 of 2720 | 99.85 | 3 of 2720 |
| HCMV | IE1 | 92 | hcmv-miR-UL112-1 | 2-8 | 1 | 10 of 4896 | 99.80 | 9 of 4896 |
| KSHV | Zta, Rta | 1144 | kshv-miR-K12-6-3p | 3-8 | 4 | 1 of 1394 | 99.93 | 1 of 1394 |

The table reports the top miRNA-IE target pairs for HSV-1, EBV, KSHV and HCMV after sorting by PV_{SH} score.
*BZLF1 and BRLF1 as well as Zta and Rta give rise to 3′ coterminal transcripts and therefore genes in each pair have the same 3′UTRs.
^{†}Rank A (resp. rank B) denotes the rank among all possible miRNA - 3′UTR pairs sorted by p-values computed for the random sequence based on the 1st order local (resp. the 3rd order global) MM. Percentile corresponds to Rank A.

Besides the IE genes, the top predicted miRNA targets include many genes involved in viral DNA replication as well as several inhibitors of apoptosis and other genes involved in immune evasion. Brief descriptions of the predicted targets in these functional groups are summarized in Tables 1 and 2 above.

Table 9 below sets forth each of the miRNAs and mRNA targets mentioned in Tables 1-8, along with representative sequences for each. The skilled artisan will appreciate that these are representative sequences only, as both miRNAs and 3′UTR targets may possess variation with their sequences, while still maintaining the sequence elements that enable recognition and binding of the miRNAs, or derivatives or analogs thereof, to their respective targets in mRNA (SID NO:=SEQ ID NO:).

TABLE 9
3′UTRs and miRNAs and representative sequences.

| | | |
|---|---|---|
| | SID | |
| 3′UTR | NO: | Representative sequence |

3′UTR targets:
Heepes simplex virus

| | | |
|---|---|---|
| RL1 | 1 | ATGGCAGGAGCCGCGCATATATACGCTTGGAGCCAGCCCGCCCTCACAGGGCGGGCCGCCTCGGGGGCGGGA |
| (ICP | | CTGGCCAATCGGCGGCCGCCAGCGCGGCGGGGCCCGGCCAACCAGCGTCCGCCGAGTCTTCGGGGCCCGGCC |
| 34.5) | | CATTGGGCGGGAGTTACCGCCCAATGGGCCGGGCCGCCCACTTCCCGGTATGGTA |
| RL2 | 2 | GGGACGCCCCCCGTGTTTGTGGGGAGGGGGGGGTCGGGCGCTGGGTGGTCTCTGGCCGCGCCCACTACACCA |
| (ICPO) | | GCCAATCCGTGTCGGGGAGGGGAAAAGTGAAAGACACGGGCACCACACACCAGCGGGTCTTTTGTGTTGGCC |
| | | CT |
| UL1 | 3 | CGATGCCTCGACGGAAACCCGTCCGGGTTCGGGGGGCGAACCGGCCGCCTGTCGCTCGTCAGGGCCGGCGGC |
| | | GCTCCTCGCCGCCCTAGAGGCTGGTCCCGCTGGTGTGACGTTTTCCTCGTCCGCGCCCCCCGACCCTCCCAT |
| | | GGATTTAACAAACGGGGGGGTGTCGCCTGCGGCGACCTCGGCGCCTCTGGACTGGACCACGTTTCGGCGTGT |
| | | GTTTCTGATCGACGACGCGTGGCGGCCCCTGATGGAGCCTGAGCTGGCGAACCCCTTAACCGCCCACCTCCT |
| | | GGCCGAATATAATCGTCGGTGCCAGACCGAAGAGGTGCTGCCGCCGCGGGAGGATGTGTTTTCGTGGACTCG |
| | | TTATTGCACCCCCGACGAGGTGCGCGTGGTTATCATCGGCCAGGACCCATATCACCACCCCGGCCAGGCGCA |
| | | CGGACTTGCGTTTAGCGTGCGCGCGAACGTGCCGCCTCCCCCGAGTCTTCGGAATGTCTTGGCGGCCG |
| UL2 | 4 | AAGGCATCGACGTCCGGGGTTTTTGTCGGTGGGGGCTTTTGGGTATTTCCGATG |
| UL5 | 5 | CCCGCCGTCCCCTTACAGTTCCACCGAACCCGGCCCGGGGGACTCACTACCCACCGCGAGATGTCCAATCCA |
| | | CAGACGACCATCGCGTATAGCCTATGCCACGCCAGGGCCTCGCTGACCAGCGCACTGCCCGACGCCGCGCAG |
| | | GTGGTGCATGTTTTTGAGTACGGCACCCGCGCGATCATGGTACGGGGCCGGGAGCGCCAGGACCGCCTGCCG |
| | | CGCGGAGGCGTTGTTATCCAGCACACCCCCATTGGGCTGTTGGTGATTATCGACTGTCGCGCCGAATTTTGT |
| | | GCCTACCGCTTTATAGGCCGGGACAGCAACCAGAAGCTCGAACGCGGGTGGGACGCCCATATGTACGCGTAT |
| | | CCGTTCGACTCCTGGGTCAGCTCCTCGCGCGGCGAAAGCGCCCGGAGCGCCACGGCCGGCATTTTGACCGTG |
| | | GTCTGGACCGCGGACACCATTTACATCACTGCAACCATTTACGGGTCGCCCCCAGAGGAGACGCCAGG |
| UL9 | 6 | GTCTCGGGACCGCACTCGTTCGGTACGTGGTCGTCCGCGGACCGGCGGCGCTGTTGCCGGAACGCACCGAGG |
| | | GGCCAAGTTGGCCCCCGGACCCGGGCCGTTTCCCACCCCCACCCCAACCCCAAAAACCGCCCCCCCCCCGTC |
| | | ACCGGTTTCCGCGACCCACCGGGCCCGGCCAGGCACGGCAGCATGGGACCCACAGACCGCCCGTGATCCTTA |
| | | GGGGCCGTGCGATGGACACCGCAGATATCGTGTGGGTGGAGGAGAGCGTCAGCGCCATTACCCTTTACGCGG |
| | | TATGGCTGCCCCCCCGCGCTCGCGAGTACTTCCACGCCCTGGTGTATTTTGTATGTCGCAACGCCGCAGGGG |
| | | AGGGTCGCGCGCGCTTTGCGGAGGTCTCCGTCACCGCGACGGAGCTGCGGGATTTCTACGGCTCCGCGGACG |
| | | TCTCCGTCCAGGCCGTCGTGGCGGCCGCCCGCGCCGCGACGACGCCGGCCGCCTCCCCGCTGGAGCCC |
| UL11 | 7 | AAACCAAAACAATGTTCTGTATACGGTCGCACGCGTGTCGTTTTTAAAAAACCCACAATCGCCGGGGTGAGG |
| | | GGGGGGGGGGGACGGTGATAGTAACGGGATCGGACGCCACACACCAGACATACACCACGGTCGGGTTAAACA |
| | | CAAACGGTTTATTAAAACGGAACCAAACAGCTACCAACGGCGGACGGTGCTGTACACGGGGTCCTCGGCGGG |
| | | CTCGGGGTCGTACCCCCCAACGGTGTCATAGATGGGATCGTCGTCGGGCAGGTGCCGCGGGTGTTGTATCTT |
| | | GGCGTACAATACGTCGGTTTGGTCGTCCGCCACCTCGTCGTAAATCGGCTCCCCGTCGGAATCTCCGTACCG |
| | | GTCGAGCTGGCCGCCGTATGAGATCGCGTAGGGGTCTTCCGCATATTCGGGAATCCCGGGCGGGCTGCCGGG |
| | | TGCGGGCCTGTGGCGGCCGTCTCGCGATCCGCGCATGGAACTGCGTACGCGCTTGAGGGCGGAATGT |
| UL13 | 8 | GAATCAGCGTTCACCCGGCGGCGCGCTCAACCACCGCTCCCCCCACGTCGTCTCGGAAATGGAGTCCACGGT |
| | | AGGCCCAGCATGTCCGCCGGGACGCACCGTGACTAAGCGTCCCTGGGCCCTGGCCGAGGACACCCCTCGTGG |
| | | CCCCGACAGCCCCCCCAAGCGCCCCCGCCCTAACAGTCTTCCGCTGACAACCACCTTCCGTCCCCTGCCCCC |
| | | CCCACCCCAGACGACATCAGCTGTGGACCCGAGCTCCCATTCGCCCGTTAACCCCCCACGTGATCAGCACGC |
| | | CACCGACACCGCAGACGAAAAGCCCCGGGCCGCGTCGCCGGCACTTTCTGACGCCTCAGGGCCTCCGACCCC |
| | | AGACATTCCGCTATCTCCTGGGGGCACCCACGCCCGCGACCCGGACGCCGATCCCGACTCCCCGGACCTTGA |
| | | CTCTATGTGGTCGGCGTCGGTGATCCCCAACGCGCTGCCCTCCCATATACTAGCCGAGACGTTCGAGC |
| UL14 | 9 | GCCGCTCGTCTCATCGCCGCGCGTCCCCCGAGACGCCCGGTACGGCGGCCAAACTGAACCGCCCGCCCCTGC |
| | | GCAGATCCCAGGCGGCGTTAACCGCACCCCCCTCGTCCCCCTCGCACATCCTCACCCTCACGCGCATCCGCA |
| | | AGCTATGCAGCCCCGTGTTCGCCATCAACCCCGCCCTACACTACACGACCCTCGAGATCCCCGGGGCCCGAA |
| | | GCTTCGGGGGGTCTGGGGGATACGGTGACGTCCAACTGATTCGCGAACATAAGCTTGCCGTTAAGACCATAA |
| | | AGGAAAAGGAGTGGTTTGCCGTTGAGCTCATCGCGACCCTGTTGGTCGGGGAGTGCGTTCTACGCGCCGGCC |
| | | GCACCCACAACATCCGCGGCTTCATCGCGCCCCTCGGGTTCTCGCTGCAACAACGACAGATAGTGTTCCCCG |
| | | CGTACGACATGGACCTCGGTAAGTATATCGGCCAACTGGCGTCCCTGCGCACAACAAACCCCTCGGTC |
| UL16 | 10 | AAATCAGTGCCCACGGGGCAGACTTTCCTCCCGCGTCTGGTTGTGTGTGTATGTGGGTGGGTGGGTGTGGGT |
| | | CGGGTCGACCCGGGGCCCCTTGGGAGAGCCATGCGAAAGAAAAGAGGACTTACGTTTGTGTTGTGGCTGGAG |
| | | GCAAACACGATGGTACTGCGCGACCCGTCCGGAAACGAGAAGGAGATGGTTTCCCCTTTAACGTGGTCCACT |
| | | CGGGCCGAACCGAACCAGCCCCGCAGGCAGGCGTCGATCTCCTCAAACACCGGCTCGGTCGCCTTGCGGATG |
| | | TGCGCCGTGTAGCCGATCTTGATCCCCCGAAAGGAGGCCAGCGACAGCGCGATGAGGGGCACCAGAAACCAG |
| | | GTCTTGCCGTGGCGCCGGGGGACGAGAAACACGGTGGCGCGCTGGCGGAAGTGGCGCACGGCCGCGTCGCTA |
| | | AACAGGGGGATCTCAAACACGAGACGCAGGAACGTGTTGACCTGCTCCGCGTGGTCCCCGAGGAGCAC |
| UL20 | 11 | CGGGGGTGGGGCGGGGGGGGGGGTATATAAGGCCTGGGATCCCACGTCCCCGGGTCTGTTGGGGACACTGGG |
| | | TTCTCCTGGAACGAGGCCGCAGCCTTCTCCCGGTGCCTTTCCCCCCCGACCGGCACCCGGCCTCTCACACAG |
| | | CATCCCCCGCCTTTTTGGGTCCGGGCCCGTCGTGTCTTTCGGTGGACCTTGGGCCGTCGGGCACGTACACGG |
| | | GTGGCCGGGCGTTGGGGTGGATCTTAGCCTCCCCGGGCCAATATCGCTAGAGACAGCCGATCTCCACGCGAC |
| | | CCCATGGCCGCTCCCAACCGCGACCCTCCGGGATACCGGTATGCCGCGGCCATGGTGCCGACCGGGTCCCTC |
| | | CTTAGCACGATCGAGGTGGCGTCGCATCGACGCCTGTTTGATTTTTTTTCCCGCGTGCGCTCCGATGCAAAC |
| | | AGCCTGTACGACGTCGAGTTCGACGCCCTGCTGGGGTCGTATTGCAACACCCTGTCGCTCGTGCGCTT |
| UL24 | 12 | GAGTGTTTCGTTCCTTCCCCCTCCCCCCGCGTCAGACAAACCCTAACCACCGCTTAAGCGGCCCCCGCGAGG |
| | | TCCGAAGACTCATTTGGATCCGGCGGGAGCCACCCGACAACAGCCCCCGGGTTTTCCCACGCCAGACGCCGG |
| | | TCCGCTGTGCCATCGCGCCCCCTCATCCCACCCCCCATCTTGTCCCCA |
| UL34 | 13 | AAAAGGACGCACCGCCGCCCTAATCGCCAGTGCGTTCCGGACGCCTTCGCCCCACACAGCCCTCCCGACCGA |
| | | CACCCCCATATCGCTTCCCGACCTCCGGTCCCGATGGCCGTCCCGCAATTTCACCGCCCCAGCACCGTTACC |
| | | ACCGATAGCGTCCGGGCGCTTGGCATGCGCGGGCTCGTCTTGGCCACCAATAACTCTCAGTTTATCATGGAT |
| | | AACAACCACCCGCACCCCCAGGGCACCCAAGGGGCCGTGCGGGAGTTTCTCCGCGGTCAGGCGGCGGCGCTG |
| | | ACGGACCTTGGTCTGGCCCACGCAAACAACACGTTTACCCCGCAGCCTATGTTCGCGGGCGACGCCCCGGCC |
| | | GCCTGGTTGCGGCCCGCGTTTGGCCTGCGGCGCACCTATTCACCGTTTGTCGTTCGAGAACCTTCGACGCCC |
| | | GGGACCCCGTGAGGCCCGGGGAGTTCCTTCTGGGGTGTTTTAATC |
| UL35 | 14 | GGCCCGGGGAGTTCCTTCTGGGGTGTTTTAATC |
| UL37 | 15 | AGCTTTATTATGTTACGCCCACCCCCGTGTGTTGTTCTCGGTGTTATGGTGTGCGGGCGGGCGGGGGGGGGG |
| | | GTGGAAGACCAAGACAGACAAACGCAGCTCGGTTTTTGGGAAGCGATCACCGCGACTCGTAGCCTAATCAGG |
| | | GGAACCGGGGCCATGGTACGGGGGCATGGGTGGCGGAAACAACACTAACCCCGGGGGTCCGGTCCATAAACA |
| | | GGCCGGGTCTCTGGCCAGCAGGGCACATATGATCGCGGGCACCCCACCGCACTCCACGATGGAACGCGGGGG |
| | | GGATCGCGACATCGTGGTCACCGGTGCTCGGAACCAGTTCGCGCCCGACCTGGAGCCGGGGGGGTCGGTATC |
| | | GTGCATGCGCTCGTCGCTGTCCTTTCTCAGCCTCATATTTGATGTGGGCCCTCGCGACGTCCTGTCCGCGGA |
| | | GGCCATCGAGGGATGTTTGGTCGAGGGGGGCGAGTGGACGCGCGCGACCGCGGGCCCTGGGCCGCCGC |
| UL39 | 16 | CCGACAAACCCCCTCCGCGCCAGGCCCGCCGCCACTGTCGTCGCCGTCCCACGCTCTCCCCTGCTGCCATGG |
| | | ATTCCGCGGCCCCAGCCCTCTCCCCCGCTCTGACGGCCCTTACGGACCAGAGCGCGACGGCGGACCTGGCGA |
| | | TCCAGATTCCAAAGTGCCCCGACCCCGAGAGGTACTTCTACACCTCCCAGTGTCCCGACATTAACCACCTGC |
| | | GCTCCCTCAGCATCCTTAACCGCTGGCTGGAAACCGAGCTTGTTTTCGTGGGGGACGAGGAGGACGTCTCCA |
| | | AGCTTTCCGAGGGCGAGCTCAGCTTTTACCGCTTCCTCTTCGCTTTCCTGTCGGCCGCCGACGACCTGGTTA |
| | | CGGAAAACCTGGGCGGCCTCTCCGGCCTGTTTGAGCAGAAGGACATTCTCCACTACTACGTGGAGCAGGAAT |
| | | GCATCGAAGTCGTACACTCGCGCGTGTACAACATCATCCAGCTGGTGCTTTTCCACAACAACGACCAG |
| UL42 | 17 | CGGGGCGGGGCCTTGGCGGCCGCCCAACTCTCGCACCATCCCGGGTTAATGTA |
| UL47 | 18 | GCTCCTCCCGATAAAAAGCGCCCCGATGGCCCTGGACGCGGCATAACTCCGACCGGCGGGTCCCGACCGAAC |
| | | GGGCGTCACCATGCAGCGCCGGACGCGCGGCGCGAGCTCCCTGCGGCTGGCGCGGTGCCTGACGCCTGCCAA |
| | | CCTGATCCGCGGCGACAACGCGGGCGTTCCCGAGCGGCGCATCTTCGGCGGGTGTCTGCTCCCCACCCCGGA |
| | | GGGGCTCCTTAGCGCGGCCGTGGGCGCCTTGCGGCAGCGCTCCGACGACGCGCAGCCGGCGTTTCTGACCTG |
| | | CACCGATCGCAGCGTCCGGTTGGCCGCGCGGCAACACAACACGGTTCCCGAGAGTTTGATCGTGGACGGGCT |
| | | CGCCAGCGACCCGCACTACGAGTACATCCGGCACTACGCTTCGGCCGCCACCCAGGCGCTGGGCGAGGTGGA |
| | | GCTGCCCGGCGGCCAGTTGAGCCGCGCCATCCTCACGCAGTACTGGAAGTACCTGCAGACGGTGGTGC |
| UL49A | 19 | ACCCGCCCTGTGTGGGGTGAGGGGTGGGGGTGGAGGGTGTCCCAGGACTTCCCCTTCCTCGCGGAAACCGAG |
| | | ACCGTTTGGGGCGTGTCTGTTTCTTGGCCCCTGGGGATTGGTTAGACCCATGGGTTGTGGTTATATGCACTT |
| | | CCTATAAGACTCTCCCCCACCGCCCACAGAGGGCCACTCACGCATCCCCAGTGGGTTTTGCGGACCCTCTCT |
| | | TCTCTCCCGGGCCGCCCCTATCGCTCGACCTCTCCACACCTGCACCACCCCCGCCGTCCGAACCCAGGCCTA |
| | | ATTGTCCGCGCATCCGACCCTAGCGTGTTCGTGGAACCATGACCTCTCGCCGCTCCGTGAAGTCGGGTCCGC |
| | | GGGAGGTTCCGCGCGATGAGTACGAGGATCTGTACTACACCCCGTCTTCAGGTATGGCGAGTCCCGATAGTC |
| | | CGCCTGACACCTCCCGCCGTGGCGCCCTACAGACACGCTCGCGCCAGA |
| UL51 | 20 | ATGCGTGTTTTCATCCAACCCGTGTGTTTTGTGTTTGTGGGATGGAGGGGCGGGTGTGATAGACCCACAGGC |
| | | ATCCAACATAAACAACTACACACAGGAAAGATGCGATACAAACGTTTTTTATTGCCCGGAACGAACCCAAAG |
| | | CTGTGGGCTAAATACCGGTAGAACCAAAACCCCCGGTCCCGCGCTCGCTCGGGGGGGCCTCCGCGTCAAACT |
| | | CGTTCGTAAACACCAGGAGCGGCGGGTTCCTGGGTTCGGCGGTTGAGTCCGGAACACCCCTGGGGTAGTTTC |
| | | GAAGCGCTTTGGTCCCGTGAAAGTTGTCCGGGGGGATCCAAGGAAGAGCGTCCGCCCCCGCAACCAGGAGCT |
| | | GGGCGACCTTGGCGCCGGCCTCGAGGGTCACAGGAACCCCCGTAAGGTTGTAAACAACAAACGCACATACGT |
| | | GCCCGGGGAGCCAGCGCGTAGGAACGACCAGGAGGCCGCGGGCGTTGAGCGACGACCGCCCCAACACA |
| UL52 | 21 | TAACGGCGTACGGCCTCGTGCTCGTGTGGTACACCGTCTTCGGTGCCAGTCCGCTGCACCGATGTATTTACG |
| | | CGGTACGCCCCACCGGCACCAACAACGACACCGCCCTCGTGTGGATGAAAATGAACCAGACCCTATTGTTTC |
| | | TGGGGGCCCCGACGCACCCCCCCAACGGGGGCTGGCGCAACCACGCCCATATCTGCTACGCCAATCTTATCG |
| | | CGGGTAGGGTCGTGCCCTTCCAGGTCCCACCTGACGCCATGAATCGTCGGATCATGAACGTCCACGAGGCAG |
| | | TTAACTGTCTGGAGACCCTATGGTACACACGGGTGCGTCTGGTGGTCGTAGGGTGGTTCCTGTATCTGGCGT |
| | | TCGTCGCCCTCCACCAACGCCGATGTATGTTTGGCGTCGTGAGTCCCGCCCACAAGATGGTGGCCCCGGCCA |
| | | CCTACCTCTTGAACTACGCAGGCCGCATCGTATCGAGCGTGTTCCTGCAGTACCCCTACACGAAAATT |
| US1 | 22 | GTCCGGTCGCCCCGACCCCCTTGTATGTCCCCAA |
| (US 1.5) | | |
| (1CP22) | | |
| US8 | 23 | GGCGCCCCATCCCGAGGCCCCACGTCGGTCGCCGAACTGGGCGACCGCCGGCGAGGTGGACGTCGGAGACGA |
| | | GCTAATCGCGATTTCCGACGAACGCGGACCCCCCCGACATGACCGCCCGCCCCTCGCCACGTCGACCGCGCC |
| | | CTCGCCACACCCGCGACCCCCGGGCTACACGGCCGTTGTCTCCCCGATGGCCCTCCAGGCTGTCGACGCCCC |
| | | CTCCCTGTTTGTCGCCTGGCTGGCCGCTCGGTGGCTCCGGGGGGCTTCCGGCCTGGGGGCCGTCCTGTGTGG |
| | | GATTGCGTGGTATGTGACGTCAATTGCCCGAGGCGCATAAAGGGCCGGTGGTCCGCCTAGCCGCAGCAAATT |
| | | AAAAATCGTGAGTCACAGCGACCGCAACTTCCCACCCGGAGCTTTCTTCCGGCCTCGATGACGTCCCGGCTC |
| | | TCCGATCCCAACTCCTCAGCGCGATCCGACATGTCCGTGCCGCTTTATCCCACGGCCTCGCCAGTTTC |
| US8A | 24 | AGGGCCGGTGGTCCGCCTAGCCGCAGCAAATTAAAAATCGTGAGTCACAGCGACCGCAACTTCCCACCCGGA |
| | | GCTTTCTTCCGGCCTCGATGACGTCCCGGCTCTCCGATCCCAACTCCTCAGCGCGATCCGACATGTCCGTGC |
| | | CGCTTTATCCCACGGCCTCGCCAGTTTCGGTCGAAGCCTACTACTCGGAAAGCGAAGACGAGGCGGCCAACG |
| | | ACTTCCTCGTACGCATGGGCCGCCAACAGTCGGTATTAAGGCGTCGACGCAGACGCACCCGCTGCGTCGGCA |
| | | TGGTGATCGCCTGTCTCCTCGTGGCCGTTCTGTCGGGCGGATTTGGGGCGCTCCTGATGTGGCTGCTCCGCT |
| | | AAAAGACCGCATCGACACGCGCGTCCTTCTTGTCGTCTCTCTTCCCCCCCATCACCCCGCAATTTGCACCCA |
| | | GCCTTTAACTAC |
| US9 | 25 | AAGACCGCATCGACACGCGCGTCCTTCTTGTCGTCTCTCTTCCCCCCCATCACCCCGCAATTTGCACCCAGC |
| | | CTTTAACTAC |
| US11 | 26 | CCCGGGCAAGTATGCCCCCCTGGCGAGCCCAGACCCCTTCTCCCCACAACATGGAGCATACGCTCGGGCCCG |
| | | CGTCGGGATCCACACCGCGGTTCGCGTCCCGCCCACCGGAAGCCCAACCCACACGCACTTGCGGCAAGACCC |
| | | GGGCGATGAGCCAACCTCGGATGACTCAGGGCTCTACCCTCTGGACGCCCGGGCGCTTGCGCACCTGGTGAT |
| | | GTTGCCCGCGGACCACCGGGCCTTCTTTCGAACCGTGGTCGAGGTGTCTCGCATGTGCGCTGCAAACGTGCG |
| | | CGATCCCCCGCCCCCGGCTACAGGGGCCATGTTGGGCCGCCACGCGCGGCTGGTCCACACCCAGTGGCTCCG |
| | | GGCCAACCAAGAGACGTCGCCCCTGTGGCCCTGGCGGACGGCGGCCATTAACTTTATCACCACCATGGCCCC |
| | | CCGCGTCCAAACCCACCGACACATGCACGACCTGTTGATGGCCTGTGCTTTCTGGTGCTGTCTGACAC |
| US12 | 27 | GTCCCGGGTACGACCATCACCCGAGTCTCTGGGCGGAGGGTGGTTCCCCCCCGTGGCTCTCGAGATGAGCCA |
| (1CP47) | | GACCCAACCCCCGGCCCCAGTTGGGCCGGGCGACCCAGATGTTTACTTAAAAGGCGTGCCGTCCGCCGGCAT |
| | | GCACCCCAGAGGTGTTCACGCACCTCGAGGACACCCGCGCATGATCTCCGGACCCCCGCAACGGGGTGATAA |
| | | TGATCAAGCGGCGGGGCAATGTGGAGATTCGGGTCTACTACGAGTCGGTGCGGACACTACGATCTCGAAGCC |
| | | ATCTGAAGCCGTCCGACCGCCAACAATCCCCAGGACACCGCGTGTTCCCCGGGAGCCCCGGGTTCCGCGACC |
| | | ACCCCGAGAACCTAGGGAACCCAGAGTACCGCGAGCTCCCAGAGACCCCAGGGTACCGCGTGACCCCAGGGA |
| | | TCCACGACAACCCCGGTCTCCCAGGGAGCCCCGGTCTCCCCGGGAGCCCCGGTCTCCCCGGGAGCCCC |

Epstein Barr virus

| | | |
|---|---|---|
| BALF2 | 28 | AGACCCCTGGGGCGGCGATGTCGGGGCTGCTGGCGGCGGCGTACAGCCAGGTGTACGCCCTGGCGGTTGAGC |
| | | TGAGCGTGTGCACCCGGCTGGACCCCCGGAGTCTGGACGTGGCTGCGGTGGTGCGCAACGCCGGCCTGCTGG |
| | | CCGAGCTGGAGGCCATCCTCCTTCCCCGTTTGAGACGGCAGAATGACCGTGCATGCAGCGCCCTGTCCCTGG |
| | | AGCTGGTGCACCTGCTAGAGAACTCGAGAGAGGCCTCTGCCGCGCTGCTCGCCCCTGGTAGAAAGGGTACCC |
| | | GGGTCCCGCCTCTCCGTACCCCCTCAGTCGCGTACTCTGTGGAGTTTTACGGGGGGCATAAAGTCGATGTAA |
| | | GTTTGTGCCT |
| BALF3 | 29 | GGTGCTAAGCGTGGTCGTGCTGCTAGCCGCCCTGGCGTGCCGTCTCGGTGCGCAGACCCCAGAGCAGCCCGC |
| | | ACCCCCCGCCACCACGGTGCAGCCTACCGCCACGCGTCAGCAAACCAGCTTTCCTTTCCGAGTCTGCGAGCT |
| | | CTCCAGCCACGGCGACCTGTTCCGCTTCTCCTCGGACATCCAGTGTCCCTCGTTTGGCACGCGGGAGAATCA |
| | | CACGGAGGGCCTGTTGATGGTGTTTAAAGACAACATTATTCCCTACTCGTTTAAGGTCCGCTCCTACACCAA |
| | | GATAGTGACCAACATTCTCATCTACAATGGCTGGTACGCGGACTCCGTGACCAACCGGCACGAGGAGAAGTT |
| | | CTCCGTTGACAGCTACGAAACTGACCAGATGGATACCATCTACCAGTGCTACAACGCGGTCAAGATGACAAA |
| | | AGATGGGCTGACGCGCGTGTATGTAGACCGCGACGGAGTTAACATCACCGTCAACCTAAAGCCCACCG |
| BALF5 | 30 | GACCCAAAGTGAGGGGGCCTGAGACTGGACCCTACTACTATTCTCTCGTTTAAACGAGAGAAGAGAGCGGCG |
| | | AGAGCAGACTCCGAATATCCCCAAAGTCAAGGGAAAGGAAGGGGGCCCTTAGCATGGGAGGCGCGGCGACGA |
| | | GCGGGATAGCAGGACGGGGGGCTGGCGAAGATTCCCAACCGGGGGATCGCTGAATCTAGTATGAAGGCTGGC |
| | | AAAGATCCCCAGTGGAGCGAAGCTAGTGCAGGGGGCTCGGCATTCCTAGGAGAAGGAGCCTCGCCTTGAGGG |
| | | CAAAGACCCCCCCAAGCCTCTCATCAGAATCTCAACCGATTTCGTCAGCCGCTTCAGACAGCCGCGGTTGTC |
| | | ATCATCATCGGGAAAGGCGGTGGGATCATGAAGCCCCCAGGGGAGCGTGGCCCGTGGATCTGTGAAACTCAC |
| | | AGTTTATTTTCTCCAAATCGCTCCTTGCAACAATGGACACGCAAGGGCGAATGCAGAAAATAGTCTGG |
| BARF0 | 31 | AATCTCTATGTCATTTATTAGGCACAAACTTACATCGACTTTATGCCCCCCGTAAAACTCCACAGAGTACGC |
| | | GACTGAGGGGGTACGGAGAGGCGGGACCCGGGTACCCTTTCTACCAGGGGCGAGCAGCGCGGCAGAGGCCTC |
| | | TCTCGAGTTCTCTAGCAGGTGCACCAGCTCCAGGGACAGGGCGCTGCATGCACGGTCATTCTGCCGTCTCAA |
| | | ACGGGGAAGGAGGATGGCCTCCAGCTCGGCCAGCAGGCCGGCGTTGCGCACCACCGCAGCCACGTCCAGACT |
| | | CCGGGGGTCCAGCCGGGTGCACACGCTCAGCTCAACCGCCAGGGCGTACACCTGGCTGTACGCCGccGcCAG |
| | | CAGCCCCGACATCGCCGCCCCAGGGGTCTCTAGACCTCGAGTCCGGGGAGAACGGTGGCCAGACGGCGCTTG |
| | | CGTCTGCCCCCGGAGCCCTGCCCTCCTCCACCCAGCAGCAGCCCGGCCGAGGCCTGCGACGCGGTGCT |
| BaRF1 | 32 | GTCAGGGTGGCTACTTGCTCAGGTTTCTGGGCATAAATTCTCCTGCCTGCCTCTGCTCTGGTACGTTGGCTT |
| | | CTGCTGCTGCTTGTGATCATGGAAACCACTCAGACTCTCCGCTTTAAGACCAAGGCCCTAGCCGTCCTGTCC |
| | | AAGTGCTATGACCATGCCCAGACTCATCTCAAGGGAGGAGTGCTGCAGGTAAACCTTCTGTCTGTAAACTAT |
| | | GGAGGCCCCCGGCTGGCCGCCGTGGCCAACGCAGGCACGGCCGGGCTAATCAGCTTCGAGGTCTCCCCTGAC |
| | | GCTGTGGCCGAGTGGCAGAATCACCAGAGCCCAGAGGAGGCCCCGGCCGCCGTGTCATTTAGAAACCTTGCC |
| | | TACGGGCGCACCTGTGTCCTGGGCAAGGAGCTGTTTGGCTCGGCTGTGGAGCAGGCTTCCCTGCAATTTTAC |
| | | AAGCGGCCACAAGGGGGTTCCCGGCCTGAATTTGTTAAGCTCACTATGGAATATGATGATAAGGTGTC |
| BARF1 | 33 | ACGCACTTGCCTATTTCACCTTGTTTTAGTGTGGCATTGGGGGGGTGGCATTGCGGGTGGATAGCCTCGCGA |
| | | CTCGTGGGAAAATGGGCGGAAGGGCACCGTGGGAAAATAGTTCCAGGTGACAGCAGCAGTGTGTGAAGATTG |
| | | TCACAGCTGCTGGTTTGGAGAAAACGGGGGTGGGCGGTGATCAGGGAGAACAATTCCCCGGGGACACCTGCA |
| | | CGAGACCCCTGGGCTCTCAGGAACTCCGCCCAGGTCTTGCCAATTGGGGTGATCCTGTAGCGCCGCGGTTTC |
| | | AGCATCACAGGTTATTTTGCCTGAAGCTTGCTGGGGCGTAAATCCCTCTCGCCTTGTTTCTCAGAGAGCATT |
| | | TCAGGCCGGTTTTGCAGTCGCTGCTGCAGCTATGGGGTCCCTAGAAATGGTGCCAATGGGCGCGGGTCCCCC |
| | | TAGCCCCGGCGGGGATCCGGATGGGTACGATGGCGGAAACAACTCCCAATATCCATCTGCTTCTGGCT |
| BBLF4 | 34 | ATAAAACAACAGACATGCAGACTCCAGGTTATGACATTTTATTTACAGCCATGGCCAATTGTAGTTGTTATT |
| | | GCCCTTAATGGGGGGGGTGGTTTCCATCATGTGTTTATTGTATGTATTGGGACTTGAAGGTGGAGGGGGGCG |
| | | GCGTGGAGCTGGGCCTCTAAGTACAGGTCGCGTAGGTCTATGGGGACCCTTGTCTTTGGTGGATTGCTGAAC |
| | | TGGGGCTGGTGGCCTGGGAGGTGCTGAGGCCCGTCCCCTGACCGGCGCGGGAGCCGGCGGCCTCGGAGGTGC |
| | | CCGGGTGCGTGGTCGGGAGAACGAAGGCGTGGGTGTCAGACCTGAAGACTGTTGGGTAGATGGCGAGACTCT |
| | | TGAAGATCGTGAGGCCTGAGAGCCGGGGGTTGCTTCATCCTCGTCGCTCTCGCTGTAGTCAGACTCGTCTGA |
| | | ATCTGAAGGATGCCACGAGGGGTCGCTATCACTGCCCTCAGATGGGTCTTCGTCACTGGGGTACTCTT |
| BDLF | 35 | GCCTCCCGCGGGGGGAGGGGGGCACGGATGAGCCCAATCCTCGCCACCTGTGCTCGTATAGTAAGCTGGAGT |
| 3.5 | | TCCATCTCCCGTTACCTGAGAGCATGGCCTCCGTGTTTGCCTGCTGGGGCTGTGGCGAGTACCACGTATGTG |
| | | ATGGATCCAGCGAGTGCACCCTGATTGAGACCCATGAGGGAGTGGTGTGCGCCCTTACAGGCAACTACATGG |
| | | GGCCGCATTTCCAGCCGGCGCTGAGGCCCTGGACCGAGATCCGACAAGACACACAGGACCAGCGGGACAAGT |
| | | GGGAGCCTGAACAAGTCCAGGGCCTGGTTAAGACTGTGGTCAATCACCTCTATCACTACTTTCTGAATGAGA |
| | | ATGTCATCTCCGGGGTCAGCGAGGCCCTCTTTGATCAGGAGGGGGCGCTGAGGCCTCACATCCCGGCCCTGG |
| | | TTTCCTTTGTGTTCCCTTGCTGCCTGATGCTGTTTAGGGGGGCCTCCTCCGAGAAGGTGGTGGATGTG |
| BDLF4 | 36 | GTGGCCTCGGGACCCCCCTCCTCGTGCACCTATTTGTTCCCGACACGGTTATGGCAGAGCTTTGCCCCAATC |
| | | GCGTGCCAAACTGCGAGGGGGCCTGGTGCCAGACTCTCTTCAGTGACCGGACGGGTCTCACGAGGGTCTGCC |
| | | GCGTGTTTGCTGCTCGGGGCATGCTGCCCGGACGGCCTAGCCATCGGGGCACGTTTACCAGTGTGCCAGTGT |
| | | ACTGCGATGAGGGCCTTCCAGAGCTCTACAACCCCTTCCACGTGGCCGCCCTTCGATTTTACGATGAAGGAG |
| | | GGCTGGTTGGGGAGCTACAGATTTATTACCTGTCTCTCTTTGAGGGGGCCAAAAGGGCTCTGACCGACGGGC |
| | | ATCTTATCAGAGAGGCCTCTGGGGTCCAGGAGTCTGCTGCGGCTATGCAGCCCATACCTATAGATCCTGGGC |
| | | CCCCCGGAGGGGCGGGTATAGAGCATATGCCGGTGGCCGCGGCCCAGGTCGAGCACCCTAAAACGTAT |
| BFRF2 | 37 | ATTTCAAGAGCTGAACCAGAATAATCTCCCCAATGATGTTTTTCGGGAGGCTCAAAGAAGTTACCTGGTATT |
| | | TCTGACATCCCAGTTCTGCTACGAAGAGTACGTGCAGAGGACTTTTGGGGTGCCTCGGCGCCAACGCGCCAT |
| | | AGACAAGAGGCAGAGAGCCAGTGTGGCTGGGGCTGGTGCTCATGCACACCTTGGCGGGTCATCCGCCACCCC |
| | | CGTCCAGCAGGCTCAGGCCGCCGCATCCGCTGGGACCGGGGCCTTGGCATCATCAGCGCCGTCCACGGCCGT |
| | | AGCCCAGTCCGCGACCCCCTCTGTTTCTTCATCTATTAGCAGCCTCCGGGCCGCGACTTCGGGGGCGACTGC |
| | | CGCCGCCTCCGCCGCCGCAGCCGTCGATACCGGGTCAGGTGGCGGGGGACAACCCCACGACACCGCCCCACG |
| | | CGGGGCACGTAAGAAACAGTAGAGGGCACGAAACATGGTGTATGCACTTTATT |
| BGLF1 | 38 | CCGGGAACAGCTTCGCAAGTTCCTCAACAAGGAGTGCCTCTGGGTGCTGAGCGATGCCTCTACGCCCCAGAT |
| | | GAAAGTCTATACGGCCACAACCGCCGTGTCAGCTGTGTACGTGCCTCAGATAGCCGGACCTCCTAAAACCTA |
| | | CATGAATGTTACCCTCATTGTGCTGAAGCCCAAGAAGAAGCCCACCTATGTGACCGTCTACATCAATGGAAC |
| | | CCTAGCCACCGTGGCCAGGCCCGAGGTTCTCTTCACTAAGGCAGTCCAGGGGCCACACAGCCTGACTCTCAT |
| | | GTACTTTGGGGTATTCTCAGATGCAGTGGGTGAGGCGGTGCCTGTGGAGATTAGGGGTAACCCTGTAGTCAC |
| | | CTGCACAGATCTGACCACGGCCCACGTCTTTACCACCTCAACCGCCGTTAAAACAGTAGAAGAACTGCAAGA |
| | | TATCACACCCTCGGAGATCATCCCACTGGGACGGGGTGGTGCCTGGTATGCAGAAGGGGCCCTGTACA |
| BGLF2 | 39 | AGCAGGTGGCACACATTACGGTGCTGGAGATTTTCCCACTGTGCCTAAACGTGATGGTGCTGGTCTCCTTGT |
| | | TGACCTCTACACGCTTGGAGTCGAAGCTCTTGGTCAAGGTGTCAATAATTTCAGTGAAAACGGCGGACGCGA |
| | | CATGTTTCTGGTGAGCCACGTAGCCTATTTGCACGTTGGAGAGATTCGAGAGGATGAGGCTGATGATGGCCA |
| | | CGACTATCCAGGTCTTGCCGTGGCGCCTGGGGATAAGAAACACGCTGGCTTTTTGCTTAAAAATGTGCAGCT |
| | | TCTCCAGCGTCATTTCTTCCAATCCGAAAGCACTTTGAAAGATGTCAAACATGGTGTCTGTAATCTCTAAAG |
| | | ATTTGATTGAGATCAGAA |
| BGLF3 | 40 | TTCTAAGCGAGATCTGGTGGCCCAGCAACTAAGAGCCTCGGTAGAAAAGAGAGCGGCTGTGAGCGCACGTGA |
| | | CAGATTTGGGAGGGACCACGCTCTGTTTGAAACACAGTTTACATCTGCTCGGGGTGCCTTAGAGTCCCTGCG |
| | | CCACGCAAGGGAGACGTTTGAGTCCAAACAGCTAATTTCTACCTATCAGAGGGTGGTCACCGCGACCAAGAC |
| | | TCAATTTCCAAAAATCAACTACAAGCAGCTAGAGCGGGTGGAGGAGCTCCGTGAGCAGGAGCTTGAGGCCAG |
| | | AGACGAGCTGCGACAGGCCCTCGAGCCATTTGAGGAACATGGATGTGAATATGGCTGCGGAGTTGAGCCCGA |
| | | CGAACTCCTCCAGCAGTGGCGAGTTGAGTGTCTCCCCAGAACCCCCTCGAGAGACCCAGGCCTTTTTGGGGA |
| | | AGGTGACTGTCATTGATTACTTCACCTTTCAGCACAAACACCTGAAGGTGACCAACATTGATGACATG |
| BGLF | 41 | TTACTTCACCTTTCAGCACAACACCTGAAGGTGACCAACATTGATGACATGACGGAGACCCTCTATGTAAA |
| 3.5 | | GCTGCCGGAGAACATGACGCGCTGTGATCACCTCCCCATTACCTGCGAGTATCTGCTGGGGCGGGGGAGCTA |
| | | CGGGGCCGTGTATGCACATGCAGATAATGCCACGGTCAAACTCTATGACTCTGTGACGGAGCTGTATCACGA |
| | | GCTCATGGTGTGTGACATGATTCAGATTGGGAAGGCCACGGCCGAGGATGGGCAGGACAAGGCCCTGGTGGA |
| | | CTACCTGTCGGCCTGCACGTCCTGCCACGCCCTGTTTATGCCCCAGTTCAGATGCAGTCTCCAGGATTATGG |
| | | CCACTGGCATGATGGTAGTATTGAGCCCCTGGTGCGGGGCTTTCAGGGCCTCAAAGATGCCGTTTACTTTCT |
| | | GAATCGGCACTGCGGCCTCTTCCATTCGGACATTAGCCCCAGCAACATCCTGGTGGATTTCACAGACA |
| BHLF1 | 42 | TGCAGTGTCCCTGCTGCCCATGGAATGCTCAGACCCCGGGTTGGTGGCACTGTTGCGCCCGGCCCTGTACAC |
| | | TACACTCTAAAAGTAACCTGTCTACTTCGCCATGCTTCTTACACTACTCACCTACATGTCAACCGCCTCTAC |
| | | CCTCCCCATGGGATGGCGGCGGTTATGTTTTCCCCATGTTGCGGGTGCCGGCCCTTACAACAGGTTTTGGCA |
| | | ACGAGAGCAATACACAATTAGGCTAAAAGCAGCCACCTATC |
| BHRF1 | 43 | TCTATACATTTTCTCAGCACTTTATATGAATCAGGGTCATTGGGCCTGCGGGGAACTGAGCCAGTAGGATAT |
| | | TAGGCAAGGGTGACACAGTGCCCATGCATTATAATTTAACCAAACAGTGGTCGTGAGTTTTAGGCCGGCCAT |
| | | GGGGGCTTACAAGAATAACATGCCAATGACCCGGCCCCCACTTTTAAATTCTGTTGCAGCAGATAGCTGATA |
| | | CCCAATGTTATCTTTTGCGGCAGAAATTGAAAGTGCTGGCCATATCTACAATTGGGTGTCCTAGGTGGGATA |
| | | TACGCCTGTGGTGTTCTAACGGGAAGTGTGTAAGCACACACGTAATTTGCAAGCGGTGCTTCACGCTCTTCG |
| | | TTAAAATAACACAAGGACAAGATACTAAAGAAATAACTGAGGTGAGTGTGGGAAGATGGGAATACTATGTGT |
| | | TATGTTAACGGGTGAGAGCCTATACTGCAGCCCAGACTCGGGGGGAGGAGGAAATGGTAAGAGTTATA |
| BLLF3 | 44 | CACCTTCATATCCCTTGTTTTACC |
| BMRF1 | 45 | CACCATGTTCTCGTGCAAGCAGCACCTGTCCCTGGGGGCCTGTGTCTTCTGTCTCGGCCTCCTGGCCAGCAC |
| | | CCCCTTCATTTGGTGCTTTGTCTTTGCCAACCTGCTCTCTCTGGAGATCTTCTCACCGTGGCAGACACACGT |
| | | GTACAGGCTTGGATTCCCGACGGCATGCCTAATGGCCGTCCTCTGGACGCTGGTACCCGCCAAGCACGCGGT |
| | | GAGGGCCGTCACTCCAGCCATCATGCTGAATATTGCCAGCGCCTTGATCTTCTTCTCCCTCAGAGTCTACTC |
| | | GACCAGCACGTGGGTTTCTGCCCCCTGTCTCTTTCTGGCCAACCTGCCTCTCTTATGCCTGTGGCCCCGGCT |
| | | GGCCATCGAGATTGTTTACATCTGCCCGGCTATACACCAAAGGTTCTTTGAACTTGGGTTGCTCTTGGCCTG |
| | | CACCATCTTTGCCCTGTCCGTGGTCTCCAGGGCCCTGGAGGTGTCGGCTGTCTTCATGTCTCCATTTT |
| BNRF1 | 46 | CCAGTCACCTTCCAGACTATGCATACACTGAATTTAGCCTGATATTGTCCCCCTAGCCCCGGGCCCAGCCCT |
| | | CCTCAGAAAACTCTGCATGGAGAAGCTGGACGTGAACCTCCCCCCCAGACCTGTGTGCTGTATTTACAAACA |
| | | CTAC |
| BOLF1 | 47 | CGGCGACTGGGGGCAAAGCCAGCGCACCCGGGGAACCGGCCCCGTGCGCGGAATCAGGACCATGGATGTGAA |
| | | TGCCCCCGGGGGCGGGAGTGGAGGCTCGGCCCTCCGCATCCTAGGCACGGCCTCGTGCAACCAGGCCCACTG |
| | | CAAGTTTGGCCGCTTTGCCGGCATCCAGTGCGTCAGCAACTGCGTCCTCTACCTGGTCAAGAGCTTCCTGGC |
| | | CGGCCGCCCCCTGACCTCCCGCCCTGAGCTGGACGAGGTCCTGGACGAGGGGGCGCGGCTGGATGCCCTCAT |
| | | GCGCCAGAGCGGCATCCTCAAGGGGCACGAGATGGCCCAGTTGACGGACGTGCCCAGCTCCGTGGTCCTGAG |
| | | GGGCGGTGGGCGCGTGCACATATACCGCTCGGCGGAGATCTTTGGCCTCGTCCTATTCCCTGCCCAGATCGC |
| | | AAACTCGGCAGTTGTTCAGTCCCTGGCCGAGGTCCTGCACGGCAGTTACAACGGGGTGGCCCAGTTCA |
| BRLF1 | 48 | ACACTTCTGAAAACTGCCTCCTCCTCTTTTAGAAACTATGCATGAGCCACAGGCATTGCTAATGTACCTCAT |
| | | AGACACACCTAAATTTAGCACGTCCCAAACCATGACATCACAGAGGAGGCTGGTGCCTTGGCTTTAAAGGGG |
| | | AGATGTTAGACAGGTAACTCACTAAACATTGCACCTTGCCGGCCACCTTTGCTATCTTTGCTGAAGATGATG |
| | | GACCCAAACTCGACTTCTGAAGATGTAAAATTTACACCTGACCCATACCAGGTGCCTTTTGTACAAGCTTTT |
| | | GACCAAGCTACCAGAGTCTATCAGGACCTGGGAGGGCCATCGCAAGCTCCTTTGCCTTGTGTGCTGTGGCCG |
| | | GTGCTGCCAGAGCCTCTGCCACAAGGCCAGCTAACTGCCTATCATGTTTCAACCGCTCCGACTGGGTCGTGG |
| | | TTTTCTGCCCCTCAGCCTGCTCCTGAGAATGCTTATCAAGCTTATGCA |
| BSLF2/ | 49 | ATGGTTAAACTGAATCTCCACCTGTGTAACCTCACTGTAATTCTATGGGAATAACAAGGGAAGAGGGAAAAG |
| BMLF1 | | AGACTGCGAAAATTCAGTCATATCGGATGCCTCACGCGAAGGGAAACGTGGGAGGCGAATGTAGCCCCTAGG |
| | | CCTGCCACGTGGGTCTCATGGGGGAATGAGGGAAAAGGCCCTAATTCAGCCACCTCCCCTGTGGCCGACTTC |
| | | TGGAACATTTGAGGAGGCACACAAAATGAGGAACGGTGATTAGGCACTGGACACACATGGCACTCATGGTAC |
| | | GGTGATAACTGACAGAGCCGTGTCTCCTGACGCCAATGCCAACTCCCCCAAACATGTCCTGTTAGCTGGTGC |
| | | GGTTATAACTGCCAGAGCTGTGTTTCCCGACGCCAATGCTAACTCCCCAAACATGTCCTGTGAGTTTTGCCC |
| | | ATAAATGACCCCATCCACTGCCACCCCTGGGTTCATTTCCTCCCGTTAGCCCAATGTAATAAGAGGAA |
| BVLF1 | 50 | CCCAGCGTCAGGAAGTACAGCCGGTCGTAGTCATCCGAGGCTGAGAACTGACGCTCCAGGATCTCCCGCGCC |
| | | GCAAGCATGGGCGAGGGGCGCCCCAGGGCAACACCGACGCCGTCCTCGAAGGCTAGACGCAGCTGTGTGCGC |
| | | GCCGCCAGCATGGCAGCCGGGTCGTGA |
| BXLF1 | 51 | GATGCAGTTGCTCTGTGTTTTTTGCCTGGTGTTGCTATGGGAGGTGGGGGCTGCCAGCCTCAGCGAGGTTAA |
| | | GCTGCACCTGGACATAGAGGGGCATGCTTCGCATTACACCATCCCATGGACCGAACTGATGGCAAAGGTCCC |
| | | AGGCCTTAGCCCAGAGGCGCTGTGGAGAGAGGCAAATGTCACCGAAGATTTGGCGTCTATGCTTAACCGCTA |
| | | CAAGTTAATTTACAAGACGTCTGGTACCCTTGGTATTGCGCTGGCCGAGCCTGTCGATATCCCTGCTGTCTC |
| | | TGAAGGATCCATGCAAGTGGATGCATCTAAGGTCCATCCCGGAGTCATTAGCGGCCTGAATTCCCCTGCCTG |
| | | CATGCTTAGTGCCCCCCTTGAGAAGCAGCTCTTCTACTATATTGGCACCATGCTGCCCAACACGCGGCCACA |
| | | CAGCTATGTCTTTTATCAGCTGCGCTGTCACTTGTCTTATGTGGCCCTGTCCATCAACGGGGACAAGT |
| BXRF1 | 52 | GCTGCTCCGCGTGGAGCTGGACGGCATCATGCGTGACCACCTGGCCAGGGCGGAGGAGATCCGCCAGGACCT |
| | | GGATGCTGTAGTGGCCTTCTCTGATGGCCTGGAGAGCATGCAGGTCAGGTCCCCCTCCACGGGAGGGCGCTC |
| | | TGCGCCAGCCCCGCCCTCCCCATCCCCAGCCCAGCCGTTCACTCGGCTCACCGGGAACGCCCAGTATGCAGT |
| | | CTCAATCTCTCCCACGGACCCCCCTCTGATGGTGGCCGGCAGCCTGGCTCAAACGCTGCTTGGTAATCTGTA |
| | | CGGGAACATCAACCAGTGGGTACCGTCCTTCGGACCCTGGTACAGGACCATGTCGGCTAATGCCATGCAGCG |
| | | GCGCGTGTTCCCTAAGCAGCTGAGGGGCAACCTGAACTTTACCAACTCCGTCTCCCTAAAGCTGATGACAGA |
| | | AGTGGTGGCGGTGCTTGAGGGCACCACCCAGGACTTTTTCTCAGACGTCAGGCACCTGCCAGACCTCC |
| BZLF1 | 53 | CTCCCGTTATTGAAACCACGCCTGCTTCACGCCTCGTTTACTAATGGAATATT |
| BZLF2 | 54 | CAGGGGTCACCTTGGATCCCCTTAATCTAGCTCACTTTCAGTGGATGCATCGTAGTCAGTCTGCTTCGCGTC |
| | | CTTTGGGAACACGGAGATCTCAGAATTGTCACTGAGAATCTCCTGTGCTTCAGCAGTAGCTTGGGAACACCG |
| | | GGCAGGTCCGTGAGAACTTTCTTCTACTCGAGGCCTTTTTGGCGTGGTGGCATTAATGTCCAGTGGGGTAAA |
| | | TGCACCTTGACTGTAATCACTGGCAAAGGGCATGCTTGGGCATGCTGTACCTGATGAGTCACACCCCACGGC |
| | | CATGCTATCTTGTAACGGCATAGGGGGAGGGGGGAATCTTGTTGGAATGGGGCGTATGGGGGCTCGGGGCTG |
| | | GGGAGATGACCATGATGGTGCAGAGGATGAGACCAGTGGCACCAATGAAAGTTGAAGACGTGGTGGGCCTGT |
| | | CTCCGATTGCAGATGTGGGAACTGGGAGACCTGATCCTGGCCATGTCCTGCAGATCCATCCCACTGAG |
| LF3 | 55 | TAGAATGACAGCCTGGTCCAAGAGTAAAAGCAGAACAGTAAACACTGCCATAAGTCCTCATGGCAGGAGAGG |
| | | CGGGGGGTATGTGCTGCGTTGGGAACTGAGTAGGCTTGATAGCAGTGACTGGTTGTAACCTATGCCTGGAAG |
| | | AATCATGGCCTACCCGAGACCCCCAACGTCTTGGGTAGGCCATACGTCTAGCCACATAGCAGGTCTCCAGAG |
| | | GGCAGACGTTAGTAACATTTGTATTGTGAGGAAAGGCCTTTAGATATAGAGGCTCTCCCAACACAATAGAAT |
| | | TTTTGCAGCTAAGTTTTCTAAGGGCACGTGCCTTTCCCCCACCCTGGAACAAACATGGGCTGCTATAGTGAG |
| | | CCAGGCTTTCTATGCCTGAAACCCAAGTTTCCTTGCCATCTAAAGCTGCAACTTTCAGTTTAGATCTGTGGT |
| | | TACATGGTGCATTTGCAGGTGTGAAATGCTTGGCCTTGAGTTACTCTAAGGCTAGTCCGATCCCCGGG |
| LMP-1 | 56 | CCTTTCTTTACTTCTAGGCATTACCATGTCATAGGCTTGCCTGACTGACTCTCCCTCCATTTACTGGGAATG |
| | | CCTTAGCTAATCACCTTAACTGGCACACACTCCCTTAGCCACACTGTCTGTCTAGGCTGAAAAGCCACATTC |
| | | ATATTCTATTTCAAAACAAGGGGAAAGGAGGACATGCGAGAATTGGCAGACACCTTTACCCAGCCCTTAACA |
| | | CACCACACAGGTAGCAAGGACCCGGGCGTTGCCAGACTCCGCCACCAACGCCCCTGCGTTGAACCCACCCCT |
| | | CCTACACACATCAGACCTCTGCACAACACAACTACCAGGCAGATGAGGCCCCTTACTTCCACAGGGTACTGG |
| | | CATACCAGCGGGGGACCACATACATCCCTGTCTCCCACCCAGTAACTCCAGCAACTTTGCTTTCCATCTTGT |
| | | GCCAATACACATTTGGATTCAGCCCAAGCCACACCTAACTCATGCCAGCAGAGGCAGGAACACCTGTT |
| LMLP- | 57 | AGGTAAGTATTATTAAATTTTAGAGACACTATCACGTGTAACTTGACGTGCAAGGATGGAAGAGAGGGGCAG |
| 2A | | GGAAACGCAAATGCCGGTTGCCCGGTATGGGGGCCCGTTTATTATGGTAAGGCTCTTCGGGCAAGATGGAGA |
| | | GGCAAACATACAGGAGGAAAGGCTATATGAGCTACTCTCTGACCCACGCTCCGCGCTCGGCCTAGACCCGGG |
| | | GCCCCTGATTGCTGAGAACCTGCTGCTAGTGGCGCTGCGTGGCACCAACAACGATCCCAGGCCTCAGCGTCA |
| | | GGAGAGGGCCAGAGAACTGGCCCTCGTTGGCATTCTACTAGGAAACGGCGAGCAGGGTGAACACTTGGGCAC |
| | | GGAGAGTGCCCTGGAGGCCTCAGGCAACAACTATGTGTATGCCTACGGACCAGACTGGATGGCAAGGCCTTC |
| | | CACATGGTCCGCGGAAATCCAGCAATTCCTGCGACTCCTGGGCGCCACGTACGTGCTTCGCGTGGAGA |
| LMP- | 58 | AGGTAAGTATTATTAAATTTTAGAGACACTATCACGTGTAACTTGACGTGCAAGGATGGAAGAGAGGGGCAG |
| 2B | | GGAAACGCAAATGCCGGTTGCCCGGTATGGGGGCCCGTTTATTATGGTAAGGCTCTTCGGGCAAGATGGAGA |
| | | GGCAAACATACAGGAGGAAAGGCTATATGAGCTACTCTCTGACCCACGCTCCGCGCTCGGCCTAGACCCGGG |
| | | GCCCCTGATTGCTGAGAACCTGCTGCTAGTGGCGCTGCGTGGCACCAACAACGATCCCAGGCCTCAGCGTCA |
| | | GGAGAGGGCCAGAGAACTGGCCCTCGTTGGCATTCTACTAGGAAACGGCGAGCAGGGTGAACACTTGGGCAC |
| | | GGAGAGTGCCCTGGAGGCCTCAGGCAACAACTATGTGTATGCCTACGGACCAGACTGGATGGCAAGGCCTTC |
| | | CACATGGTCCGCGGAAATCCAGCAATTCCTGCGACTCCTGGGCGCCACGTACGTGCTTCGCGTGGAGA |

| | | | |
|---|---|---|---|
| | SID | Representative sequence | Representative sequence |
| 3′UTR | NOs | (FIX strain) | (conserved among six strains) |

Human cytomegalovirus

| | | | |
|---|---|---|---|
| IE1 | 59/60 | ACTATTGTATATATATCAGTTACTGTTATGGATC | ACTATTGTATATATATCAGTTACTGTTATGGATC |
| (UL123) | | CCACGTCACTATTGTATACTCTATATTATACTCT | CCACGTCACTATTGTATACTCTATATTATACTCT |
| | | ATGTTATACTCTGTAATCCTACTC | ATGTTATACTCTGTAATCCTACTC |
| 1E2 | 61/62 | GTGAAAAACTGGAAAGAGAGACATGGACTCTTGT | GTGAAAAACTGGAAAGAGACATGGACTCTTGTAC |
| (UL122) | | ACATAGTGATTCCCCGTGACAGTATTAACGTGTG | ATAGTGATTCCCCGTGACAGTATTAACGTGTGGT |
| | | GTGAGAAGGCTGTTT | GAGAAtGCTGTTT |
| RL1 | 63/64 | ACGTGGTAGGGGGATCTACCAGCCCAGGGATCGC | ACGgGGTAGGGGGATCTACCAGCCCAGGGaTCGC |
| | | GTCTTTCGCCGCCACGCTGCTTCACCGATATCC | GTaTTTCGCCGCCACGCTGCTTCACCGATATCC |
| RL10 | 65/66 | CAAGGAAGGCGAGAACGTGTTTTGCACCATGCAG | caAGGAAGgCGAGAACGTGTTTTGCACCATGCAG |
| | | ACCTACAGCACCCCCCTCACGCTTGTCATAGTCA | ACCTACAGCAcCcCCCTCACGCTTGTCATAGTCA |
| | | CGTCGCTGTTTTTGTTCACAACTCAGGGAAGTTC | CGTCGCTGTTTTTgTtcacaactcagggaagttc |
| | | ATCGAACGCCGTCGAACCAACCAAAAAACCCCTA | atcgaacgccgtcgaaccaaccaaaaaaccccta |
| | | AAGCTCGCCAATTACCGCGCCACCTGCGAGGACC | aagctcgccaattaccgcgccacctgcgaggacc |
| | | GTACACGTACTCTGGTTACCAGGCTTAACACTAG | gtacacgtactctggttaccaggcttaacactag |
| | | CCATCACAGCGTAGTCTGGCAACGTTATGATATC | ccatcacagcgtagtctggcaacgttatgatatc |
| | | TACAGCAGATACATGCGTCGTATGCCGCCACTTT | tacagcagatacatgcgtcgtatgccgccacttt |
| | | GCATCATTACAGACGCCTATAAAGAAACCACGCA | gcatcattacagacgcctataaagaaaccacgca |
| | | TCAGGGTGGCGCAACTTTCACGTGCACGCGCCAA | tcagggtggcgcaactttcacgtgcacgcgccaa |
| | | AATCTCACGCTGTACAATCTTACGGTTAAAGATA | aatctcacgctgtacaatcttacggttaaagata |
| | | CGGGAGTCTACCTCCTGCAGGATCAGTATACCGG | cgggagtctacctcctgcaggatcagtataccgg |
| | | TGATGTCGAGGCTTTTTACCTCATCATCCACCCA | tgatgtcgaggctttttacctcatcatccaccca |
| | | CGTAGCTTCTGCCGAGCTTTGGAAACGCGTCGAT | cgtagcttctgccgagctttggaaacgcgtcgat |
| | | GCTTTTATCCGGGACCAGGGAGAG | gcttttatccgggaccagggagag |
| UL3 | 67/68 | CGACGACGCATACCCGTCGTTCGGCACCCTACCC | cgACGaCGCATAcCCGTCGTTCGGCAcCCTACCC |
| | | GCTTCGCACGCTCAGTACGGCTTTCGACTACTAC | GCtTCGCACGCTCAGTACGGCTTTCGAcTaCTaC |
| | | GCGGCATATTTTTGATTACGCTCGTCATCTGGAC | GCGGCATATTTTTgattAcGCTcGTcATcTGGAC |
| | | CGTAGTGTGGCTCAAACTGCTTCGAGACGCTCTT | CGtAGTGTGGCTCAAaCTGCTTCGAGACGCTCTT |
| | | TTATAAAAACATACGCAGAAAACATTTATGTTCC | TTaTAAAAacatACGcAGAAAAcaTtTaTGTTcc |
| | | GTGATCTCCTGTGGTAACATAGCAACAGGAACCT | gTgATctcctgtggtAACAtagcaacAggAAcct |
| | | GCACTTTCCTTGAATTATGTTCTCATAAACTGTA | gcACTTtccttgaattatgttctcataaactgta |
| | | CCGTCCTGGAGTACGCTATGTATCACGCGTCTTT | ccgtcctggagtacgctatgtatcacgcgtcttt |
| | | TCATGGAGCGCACTGTATGCCGACACACGGAGAT | tcatggagcgcactgtatgccgacacacggagat |
| | | AACGAAGGAAATTCCACTCGCAGATCTGCCTTGT | aacgaaggaaattccactcgcagatctgccttgt |
| | | CTGGAGATGGGGTAGGAATACAACGGCGTTTAAA | ctggagatggggtaggaatacaacggcgtttaaa |
| | | GTAAAGACAGATGAGGCACATGGTGAA | gtaaagacagatgaggcacatggtgaa |
| UL16 | 69/70 | ACGGATAACCGCAAAGGCCACGTGCAACGTTCAC | ACGGATAACCGCAAAGGCCACGTGCAACGTTCAC |
| | | GCTGCTATAAGAAGGCCATGTCCCCCGTGGACGG | GCTGCTATAAGAAGGCCATGTCCcCCGTGGACGG |
| | | GTCTCTTTGACACGAGCGCGGCACGCCGTTGCCA | GTCTCTTTGACACGAGCGCGGCACCCGTTGCCAC |
| | | CGAGCATGGATCACGCGCTCTTCACACACTTCGT | GAGCATGGATCACGCGCTCtTCACACACTTCGTC |
| | | CGGCCGGCCCCGTCACTGTCGGTTGGAAATGTTG | GGCCGgCCCCGTCACTGTCGGTTGGAAATGTTGA |
| | | ATTCTGGACGAACAGGTGTCTAAGAGATCCTGGG | TTCTGGACGAACAGGTGTCTAAGAGATCCTGGGA |
| | | ACACCACGGTTTACCACAGGCGCCGCAGACATCT | CACCACGGTTTACCACAGGCGCCGCAaACATCTA |
| | | ACCTCGACGCCGCGCTCCGTGCGGCCCCCAGAGG | CCTCGACGtCGCGCTCCGTGCGGCCCCCAGAGGC |
| | | CCCGCCGAGATTCCCAAAAGAAGAAAAAAGGCGG | CCGCCGAGATTCCCAAAAGAAGAAaAAAGGCGGC |
| | | CCGTCCTTCTATTTTGGCACGATTTGTGCTGGCT | CGTCCTTCTgTTTTGGCACGATTTGTGCTGGCTG |
| | | GTTTCGACGACTTTTCTTTCCTCGGGAGGACTCG | TTTCGACGACTTTTCTTTCCTCGGGAGGACTCgG |
| | | GAGCCACTGATGTCGGATCCGGCACGGTCTCCCG | AGCCACTGATGTCGGATCCGGCACGGTCTCCCGA |
| | | AAGAGGAGGAGThAACAACACACGGCTAAGAGGA | AGAGGAGGAGTAAACAACACACGGCTAAGAGGAT |
| | | TACATCATCAAAGAAGATAGGAGGGGTCAAAACG | ACATCATCAAAGAAGATAGGAGGGGTCAAAACGt |
| | | CGGACTGAAAGTATATAACGCCGA | GGACTGAAAGTATATAACGCcGA |
| UL17 | 71/72 | ACAACACACGGCTAAGAGGATACATCATCAAAGA | ACAACACACGGCTAAGAGGATACATCATCAAAGA |
| | | AGATAGGAGGGGTCAAAACGCGGACTGAAAGTAT | AGATAGGAGGGGTCAAAACGcGGACTGAAAGTAT |
| | | ATAACGCCGATCATGTCCGAGGAACTGTT | ATAACGCcGATCATGTCCGAGGAACTGTT |
| UL20 | 73/74 | CGGACTTTGGACTGAGCCCCAAGCGGTACGGACT | CGgACTTTGgACtcTGAGCCCCAAGCGGTACGgA |
| | | ATATATTTTCCACAAGTCTACACTGAACTTGAGC | CTAcATATTTTCCAtAAaTCTAtACTGAACTTaA |
| | | ACACAAATACTGACAATAGACTGGATATATAGAC | GCACAaAaATACTGACAATgGACTGgATATAcAG |
| | | TTTTATATGATCCCTGTACAGATGTA | ACTTTTATATaATCCcTGTACAGATGTA |
| UL26 | 75/76 | CAAAACAGGAAGGAAAAAAACACACACATGAAAA | CAAAAtAGGAAGgAAAAaaaccacACgtgaAaaA |
| | | ACCCGGAGAAGACAGAGAGGACGAGCGTCCACAC | AAAAacCCGGAGAAGACAGAGagGACGAGCGTCC |
| | | ACCGCTTTGGTCGTAGACGTACTTTTTAT | ACACACCGCTTTGGTCGTAGACGcATTTTTAT |
| UL29 | 77/78 | GTCATCAGTGTACACACGTCCAGAAATAGGGCGA | GTCATCAGTGTACACgCCCAGAAATAGgGCGACG |
| | | CGGTGTTTTTATAACCGAAAGTAGCGTGTTTGAG | GTGTTTTTATAACCGAAAGTAGCGTGTTTGAGAC |
| | | ACACGCGCTTATAGTCGGTTTTTTCACCGTCGTC | ACGCGCTTcTggTCGGTTTTTTCACCGTCGTCGC |
| | | GCTCTAGGTTTGATTTTCGCGCTCTTGTGTCTCC | TCTAGGTTTGATTTTCGCGCTCTTGTGTCTCCCG |
| | | CGACAGGCTCGTCGTGGGCTACTTTGACTCGCTA | ACAGGCTCGTCGTGGGCTACTTTGACTCGCTcTC |
| | | TCGTCGCTCTATCTGCGCGGGCAGCCCAAGTTCA | GTCGCTCTATCTGCGCGGGCAGCCCAAGTTCAGC |
| | | GCAGCATCTGGCGCGGTCTGCGTGATGCCTGGAC | AGCATCTGGCGCGGTCTGCGTGATGCCTGGACCC |
| | | CCACAAGCGCCCGAAGCCGCGCGAGCGTGCGAGC | ACAAGCGCCCGAAGCCGCGCGAGCGTGCGAGCGG |
| | | GGGGTTCACCTGCAGCGCTACGTACGCGCCACGG | GGTTCACCTGCAGCGCTACGTgCGCGCCACGGCG |
| | | CGGGTCGTTGGCTCCCGCTGTGCTGGCCGCCGCT | GGTCGTTGGCTCCCGCTGTGCTGGCCGCCGCTGC |
| | | GCACGGCATCATGCTGGGCGACACTCAGTACTTT | ACGGCATCATGCTGGGCGACACTCAGTACTTTGG |
| | | GGGGTGGTGCGCGATCACAAGACCTACCGGCGCT | GGTGGTGCGCGATCACAAGACCTACCGGCGCTTC |
| | | TCTCGTGCCTGCGCCAGGCTGGCCGCTTGTACTT | TCGTGCCTaCGCCAGGCTGGCCGCTTGTACTTTA |
| | | TATCGGCCTCGTCAGTGTGTACGAATGCGTGCCG | TCGGCCTCGTCAGTGTGTACGAATGCGTGCCGGA |
| | | GACGCAAACACGGCGCCCGAGATC | cGCAAACACGGCGCCCGAGATCtg |
| UL31 | 79/80 | CCCTCCGTCCGTCCTCCTTTCCCGACACGTCACT | CCCTCCGTCCGTCCTCCTTTCCCGACACGTCACT |
| | | ATCCGATGATTTCATTAAAAAGTACGTCTGCGTG | ATCCGATGaTTTCATTAAAAAGTACGTCTGCGTG |
| | | TGTGTTTCTTAACTATTCCTCCGTGTTCTTAATC | TGTGTTTcTtaactattcctccgtgttcttaatc |
| | | TTCTCGATCTTTTGAAGGATGTTCTGCACGGCGT | ttctcgatcttttgaaggatgttctgcacggcgt |
| | | CCGACGGCGTTTTGGCGCCCCCCATGCCGGCAGA | ccgacggcgttttggcgccccccatgccggcaga |
| | | ACCCGGTTGCGGCCCCGTACCGCTCTTCTGGGGC | acccggttgcggccccgtaccgctcttctggggc |
| | | GACGATAGGTCGAAAGCCACCGTTTTCATGCCCG | gacgataggtcgaaagccaccgttttcatgcccg |
| | | TCGTGCTCTTGACGGGGGAACCTACGGCGGCGGT | tcgtgctcttgacgggggaacctacggcggcggt |
| | | CCCCGTCGAGCGGCGTGATTGCAAAGCCGCGCTC | ccccgtcgagcggcgtgattgcaaagccgcgctc |
| | | GCCCCCGGTTTCAGGATGGAGGGGGAGGCCACAG | gcccccggtttcaggatggagggggaggccacag |
| | | GCGGCGCATTCGATACGCTGCTTTTGGCCGTAGA | gcggcgcattcgatacgctgcttttggccgtaga |
| | | CGACGGTGGGTAAACGGTGGTTACCGCGGGATAC | cgacggtgggtaaacggtggttaccgcgggatac |
| | | GTCGGCGTGGTCGAGGCGGCCCGGCTGCTGCCGG | gtcggcgtggtcgaggcggcccggctgctgccgg |
| | | ACAGGCGACCCGGCGCGCTACCGCTCACGGGGAC | acaggcgacccggcgcgctaccgctcacggggac |
| | | CGAGGGCGGTCGACCTACCACCGC | cgagggcggtcgacctaccaccgc |
| UL32 | 81/82 | TTAAGAAACACACACGCAGACGTACTTTTTAATG | Ttaagaaacacacacgcagacgtactttttaatg |
| | | AAATCATCGGATAGTGACGTGTCGGGAAAGGAGG | aaaccatcggatagtgacgtgtcgggaaaggagg |
| | | ACGGACGGAGGGTCAGGGATGGGGAGATGTGAGA | acggacggagggtcagggatggggagacgtgaga |
| | | AAGTTGTCCGCGGGCAATTGCATGTCGCCCAGAA | aagttgtccgcgggcaattgcatgtcgcccagaa |
| | | AGAACGTGGTTGCTCCGGCGGCGTGCATCTGCCG | agaacgtggttgctccggcggcgtgcatctgccg |
| | | AAACACCGTGTGGTGATTGTACGAGTACACGTTA | aaacaccgtgtggtggttgtacgagtacacgtta |
| | | CCGTCGCCCTCGGTGATTTGATACAACGTGGCGA | ccgtcgccctcgqtgatttgatacaacgtgqcga |
| | | TGGGGGTGCCCTGCGGGATCACGATGGAACGCGT | tgggggtgccctgcgggatcacgatggaacgcgt |
| | | GCGCGTCCACAGCGTGACTTTGAGCGGCTCGCCG | gcgcgtccacagcgtgactttgagcggctcgcca |
| | | CCGCGCCACACGCTGAGCCCCGTGTAAAAGGCGT | ccgcgccacacgctgagccccgtgtaaaaggcgt |
| | | CCTCGTGTGGCAAGTTGGCCACCAAGAAACACCG | cctcgtgtggcaagttggccaccaagaaacaccg |
| | | GTCTGTGATCTGCACGTAGCGCAAGTCCAACTCC | gtctgtgatctgcacgtagcgcaagtccaactcc |
| | | ACCGTCTGCCGCGGTTGCACTCCGAAGTGGATAT | accgtctgccgcggttgcaccccgaagtggatat |
| | | CGTAAGGCGCGTGCACCGTGAGCGAAAACACGTT | cgtaaggcgcgtgcaccgtgagcgaaaacacgtt |
| | | GGGCTCGTTGAGAAGCGGACAGTT | gggctcattgagaagcggacagTT |
| UL33 | 83/84 | GCTTTCCTGTTACTTTAT | GCTTTCCTGTTACTTTAT |
| UL34 | 85/86 | CGTCACTGGAGAAC | CGTCACTGGAGAAC |
| UL37 | 87/88 | CGTCAACGCTGATAGTGTCTATAAAGGCCGTGCC | CGTCAACGCTGATAGTGTCTATAAAGGCCGTGCC |
| | | GCCGCGCCGTAGTTCTCCGAAGGCGGACGGAGGA | GCCGCGCCGTAGTTCTCCGAAGGCGGACGgAGGA |
| | | GTCTGTCGACCGCAGCGGTGGCTGGAGAAGCGCA | GTCTGTCGACCGCAGCGGTGGCTGGAGAAGCGCA |
| | | GCGTCGGCGAGCGAAGGTAGAGGAGTCCGTCATG | GCGTCGGCGAGCGAAGGTAGAGGAGTCCGTCATG |
| | | GACGACCTACGGGACACGCTGATGGCCTACGGCT | GACGACCTACGGGACACGcTGATGGCCTACGGCT |
| | | GCATCGCCATCCGAGCCGGGGACTTTAACGGTCT | GCATCGCCATcCGAGCCGGGGACTTTAACGGTCT |
| | | CAACGACTTTCTGGAGCAGGAATGCGGCACCCGG | CAACGACTTTCTGGAGCAgGAATGCGGCACCCGG |
| | | CTGCACGTGGCCTGGCCTGAACGCTGCTTCATCC | CTGCACGTGGCCTGGCCtGAACGCTGCTTCATCC |
| | | AGCTCCGTTCGCGCAGCGCCCTGGGGCCTTTCGT | AGCTCCGTTCGCGCAgCGCCCTGGGGCCtTTCGT |
| | | GGGCAAGATGGGCACCGTCTGTTCGCAAGGTAAG | GGGCAAGATGGGCACCGTCTGTTCGCAAGGTAAG |
| | | CCCCACGTCGTTGAAGACACCTGGAAAGAGGACG | CCCCACGTCGTTGAAGACACCTGGAAAGAGGACG |
| | | TTCGCTCGGGCACGTTCTTTCCAGGTGTTTTCAA | TTCGCTCGGGCACGTTCTTTCCAGGTGTTTTCAA |
| | | CGTGCGTGGATTTTTTCTCTCTACCAGGTGCTTA | CGTGcGTGGATTTTTtctCTCtACCAGGTGCTTA |
| | | CGTCTGCTGTCAGGAGTACCTGCACCCCTTTGGC | CGTcTGCTGTCAGGAgTACCTGCACCCCTTtGGC |
| | | TTCGTCGAGGGTCCGGGCTTTATG | TTCGTCGAGGGTCCGGgCtttatg |
| UL38 | 89/90 | AAGGAGAACTTTGCTGCTAGATGACCATGTTCAG | AAGGAGAACTTTGCTGCTAGATGACCATGTCAGC |
| | | CTTTTTTTTTGTAGTATTTTTTCATAGTTGCTAT | TTTTTTTTTGTAGTATTTTTTcATAGTTGCTATA |
| | | ACCTCAGTTATCCCCCCTATTAGCCCCACATGCT | CCTCAGTTATCCCCCCTATTAGCCCCACATGCTG |
| | | GCTT | CTT |
| UL40 | 91/92 | TAATGATAACTGCACATCCTCACGAGTGCCCTTA | Taatgataactgcacatcctcacgagtgccttac |
| | | CCTATCATCACACTAAG | ctatcatcacactaag |
| UL43 | 93/94 | GCCGCGGACGCCGTCGGTACCGTCTCCACCACAG | gCCGCGGACGCCGTCGGTACCGTCTCCACCCAGT |
| | | TTGCCACCGTCGCCGTCACTGCCACCGACATGGA | TaCCACCGTCGCCGTCACTGCCACCGACATGGAG |
| | | GCCCACGCCGATGCTCCGCGAGCGGGATCACGAC | CCCACGCCGATGCTCCGCGAcCGGGATCACGACG |
| | | GACGCGCCCCCCACCTACGAGCAAGCCATGGGCC | ACGCGCCCCCCACCTACGAGCAgGCCATGGGtCT |
| | | TGTGCCCAACGACGGTTTCCACGCCACCGCCGCC | GTGCCCgACGACGGTTTCCACaCCACCGCCGCCA |
| | | ACCACCCGATTGCAGCCCACCGCCCTATCGACCC | CCACCcGAcTGCAGCCCACCGCCCTATCGACCCC |
| | | CCGTACTGCCTGGTTAGTTCGCCGTCGCCGCGAC | CGTACTGCCTGGTTAGTTCGCCGTCGCCGCGACA |
| | | ACACGTTCGACATGGATATGATGGAAATGCCCGC | CACGTTCGACATGGAtATGATGGAAATGCCCGCC |
| | | CACCATGCATCCCACCACGGGGGCGTACTTTGAC | ACCATGCATCCCACCACGGGGGCGTACTTTGACA |
| | | AACGGCTGGAAATGGACTTTTGCTCTCTTAGTGG | ACGGCTGGAAATGGACTTTTGCTCTCTTAGTGGT |
| | | TCGCTATATTAGGGATCATTTTCTTGGCCGTGGT | cGCTATATTAGGGATCATTTTCTTGGCCGTGGTG |
| | | GTTCACCGTGGTGATTAACCGGGACAGTGCCAAT | TTCACCGTGGTGATTAACCGGGACAaTtCCAcTa |
| | | ACAACAACGGGGGTTTCCTCATCATCGGGGTAAC | CAACGGGtacAtCATCGGGgTAACGGGaAaTAGA |
| | | GGGGATAGAGCATGTGCTTGACTGTACCATCATT | gCATGTGCTTGACTGTACCATCATTGCTGCTACG |
| | | GCTGCTACGGAATAATAACTACGC | GAATAATAACTacgctacgacct |
| UL44 | 95/96 | AGCGCGTGCCCGGGAACGCGGCCCGCGCGCACGG | AGCGtGgGCCgcGtgcCtgGGaacGCGCGCACGG |
| | | CGCGGTCCCGCGATGGAGAAAACGCCGGCGGAGA | CGCGGTCCCGtGATGGAGAAAACGCCGGCGGAGA |
| | | CGACGGCGGTTTCAGCTGGCAACGTGCCACGTGA | CGACGGCGqTTTCAGCTGGCAACGTGCCACGTGA |
| | | CTCAATCCCGTGTATAACTAACGTGTCCGCGGAC | CTCAATtCCGTGTATAACTAACGTGTCCGCGGAC |
| | | ACCCGCGGCCGTACCCGCCCCAGCAGACCAGCCA | ACCCGCGGCCGTACCCGtCCCAGCAGACCAGCCA |
| | | CCGTTCCTCAGCGACGTCCCGCGCGGATCGGACA | CCGTcCCTCAGCGACGTCCCGCGCGGATCGGACA |
| | | CTTTAGGCGGCGCAGCGCCAGCCTTAGCTTTCTT | CTTTAGGCGGCGCAGCGCCAGCCTTAGCTTTCTT |
| | | GACTGGCCGGACGACAGCGTCACAGAGGGCGTTC | GACTGGCCGGACGaCAGCGTCACAGAGGGCGTTC |
| | | GGACGACCTCCGCGTCGGTCGCCGCCTCCGCGGC | GGACGACCTCCGCGTCGGTCGCCGCCTCCGCGGC |
| | | CCGTTTCGACGAAATCCGGCGACGCCGCCAGAGC | cCGTTTCGACGAAATCCGGCGgCGCCGcCAGAGC |
| | | ATTAACGACGAGATGAAGGAACGCACGCTGGAGG | ATcAACGACGAGATGAAGGAACGtACGCTGGAGG |
| | | ACGCGCTGGCTGTCGAGCTGGTCAACGAGACCTT | ACGCGCTGGCTGTCGAGCTGGTcAACGAGACCTT |
| | | CCGCTGCTCTGTCACCGCCGACGCCCGCAAGGAC | CCGCTGCTCTGTCACCgCCGACGCcCGCAAGGAC |
| | | CTGCAGAAGCTGGTTCGTCGCGTCAGTGGCACGG | CTGCAGAAGCTGGTTCGTCGCGTCAGcGGCACGG |
| | | TGCTGCGTCTCAACTGGCCGAACG | TGCTGCGTCTCAgCTGGCCgAACG |
| UL45 | 97/98 | TCGGGGGCCCGCTGGCTCGGCGCGGCTGTATTAT | TCGGGGGCCCGCTGGCTCGGCGCGGCTGTATTAT |
| | | TAGACGCCGGGCGTCTTCGCAGCGTTCCCGGTCG | TAGACGCCGGGCGTCTTCGCAGCGTTCCCGGTCG |
| | | TCGTGTGTGCTCTCTATAAAACTTTCGCTCGCTC | TCGTGTGTGCTCTCTATAAAACTTTCGCTCGCTC |
| | | GCGCCCGCTCCTTAGTCGAGACTTGCACGCTGTC | GCGCCCGCTCCTTAGTCGAGACTTGCACGCTGTC |
| | | CGGGATGGATCGCAAGACGCGCCTCTCGGAGCCG | CGGGATGGATCGCAAGACGCGCCTCTCGGAGCCg |
| | | CCGACGCTGGCGCTGCGGCTGAAGCCGTACAAGA | CCGACGCTGGCGCTGCGGCTGAAGCCGTACAAGA |
| | | CGGCTATCCAGCAGCTGCGATCTGTGATCCGTGC | CGGCTATCCAGCAGCTGCGATCTGTGATCCGTGC |
| | | GCTCAAGGAGAACACCACGGTTACCTTCTTGCCC | GCTCAAGGAGAACACCACGGTTACCTTCTTGCCC |
| | | ACGCCGTCGCTTATCTTGCAAACGGTACGCAGTC | ACGCCGTCGCTTATCTTGCAAACGGTACGCAGTC |
| | | ACTGCGTGTCAAAAATCACTTTTAACAGCTCATG | AcTGCGTGTCAAAAATCACTTTTAACAGCTCATG |
| | | CCTCTACATCACTGACAAGTCGTTTCAGCCCAAG | cCTCTACATCACtGACAAGTCGTTTCAGCCCAAG |
| | | ACCATTAACAATTCCACGCCGCTGCTGGGTAATT | ACCATTAACAATTCCACGCCGCTGCTgGGtAATT |
| | | TCATGTACCTGACTTCCAGCAAGGACCTGACCAA | TcATGTACCTGACtTCCAGCAAGGACCTGACCAA |
| | | GTTCTACGTGCAGGACATCTCGGACCTGTCGGCC | GTTCTACGTGCAGGACATCTCGGACCTgTCGGCC |
| | | AAGA | AAGATCTCCATGTGCGCGCCCGAT |
| UL50 | 99/100 | CGAGTTCCACCAGGCTCTGTGCCGTCTCTTCGCG | tGAGTTCCACCAGGCTCTGcGCCGTCTCTTCGCG |
| | | CCCCTCTGCGTTCACGAGGACCATTTCCATGTGC | CCCCTCTGCGTTCACGAGGACCATTTCCATGTGC |
| | | AGCTGGTGATCGGCCGCGGTGCGCTGCAGCCGGA | AGCTGGTGATCGGCCGCGGTGCGCTGCAGCCGGA |
| | | GGAAGCGGCGGTAGAAACGTCGCAGCCACCGGCG | GGAAGCGGCGGTAGAAACGTCGCAGCCACCGGCG |
| | | CAGTTTGCGGCGCAGACGTCGGCGGTCCTCCAGC | CAGTTTGCGGCGCAGACGTCGGCGGTCCTCCAGC |
| | | AGCAGCTGGTGCATCACGTGCCACGTTCTTGCGT | AGCAGCTGGTGCATCACGTGCCACGTTCTTGCGT |
| | | CCTTCATCTCTTCGTGACGGATAAGCGCTTTCTG | CCTTCATCTCTTCGTGACGGATAAGCGCTTTCTG |
| | | AATCGCGAGCTGGGCGACCGTCTCTACCAACGCT | AATCGcGAGCTGGGCGACCGTCTCTACCAACGCT |
| | | TCCTGCGCGAATGGCTGGTGTGTCGGCAGGCCGA | TCCTGCGCGAATGGCTGGTGTGTCGGCAaGCCGA |
| | | GCGGGAGGCGGTGACGGCGCTCTTTCAGCGTATG | GCGGGAGGCGGTGACGGCGCTcTTTCAGCGTATG |
| | | GTTATGACCAAGCCCTACTTTGTGTTTCTCGCTT | GTTATGACCAAGCCCTACTTTGTGTTTCTCGCTT |
| | | ACGTCTACAGCATGGACTGTCTGCACACCGTGGC | ACGTCTACAGCATGGACTGTCTGCACACCGTGGC |
| | | CGTCCGCACGATGGCCTTTCTGCGTTTCGAACGC | CGTCCGCACGATGGCCTTTCTGCGTTTCGAACGC |
| | | TACAACACCGACTACCTGCTGCGCCGTCTGCGGC | TACgACgCCGACTACCTGCTGCGCCGTCTGCGGC |
| | | TCTACCCGCCCGAGCGGCTGCACG | TCTACCCGCCCGAGCGGCTGCACG |
| UL51 | 101/102 | ATCGGCGGTGGCGTCGGTGCGATGGAGATGAACA | ATCGGCGGTGGCGTCGGTGCGATGGAGATGAACA |
| | | AGGTTCTCCATCAGGATCTGGTGCAGGCCACGCG | AGGTTCTCCATCAGGATCTGGTGCAGGCCACGCG |
| | | GCGTATCCTCAAGTTGGGTCCCAGCGAGCTGCGC | GCGTATCCTCAAGTTGGGTCCCAGCGAGCTGCGC |
| | | GTCACCGATGCCGGCCTCATCTGTAAAAACCCCA | GTCACCGAcGCCGGCCTcATCTGTAAAAAcCCCA |
| | | ATTACTCGGTGTGCGACGCCATGCTCAAGACAGA | ATTACTCGGTGTGCGACGCCATGCTCAAGACAGA |
| | | CACGGTCTATTGTGTCGAGTATCTGCTCAGCTAC | CACGGTCTATTGTGTCGAGTATCTgCTCAGCTAC |
| | | TGGGAGAGCCGCACAGACCACGTGCCTTGTTTTA | TGGGAGAGCCGCACAGACCACGTGCCTTGTTTTA |
| | | TCTTTAAAAACACTGGCTGTGCCGTCTCCCTCTG | TCTTTAAAAACACTGGCTGtGCCGTCTCCCTCTG |
| | | CTGTTTTGTGCGAGCGCCCGTCAAGCTCGTTTCG | CTGTTTTGTgCGAGCGCCCgTCAAGCTCGTcTCG |
| | | CCGGCGCGCCACGTAGGTGAGTTCAATGTGCTTA | CCGGCGCGCCACGTAGGTGAGTTCAATGTGCTTA |
| | | AGGTGAACGAGTCGCTCATCGTCACGCTCAAGGA | AGGTGAACGAGTCGCTCATCGTCACGCTCAAGGA |
| | | CATCGAGGAGATCAAGCCCTCGGCCTACGGAGTG | CATCGAGGAGATCAAGCCCTCGGCCTACGGAGTG |
| | | CTGACGAAGTGCGTGGTGCGCAAATCCAATTCGG | CTGACGAAGTGCGTGGTGCGCAAATCCAATTCGG |
| | | CGTCGGTCTTCAACATCGAGCTCATCGCCTTCGG | CGTCGGTCTTCAACATCGAGCTCATCGCCTTCGG |
| | | ACCCGAAAACGAGGGCGAGTACGA | ACCCGAAAACGAGGGCGAGTACGA |
| UL52 | 103/104 | CGTGAGCGGCGTGCGCACGCCGCGCGAACGACGC | CGTGAGCGGCGTGCGCACGCCGCGCGAACGACGC |
| | | TCGGCCTTGCGCTCCCTGCTCCGCAAGCGCCGCC | TCgGCCTTGCGCTCCCTGCTCCGCAAGCGCCGCC |
| | | AACGCGAGCTGGCCAGCAAAGTGGCGTCAACGGT | AACGCGAaCTGGCCAGcAAAGTGGCGTCgACGGT |
| | | GAACGGCGCTACGTCGGCCAACAACCACGGCGAA | GAACGGCGCTACGTCGGCCAACAACCACGGCGAA |
| | | CCGCCGTCGCCGGCCGACGCGCGCCCGCGCCTCA | cCGCCGTCgCCGGCCGACGCGCGCCCGCGCCTCA |
| | | CGCTGCACGACTTGCACGACATCTTCCGCGAGCA | CGCTGCACGACcTGCACGACATCTTCCGCGAGCA |
| | | CCCCGAACTAGAGCTCAAGTACCTCAACATGATG | CCCCGAACTgGAGCTCAAGTAcCTcAACATGATG |
| | | AAGATGGCCATCACGGGCAAAGAGTCCATCTGCT | AAGATGGCCATcACGGGCAAAGAGTCCATCTGCT |
| | | TACCCTTCAATTTCCACTCGCACCGGCAGCACAC | TACCCTTCAATTTCCACTCGCAcCGGCAGCACAC |
| | | CTGCCTCGACATCTCGCCGTACGGCAACGAGCAG | CTGCCTCGACATCTCGCCGTACGGCAACGAGCAG |
| | | GTCTCGCGCATCGCCTGCACCTCGTGCGAGGACA | GTCTCGCGCATCGCCTGCACCTCGTGCGAGGACA |
| | | ACCGCATCCTGCCCACCGCCTCCGACGCCATGGT | ACCGCATCCTGCCCACCGCCTCCGACGCCATGGT |
| | | GGCCTTCATCAATCAGACGTCCAACATCATGAAA | GGCCTTCATCAATCAGACGTCCAACATCATGAAA |
| | | AATAGAAACTTTTAT | AATAGAAACTTTTAT |
| UL54 | 105/106 | GAAACAGCGGCGGCGGTGGTGACTGGGGACGGTG | GAAACAGCGGCGGCGGTGGTGACTGGGGACGGTG |
| | | ATGATGCTGCTGAGACTGAGACTGGTGGTGAGAG | ATgATGCTGCTGAGACTGAGaCTGGTGGTGAGAG |
| | | TAGTGGTGGGGCTGCGTCGCCTGCGACGGCGGGT | TAGTGGTGGGGCTGCGTCGCCTGCGACGGCGGgT |
| | | GGAGATGAGGCGGCGTGGACTGGGACGAGGAGGA | GGAGATGAGGCGGCGTGGACTGGGACGAGGAGGA |
| | | GGGGCCGCAGCCGTTGGTGGAAACTACGTGCAAC | GGGGCCGCAGCCGTTGGTGGAAacTACGTGCAAC |
| | | GGCGACGCGGTTAAGGGAGACCGTATCGCGTAGG | GGCGACGCGGTTAaGGGAGACCGTATCGCGTAGG |
| | | ACGACGTGGCCTCCTCGTATAGGTTGTTGCCGCT | AcGACGTGGCCTCCTCGTATAGGTTGcTGCCGCT |
| | | GGACTGACACAGCTCCTGAATGAGCTCTTTGTAG | GGACTGACACAGCTCCTGAATGAGCTCTTTGTAG |
| | | CGCTCAAAGGACTCGCTCACGTCGTTGGGAATGT | CGCTCAAAGGACTCGCTCACGTCGTTGGGAATGT |
| | | CCATCTCGTCAATCTTGCGTTGCAAAATAGTCAC | CCATCTCGTCAATCTTGCGTTGCAAAATAGTCAC |
| | | GTCGATCTTGACGCTGCTGGCCGAGACGGCGTGA | GTCGATCTTGACGCTGCTGGCCGAGACGGCGTGA |
| | | CACAGCACGCTGATAACGACGTGGTCGCGCACGA | CACAGCACGCTGATAACGACGTGGTCGCGCACGA |
| | | TGTTGAGCGTGACGCTGTAGTCTTCGCGCGCCGC | TGTTGAGCGTGACGCTGTAGTCTTCGCGCGCCGC |
| | | CGTGAGCATCTGCGTGATGCAGTCGCAGGGGATG | CGTGAGCATCTGCGTGATGCAGTCGCAGGGGATG |
| | | TGCACGTCGGGGTTTTCGAAGATG | TGCACGTCGGgGTTTTCGAAGatg |
| UL57 | 107/108 | CCGCCAGCAAACGCCGCGACAACGGCCGCCGCAG | CCGCCAGCAaACGCCGCGACAACGGCCGCCGCAG |
| | | CCACGAGCATCGCAACAACAGCAGCAACAGTCGC | CCACGAGCgTtGCAACAACAGCAgCAACAGTCGC |
| | | AGCCCCCGTGGCCGCTTTTCAGACCGCAACAACA | AGCCCCCGTGGCCGCTTTTCAGACCGCAACAACA |
| | | GCAGCAACAGCAGCCACCGACACAGCAGCACCAG | GCAGCAACAGCAGCCACCGACACAGCAGCACCAG |
| | | GCGACACCGTATCAGCTACCGCCGCAACAGCGGC | GCGAtACCGTATCAGCTACCGCCGCAACAGCGGC |
| | | GACAGACGGCGTCGCATCATCAACAGCAGCAACA | GACAGACGGCGTCGCATCATCAaCAGCAGCAACA |
| | | GCCCCGAAGGTTAGCGCCGCGGCACCAGAGACAG | GCCCCGAAGGTTaGCGCCGCGGCACCAGAGACAG |
| | | AGACCGCCGCCGCGCTGGCAAACTCCGACATTCG | AGACCGCCGCCGCGCTGGCAAaCTCCGACATTCG |
| | | CGTCGGCGCCCGGGCCGCCTGAGGAAGGGGAGGA | CGTCGGCGCCCGGGCCGCCTGAGGAAGGGGAGGA |
| | | GTGTCAGACACAGCCGGTCATCTCCGAGCCCCCG | GTGTCAGACACAGCCGGTCATCTCCGAGCCCCCG |
| | | TCGCCCGAGGCGGAGGAGCCGGCGGCGGCGGTGG | TCGCCCGAGGCGGAGGAGCCGGCGGCGGCGGTGG |
| | | TGGAGGAGGTTGCGCCGCAAGCGGCGGCAACAGC | TGGAGGAGGTTGCGCCGCAaGCGGCGGCAACAGC |
| | | TTCGGGAGCAGAACCCGCGTCGTCGACGACGTCG | tTCGGGAGCAGAACCCGCGTCGTCGACGACGTCG |
| | | TTATATATTAACGTCAACGTCAGTCGGCATAGCG | TTATATATTAACGTCAACGTCAGTCGGCATAGCG |
| | | AGCGGCCCGCGAGTTATTTGTGCA | AGCGGCCCGCGAGTTATTTGTgca |
| UL60 | 109/110 | AACGGACTGATGACGTAGCTCGCTTCGCTCGCTA | AACGGACTGATGACGTAGCTCGCTTCGCTCGCTA |
| | | CGTCATCAGAGATGATTTCCGCCGGAGGTGGCGC | CGTCATCAGAGATGATTTCCGCCGGAGgTGaCGc |
| | | ACGCATACGTGACGTAGCTCGCTACGCTCGCTAC | ACGCATACGTGACGTAGCTCGCTACGCTCGCTAC |
| | | GTCATCGTATGTCCGGAATTCCACGGGATGACGT | GTCAcCGTATGTCCGGAATTCCACaGGATGACGT |
| | | ATATCCGGAGTGGGTGTGGTCACGCGAGTGTGAC | ATATCCGGAGTGGGTGTGGctACGCGAGTGTGAC |
| | | GTAGGCTTGTCAGGGGTCACGTGAGAAGCGGCGG | GTagGCTTGtCAGGGGTCACGTGAGAAGCGGCGG |
| | | CGTTAAGTTTACTAGGCCAAAACAGAGGAAGGGG | CGTTAAGTTTACTAGGcCAAAACAGAGGAAGGGG |
| | | GCGGATACCCTAAGTAAGGGGGCGTGCACGTAGC | GCGGATACCCTAgGTAAGGGGGCGTGCACGTAGC |
| | | CCTGTAGACACTCCCCCCTAGGGTCCAGTAGCTT | CCTGTAGACACTCCCCCCTAGGGTCCAGTAGCTT |
| | | ATGACGCGTATCCGGGAGTAGCGTCTACGTCAGC | ATGACGCGTATCCGGGAGTAGCGTCTACGTCAGC |
| | | AGGTGTATATTTCCGGTAAACGGAGAAGCCTGTA | AGGTGTATATTTCCGGTAgACGGAGAAGCCTGTA |
| | | CGTACACCGAGGACGGTGGAACCCTAACGGGTTC | CGTACACCGAGGACGGTGGAACCCTAACGGGTTC |
| | | CACCTATCTGAAATTTCCGTACAAGGGGTGGAGT | CACCTATCTGAAATTTCCGTACAAGGGGTGGAGT |
| | | CTAGGGAGGGGTCATTGTATATTCGTTTCTGTGA | CTAGGGAGGGgTCATTGTATATCCGTTTCTgTGA |
| | | TTGGTAGATAAGGTAGCGTACCTA | TTGGTAGATAAGGTgGCGTACCTA |
| UL61 | 111/112 | GGCGGGAAGCAGGCGGGAGCGGGCGCAGCGTGCG | ggcgggaagcaggcgggagcgggcgcagcgtgcg |
| | | GACCGCAGCACGGCCGGAACCCTGCCGCGGACTG | gaccgcagcacggccggaaccctgccgcggactg |
| | | CGCCGGGGGGCGGCGGGCACGCCGGGTTTTATAG | cgccggggggcggcgggcacgccgggttttatag |
| | | GTTTTCAGATGCCCCGCCTAGGTGGGCGGAGCGG | gttttcagatgccccgcctaggtgggcggagcgg |
| | | TAATTTTCCACCGCCGCGGCCCATGCCCGGCACG | taattttccaccgccgcggcccatgcccggcacg |
| | | GGGCTCGCGCTCCCTAGGTGCGGCCGCCCAGTGG | gggctcgcgctccctaggtgcggccgcccagtgg |
| | | AAAAACACCGGCGCATGCGCACGGCGCACATCCA | aaaaacaccggcgcatgcgcacggcgcacatcca |
| | | GTGGAATTTTACCGACGCATGCGCACTGACCGCC | gtggaattttaccgacgcatgcgcactgaccgcc |
| | | TCCAGTGGAAAAATACTGGCGCATGCGCACGACA | tccagtggaaaaatactggcgcatgcgcacgaca |
| | | CACACCCGGTGGAATTTTACCGGCGCATGCGCAG | cacacccggtggaattttaccggcgcatgcgcag |
| | | GGCGACCCTCCCGCGGTCCCTGGCTCGCGCATGC | ggcgaccctcccgcggtccctggctcgcgcatgc |
| | | GCACCGGGGCCCCTGGTTCACCCCTCCTTATATA | gcaccggggcccctggttcacccctccttatata |
| | | TAGGTTTTCCATGCGGCATCCCCGGCGCATGCGC | taggttttccatgcggcatccccggcgcatgcgc |
| | | ACTCGAGTCCCCATCCCATAATCCGCGTGGCAAC | actcgagtccccatcccataatccgcgtggcaac |
| | | GCCCTGACAACCAAAAACTCGCCC | gccctgacaaccaaaaactcgccc |
| UL67 | 113/114 | GGTTATAGCATCATCTAGTTTGTTCATTTCATAC | GGTTATAGCATCATCTAGTTTGTTCATTTCATAC |
| | | CTGTTGAGAACGTTTATGTTCTAGCAATTGATTT | CTGTTGAGAACGTTTATGTTCTAGCAATTGATTT |
| | | CGCGTCATAGGGCTGTGACGGTGATTCTTCAGAG | CGCGTCATAGGGCTGTGACGGTGATTCTTCAGAG |
| | | AATCAGAAAAAAAAAAGAGGCTCAACGAGCACCA | AATCAGgAAAAAAAAAaaGAGGCTCAACGAGCAC |
| | | GAGACTAAGTCGGAAAACTCGCGCCCGCTTCCCC | CAgAGaCTAAGTCGGAAAACTCGCGCCCGCTTCC |
| | | GGACGGTTTCAGCTTAGCCTCTGGCCTGCGATGG | CCGGACGGTTTCgGCTTAGCCTCTGGCCTGCGAT |
| | | TTTTTTTAT | GGTTTTTTTAT |
| UL69 | 115/116 | AAAGAGAGTGAGGGGTGTTGTGCGTGATTGCTGT | AAAGAGAGTGAaGGGTGTTGTGCGTGAtgaTTGC |
| | | CCCTTATCCCGTTACAAAGAAAAAAGAAAAAATG | TGTCCCTTATCCCGTTACAAAGAAAAgaaaaAAT |
| | | GTGTTACACACTCCTTGGTACTACTATGACTCGT | GGTGTTACACACTCCTTGGTACTACTATGACcCG |
| | | GGTGAGATATCCGATGATGATAATGATGTACGCG | TGGTGAGATATCCGATGATGATAATaatGATGTA |
| | | TGCCTGAGCTTGGTGTTTTTTTTTCTCTCTGTGA | CGCGTGCCTGAGCTTGGTGTTTTTTCTCTCTGTG |
| | | GCTTTTTTCCCCATAAGCTGTGTACTGTTCGTGT | AGCTTTTTTCCCCATAAGCTGTGTACTGTTCGTG |
| | | CCGGACCCCATACACGGTTTCCGTTAATGACGGC | TCCGGACCCCATACACGGTTTcCGTTAATGACGG |
| | | CCCCTCCTTTTCCCCCACCGTAAAAAAAAAAAAC | CCCCCTCCTTTTCCCCcACCGTAAAAAaaaaaac |
| | | AAAGCACAATACACATGTGGTTTTTTGGTTCGAA | AAAGCACAATACACATGTGGTTTTTTGGTTCGAA |
| | | TCGAGCTTGGCGTTTAT | TCGAGCTTGGCGTTTAT |
| UL78 | 117/118 | GCGGCGGCGCTGTACGGCAGCGGGGAGAAAAGTG | GCGGCGGCGcTGTACGgCAGCGGGGAGAAAAGTG |
| | | GCAGATAAATCACGTTAGGTTCACACGTCGTTAG | GCAGATAAATcACGTcAGGTTCACACGTCGtTAG |
| | | CCAGCGTCGGCATATGAAGGGCGCGGGCGGCCAG | CCAGCGTCGGCATATGAAGGGCGCGGGCGGCCAG |
| | | TACGGCCTCTGGGCTGAGACAGGACGAGGCAGGG | TACGGCCTCTGGGcTGAGACAGGACGAGGCAGGG |
| | | TGAGAAAGAGGAGGATGGGGGGGACCGGGGTGGT | TGAGAAAGAGGAGGATGGGGGGGACCGGGGTGGT |
| | | GGTGCTGCTGCTGTTGTGGGTGCGGACGGTGCGG | GGTGCTGCTGCTGTTGTGGGTGcGGACGGTGCGG |
| | | GTGCCGGGACAGCGTGCCGGCGAACGTTCTGTAA | gTGCCGGGACAaCGTGCCGGCGAACGTTCTGTAA |
| | | TCTTCCAT | TCTTCCAT |
| UL79 | 119/120 | ACCTAACGTGATTTATCTGCCACTTTTCTCCCCG | ACCTaACGTgATTTATCTGCCACTTTTCTCCCCG |
| | | CTGCCGTACAGCGCCGCCGCTCATAATGCCGTCA | CTGcCGTACAgCGCCGCCGCTCATAATGCCGTcA |
| | | CCGTCGCGTCGGACGCGACGGTGTTTTCGCCGTC | CCGTCGCgTCgGaCGCGACGGTGTTTTCGCCGTC |
| | | GATGCAGAGGACGGAGGAACTTTCGGCCGAAACA | GaTGCAGAGGACGGAGGAACTtTCGGCCGAAACa |
| | | TCGATCGTAGTCCCAGGACACATTTCGGAAGCCA | TCGATCGTAGTCCCAGGACACATTTCGGAAGCCA |
| | | TGCCTTCCGCGTGCTTCACCAACGTGGCTTTCTC | TgCCTTCCGCGTGCTtcACCAACGTGGCTTTCTC |
| | | CGACGTGGTTGTCGTTACCACAACGGCCGCCGAC | cGACGTGGttGTCGTTACCACAACgGcCGCCGAc |
| | | GTCGCGTCGGCGTAACAACGGCTGGAGGACTTTT | GTCGCGTCgGCGTAACAACGGCTGGAGGACTTTT |
| | | TCACCGCCTCGGCGACGTCTCGAACGGACGTAGA | TCACCGCcTCGGCGACGTCTCGaACGGACGTAGA |
| | | AAAGTAACACACGGCCAGCTCCACGCTATACATA | AAAGTAACACaCGGCCAGCTCCACGCTATACATA |
| | | GCCCGTTTCAACGCCTGCACCAACCGACGTACGA | GCCCGtTTCAACGCCTGCACCAACCGACGTACGA |
| | | AATGACCGTGGCAGCTTTGCTGACATCTCTCGAC | AATGACCGTGGCAGCTtTGcTGACATcTCTCGAC |
| | | CAGATAATCAAAGGAGTCATCCAGATCCTTGGTG | CAGATAATCAAAGGAGTCATCCAGATCCTTGGTG |
| | | GGCTCGCGGGAGAAGAACGCAATGATAAAGAGCG | GGCTCGCGGGAgAAGAACGCAATGATAAAGAGCG |
| | | GCAGAATGCCAAGACGCATGGTGA | GCAGAATGCCAAGACGCATGGTGA |
| UL80 | 121/122 | GAGAGACGCTATATTTAGGGCTTCCCTCTCTTTT | GAGAGACGCTATATTTAGGGcTTCCCTCTCTTTT |
| | | TTTTTTCTACACCGTGATACCCT | TTTTttCTACAcCgTGATACCCT |
| UL86 | 123/124 | GGCCGTCCGGTGAGGAGGACGGCGACGACCGCAG | GGCCGTCCGGTGAGGAGGACGGCGACGACCGCAG |
| | | GTTAGCGGCGAGTCACCTAGACGCAAACGCGGGC | GTTAaCGGCGAaTCACCTAGACGCAAACGCGGGC |
| | | CCGGACGCGCCACGCTCGCTCTGACGCCGCGCCC | CCGGACGCGCCACGCTCGCTCTGACGCCGCGCCC |
| | | GGTGCAGACGTTGTTCGTCTCTGCTTCTCCTCCG | GGTGCAGACGTTGTTCGTCTCtGCtTCTCCTCCG |
| | | TCGCGGCCAGGATTTCACCGCCGCTATGGCGGCC | TCGCGGCCAgGATTTCACCGCCGCTATGGCGGCC |
| | | ATGGAGGCCAACATCTTCTGCACTTTCGACCACA | ATGGAGGCCAACATCTTCTGcACTTTCGACCACA |
| | | AGCTCAGCATCGCCGACGTAGGCAAACTGACCAA | AGCTCAGCATCGCCGACGTAGGCAAACTGACCAA |
| | | GCTAGTAGCGGCCGTTGTGCCCATTCCGCAGCGT | GCTAGTAGCGGCcGTtGTGCCCATTCCGCAgCGT |
| | | CTACATCTCATCAAGCACTACCAGCTGGGCCTAC | CTACATCTCATCAAaCACTACCAGCTGGGCCTAC |
| | | ACCAGTTCGTAGATCACACCCGCGGCTACGTACG | ACCAGTTCGTAGATCACACCCGCGGCTACGTaCG |
| | | ACTGCGCGGCCTGCTGCGCAATATGACGCTGACG | ACTGCGCGGCCTGCTGCGCAATATGACGCTGACG |
| | | TTGATGCGGCGCGTAGAAGGCAACCAGATCCTCC | TTGATGCGGCGCGTAGAAGGCAACCAGATCCTCC |
| | | TACACGTACCGACGCACGGACTGCTCTACACCGT | TACACGTACCgACGCACGGACTGCTCTACACCGT |
| | | CCTCAACACGGGACCCGTGACTTGGGAGAAGGGC | CCTCAACACGGGACCCGTGACTTGGGAGAAGGGC |
| | | GACGCGCTATGCGTGCTGCCGCCG | GACGCGCTATGCGTGCTGCCGCCG |
| UL87 | 125/126 | TGGAAGCCGCGGCCGCTGCCGCCGCGGCGTTTCG | TGGAAGCCGCGGCCGCTGCCGCCGCGGCGTTTCG |
| | | TCCGGAGGAGCGTCCGACGCCGGGTTGGCACGAC | TCCGGAGGAGCGTCCGACGCCGGGTTGGCACGAC |
| | | GCGGCGTTGTTAATGGACGACGGTACGGTGCGCG | GCgGCGTTGTTAATGGACGACGGTACGGTGCGCG |
| | | AGCACGCGTTTCGCAACGGACCGCTGTCGCAACT | AGCACGCGTTTCGCAACGGACCGCTGTCGCAACT |
| | | GATTCGCCGTGTGTTACCGCCGCCGCCCGACGCC | GATTCGCCGTGTGTTACCGCCGCCGCCCGACGCC |
| | | GAAGACGACGTGGTTTTTGCTTCCGAGCTGTGTT | GAAGAcGACGTGGTTTTTGCtTCcGAgCTGTGTT |
| | | TTTAT | TTTAT |
| UL91 | 127/128 | GGCACGTCCAGAACGCGTTTACCGAGGAGATCCA | GGCACGTCCAGAACGCGTTTACCGAGGAGATCCA |
| | | GTTACACTCGCTCTACGCGTGCACGCGCTGCTTT | GTTACAtTCgCTCTACGCGTGCACGCGCTGCTTT |
| | | CGCACGCACCTGTGTGATCTGGGCAGCGGCTGCG | CGCACGCACCTGTGTGATCTGGGCAGCGGCTGCG |
| | | CGCTCGTCTCCACGCTCGAGGGCTCCGTCTGCGT | CGCTCGTCTCCACGCTCGAGGGCTCCGTCTGCGT |
| | | CAAGACGGGCCTGGTATACGAAGCTCTCTATCCG | CAAGACGGGCCTGGTATACGAggCTCTcTATCCG |
| | | GTGGCGCGTAGCCACCTGTTGGAACCCATCGAGG | GTGGCGCGTAGCCACCTGTTGGAACCcATgGAGG |
| | | AGGCCGCACTGGACGACGTCAACATCATCAGCGC | AGGcCtCACTGGACGACGTCAACATCATCAGCGC |
| | | CGTGCTCAGCGGCGTGTACAGCTACCTCATGACG | CGTGCTCAGCGGCGTGTACAGCTACCTCATGACG |
| | | CACGCCGGCCGTTACGCCGACGTGATCCAAGAGG | CAcGCaGGCCGTTACGCCGACGTGATCCAaGAGG |
| | | TGGTCGAGCGCGACCGCCTCAAAAAGCAGGTGGA | TGGTCGAGCGCGACCGCCTCAAAAAGCAGGTGGA |
| | | GGACAGTATTTACTTCACCTTTAATAAGGTTTTC | GGACAGTATTTACTTCACCTTTAATAAGGTTTTC |
| | | CGTTCTATGCATAACGTCAATCGTATTTCGGTGC | CGTTCTATGCATAACGTCAAcCGTATTTCGGTGC |
| | | CCGTCATCAGCCAACTTTTTAT | CCGTCATCAGCCAACTTTTTAT |
| UL92 | 129/130 | GGCGCGGTTCGCTGACGATGAGCAATTGCCTCTA | gGCGCGGTTCGCTGAcGATGAGCAATTGCCTCTA |
| | | CACCTGGTGCTCGACCAGGAGGTGCTGAGTAACG | CActTGGTGCTCGACCAGGAGGTGcTGAGTAACG |
| | | AGGAGGCCGAGACGCTGCGCTACGTCTACTATCG | AGGAGGCCGAGACGCTGCGCTACGTCTACTATCG |
| | | TAATGTAGACAGCGCTGGCCGATCCGCGGGCCGC | TAATGTAGACAGCGCTGGCCGATCCgCGGGCCGC |
| | | GTTCCGGGCGGAGATGAGGACGACGCACCGGCCT | GcTCCgGGcGGAGATGAGGACGACGCACCGGCCT |
| | | CCGACGACGCCGAGGACGCCGTGGGCGGCGATCG | CCGACGACGCCGAGgACGCCGTGGGCGGCGATCG |
| | | CGCTTTTGACCGCGAGCGGCGGACTTGGCAGCGG | CGCTTTTGAcCGCGAGCGGCGGACTTGGCAGCGg |
| | | GCCTGTTTTCGTGTACTACCGCGCCCACTGGAGT | GCCTGTTTTCGTGTAcTACCGCGCCCACTGGAGT |
| | | TGCTCGATTACCTACGTCAAAGCGGTCTCACTGT | TGCTcGATTACCTACGTCAAAGCGGTCTCACTGT |
| | | GACGTTAGAGAAAGAGCAGCGCGTGCGCATGTTC | GACGTTAGAGAAAGAGCAGCGCGTGCGCATGTTC |
| | | TATGCCGTCTTCACTACGTTGGGTCTGCGCTGCC | TATGCCGTCTTCACTACGTTgGGTCTGCGCTGCC |
| | | CCGATAATCGGCTCTCAGGCGCGCAGACGCTACA | CCGATAATCGGCTCTCAGGCGCGCAGACGCTACA |
| | | CCTGAGACTGGTCTGGCCCGACGGCAGCTATCGT | CCTGAGACTGGTCTGGCCCGACGGCAGCTATCGT |
| | | GACTGGGAGTTTTTAGCGCGTGACCTGTTACGAG | GACTGGGAgTTTTTAGCGCGTGACCTGTTACGAG |
| | | AAGAAATGGAAGCGAATAAGCGCG | AAGAAATGGAAGCGAAtAAGCGCG |
| UL95 | 131/132 | CGTCGGTCAACAAACAGCTCTTAAAGGACGTGAT | CGTCGGTCAACAAACAGCTCTTAAAGGACGTGAT |
| | | GCGCGTCGACCTTGAGCGACAGCAGCATCAGTTT | GCGCGTCGACCTTGAGCGACAGCAGCATCAGTTT |
| | | CTGCGGCGTACCTACGGACCGCAGCACCGGCTCA | CTGCGGCGTACCTACGGACCGCAGCACCGGCTCA |
| | | CCACGCAGCAGGCTTTGACGGTGATGCGTGTGGC | CCACGCAGCAGGCTTTGACGGTGATGCGTGTGGC |
| | | CGCTCGGGAACAGACCCGATACAGTCAGCGAACG | CGCTCGGGAACAGACCCGATACAGTCAGCGAACG |
| | | ACGCAGTGCGTGGCCGCACACCTGTTGGAGCAAC | ACGCAGTGCGTGGCCGCACACCTGTTGGAGCAAC |
| | | GGGCGGCCGTGCAGCAAGAGTTGCAACGCGCCCG | GGGCGGCCGTGCAGCAAGAGTTGCAACGCGCCCG |
| | | ACAGCTGCAATCCGGTAACGTGGACGACGCGCTG | ACAGCTGCAATCCGGTAACGTGGACGACGCGCTG |
| | | GACTCTTTAACCGAGCTGAAGGACACGGTAGACG | GACTCTTTAACCGAGCTGAAGGACACGGTAGACG |
| | | ACGTGAGAGCCACCTTGGTGGACTCGGTTTCGGC | AcGTGAGAGCCACCTTGGTGGACTCGGTTTCGGc |
| | | GACGTGCGATTTGGACCTGGAGGTCGACGACGCC | GACGTGCGATTTGGACCTGGAGGTcGACGACGCC |
| | | GTCTAACAGGTATAGCAATCCCCGTCACGCCTCT | GTCTAACAGGTATAGCAATCcCCGTCACGCCTCT |
| | | GTTCAGATTTTAT | GTTCAgATTTTAT |
| UL97 | 133/134 | CCGGGACGCGGAACGTGACGGTTGCTGAGGGGAA | CCGGGACGCGGAACGTGACGGTTGCtGAGGGGAA |
| | | AGGCAACAGAGAAGGTACAAACCCACCGGCGGGG | AGGcaACAGAGAAGGTACAAACCCACCGGCGGGG |
| | | AAAATACCGAGGCGCCGCCATCATCATGTGGGGC | AAAATACcGAGGCGCCGCCATCATCATGTGGGGC |
| | | GTCTCGAGTTTGGACTACGACGACGATGAGGAGC | GTCTCGAGTTTGGACTACGACGACGATGAGGAGC |
| | | TCACCCGGCTGCTGGCGGTTTGGGACGATGAGCC | TCACCCGGCTGCTGGCGGTTTGGGACGATGAGCC |
| | | CCTCAGTCTCTTTCTCATGAACACCTTTTTGCTG | cCTCAGTCTcTTTCTcATGAACACCTTTTTGCTG |
| | | CACCAGGAGGGCTTCCGTAATCTGCCCTTTACGG | CACCAGGAGGGCTTCCGTAATCTGCCCTTTACGG |
| | | TGCTGCGTCTGTCTTACGCCTACCGCATCTTCGC | TGCTGCGTtTGTCTTACGCCTACCGCATCTTCGC |
| | | CAAGATGCTGCGGGCCCACGGTACGCCAGTAGCC | CAAGATGcTGCGGGCCCACGGTACGCCAGTAGCC |
| | | GAGGACTTTATGACGCGCGTGGCCGCGCTGGCTC | GAGGACTTTATGACGCGCGTGGCCGCGcTGGCTC |
| | | GCGACGAGGGTCTGCGCGACATTTTGGGTCAGCG | GCGACGAGGGTCTGCGCGACATTTTGGGTCAGCG |
| | | GCACGCCGCCGAAGCCTCACGCGCCGAGATCGCC | GCACGCCGCCGAAGCcTCgCGCGCCGAGATCGCC |
| | | GAGGCCCTGGAGCGCGTGGCCGAGCGGTGCGACG | GAGGCCCTGGAGCGCGTGGCCGAGCGGTGCGACG |
| | | ACCGGCACGGCGGCTCGGACGACTACGTGTGGCT | ACCGGCACGGCGGCTCGGACGACTACGTGTGGCT |
| | | CAGCCGGTTGCTGGATTTGGCGCC | tAGCCGGTTGCTGGATTTgGCGCC |
| UL98 | 135/136 | AAGATGCTCTGGGTCGCCAGGTGTCTCTACGCTC | AAGATGCTCTGGGTCGCCAGGTGTCTCTACGCTC |
| | | CTACGACAACATCCCTCCGACTTCCTCCTCGGAC | CTACGACAACATCCCTCCGACTTCCTCCTCGGAC |
| | | GAAGGGGAGGACGATGACGACGGGGAGGATGACG | GAAGGGGAGGACGATGACGACGGGGAGGATGACG |
| | | ATAACGAGGAGCGGCAACAGAAGCTGCGGCTCTG | ATAACGAGGAGCGGCAACAGAAGCTGCGGCTcTG |
| | | CGGTAGTGGCTGCGGGGGAAACGACAGTAGTAGC | CGGTAGTgGCTGCGGGGGAAACGACAgTAGTAGC |
| | | GGCAGCCACCGCGAGGCCACCCACGACGGCTCCA | GGCAGCCACCGCGAGGCCaCCCACGACgGCtCCA |
| | | AGAAAAACGCGGTGCGCTCGACGTTTCGCGAGGA | AGAAAAAcGCGGTGCGCTCGACGTTTCGCGAGGA |
| | | CAAGGCTCCGAAACCGAGCAAGCAGTCAAAAAAG | CAAGGCTCCGAAACCGAGCAAGCaGTCAAAAAAG |
| | | AAAAAGAAACCCTCAAAACATCACCACCATCAGC | AAAAAGAAACCCTCAAAACaTCACCACCATCAGC |
| | | AAAGCTCCATTATGCAGGAGACGGACGACCTAGA | AAAGCTCCATTATGCAGGAGACGGACGACcTAGA |
| | | CGAAGAGGACACCTCAATTTACCTGTCCCCGCCC | CGAAGAGGACACCTCAATTTACCTGTCCCCGCCC |
| | | CCGGTCCCCCCCGTCCAGGTGGTGGCTAAGCGAC | CCGGTCCCCCCCGTCCAGGTGGTGGCTAAGCGAC |
| | | TGCCGCGGCCCGACACACCCAGGACTCCGCGCCA | TGCCGCGGCCCGACACACCCAGGACTCCGCGCCA |
| | | AAAGAAGATTTCACAACGTCCACCCACCCCCGGG | AAAGAAGATTTCACAACGTCCAcCCACCCCCGGG |
| | | ACAAAAAAGCCCGCCGCCTCCTTG | ACAAAAAAGCCCGCCGCCtCCTTG |
| UL100 | 137/138 | CCCCGCCGCCACCCGCACCAGACTTGGAGACATG | CCCCGCCGCCACCCGCACCAGACTTGGAGACATG |
| | | GACATAAAAAAGAGACACGCAGACCGTGGGTCGG | GACATAAAAAAGAGACACGCAGACCGTGGGTCGG |
| | | GAGCACATACTTTTTTTTTAT | GAGCACATACTTTTTTTTTtAT |
| UL103 | 139/140 | GAAGCGAACTAGACACGCATATCATAGAAAAAAA | GAAGCGAACTAGACACGCATATCATAGaaaaaaa |
| | | AAAAACACGCAACACGTAGTGAGCTTTGACGTCC | aacacgcaacacgtagtgagctttgacgtccctt |
| | | CTTTTACTAGTATCCACGTCACACGCTGAGAACT | ttactagtatccacgtcacacgctgagaactttg |
| | | TTGACGCACTTTTTTTTTACTAGTATCCACGTCA | acgcacttttttttactagtatccacgtcactta |
| | | CTTACCCGCGTAGTTCCCCTACGTGACTCGTTAA | cccacgtagttctcctacgtgactcgttaagcgt |
| | | GCGTTGAGCCGGAAAAACCTCAGGCCCTCGGAAG | tgagccggaaaaaccgcaggccctcggaagccac |
| | | CCACCCGCTTAGCAGCGTGTTGCGCGTCAACCGC | ccgcttagcagcgtgttgcgcgtcaaccgccagc |
| | | CAGCGAACGCACCCACTCGTCGCGCTCCTCGAGC | gagcgcacccactcgtcgcgctcctcgagccaag |
| | | CAAGTCGCCGACGAAGAAGAACAAGACGGAGGAG | ttgccgacgaagaagaacaagacggaggagacac |
| | | ACACCGTCGCCGTGCCCGAAGAGGACGAAGTGAC | cgtcgccgtgcccgaagaggacgaagtgacggac |
| | | GGACGGCAAGGCGGAGGAGAGAACGGAAGAAGAA | ggcaaggcggaggagagaacggaagaagaagaac |
| | | CAAGCGGTGGTAGAAGCGGTGGAGGACGACAATA | aagtggtggtggaagcggtggaggacgacaataa |
| | | ACTCTCGCGCCCAGACCTCCACGCAAGCCGTGAG | ctctcgcgcccagacctccacgcaagccgtgagc |
| | | CATGGCAAAAGCCTTGTCCACATAGACGCCGTAG | atggcaaaggccttgtccacatagacgccgtagc |
| | | CCGATATCGGCCGCTAACGCCGTA | cgatatcggccgccaacgccgtat |
| UL105 | 141/142 | CACAACACCGTGTAAGGAAAACGTGACTTTAT | CACAACACCGTGTAAggAAAACGTGACTTTAT |
| UL107 | 143/144 | GGCATCCTCTCTGCCACACGCGCAGTCACGGATA | GGCATCCTCTCTGCCACACGCGCAGTCACGGATA |
| | | GGATCAGTGCGTATTCATTATAAAAAAAACACAA | GGATcAGTGCGTATTCATTATAAAAAAAAaCACA |
| | | ACAACCCATATATGTGAAGCAGAATGATGACCGA | AACAACCCATATATGTGAAGCAGAATGATGACCG |
| | | CCGCACGGAGCGACGCCGTCGACTGACCCACGCG | ACCgCACGGAGCGACGCCGTCGACTGACCCACGC |
| | | GGATGTACGCCGTCCGCGAACAACCAAAGGACGA | GGcATGTACGCCGTCCGCGAACaACCAAAGGACG |
| | | CCCGTCTCCCCCCGCATCCGGGTTTTTCTCTTGG | ACCCGTCTCCCCCCGCAcCCGGGTTTTTtCTCTT |
| | | TCGAACCCGGCTTGCGACGACGGGTTGTTGCTTT | GGTCGAACCCGGCTTGCGACGACGGGTtGTTcCT |
| | | ACCGGACGACGGTCAGCCGCGGGGTTGATACCCA | TTACCGGACGACGGTCAGCCGCGGGGTTGATACC |
| | | GCGACGGCGTCGCTCCCACCCGGGTTTCTTCTCT | CAGCGACGGCGTCGCTCCCACCCGGGTTTCTTCT |
| | | TGTAGGTACCACTCGTAGACTGTCAGCCTTACGA | CTTGcAGgTACCACcCGTcGACTGTCAGCCTcgC |
| | | GGAGACACCGCGGACCGGGGAAACGGATAAGTTT | GAgGAGACACCGCGGACCgGGGAAACGGATAAGT |
| | | ACGAACAGAAATCTCAAGAGAAAGATGCTGACCC | TTaCGAACAGAAATCtCAAAagAcGCTGACCCGa |
| | | GATAAGTACCGTCACGGAGACACGGTGGTTTTTA | tAAGTACcGTcACGgaGAcACGGTGGTTTTTAT |
| | | T | |
| UL112- | 145/146 | AAAACAGAGCCGAGACCGGAAAAATTATGAAACA | AAAACaGAGCCGAGACCGGAAAAAtTATGAAACA |
| 113 | | GGACGCGCTTGGACATTTGGGTTTCCACCCCTTT | GGACGCGCTTGGACATTTGGGTTTCCACCCCtTT |
| | | CGGTGTGTGTCTATATATATTGTGGTCACTGATT | cGGTGTGTGTCTATATATATTgtGGTcACTGATT |
| | | TTTTTTTAC | TTTTTTtac |
| UL117 | 147/148 | AGCGGCGGCGGCGATGGCGGGGCTGGTTGCTTTT | AGCGGCGGCGGCGATGGCGGGGCTGGTTGCTTTT |
| | | CCTGGCCCTGTGCTTTTGCTTACTGTGTGAAGCG | CCcGGCcCTGTGCTTTTGCTTACTGtGTGAAGCG |
| | | GTGGAAACCAACGCGACCACCGTTACCAGTACCA | GTGGAAACCAACgCGACCACCGTTACCaGTACCA |
| | | CCGCTGCCGCCGCCACGACAAACACTACCGTCGC | CCGCTGCCGCCGCCACGACAAACACTACCGTCGC |
| | | CACCACCGGTACCACTACTACCTCCCCTAACGTC | CACCACCGGTACCACTACTACCTCCCCtAACGTC |
| | | ACTTCAACCACGAGTAACACCGTCATCACTCCCA | ACTTCAACCACGagtAaCaCCgtcaccactccca |
| | | CCACGGTTTCCTCGGTCAGCAATCTGACATCCAG | ccacggtttcctcgGTCagcAATctgAcgTCCAg |
| | | CGCCACGTCGATTCCCATCTCAACGTCAACGGTT | CaCcaCgtCGAttcccatctcaaCGTCAACgGTT |
| | | TCTGGAACAAGAAACACAAGGAATAATAATACCA | TCTGgaaCAAgAAAcACAgGGAATAAtaaTACCA |
| | | CAACCATCGGTACGAACGTTACTTCCCCCTCCCC | CAACCaTCGGTACGAACGcTACTTCCCCCTCCCC |
| | | TTCTGTATCCATACTTACCACCGTGACACCGGCC | TTCTGTATCCATACTTACCACCGtGACACCGGCC |
| | | GCGACTTCTACCACCTCCAACAACGGGGATGTAA | GCaACTTCTACcAtCTCCgtcgACGGtGtcGTcA |
| | | CATCCGACTACACTCCAACTTTTGACCTGGAAAA | CggcgTCaGACTACACTCCgACTTTTgacGAtCT |
| | | CATTACCACCACCCGCGCTCCCACGCGTCCTCCC | GGAAAACATTACCACCACCCGCGCTCCCACGCGT |
| | | GCCCAGGACCTTTGTAGCCATAAC | CCTCCCGCCCAGGACCTgTGTAGC |
| UL120 | 149/150 | CGCGGCCCCCTGCCACATATAGCTCGTCCACACG | CGCGgCCcCctGCCACATATAGCTCGTCCACaCg |
| | | CCGTCTCGTCACACAGCAACATGTGTCCCGTGCT | CCGTCTcGTCACACaGCAACATGTGTcCCGtgCT |
| | | GGCGATCGTACTCGTGGTTGCGCTCTTGGGCGAC | GGCGATcGtaCTCgtgGttgCGCTcTTggGcgAC |
| | | ACGCACCCGGGAGTGGAAAGTAGCACCACAAGCG | AcGCACCCGgGagTGgaAAGTAGCACcACAAGcG |
| | | CCGTCACGTCCCCTAGTAATACCACCGCCACATC | CCGTcACgTCCCCtagTAATAcCACCGcCACaTc |
| | | CACTACGTCAATAAGTACCTCTAACAACGTCACT | cACTACGTCaATAAgTACCtCtAAcAACGTCACT |
| | | TCTGCTGTCACCACCACGGTACAAACCTCTACCT | TCtgCtGTCAcCACCACGGTACAAACCTCTAccT |
| | | CGTCCGCCTCCACCTCCGTGATAGCCACGACGCA | cgTCCGCCtCcACcTCCGTGatAgCCACGACGCA |
| | | GAAAGAGGGGCGCCTGTATACTGTGAATTGCGAA | GAAAGAGGGGCgCCTGTATAcTGTGAATTGCGAA |
| | | GCCAGCTACAGCTACGACCAAGTGTCTCTAAACG | GCCAGCTACAGCtACGACCAaGTGTCTCTaAACG |
| | | CCACCTGCAAAGTTATCCTGTTGAATAACACCAA | CCACCTGcAAAGTtatCCTGTTGAAtAAcACCaa |
| | | AAATCCAGACATTTTATCAGTTACTTGTTATGCA | AAATCCaGACATTTTaTCagTTACtTGTTATGCA |
| | | CGGACAGACTGCAAGGGTCCCTTCACTCAGGTGG | CGGACagACTGCAAgGGTCCcTTCACTCAGGTGG |
| | | GGTATCTTAGCGCTTTCCCCCCCGATAATGAAGG | GGTATCTTAGCGCtTTccCccCCgataAtgAAGG |
| | | TAAGTAGCACCTACCTTTCTGTTC | TAAgtagcacctacctttctgttc |
| UL137 | 151/152 | TGTTACCCCGCCAGCACCTCCGCCGGCAACCGCG | tgttaccccgccagcacctccgccggcaaccgcg |
| | | TCGTCGTTGCTATCGTCGCCGGTTTCGGGCGATG | tcgtcgttgctatcgtcgccggtttcgggcgatg |
| | | ACAGCGCCGGCGGCGCGGGTCTCGTCTCGTCCAC | acagcgccggcggcgcgggtctcgtctcgtccac |
| | | CATTTCCACCGTGTCGAAGCGACAGCCGCTGCCG | catttccaccgtgtcgaagcgacagccgctgccg |
| | | TAGTACATGGCCCCGTTCAACGGCCGGCGGGCCG | tagtacatagctccgttcaacggccggcgggccg |
| | | GGTCGCCGAGTTCCGGGTCGGGCACATCCATGGC | ggtcgccgagttccgggtcgggcacatccatggc |
| | | TCGCCGTCTGCTTCTCTGCCGCTCGTGGTGCCGA | ttgccgtctccttctctgccgctcgtggtgccga |
| | | CGGCACTTCTCAGGATAATGACAGCCGCAAAATA | cggcacttctcgggataatgacagccgcaaaata |
| | | GATCGTGGAGCATGTCTCGCCAACTGTCCTGGTG | gatcgtggagcatgtctcgccaactgtcctggtg |
| | | GTAATATCTTAAGTACGCGATGAGCGCGCCGATG | gtaatatcttaagtacgcgatgagcgcgccgatg |
| | | GCCATAATCATAAGCGTAAGCAAAACGGCACAGA | gccataatcataagcgtaagcaaaacggcacaga |
| | | TAACGTGAAACACCGCGGTCATCCAAGTCGGGCG | taacgtgaaacaccgcggtcatccaagtcgggcg |
| | | GCGTCGGGGACGCGGTGGGTCGGTTTCTCTTACG | gcgtcggggacgcggtgggtcggtttctcttacg |
| | | CCGGCGTCACTCAGCCACCACACCCGTAGTCGAC | ccggcgtcactcagccaccacacccgtagccgac |
| | | ATTCCCAGAACCGGTGAATGCGAC | attcccagaaccggtgaatgcgac |
| UL141a | 153/154 | GCTGCCCGCGACTCCTCGAATATTCTTCCTCTTC | gctgcccgcgactcctcgaatattcttcctcttc |
| | | GTTCCCCTTCGCCACCGCTGACATTGCCGAAAAG | gttccccttcgccaccgctgacattgccgaaaag |
| | | ATGTGGGCCGAGAATTATGAGACCACGTCGCCGG | atgtgggccgagaattatgagaccacgtcgccgg |
| | | CGCCGGTGTTGGTCGCCGAGGGAGAGCAAGTTAC | cgccggtgttggtcgccgagggagagcaagttac |
| | | CATCCCCTGCACGGTCATGACACACTCCTGGCCC | catcccctgcacggtcatgacacactcctggccc |
| | | ATGGTCTCCATTCGCGCACGTTTCTGTCGTTCCC | atggtctccattcgcgcacgtttctgtcgttccc |
| | | ACGACGGCAGCGACGAGCTCATCCTGGACGCCGT | acgacggcagcgacgagctcatcctggacgccgt |
| | | CAAAGGCCATCGGCTGATGAACGGACTCCAGTAC | caaaggccatcggctgatgaacggactccagtac |
| | | CGCCTGCCGTACGCCACTTGGAATTTCTCGCAAT | cgcctgccgtacgccacttggaatttctcgcaat |
| | | TGCATCTCGGCCAAATATTCTCGCTGACTTTCAA | tgcatctcggccaaatattctcgctgactttcaa |
| | | CGTATCGACGGACACGGCCGGCATGTACGAATGC | cgtatcgacggacacggccggcatgtacgaatgc |
| | | GTGCTGCGCAACTACAGCCACGGCCTCATCATGC | gtgctgcgcaactacagccacggcctcatcatgc |
| | | AACGCTTCGTAATTCTCACGCAACTGGAGACGCT | aacgcttcgtaattctcacgcaactggagacgct |
| | | CAGCCGGCCCGACGAACCTTGCTGCACGCCGGCG | cagccggcccgacgaaccttgctgcacgccggcg |
| | | TTAGGTCGTTACTCGCTGGGAGAC | ttaggtcgttactcgctgggagac |
| UL151 | 155/156 | AGAAGGGGAGGACGACGTTCTCGCCACAATCCGC | ctggaacgtcgtacgctgccgcggcacaggcttt |
| | | AACACGTTGTCCGCCCCAACCTCACCTGCTGCGG | cgcgcacacgattccgaggacggcgtctctgtct |
| | | CTACCACGCATCGACTGTCGTTCCCTGGAGAATC | cgcgtcagcacttggtttttttactcggaggcca |
| | | GACCTTCTGCCTCACCGCTGTTTCCGAGTGCTCA | cggccgccgtgtacagttagaacgtccatccgcg |
| | | CAACGTCGAACATCAACGGCTGCATTAACGCCGC | ggagaagcccaagctcgaggcctattgccacgca |
| | | CGCCGCCAGCGGTAGCTGCTGCGTTCTCTTTTTC | tccggatcacccccatctccacatctccacgccc |
| | | GTCCACGGTCTCCGAGACCGGCACTTTTCCGCAG | aaaaccaccccagcccaccatatccaccgcatcg |
| | | AGCACAACAGGCCGCACACGTGTCGACGACACCG | cacccacatgctacgactcgcccacatcacacgc |
| | | CCGTCGTTACCGCCGGAGACCCGCGCTCTCCTGT | tctttcctatcccttctacaccctcagccacggt |
| | | GACACACGTAACTCTCCTCCAGATATTCCGTCTG | tcacaatccccgaaactacgccgtccaacttcac |
| | | CGTAGCTCGCTGCTGACGAGCAGGTCCGGCGGCG | gccgaaacgacccgcacatggcgctgggcacgac |
| | | CTCTCCGCGGAGGTGAGCACGAGGCCATCCCCAA | gcggtgaacgtggcgcgtggatgccggccgagac |
| | | AGTCGCGTCGCTGTTCTGGACGCTGCTCAAAGCA | atttacatgtcccaaggataaacgtccctggtag |
| | | ACACAGATAGTTGACATGACTCACAAAACACCGA | acggggtagggggatctaccagcccagggatcgc |
| | | GTGCCGACTCTCACCGCAACCCAC | gtatttcgccgccacgctgcttca |
| UL151a | 157/158 | ACGCCGTGCACCACAAACTCTGCGGCGCGATGAT | acgccgtgcaccacaaactctgcggcgcgatgat |
| | | ATCTTCGTCGTGTTCCACCACTTGCACACCGCTG | atcttcgtcgtgttccaccacttgcacaccgctg |
| | | ATTATGGACTTGCCGTCGCTGTCCGTGGAACTAT | attatggacttgccgtcgctgtccgtggaactat |
| | | CTGCAGGACACAAGAAAAAAGAAACACCAACCGA | ctgcaggacacaagaaaaaagaaacaccaaccga |
| | | GGGTGGGTGGGGCGGTGAAGAAGGGGAGGACGAC | gggtgggtggggcggtgaagaaggggaggacgac |
| | | GTTCTCGCCACAATCCGCAACACGTTGTCCGCCC | gttctcgccacaatccgcaacacgttgtccgccc |
| | | CAACCTCACCTGCTGCGGCTACCACGCATCGACT | caacctcacctgctgcggctaccacgcatcgact |
| | | GTCGTTCCCTGGAGAATCGACCTTCTGCCTCACC | gtcgttccctggagaatcgaccttctgcctcacc |
| | | GCTGTTTCCGAGTGCTCACAACGTCGAACATCAA | gctgtttccgagtgctcacaacgtcgaacatcaa |
| | | CGGCTGCATTAACGCCGCCGCCGCCAGCGGTAGC | cggctgcattaacgccgccgccgccagcggtagc |
| | | TGCTGCGTTCTCTTTTTCGTCCACGGTCTCCGAG | tgctgcgttctctttttcgtccacggtctccgag |
| | | ACCGGCACTTTTCCGCAGAGCACAACAGGCCGCA | accggcacttttccgcagagcacaacaggccgca |
| | | CACGTGTCGACGACACCGCCGTCGTTACCGCCGG | cacgtgtcgacgacaccgccgtcgttaccgccgg |
| | | AGACCCGCGCTCTCCTGTGACACACGTAACTCTC | agacccgcgctctcctgtgacacacgtaactctc |
| | | CTCCAGATATTCCGTCTGCGTAGC | ctccagatattccgtctgcgtagc |
| UL153 | 159/160 | CATTCCCCTGGGAATTCATGCTGTATGGGCGGGT | cattcccctgggaattcatgctgtatgggcgggt |
| | | ATAGTGGTATCTGTGGCACTTATAGCCTTATACA | atagtggtatctgtggcacttatagccttataca |
| | | TGGGTAGCCGTCGCGTCCCCAGAAGACCGCGTTA | tgggtagccgtcgcgtccccagaagaccgcgtta |
| | | TACAAAACTTCCCAAATACGACCCAGATGAATTT | tacaaaacttcccaaatacgacccagatgaattt |
| | | TAGACTAAAACCTAACATGCACATC | tagactaaaacctaacatgcacatc |
| US7 | 161/162 | TAAACTGTTAGGTTCGTTATAAGCGTGGATGGTC | taaactgttaggcttgttataagcgtggatgatc |
| | | ATATATAAACCGTATGCACAAAAGGTATGTGTGA | atatataaaccgtatgcacaaaaggtatgtgtga |
| | | ATGGAAATACATGATGAATGTCATCATCACGCAA | atggaaatacatgatgaatgtcatcgtcacgcaa |
| | | AGCAGCCGTGGGAATGGTAAAGACATCGTCACAC | agcagccgtgggaatggtaaagacatcgtcacac |
| | | CTATCATAAAGAATGCAACGCTTTCAGGATAGGT | ctatcataaagaatgcaacgctttcaggataggt |
| | | GTGGCGAAAGCCTCCTCCGTTCCGTATTCTATCG | gtggcgaaagcctcctccgttccgtattctatcg |
| | | TAACAAATATATGGAGTTTGTGTAATGCGTACTT | taacaaatatatagagtttatgtaatgcgtactt |
| | | CATGCCCCGATGAACGCTCTCGTCAGGCTTGTCA | catgccccgatgaacgctctcgtcaggcttgtca |
| | | TGGTCTGTAAAAGCTGCATGAAAAACACGACGAA | tggtccgtaaaagttgcatgaaaaacacgacgaa |
| | | AGCGTTCAGTGTTGGATCAGACTCCCACGTTAAT | agcgttcagtgttggatcagactcacgtcacacg |
| | | TAAGGGCGGCCGGATCCATGTTTAAACAGGCGCG | ttacatcatacaacgtagggcggtattgttgaga |
| | | CCTAGCTTC | acatatataatcgccgtttcgtaagtacgtcgat |
| | | | atcgctccttcttcactatggacctcttgatccg |
| | | | tctcggttttctgttgatgtgtgcgttgccgacc |
| | | | cccggtgagcggtcttcgcgtgac |
| US10 | 163/164 | AATGATTTGTTATGATGTCATTGTTGTTTACTGA | aatgatttgttatgatgtcattgttgtttactga |
| | | AAAGGAATGTGCTTTCCCGGCATGGGCCCGATTC | aaaggaatgtgctttcccggcatgggcccgattc |
| | | CGAGAAATGGTATGATGAATCATGTGGTCAGGCG | cgagaaatggtatgatgaatcatgtggtcaggcg |
| | | CTGCTCTCAACGTCCATATAAACGTGGGTTTCGG | ctgctctcaacgtccatataaacgtgggtttcgg |
| | | TGACCACAACCACGTCGGGGCTGACGCGGATCGG | tgaccacaaccacgtcggggctgacgcggatcgg |
| | | ACATCATACTGACGTGAGGCGCTCCGTCACCTCT | acatcatactgacgtgaggcgctccgtcacctct |
| | | CGGGCCGAACCCCGTCAGCACCCCGCGTCACTTA | cgggccgaaccccgtcagcaccccgcgtcactta |
| | | CAAATCACGTTCGTCGTGACGGGGGTTTCCCCTG | caaatcacgttcgtcgtgacgggggtttcccctg |
| | | ACACGTAATACTCGCGTCACGTCGGGACGATATA | acacgtaatactcgcgtcacgtcgggacgatata |
| | | AAGAGGCACGGTGTTTCGGCTCCCGCACACAGAC | aagaggcacggtgtttcggctcccgcacacagac |
| | | GACGCGCCGGGCGGCTTCCTGCGGCCGGCCGCGG | gacgcgccgggcggcttcctgcggccggccgcgg |
| | | TGCCGGCGGCTATGATCCTGTGGTCCCCGTCCAC | tgccggcggctatgatcctgtggtccccgtccac |
| | | CTGTTCCTTCTTCTGGCACTGGTGTCTGATCGCA | ctgttccttcttctggcactggtgtctgatcgca |
| | | GTAAGTGTACTCTCGAGCCGCTCCAAGGAGTCGC | gtaagtgtactctcgagccgctccaaggagtcgc |
| | | TCCGGTTGTCGTGGTCCAGCGACG | tccggttgtcgtggtccagcgacg |
| US12 | 165/166 | AAAAAAAACGTTTCTATCACCTAATCTGTCGTAC | aaaaaaaacgtttctatcacctaatctgtcgtac |
| | | TGTCCTTTGTCCCCCGCACCCTAAAACACCGTGT | tgtcctttgtcccccgcaccctaaaacaccgtgt |
| | | TCTCCCGACGTCACTAGATCACCACCCTGTTCCC | tctcccgacgtcactagatcaccaccctgttccc |
| | | CATGACGTGCAAGACTACATGCTATAAGACAGCC | catgacgtgcaagactacatgctataagacagcc |
| | | TTACAGCTTTTGAGTCTAGACAGGGGAACAGCCT | ttacagCttTtGagtctagaCaggggaaCagcCt |
| | | TCCCTTGTAAGACAGAATGAATCTTGTAATGCTT | tcccTtGtaAgacagAatgaatCttgtaatGCtt |
| | | ATTCTAGCCCTCTGGGCCCCGGTCGCGGGTAGTA | aTtctagccctctGGGccccgGtcgcggGtaGta |
| | | TGCCTGAATTATCCTTGACTCTTTTCGATGAACC | tgcCtgaattatccttgactcttttcGatgaaCc |
| | | TCCGCCCTTGGTGGAGACGGAGCCGTTACCGCCT | tccgcccttggTGgagaCggaGccGttacCgcct |
| | | CTGCCCGATGTTTCGGAGTACCGAGTAGAGTATT | ctgccCGatGtttcGgagtaccgagtAgagtatt |
| | | CCGAGGCGCGCTGCGTGCTCCGATCGGGCGGTCG | ccgagGCgcgcTgcgtgctcCGatcggGcggtcg |
| | | ATTGGAGGCTCTGTGGACCCTGCGCGGGAACCTG | AttggagGctcTgtggaCcctgcGcgggaacctG |
| | | TCCGTGCCCACGCCGACACCCCGGGTGTACTACC | TccGtgcccaCgccgacaccccGggtgtaCTacc |
| | | AGACGCTGGAGGGCTACGCGGATCGAGTGCCGAC | aGacgctGgagggctacgcGgaTcGagtGCCgac |
| | | GCCGGTGGAGGACGTCTCCGAAAG | GccggtggaGgAcgtctccGaAaG |
| US14 | 167/168 | GCTCCGCTGGTTTATAAGAAGACTCCACCGAGAC | GctCCGCTGGTTTATAAGAAGACTCCACCGAGAC |
| | | GCTCACCCGTTCACTCGGGCGCATCACCCGCCTC | GCTCACCCGTTCACTCGGGcGCATCACCCGCCTC |
| | | ATGGACTCGCCGCTACCGTCGCTACATTCGCCGC | ATGGACtCGCCGCTaCCGTCGCTACATTCGCCGC |
| | | AATGGGCTTCCCTCCTGCAGCTGCACCACGGCCT | AATGGGCTTCcCTCCTGCAGCTGCACCACGGCCT |
| | | TATGTGGCTGCGCCGTTTTGCTGTCCTCGTCCGG | TATGTGGCTGCGCCGTTTTGCTGTCCTCGTCCGG |
| | | GTCTACGCCCTAGTGGTCTTTCACATCGCCATCA | GTCTACGCCCTAGTGGTCTTTCACATCGCCATCA |
| | | GTACGGCTTTCTGCGGAATGATTTGGCTGGGTAT | GTACGGCTTTCTGCGGAATGATTTGGCTGGGtAT |
| | | CCCCGATTCCCACAACATATGTCAACATGAATCT | CCCCGATTCCCACAACATATGTCAACATGAATCT |
| | | TCCCCTCTGCTGCTGGTTTTTGCCCCCTCCCTTC | TCCCCTCTGCTGCTGGTTTTTGCCCCCTCCCTTC |
| | | TCTGGTGTTTGGTCTTGATACAGGGCGAAAGGCA | TCTGGTGTTTGGTCTTGATACAGGGCGAAAGGCA |
| | | CCCCGACGACGTGGTATTGACCATGGGCTACGTA | CCCCGACGACGTGGTATTGACCATGGGCTACGTA |
| | | GGCCTCCTCTCCGTTACCACGGTTTTCTACACCT | GGCCTCCTCTCCGTTACCACGGTTTTCTACACCT |
| | | GGTGCTCCGACCTGCCCGCCATCCTCATCGACTA | GGTGCTCCGACCTGCCCGCCATCCTCATCGACTA |
| | | CACACTGGTCCTCACGCTGTGGATAGCTTGCACC | CACACTGGTCCTCACGCTGTGGATAGCTTGCACC |
| | | GGCGCTGTCATGGTTGGGGACAGC | GGCGCTGTCATGGTTGGGGACAGc |
| US24 | 169/170 | GCGTCGAGCGGAGGACGCGG | gCGTCGAGCGGAGgACGCgG |
| US26 | 171/172 | AAACAACGTCAACAGTTTACGAGTACAAAACAGG | AAACAACaTCAACAGTTTACGAGTACAAAACAGG |
| | | AAAGGAACACA | AAAGGAAtACA |
| US27 | 173/174 | TTCGATCCTCTCTCACGCGTCCGCCGCACATCTA | TTCGATCCTCTCTCACGCGTCCGCCGCACATCTA |
| | | TTTTTGCTAATTGCACGTTTCTTCGTGGTCACGT | TTTTTGCTAATTGCACGTTTCTTCGTGGTCACGT |
| | | CGGCTCGAAGAGGTTGGTGTGAAAACGTCATCTC | CGGCTCGAAGAGGTTGGTGTGAAAACGTCATCTC |
| | | GCCGACGTGGTGAACCGCTCATATAGACCAAACC | GCCGACGTGGTGAACCGCTCATATAGACCAAACC |
| | | GGACGCTGCCTCAGTCTCTCGGTGCGTGGACCAG | GGACGCTGCCTCAGTCTCTCGGTGCGTGGACCAG |
| | | ACGGCGTCCATGCACCGAGGGCAGAACTGGTGCT | ACGGCGTCCATGCACCGAGGGCAGAACTGGTGCT |
| | | ATCATGACACCGACGACGACGACCGCGGAACTCA | AtCATGACaCCGACGACGACGACCGCGGAACTCA |
| | | CGACGGAGTTTGACTACGATGAAGACGCGACTCC | CGACGGAGTTTGACTACGATGAAGaCGCGACTCC |
| | | TTGTGTTTTCACCGACGTGCTTAATCAGTCAAAG | TTGTGTTTTcACCGACGTGCTTAATCAgTCAAAG |
| | | CCAGTTACGTTGTTTCTGTACGGCGTTGTCTTTC | CCaGTtACGTTGTTTCTGTACGGCGTTGTCTTTc |
| | | TCTTCGGTTCCATCGGCAACTTCTTGGTGATCTT | TcTTCGGTTCCATCGGCAACTTcTTGGTGATCTT |
| | | CACCATCACCTGGCGACGTCGGATTCAATGCTCC | CACCATCACCTGGCGACGTCGGATTCAATGCTCC |
| | | GGCGATGTTTACTTTATCAACCTCGCGGCCGCCG | GGCGATGTTTACTTTATCAACCTCGCGGCCGCCG |
| | | ATTTGCTTTTCGTTTGTACACTACCTCTGTGGAT | ATTTGCTTTTCGTTTGTACACTACCTCTGTGGAT |
| | | GCAATACCTCCTAGATCACAACTC | GCAATACCTCCTAGATCACAACTC |
| US28 | 175/176 | TAAAAAAGCGCTACCTCGGCCTTTTCATACAAAC | TAAAAAAGCGCTACCTCGGtCTTTTCgTACAAAC |
| | | CCCGTGTCCGCCCCTCTTTTCCCCGTGCCCGATA | CCCGTGTCCGCCCCTcTTTTCCCCGTgCCCGATA |
| | | TACACGATATTAAACCCACGACCATTTCCGTGCG | TACACGATATTAAACCCACGACCATTTCCGTgCG |
| | | ATTAGCGAACCGGAAAAGTTTATGGGGAAAAAGA | ATTAGCGAACCGGAAAAGTTTATGGGGAAAAAGA |
| | | CGTAGGAAAGGATCATGTAGAAAAACATGCGGTG | CGTAGGAAAGGATCATGTAGAAAAACATGCGGTG |
| | | TTTCCAATGGTGGCTCTACAGTGGGTGGTGGTGG | TTTCCgATGGTGGCTCTACAGTGGGTGGTGGTGG |
| | | CTCACGTTTGGATGTGCTCGGACCGTGACGGTGG | CTCACGTTTGGATGTGCTCGGACCGTGACGGTGG |
| | | GTTTCGTCGCGCCCACGGTCCGGGCACAATCAAC | GTTTCGTCGCGCCCACGGTCCGGGCACAATCAAC |
| | | CGTGGTCCGCTCTGAGCCGGCTCCGCCGTCGGAA | CGTGGTCCGCTCTGAGCCGGCTCCGCCGTCGaAA |
| | | ACCCGACGAGACAACAATGACACGTCTTACTTCA | ACCCGACGAGACAACAATGACACGTCTTACTTCA |
| | | GCAGCACCTCTTTCCATTCTTCCGTGTCCCCTGC | GCaGCACCTCTTTCCATTCTTCCGTGTCCCCTGC |
| | | CACCTCAGTGGACCGTCAATTTCGACGGACCACG | CACCTCAGTGGACCGTCAATTTCGACGGCCCACG |
| | | TACGACCGTTGGGACGGTCGACGTTGGCTGCGTA | TACGACCGTTGGGACGGTCGACGTTGGCTGCGcA |
| | | CCCGCTACGGGAACGCCAGCGCCTGCGTGACGGG | CCCGCTACGGGAACGCCAGCGCCTGCGTGACGGG |
| | | CACCCAATGGAGCACCAACTTTTT | CACCCaATGGAGCACCAACTTTTt |
| New | 177/178 | AAAATGATAATGATGATAATAACGATTACGACCG | AAAATGATAATGATGATAATAACGATTACGaCCG |
| ORF1 | | CTAAAACCCAGAGGGCGTGTGTAGCCACGTGTTG | CTAAAACCCAGAGGGCGTGTGTaGCCACGTGTTG |
| | | GTGCTGTGGGCTTGGTTGTAACGGTGTTTCCGCT | GTgCTGTGGGCTTGGTTGTAACGGTGTTTCCGCT |
| | | GCTGTGGCTTCAAAACCAACGTGATGTTCTACGT | gCTGTGGCTtCaAAACCaACGTGAtGTTCTACGT |
| | | GACTGTTAGGGGTGGTGGATTTTTTGGGACTGGA | GacTgTTAGGGGTGgTGGATTtTTTGGGAcTGGa |
| | | GTGTTTATGATGGGTAGTGCTTATCGTCGTCTTC | GTGTttATGATGGGTAGTGCTTaTCGTCGTCTTC |
| | | TTGGCGGTGGTGGTTGTTCTCGTGGTGGTTGTTT | TTGGcGGtgGtGGTtGTtCTCGTGGTGGTTGTTt |
| | | TTTGTGTTGTGGTAGTTGTCGTTCTCGTAGTCGT | TtTgTGTTGTgGTAGTTGTCGTTCTCGtaGTCGT |
| | | AGTGGGCTTTTTGGTGGTGGTAGTGGGGAATGTA | AGTGGGcTTTTTGGTGGTGGTAGTGGGgAaTGTa |
| | | CCGTTTTCGTTCACTGTCAGATTGTAACATGTGT | CCGTTTTcGtTcACtgtcAgATtgTAACATGTGT |
| | | CTAAAGTCCATCGAAAACCATGGTTATGTTGTTG | CTAAAGTCCATcgaaaaCCaTGGtTaTGttgtTg |
| | | GTGACGCCAATCGTCTAGCGATGTCATAGTACGA | gTGacgCcaATcgtCtAgcGatGTCATaGTaCGA |
| | | TAGGTAGTACTATACTGCGCGGTAACGTTAATGA | TAGgtagtacTatactgcgcggtaacgttaatga |
| | | GGAGGAGGCTGTAATTACTCAGACATGAAAAATT | ggaggaggctgtaattactcagacatgaaaaatt |
| | | AAAGCGCGTGCTGTTAAACGTTGT | aaagcgcgtgctgttaaacgttgt |
| New | 179/180 | TTTTCTCCCCCATCCGACAAAACCGTGTCCCTTA | AACACCGTTtGACtGCACCCCAACCGGCGCCATC |
| ORF3 | | AAATTCCCCACCTTTCTCTGTTCAAATGGCCCCG | TTGGTGACCttcTCGACGGTTCTCTCGCTCGTCA |
| | | AAACTGTAAAACACCGTTTGACCGCACCCCAACC | TGCCGTTCTGAGCTCCGACATGGCGGACGAGAGA |
| | | GGCGCCATCTTGGTGACCTCGACGGTTCTCTCGC | AAATGGtGTCGAGAGCcgAGGAGCGTTTTcGCTC |
| | | TCGTCATGCCGTTCTGAGCTCCGACATGGCGGAC | CAGGCGGGTAAAAaAATAGCACGATAACTTTTCT |
| | | GAGAGAAAATGGCGTCGAGAGCCTAGGAGCGTTT | GTGCTTTTTTGAGACGTTTTtGAAGAGCTTTTTT |
| | | TCGCTCCAGGCGGGTAAAAAAATAGCACGATAAC | tCTGCTCAGAGCGAAAAAATGATAGCCCTGAAAA |
| | | TTTTCTGTGCTTTTTTTGAGACGTTTTAGAAGAG | TCTCGACGAGTCTGGCCGAGCGGCGCCATCTTGG |
| | | CTTTTTTCTGCTCAGAGCGAAAAAATGATAGCCC | AGGAGGGGCGAGTCGCGGGCACCgCCTCGGTACC |
| | | TGAAAATCTCGACGAGTCTGGCCGAGCGGCGCCA | CCCcTGGCcGAGGCGAGTCCGCGgTCGCCGCCTG |
| | | TCTTGGAGGAGGGGCGAGTCGCGGGCACCGCCTC | TTCCGTGATGCTACCTAGAGGGCgccgtcgaggc |
| | | GGTACCCCCTGGCTGAGGCGAGTCCGCGGTCGCC | gactcttcctgttttcgccctgagggctaacggt |
| | | GCCTGTTCCGTGATGCTACCTAGAGGGCGCTGTC | cgctgacgtcaaaccatctcgtgctcgctgagtc |
| | | GAGGCGACTCTTCCTGTTTTCGCCCTGAGGGCTA | acatccggttgttgacaagcgatggaggaccgca |
| | | ACGGTCGCTGACGTCAAACCATCT | cccaaagtgcgccctctagtcatc |

| | | |
|---|---|---|
| | SID | |
| 3′UTR | NO | Representative sequence |

Kaposi's sarcoma-associated herpesvirus

| | | |
|---|---|---|
| ORF6 | 181 | TTGTGTACCCGTAACGATGGCAAAGGAACTGGCGGCGGTCTATGCCGATGTGTCAGCCCTAGCCATGGACCT |
| (HHV8 | | CTGTCTTCTTAGTTACGCAGACCCGGCAACACTGGACACTAAAAGTCTGGCCCTCACTACAGGGAAGTTTCA |
| gp03) | | GAGCCTTCACGGCACACTACTCCCCCTCCTCAGACGACAAAACGCACACGAATGCTCAGGTCTGTCACTAGA |
| | | ATTGGAGCACTTTTGGAAAACGTGGCTGATGCTCTGGCCACGTTGGGAGTGTGCACTAGCAGAAAACTGTCT |
| | | CCAGAAGAGCATTTTTCCCTCCTGCATTTGGACACAACATGCAACAAGCAACCGGAGCGTTAGGTTTAATTT |
| | | TTACGGAAATTGGGCCTTGGAGTTAAAGCTGTCACT |
| ORF7 | 182 | ATTGGCCACCCTGGGGACTGTCATCCTGTTGGTCTGCTTTTGCGCAGGCGCGGCGCACTCGAGGGGTGACAC |
| (HHV8 | | CTTTCAGACGTCCAGTTCCCCCACACCCCCAGGATCTTCCTCTAAGGCCCCCACCAAACCTGGTGAGGAAGC |
| gp04) | | ATCTGGTCCTAAGAGTGTGGACTTTTACCAGTTCAGAGTGTGTAGTGCATCGATCACCGGGGAGCTTTTTCG |
| | | GTTCAACCTGGAGCAGACGTGCCCAGACACCAAAGACAAGTACCACCAAGAAGGAATTTTACTGGTGTACAA |
| | | AAAAAACATAGTGCCTCATATCTTTAAGGTGCGGCGCTATAGGAAAATTGCCACCTCTGTCACGGTCTACAG |
| | | GGGCTTGACAGAGTCCGCCATCACCAACAAGTATGAACTCCCGAGACCCGTGCCACTCTATGAGATAAGCCA |
| | | CATGGACAGCACCTATCAGTGCTTTAGTTCCATGAAGGTAAATGTCAACGGGGTAGAAAACACATTTACTGA |
| | | CAGAGACGATGTTAACACCACAGTATTCCTCCAACCAGTAGAGGGGCTTACGGATAACATTCAAAGGTACTT |
| | | TAGCCAGCCGGTCATCTACGCGGAACCCGGCTGGTTTCCCGGCATATACAGAGTTAGGACCACTGTCAATTG |
| | | CGAGATAGTGGACATGATAGCCAGGTCTGCTGAACCATACAATTACTTTGTCACGTCACTGGGTGACACGGT |
| | | GGAAGTCTCCCCTTTTTGCTATAACGAATCCTCATGCAGCACAACCCCCAGCAACAAAAATGGCCTTAGCGT |
| | | CCAAGTAGTTCTCAACCACACTGTGGTCACGTACTCTGACAGAGGAACCAGTCCCACTCCCCAAAACAGGAT |
| | | CTTTGTGGAAACGGGAGCGTACACGCTTTCGTGGGCCTCCGAGAGCAAGACCACGGCCGTGTGTCCGCTGGC |
| | | ACTGTGGAAAACCTTCCCGCGCTCCATCCAGACTACCCACGAGGACAGCTTCCACTTTGTGGCCAACGAGAT |
| | | CACGGCCACCTTCACGGCTCCTCTAACGCCAGTGGCCAACTTTACCGACACGTACTCTTGTCTGACCTCGGA |
| | | TATCAACACCACGCTAAACGCCAGCAAGGCCAAACTGGCGAGCACTCACGTCCCTAACGGGACGGTCCAGTA |
| | | CTTCCACACAACAGGCGGACTCTATTTGGTCTGGCAGCCCATGTCCGCGATTAACCTGACTCACGCTCAGGG |
| | | CGACAGCGGGAACCCCACGTCATCGCCGCCCCCCTCCGCATCCCCCATGACCACCTCTGCCAGCCGCAGAAA |
| | | GAGACGGTCAGCCAGTACCGCTGCTGCCGGCGGCGGGGGGTCCACGGACAACCTGTCTTACACGCAGCTGCA |
| | | GTTTGCCTACGACAAACTGCGGGATGGCATTAATCAGGTGTTAGAAGAACTCTCCAGGGCATGGTGTCGCGA |
| | | GCAGGTCAGGGACAACCTAATGTGGTACGAGCTCAGTAAAATCAACCCCACCAGCGTTATGACAGCCATCTA |
| | | CGGTCGACCTGTATCCGCCAAGTTCGTAGGAGACGCCATTTCCGTGACCGAGTGCATTAACGTGGACCAGAG |
| | | CTCCGTAAACATCCACAAGAGCCTCAGAACCAATAGTAAGGACGTGTGTTACGCGCGCCCCCTGGTGACGTT |
| | | TAAGTTTTTGAACAGTTCCAACCTATTCACCGGCCAGCTGGGCGCGCGCAATGAGATAATACTGACCAACAA |
| | | CCAGGTGGAAACCTGCAAAGACACCTGCGAACACTACTTCATCACCCGCAACGAGACTCTGGTGTATAAGGA |
| | | CTACGCGTACCTGCGCACTATAAACACCACTGACATATCCACCCTGAACACTTTTATCGCCCTGAATCTATC |
| | | CTTTATTCAAAACATAGACTTCAAGGCCATCGAGCTGTACAGCAGTGCAGAGAAACGACTCGCGAGTAGCGT |
| | | GTTTGACCTGGAGACGATGTTCAGGGAGTACAACTACTACACACATCGTCTCGCGGGTTTGCGCGAGGATCT |
| | | GGACAACACCATAGATATGAACAAGGAGCGCTTCGTAAGGGACTTGTCGGAGATAGTGGCGGACCTGGGTGG |
| | | CATCGGAAAAACGGTGGTGAACGTGGCCAGCAGCGTGGTCACTCTATGTGGCTCATTGGTTACCGGATTCAT |
| | | AAATTTTATTAAACACCCCCTAGGTGGCATGCTGATGATCATTATCGTTATAGCAATCATCCTGATCATTTT |
| | | TATGCTCAGTCGCCGCACCAATACCATAGCCCAGGCGCCGGTGAAGATGATCTACCCCGACGTAGATCGCAG |
| | | GGCACCTCCTAGCGGCGGAGCCCCAACACGGGAGGAAATCAAAAACATCCTGCTGGGAATGCACCAGCTACA |
| | | ACAAGAGGAGAGGCAGAAGGCGGATGATCTGAAAAAAAGTACACCCTCGGTGTTTCAGCGTACCGCAAACGG |
| | | CCTTCGTCAGCGTCTGAGAGGATATAAACCTCTGACTCAATCGCTAGACATCAGTCCGGAAACGGGGGAGTG |
| | | ACAGTGGATTCGAGGTTATTGTTTGATGTAAATTTAGGAAACACGGCCCGCCTCTGAAGCACCACATACAGA |
| | | CTGCAGTTATCAACCCTACTCGTTGCACACAGACACAAATTACCGTCCGCAGATCATGGATTTTTTCAATCC |
| | | ATTTATCGACCCAACTCGCGGAGGCCCGAGAAACACTGTGAGGCAACCCACGCCGTCACAGTCGCCAACTGT |
| | | CCCCTCGGAGACAAGAGTATGCAGGCTTATACCGGCCTGTTTCCAAACCCCGGGGCGACCCGGCGTGGTTGC |
| | | CGTGGACACCACATTTCCACCCACCTACTTCCAGGGCCCCAAGCGGGGAGAAGTATTCGCGGGAGAGACTGG |
| | | GTCTATCTGGAAAACAAGGCGCGGACAGGCACGCAATGCTCCTATGTCGCACCTCATATTCCACGTATACGA |
| | | CATCGTGGAGACCACCTACACGGCCGACCGCTGCGAGGACGTGCCATTTAGCTTCCAGACTGATATCATTCC |
| | | CAGCGGCACCGTCCTCAAGCTGCTCGGCAGAACACTAGATGGCGCCAGTGTCTGCGTGAACGTTTTCAGGCA |
| | | GCGCTGCTACTTCTACACACTAGCACCCCAGGGGGTAAACCTGACCCACGTCCTCCAGCAGGCCCTCCAGGC |
| | | TGGCTTCGGTCGCGCATCCTGCGGCTTCTCCACCGAGCCGGTCAGAAAAAAAATCTTGCGCGCGTACGACAC |
| | | ACAACAATATGCTGTGCAAAAAATAACCCTGTCATCCAGTCCGATGATGCGAACGCTTAGCGACCGCCTAAC |
| | | AACCTGTGGGTGCGAGGTGTTTGAGTCCAATGTGGACGCCATTAGGCGCTTCGTGCTGGACCACGGGTTCTC |
| | | GACATTCGGGTGGTACGAGTGCAGCAATCCGGCCCCCCGCACCCAGGCCAGAGACTCTTGGACGGAACTGGA |
| | | GTTTGACTGCAGCTGGGAGGACCTAAAGTTTATCCCGGAGAGGACGGAGTGGCCCCCATACTCAATCCTATC |
| | | CTTTGATATAGAATGTATGGGCGAGAAGGGTTTTCCCAACGCGACTCAAGACGAGGACATGATTATACAAAT |
| | | CTCGTGTGTTTTACACACAGTCGGCAACGATAAACCGTACACCCGCATGCTACTGGGCCTGGGGACATGCGA |
| | | CCCCCTTCCTGGGGTGGAGGTCTTTGAGTTTCCTTCGGAGTACGACATGCTGGCCGCCTTCCTCAGCATGCT |
| | | CCGCGATTACAATGTGGAGTTTATAACGGGGTACAACATAGCAAACTTTGACCTTCCATACATCATAGCCCG |
| | | GGCAACTCAGGTGTACGACTTCAAGCTGCAGGACTTCACCAA |
| ORF8 | 183 | CAGTGGATTCGAGGTTATTGTTTGATGTAAATTTAGGAAACACGGCCCGCCTCTGAAGCACCACATACAGAC |
| (HHV8 | | TGCAGTTATCAACCCTACTCGTTGCACACAGACACAAATTACCGTCCGCAGATCATGGATTTTTTCAATCCA |
| gp05) | | TTTATCGACCCAACTCGCGGAGGCCCGAGAAACACTGTGAGGCAACCCACGCCGTCACAGTCGCCAACTGTC |
| | | CCCTCGGAGACAAGAGTATGCAGGCTTATACCGGCCTGTTTCCAAACCCCGGGGCGACCCGGCGTGGTTGCC |
| | | GTGGACACCACATTTCCACCCACCTACTTCCAGGGCCCCAAGCGGGGAGAAGTATTCGCGGGAGAGACTGGG |
| | | TCTATCTGGAAAACAAGGCGCGGACAGGCACGCAATGCTCCTATGTCGCACCTCATATTCCACGTATACGAC |
| | | ATCGTGGAGACCACCTACACGGCCGACCGCTGCGAGGACGTGCCATTTAGCTTCCAGACTGATATCATTCCC |
| | | AGCGGCACCGTCCTCAAGCTGCTCGGCAGAACACTAGATGGCGCCAGTGTCTGCGTGAACGTTTTCAGGCAG |
| | | CGCTGCTACTTCTACACACTAGCACCCCAGGGGGTAAACCTGACCCACGTCCTCCAGCAGGCCCTCCAGGCT |
| | | GGCTTCGGTCGCGCATCCTGCGGCTTCTCCACCGAGCCGGTCAGAAAAAAAATCTTGCGCGCGTACGACACA |
| | | CAACAATATGCTGTGCAAAAAATAACCCTGTCATCCAGTCCGATGATGCGAACGCTTAGCGACCGCCTAACA |
| | | ACCTGTGGGTGCGAGGTGTTTGAGTCCAATGTGGACGCCATTAGGCGCTTCGTGCTGGACCACGGGTTCTCG |
| | | ACATTCGGGTGGTACGAGTGCAGCAATCCGGCCCCCCGCACCCAGGCCAGAGACTCTTGGACGGAACTGGAG |
| | | TTTGACTGCAGCTGGGAGGACCTAAAGTTTATCCCGGAGAGGACGGAGTGGCCCCCATACTCAATCCTATCC |
| | | TTTGATATAGAATGTATGGGCGAGAAGGGTTTTCCCAACGCGACTCAAGACGAGGACATGATTATACAAATC |
| | | TCGTGTGTTTTACACACAGTCGGCAACGATAAACCGTACACCCGCATGCTACTGGGCCTGGGGACATGCGAC |
| | | CCCCTTCCTGGGGTGGAGGTCTTTGAGTTTCCTTCGGAGTACGACATGCTGGCCGCCTTCCTCAGCATGCTC |
| | | CGCGATTACAATGTGGAGTTTATAACGGGGTACAACATAGCAAACTTTGACCTTCCATACATCATAGCCCGG |
| | | GCAACTCAGGTGTACGACTTCAAGCTGCAGGACTTCACCAA |
| ORF9 | 184 | TGACTCAGACGCGGAAACAGCGCCTAGAAAGTTTCCTCTTGCGCTATGTGGGACAACTAGAGTCCAACCTGG |
| (HHV8 | | CAAGCAGTGGAGCAAGACGCCAGACAGCCGATCTCGAAAAAAATAATGCAGACAGAGGCAACGTTCATCCTA |
| gp06) | | GGTGACTGGGAGATAACGGTGTCTAACTGCCGGTTTACTTGCAGCAGCCTAACATGTGGCCCCCTTTACAGA |
| | | TCTAGCGGCGACTACACGCGGCTAAGAATCCCCTTCTCTCTGGATCGACTAATACGTGACCATGCCATCTTT |
| | | GGGCTAGTGCCAAATATTGAGGATCTGTTAACCCATGGGTCATGCGTCGCCGTAGTGGCCGACGCAAACGCC |
| | | ACAGGCGGCAACGCGCGACGCATCGTCGCGCCTGGCGTGATAAACAATTTTTCAGAACCCATCGGCATTTGG |
| | | GTACGCGGCCCTCCGCCGCAAACGCGCAAGGAAGCTATTAAGTTCTGCATATTTTTTGTCAGTCCCCTGCCC |
| | | CCGCGGGAGATGACCACATATGTGTTCAAGGGCGGCGATTTGCCTCCCGGAGCAGAGGAACCCGAAACACTA |
| | | CACTCCGCCGAGGCACCCCTACCGTCGCGCGAGACGCTGGTAACTGGACAGCTGCGATCCACCTCGCCGCGA |
| | | ACGTATACGGGATACTTTCACAGTCCTGTCCCGCTCTCTTTTTTGGACCTCCTGACATTCGAGTCCATTGGG |
| | | TGTGACAACGTGGAAGGTGACCCCGAGCAATTGACACCCAAGTACTTGACGTTCACGCAGACGGGAGAAAGA |
| | | CTTTGCAAAGTAACCGTTTACAACACCCATTCGACAGCATGCAAGAAGGCCCGTGTTCGTTTCGTCTACAGA |
| | | CCGACGCCGTCCGCCCGTCAGCTTGTCATGGGTCAGGCTTCACCCCTCATAACAACCCCTCTGGGAGCCAGG |
| | | GTATTCGCAGTCTATCCAGACTGTGAGAAAACTATCCCACCTCAGGAAACCACCACCCTGAGGATTCAATTG |
| | | CTGTTCGAGCAGCATGGTGCCAACGCCGGAGACTGCGCCTTTGTCATCATGGGGCTCGCCCGTGAAACAAAG |
| | | TTTGTCTCATTTCCCGCAGTACTCCTTCCGGGCAAGCACGAACACCTTATTGTATTCAACCCACAGACACAT |
| | | CCTCTGACCATTCAACGGGACACAATAGTGGGCGTGGCAATGGCTTGCTATATCCACCCCGGTAAGGCAGCC |
| | | AGCCAGGCACCATACAGCTTCTACGACTGCAAGGAAGAGAGCTGGCACGTGGGGCTCTTCCAGATCAAACGC |
| | | GGACCGGGAGGGGTCTGTACACCACCTTGCCACGTAGCGATTAGGGCCGACCGCCACGAGGAACCCATGCAA |
| | | TCGTGACTGTCCGAGCACATATGGCGCAGGAGTCAGAGCAGTGCTCCCGTGCGTTTGCAGTGTGCAGTAGTA |
| | | AACGACAGCTCGGGCGCGGCGAGCCCGTGTGGGATTCCGTCATTCACCCGAGCCACATCGTCATCTCTAATC |
| | | GAGTACCCCTCTTACTAAGAGAACAGCACATATGTCTCCCTTCGTGCCCCAGCGTCGGCCAGATCCTCCACA |
| | | GAGCCTACCCCAACTTTACATTTGACAACACGCACCGCAAGCAGCAAACGGAGACCTACACTGCATTCTACG |
| | | CTTTTGGGGACCAAAATAACAAGGTTAGGATCTTGCCCACTGTTGTGGAAAGCTCCTCGAGCGTGCTGATTT |
| | | TTAGACTGCGTGCATCGGTCTCTGCGAACATCGCCGTGGGAGGGCTCAAAATAATAATACTTGCTCTCACCC |
| | | TGGTGCATGCCCAAGGAGTGTACCTGCGTTGCGGTAAGGACCTTTCTACACCACACTGCGCACCGGCTATTG |
| | | TTCAGCGTGAGGTGCTGAGCAGCGGGTTTGAGCCGCAGTTTACCGTAACTGGCATTCCAGTGACATCCTCGA |
| | | ACTTAAACCAATGCTACTTTCTGGTAAGAAAGCCAAAAAGCCGGCTGGCAAAGCCGTTTGCACGCCTGTCCG |
| | | CGGAGACGACTGAGGAGTGTCGCGTCAGGTCTATCCGCCTTGGGAAGACACACCTGCGGATATCGGTGACTG |
| | | CGCCTGCGCAGGAAACGCCCGTCTGGGGGCTCGTGACCACGAGCTTCAGCCTTACCCCCACCGCACCGCTGG |
| | | CCTTTGATCGTAACCCGTACAATCACGAGACATTTGCCTGTAATGCCAAGCACTACATCCCAGTCATCTACA |
| | | GCGGACCAAAAATTACGCTGGCCCCGCGCGGCCGCCAGGTAGTCTGGCACAACAACAGCTACACGTCCTCCC |
| | | TGCCATGCAAAGTCACAGCCATCGTGTCAAACCACTGCTGTAACTGTGACATATTTTTAGAGGACTCGGAAT |
| | | GGCGCCCAAACAAGCCAGCACCCCTGAAACTGGTGAACACGAGTGATCATCCCGTCATATTGGAGCCGGACA |
| | | CACACATTGGAAACGCCCTCTTCATCATCGCACCCAAGGCCCGAGGTTTACGCAGACTGACTCGCTTAACCA |
| | | CAAAAACCATTGAACTTCCTGGCGGGGTAAAGATAGACAGCAGGAAATTACAAACATTCAGAAAAATGTATG |
| | | TTGCCACCGGACGCAGTTAGGTGTCCGGTTCCCACCCACACATTTGTCTTTATTGCTTTCA |
| ORF16 | 185 | CGCGTAATTCGAGGTCCCCGGAAGAGTAGAGGGTTGCATGTTATACAAACAACATAAACATTAAATGAACAT |
| (HHV8 | | TGTTCAAAACGTATGTTTATTTTTTTTCAAACAGGGGAGTAGGGTAGGAAGGGTACGTCTAATACGTAACTG |
| gp17) | | TTCGCTACTGCTTGTTCAGGAGCTCCTCGCAGAACATCTTGCGAATTTTAGATTTTGGACTAGAGCGACTGC |
| | | TGGCTTCAACGCGGTTCGATGTAGGGTTCGGCGTAGGAGCGTCTTTCTCCACCGCCGCGCATGGTGTATGCG |
| | | TGGTCTCCGGTGCCTGTTGTTGGATGCTCTGCGTGCTGGAGGCGGGGGTGGGTTCAGCGGGTGGTGCGCCAA |
| | | CTACCGCGAGTCCTGTAGAGACTGGCGGGTGGCTCACATGTGGCTGAGCAAAAAGGATGGGCGCCGCTTGCT |
| | | GGAACTGACCGTGTGGCGCCTGCACGTAAATGGGTGGGTGTACGTAGGTTCCTCCGTGCTCCTTCATTGTCG |
| | | GGAATTGACACGGGACCGCTGAATTGGCGTGGGGCCTGTAGTGTGGATCTACTGCGGCTGCTGCTGCAGAGG |
| | | AGGACGGCGGTGGCCCTGCGTGCCAACCGTTCAGTTTCATCTCTTTGAGTTCAGACTGTATTTCCGCTATGT |
| | | TCTTTGACATGGACAAGATATCCTTGTGATACGCCGGCTCCTCTCCTGGAAAGAGGTGTCCTTCGTCGTCCT |
| | | CTGCGCCGCGCTTGCGCTTCCCCGTCCTATATCCAGGCAGCTGTGGCGAGTAATACCATGGATCGTATGGGT |
| | | TCTTGTAAGCGTAGCCGTATGGTGGCGCTGGGTTTGAAACATACGAAGGTAGGTGATGGTCGGTGGGGAACA |
| | | TCTGGCCCCCACACCCCATTAGGCCTGGCCCTGAAAGTGTATGTGACATTTTTGCCGCTGTGGTCTTCATTC |
| | | CATCGATGCTGCTTTGTAGCATGCTCAGGAAGGCGGATTTGGGGATGGATATGATATCCTCTTGACCAGAGC |
| | | TGTTCATGGCTGGTCTGGGTGGTGTGACGGCTTGGATGCCGACCGGGAATTGGCTGGCCTTTAAATACGCCG |
| | | GGCTCAATATGCTGGCCACACCTCTGTCAGTTTTCAATAGGTCGAGGCGGTCCCGTATGAAGCTGGCATCTA |
| | | TAGCTTTTGCCATTAAGGTCTCCAGGGGACTGACGAAATTTGGTGTGGAAAGGTCCTCCAGCCTGCAGCTAC |
| | | TTACGTGCTGGAGGATGTGGGCGCGCTCCGACTTAGATACTGATGAGAATCTGGAAACCACCCACTCGGCGT |
| | | CGTGTCCGTACACGGCCACTGTGCCGCGTCGGCGCCCCAGGGCGCATAGTGATACGTGTTGAAACACGGGAC |
| | | CGCTGGGAGTCTGGGATAACTCGCGGGGATGTATAGACGATAAAGACAGCCCCGGGAGCCACGTGTGGAGTA |
| | | TCTCCAACAGTGGTTCCTTAGGGAGATTTTTCACGGGGGCTCTGGCCACGTGGGAGGTGTCCGCCAGCCTGG |
| | | ATGCCAGCTCTAGGAAGGCTGGCGACGTGATGGCTCCGGTGCAGAAAATACCGTGGGACACTTGAAATAGAC |
| | | CCAGTGTCCAGCCCACTTCTGTCTCTGGTAGGTGTTCGATTGTTATTGGAAGGGGTTCTGTGACTGGGAGAT |
| | | AATCCGTCACCTGATCCGGATCGAGATAGAGCTCTTGCTCCAGCTTGGGGCAGGACACAACATCTACAAACC |
| | | CTCCGACGTACAGGCCCTGTGCCATGCTCGGAAAATACGTGTGTGAGACCGAGCCGCTGAGCCCGGGGCTTA |
| | | GGAGGCTCATGTGGCGCTTTTTGCAAAATAAGAATTTAAATACATTCCACGCCCAAGAGCTGCGTTTTATTC |
| | | ATTTGGTTCTCTGCAGGATGTACAATTTCGGTCTAAATGTGTACCTGTTAAGGGAGGCTACTGCCAATGCCG |
| | | GGACCTACGACGAGGTGGTCCTGGGACGCAAGGTTCCTGCGGAGGTGTGGAAGCTCGTGTACGATGGGCTCG |
| | | AGGAGATGGGCGTGTCAAGTGAGATGCTGCTGTGTGAGGCATACCGGGACAGCCTCTGGATGCACTTGAACG |
| | | ATAAGGTGGGGCTCTTGAGGGGCCTGGCGAATTATCTGTTTCACCGGCTAGGGGTCACCCACGACGTTCGCA |
| | | TCGCCCCGGAAAACCTGGTGGACGGAAACTTTTTGTTTAATCTGGGAAGTGTGCTCCCCTGCAGGCTGCTCC |
| | | TTGCGGCGGGCTACTGCCTCGCCTTTTGGGGCAGCGATGAACACGAACGCTGGGTGCGCTTCTTCGCCCAGA |
| | | AGCTTTTCATTTGCTACCTGATAGTCTCCGGGCGTCTTATGCCACAGAGGTCTCTGCTAGTTTGGGCCAGCG |
| | | AAACGGGCTATCCCGGTCCGGTGGAGGCAGTCTGTCGCGACATCCGCTCCATGTACGGCATACGAACGTATG |
| | | CGGTCTCGGGTTATCTTCCGGCTCCGTCCGAAGCGCAGCTGGCCTACCTTGGTGCGTTTAACAACAACGCGG |
| | | TTTAAACGACCGCGAGGACCACCGGCAGGCAGCCAAGAACCATAAAGTACGCTCTATCGTAGTCATCGCCGC |
| | | CGCCAAACTGGGACTTGATAATCTCCTGGAGAAGGGTGGGTGGGGATGGGTGTGAAAGCAGGACGTCCAGGC |
| | | CCTCTTCTGTTGCCAGGCGGAGGGCTGTTCTCGCCTGGAGCAGCGCCAGTGGATCTCGGAATGTAAGCTGCT |
| | | GGTTCAGGATTTCGAATATCTCATTAAACCTACTGCCTGTCAGATTTACAAATGGTCCGGGTTGTTTGTGGG |
| | | ACACGGTCGATCGCGCCTCGAGGGCGGCCAGTATTATGCCAGGGAAGATGAAGGACACGGGGGCGTTTGGAT |
| | | TAGCCTGCAGTGTGGGGATTATGTAGTGCTCCGATATGAACGAAAATAGCTGGCCCCTTTTCAGCATGGGGG |
| | | CGTTTGGATCCGGTAGGGCACCGGGCTGAAATTTGGGTCCCAGCAGGGATACCAGGTTCAAGCGGCGGTTTG |
| | | GGTGCCCTCGCGCGACTTGCCCAAACTCCAGCAATCCATACGCGAGGATAAACACCTCCAGCGCAACAATCC |
| | | CCGCTCGCAGGTTCCACTGGTATGCGGAAAATGGTGGTATATCGGACCCAAACATGGCGCTCGTAATGGCGA |
| | | ATACCAAGTCCATGGCGGGCGCTGTCCCTGGCGCGCCCGTACCCTTGTTGTGGGGAAATAATCCAGCCTTAG |
| | | CCATCATTGCGTGAAGCTTGTGGCGCTGGAAGAAGGCTGTCGGATAGCGGCTCTCCTTATTGAGAGGCGCCA |
| | | GCGAGGCGCGCTCCTGGGGGTTTGAGTATGTGAAGCTGAAGTCCCCAGGACCGCTTTCCTGTTTTAGCTGAG |
| | | TGATTAGCAGGTCTAGCTTTTGAGGCAGGTCTGCTAACAGGTCATCGGGAGTAGCGGGCAGTTGCCTGGATG |
| | | TCTTTTGACAAAAGTACGCGTTGACGAGGCAAAGCGCGGCCTGGGTGTCCGTGAGATGCCTGGCGTCGGCGA |
| | | AAAAGTCAGCGGTGGTCGAGGCGACCGTCGTCAGGGTGTGAGAGATGAGTTTGAGCGATGTGGAATTCTGAA |
| | | AGTTAACAGTCCCCTTTAGTTCTTTAGGGAAGACGCGCCGCTGCATGGCGTTGTCCGTGAGGCTGATGAACC |
| | | ACGGCCCAAAGGATGGCAACCACTGATTCTGGTTCATGTACAGGGTGGGCATGAGCTCGCCGCGCAGGTCCC |
| | | TGTCAACGGAGAAGTGAGGGTCCCCGGGGACGATCGCCACGGTGAAGTTACGGTGGCTGGCCTGCGGGGGGG |
| | | ATGTCACTAAGGGAGGCTCATGGGAACGGCTTTGGGGCATGTCTATGTTGTCAGACCATGTCATGTTGCCTA |
| | | TCATCTGTTTCACCGCGTCGATATCTGCGTTAATGACGCGGACGCGTGAGTCATGGACCTGAACAAGCCGGT |
| | | CCAGCTCTAGGGAAAGCAGGTGTGCCTTTGTCTTTCGTTCTCGATTTCGCACGAGTTGGCTGCGCAGTCCAA |
| | | GGGCGACCCTTCTTGTTTCTTCCATGGTGGGCTTGTG |
| ORF18 | 186 | ACGACCGCGAGGACCACCGGCAGGCAGCCAAGAACCATAAAGTACGCTCTATCGTAGTCATCGCCGCCGCCA |
| (HHV8 | | AACTGGGACTTGATAATCTCCTGGAGAAGGGTGGGTGGGGATGGGTGTGAAAGCAGGACGTCCAGGCCCTCT |
| gp19) | | TCTGTTGCCAGGCGGAGGGCTGTTCTCGCCTGGAGCAGCGCCAGTGGATCTCGGAATGTAAGCTGCTGGTTC |
| | | AGGATTTCGAATATCTCATTAAACCTACTGCCTGTCAGATTTACAAATGGTCCGGGTTGTTTGTGGGACACG |
| | | GTCGATCGCGCCTCGAGGGCGGCCAGTATTATGCCAGGGAAGATGAAGGACACGGGGGCGTTTGGATTAGCC |
| | | TGCAGTGTGGGGATTATGTAGTGCTCCGATATGAACGAAAATAGCTGGCCCCTTTTCAGCATGGGGGCGTTT |
| | | GGATCCGGTAGGGCACCGGGCTGAAATTTGGGTCCCAGCAGGGATACCAGGTTCAAGCGGCGGTTTGGGTGC |
| | | CCTCGCGCGACTTGCCCAAACTCCAGCAATCCATACGCGAGGATAAACACCTCCAGCGCAACAATCCCCGCT |
| | | CGCAGGTTCCACTGGTATGCGGAAAATGGTGGTATATCGGACCCAAACATGGCGCTCGTAATGGCGAATACC |
| | | AAGTCCATGGCGGGCGCTGTCCCTGGCGCGCCCGTACCCTTGTTGTGGGGAAATAATCCAGCCTTAGCCATC |
| | | ATTGCGTGAAGCTTGTGGCGCTGGAAGAAGGCTGTCGGATAGCGGCTCTCCTTATTGAGAGGCGCCAGCGAG |
| | | GCGCGCTCCTGGGGGTTTGAGTATGTGAAGCTGAAGTCCCCAGGACCGCTTTCCTGTTTTAGCTGAGTGATT |
| | | AGCAGGTCTAGCTTTTGAGGCAGGTCTGCTAACAGGTCATCGGGAGTAGCGGGCAGTTGCCTGGATGTCTTT |
| | | TGACAAAAGTACGCGTTGACGAGGCAAAGCGCGGCCTGGGTGTCCGTGAGATGCCTGGCGTCGGCGAAAAAG |
| | | TCAGCGGTGGTCGAGGCGACCGTCGTCAGGGTGTGAGAGATGAGTTTGAGCGATGTGGAATTCTGAAAGTTA |
| | | ACAGTCCCCTTTAGTTCTTTAGGGAAGACGCGCCGCTGCATGGCGTTGTCCGTGAGGCTGATGAACCACGGC |
| | | CCAAAGGATGGCAACCACTGATTCTGGTTCATGTACAGGGTGGGCATGAGCTCGCCGCGCAGGTCCCTGTCA |
| | | ACGGAGAAGTGAGGGTCCCCGGGGACGATCGCCACGGTGAAGTTACGGTGGCTGGCCTGCGGGGGGGATGTC |
| | | ACTAAGGGAGGCTCATGGGAACGGCTTTGGGGCATGTCTATGTTGTCAGACCATGTCATGTTGCCTATCATC |
| | | TGTTTCACCGCGTCGATATCTGCGTTAATGACGCGGACGCGTGAGTCATGGACCTGAACAAGCCGGTCCAGC |
| | | TCTAGGGAAAGCAGGTGTGCCTTTGTCTTTCGTTCTCGATTTCGCACGAGTTGGCTGCGCAGTCCAAGGGCG |
| | | ACCCTTCTTGTTTCTTCCATGGTGGGCTTGTG |
| ORF21 | 187 | CCTTCTTGGCGGCCCTTGCATGCTGGCGATGCATATCGTTGACATGTGGAGCCACTGGCGCGTTGCCGACAA |
| (HHV8 | | CGGCGACGACAATAACCCGCTCCGCCACGCAGCTCATCAATGGGAGAACCAACCTCTCCATAGAACTGGAAT |
| gp22) | | TCAACGGCACTAGTTTTTTTCTAAATTGGCAAAATCTGTTGAATGTGATCACGGAGCCGGCCCTGACAGAGT |
| | | TGTGGACCTCCGCCGAAGTCGCCGAGGACCTCAGGGTAACTCTGAAAAAGAGGCAAAGTCTTTTTTTCCCCA |
| | | ACAAGACAGTTGTGATCTCTGGAGACGGCCATCGCTATACGTGCGAGGTGCCGACGTCGTCGCAAACTTATA |
| | | ACATCACCAAGGGCTTTAACTATAGCGCTCTGCCCGGGCACCTTGGCGGATTTGGGATCAACGCGCGTCTGG |
| | | TACTGGGTGATATCTTCGCATCAAAATGGTCGCTATTCGCGAGGGACACCCCAGAGTATCGGGTGTTTTACC |
| | | CAATGATTGTCATGGCCGTCAAGTTTTCCATATCCATTGGCAACAACGAGTCCGGCGTAGCGCTCTATGGAG |
| | | TGGTGTCGGAAGATTTCGTGGTCGTCACGCTCCACAACAGGTCCAAAGAGGCTAACGAGACGGCGTCCCATC |
| | | TTCTGTTCGGTCTCCCGGATTCACTGCCATCTCTGAAGGGCCATGCCACCTATGATGAACTCACGTTCGCCC |
| | | GAAACGCAAAATATGCGCTAGTGGCGATCCTGCCTAAAGATTCTTACCAGACACTCCTTACAGAGAATTACA |
| | | CTCGCATATTTCTGAACATGACGGAGTCGACGCCCCTCGAGTTCACGCGGACGATCCAGACTAGGATCGTAT |
| | | CAATCGAGGCCAGGCGCGCCTGCGCAGCTCAAGAGGCGGCGCCGGACATATTCTTGGTGTTGTTTCAGATGT |
| | | TGGTGGCACACTTTCTTGTTGCGCGGGGCATTACCGAGCACCGATTTGTGGAGGTGGACTGCGTGTGTCGGC |
| | | AGTATGCGGAACTGTATTTTCTCCGCCGCATCTCGCGTCTGTGCATGCCCACGTTCACCACTGTCGGGTATA |
| | | ACCACACCACCCTTGGCGCTGTGGCCGCCACACAAATAGCTCGCGTGTCCGCCACGAAGTTGGCCAGTTTGC |
| | | CCCGCTCTTCCCAGGAAACAGTGCTGGCCATGGTCCAGCTTGGCGCCCGTGATGGCGCCGTCCCTTCCTCCA |
| | | TTCTGGAGGGCATTGCTATGGTCGTCGAACATATGTATACCGCCTACACTTATGTGTACACACTCGGCGATA |
| | | CTGAAAGAAAATTAATGTTGGACATACACACGGTCCTCACCGACAGCTGCCCGCCCAAAGACTCCGGAGTAT |
| | | CAGAAAAGCTACTGAGAACATATTTGATGTTCACATCAATGTGTACCAACATAGAGCTGGGCGAAATGATCG |
| | | CCCGCTTTTCCAAACCGGACAGCCTTAACATCTATAGGGCATTCTCCCCCTGCTTTCTAGGACTAAGGTACG |
| | | ATTTGCATCCAGCCAAGTTGCGCGCCGAGGCGCCGCAGTCGTCCGCTCTGACGCGGACTGCCGTTGCCAGAG |
| | | GAACATCGGGATTCGCAGAATTGCTCCACGCGCTGCACCTCGATAGCTTAAATTTAATTCCGGCGATTAACT |
| | | GTTCAAAGATTACAGCCGACAAGATAATAGCTACGGTACCCTTGCCTCACGTCACGTATATCATCAGTTCCG |
| | | AAGCACTCTCGAACGCTGTTGTCTACGAGGTGTCGGAGATCTTCCTCAAGAGTGCCATGTTTATATCTGCTA |
| | | TCAAACCCGATTGCTCCGGCTTTAACTTTTCTCAGATTGATAGGCACATTCCCATAGTCTACAACATCAGCA |
| | | CACCAAGAAGAGGTTGCCCCCTTTGTGACTCTGTAATCATGAGCTACGATGAGAGCGATGGCCTGCAGTCTC |
| | | TCATGTATGTCACTAATGAAAGGGTGCAGACCAACCTCTTTTTAGATAAGTCACCTTTCTTTGATAATAACA |
| | | ACCTACACATTCATTATTTGTGGCTGAGGGACAACGGGACCGTAGTGGAGATAAGGGGCATGTATAGAAGAC |
| | | GCGCAGCCAGTGCTTTGTTTCTAATTCTCTCTTTTATTGGGTTCTCGGGGGTTATCTACTTTCTTTACAGAC |
| | | TGTTTTCCATCCTTTATTAGACGGTC |
| ORF25 | 188 | CTAACCCTTCTAGCGTTGGCTAGTCATGGCACTCGACAAGAGTATAGTGGTTAACTTCACCTCCAGACTCTT |
| (HHV8 | | CGCTGATGAACTGGCCGCCCTTCAGTCAAAAATAGGGAGCGTACTGCCGCTCGGAGATTGCCACCGTTTACA |
| gp26) | | AAATATACAGGCATTGGGCCTGGGGTGCGTATGCTCACGTGAGACATCTCCGGACTACATCCAAATTATGCA |
| | | GTATCTATCCAAGTGCACACTCGCTGTCCTGGAGGAGGTTCGCCCGGACAGCCTGCGCCTAACGCGGATGGA |
| | | TCCCTCTGACAACCTTCAGATAAAAAACGTATATGCCCCCTTTTTTCAGTGGGACAGCAACACCCAGCTAGC |
| | | AGTGCTACCCCCATTTTTTAGCCGAAAGGATTCCACCATTGTGCTCGAATCCAACGGATTTGACCTCGTGTT |
| | | CCCCATGGTCGTGCCGCAGCAACTGGGGCACGCTATTCTGCAGCAGCTGTTGGTGTACCACATCTACTCCAA |
| | | AATATCGGCCGGGGCCCCGGATGATGTAAATATGGCGGAACTTGATCTATATACCACCAATGTGTCATTTAT |
| | | GGGGCGCACATATCGTCTGGACGTAGACAACACGGATCCACGTACTGCCCTGCGAGTGCTTGACGATCTGTC |
| | | CATGTACCTTTGTATCCTATCAGCCTTGGTTCCCAGGGGGTGTCTCCGTCTGCTCACGGCGCTCGTGCGGCA |
| | | CGACAGGCATCCTCTGACAGAGGTGTTTGAGGGGGTGGTGCCAGATGAGGTGACCAGGATAGATCTCGACCA |
| | | GTTGAGCGTCCCAGATGACATCACCAGGATGCGCGTCATGTTCTCCTATCTTCAGAGTCTCAGTTCTATATT |
| | | TAATCTTGGCCCCAGACTGCACGTGTATGCCTACTCGGCAGAGACTTTGGCGGCCTCCTGTTGGTATTCCCC |
| | | ACGCTAACGATTTGAAGCGGGGGGGGGGTATGGCGTCATCTGATATTCTGTCGGTTGCAAGGACGGATGACG |
| | | GCTCCGTCTGTGAAGTCTCCCTGCGTGGAGGTAGGAAAAAAACTACCGTCTACCTGCCGGACACTGAACCCT |
| | | GGGTGGTAGAGACCGACGCCATCAAAGACGCCTTCCTCAGCGACGGGATCGTGGATATGGCTCGAAAGCTTC |
| | | ATCGTGGTGCCCTGCCCTCAAATTCTCACAACGGCTTGAGGATGGTGCTTTTTTGTTATTGTTACTTGCAAA |
| | | ATTGTGTGTACCTAGCCCTGTTTCTGTGCCCCCTTAATCCTTACTTGGTAACTCCCTCAAGCATTGAGTTTG |
| | | CCGAGCCCGTTGTGGCACCTGAGGTGCTCTTCCCACACCCGGCTGAGATGTCTCGCGGTTGCGATGACGCGA |
| | | TTTTCTGTAAACTGCCCTATACCGTGCCTATAATCAACACCACGTTTGGACGCATTTACCCGAACTCTACAC |
| | | GCGAGCCGGACGGCAGGCCTACGGATTACTCCATGGCCCTTAGAAGGGCTTTTGCAGTTATGGTTAACACGT |
| | | CATGTGCAGGAGTGACATTGTGCCGCGGAGAAACTCAGACCGCATCCCGTAACCACACTGAGTGGGAAAATC |
| | | TGCTGGCTATGTTTTCTGTGATTATCTATGCCTTAGATCACAACTGTCACCCGGAAGCACTGTCTATCGCGA |
| | | GCGGCATCTTTGACGAGCGTGACTATGGATTATTCATCTCTCAGCCCCGGAGCGTGCCCTCGCCTACCCCTT |
| | | GCGACGTGTCGTGGGAAGATATCTACAACGGGACTTACCTAGCTCGGCCTGGAAACTGTGACCCCTGGCCCA |
| | | ATCTATCCACCCCTCCCTTGATTCTAAATTTTA |
| ORF26 | 189 | CGATTTGAAGCGGGGGGGGGGTATGGCGTCATCTGATATTCTGTCGGTTGCAAGGACGGATGACGGCTCCGT |
| (HHV8 | | CTGTGAAGTCTCCCTGCGTGGAGGTAGGAAAAAAACTACCGTCTACCTGCCGGACACTGAACCCTGGGTGGT |
| gp27) | | AGAGACCGACGCCATCAAAGACGCCTTCCTCAGCGACGGGATCGTGGATATGGCTCGAAAGCTTCATCGTGG |
| | | TGCCCTGCCCTCAAATTCTCACAACGGCTTGAGGATGGTGCTTTTTTGTTATTGTTACTTGCAAAATTGTGT |
| | | GTACCTAGCCCTGTTTCTGTGCCCCCTTAATCCTTACTTGGTAACTCCCTCAAGCATTGAGTTTGCCGAGCC |
| | | CGTTGTGGCACCTGAGGTGCTCTTCCCACACCCGGCTGAGATGTCTCGCGGTTGCGATGACGCGATTTTCTG |
| | | TAAACTGCCCTATACCGTGCCTATAATCAACACCACGTTTGGACGCATTTACCCGAACTCTACACGCGAGCC |
| | | GGACGGCAGGCCTACGGATTACTCCATGGCCCTTAGAAGGGCTTTTGCAGTTATGGTTAACACGTCATGTGC |
| | | AGGAGTGACATTGTGCCGCGGAGAAACTCAGACCGCATCCCGTAACCACACTGAGTGGGAAAATCTGCTGGC |
| | | TATGTTTTCTGTGATTATCTATGCCTTAGATCACAACTGTCACCCGGAAGCACTGTCTATCGCGAGCGGCAT |
| | | CTTTGACGAGCGTGACTATGGATTATTCATCTCTCAGCCCCGGAGCGTGCCCTCGCCTACCCCTTGCGACGT |
| | | GTCGTGGGAAGATATCTACAACGGGACTTACCTAGCTCGGCCTGGAAACTGTGACCCCTGGCCCAATCTATC |
| | | CACCCCTCCCTTGATTCTAAATTTTA |
| ORF28 | 190 | AACGGGGTGTGTGCTATAATGGATGGCTATGGGGGGGCTGTAGATAATTGAGCGCTGTGCTTTTATTGTGGG |
| (HHV8 | | GATATGGGCTTGTACATGTGTCTATCATCGGTAGCCATAAAATGGGCCATGACAACTGCCACAAGTAAGTCG |
| gp29) | | TCCGACATGTGCTTTTGCTTGGCGCTGTATGACTGCCCTCCATCCCTAAGCGGGACGCACTTGATCGCGCGG |
| | | ACCTGTTCTACCAGGTAGGTCACCGGGTCAAATGATATTTTGATGGTGTTGGACACCACCGTCTGGCTGGCG |
| | | CTCAGGGTGCCGGAGTTCAGAGCGTAGATGAATGTCTCAAACGCGGAGGATTTCTCGCCTCCCAACATGTAA |
| | | ATTGGCCACTGCAGGGCGCTGCTCTTGTCAGTATAGTGTAGAAAATGTATGGGGAGCGGGCATATTTCGTTA |
| | | AGGACGGTTGCAATGGCCACCCCAGAATCTTGGCTGCTGTTGCCTTCGACCGCCGCGTTCACGCGCTCAATT |
| | | GTGGGGTGGAGCACAGCGATCGCCTTAATCATCGTGCATGCGCAGGACGCTATCTCGTAAGCAGCTGCGCCA |
| | | GTGAGGTCGCGCAGGAAGAAATGCTCCATGCCCAATATGAGGCTTCTGGTGGGAGTCTGAGTACTCGTGACA |
| | | ACGGCGCCCACGCCAGTACCGGACGCCTCCGTGTTGTTCGTATACGCGGGGTCGATGTAAACAAACAGCTGT |
| | | TTTCCAAGGCACTTCTGAACCTGCTGGGCGGTGGTGTCTACCCGACACATGTCAAACTGTGTCAGCGCTGCG |
| | | TCACCCACCACGCGGTAAAGCGTAGCATTTGACGACGCTGCTCCCTCGCCCATTAGTTCGGTGTCGAATGCC |
| | | CCCTCCATAAAGAGGTTGGTGGTGGTTTTGATGGATTCGTCGATGGTGATGTACGTCGGAATGTGCAGTCTG |
| | | TAACAAGGACAGGACACTAGTGCGTCTTGCAGGTGGAAATCTTCGCGGTGGTCCGCACACACGTAACTGACC |
| | | ACATTCAGCATCTTTTCCTGGGCGTTCCTGAGGTTAAGCAGGAAACTCGTGGAGCGGTCTGACGAGTTCACG |
| | | GATGATATAAATATAAGCTTGGCGTCTTTCTGAAGCATGAAACCCAGAATAGCCGGCAGTGCATCCTTTTT |
| ORF32 | 191 | CCGGAGGCGCAAACTTCGGAATTTCCTAAACAAGGAATGCATATGGACTGTTAACCCAATGTCAGGGGACCA |
| (HHV8 | | TATCAAGGTCTTTAACGCCTGCACCTCTATCTCGCCGGTGTATGACCCTGAGCTGGTAACCAGCTACGCACT |
| gp33) | | GAGCGTGCCTGCTTACAATGTGTCTGTGGCTATCTTGCTGCATAAAGTCATGGGACCGTGTGTGGCTGTGGG |
| | | AATTAACGGAGAAATGATCATGTACGTCGTAAGCCAGTGTGTTTCTGTGCGGCCCGTCCCGGGGCGCGATGG |
| | | TATGGCGCTCATCTACTTTGGACAGTTTCTGGAGGAAGCATCCGGACTGAGATTTCCCTACATTGCTCCGCC |
| | | GCCGTCGCGCGAACACGTACCTGACCTGACCAGACAAGAATTAGTTCATACCTCCCAGGTGGTGCGCCGCGG |
| | | CGACCTGACCAATTGCACTATGGGTCTCGAATTCAGGAATGTGAACCCTTTTGTTTGGCTCGGGGGCGGATC |
| | | GGTGTGGCTGCTGTTCTTGGGCGTGGACTACATGGCGTTCTGTCCGGGTGTCGACGGAATGCCGTCGTTGGC |
| | | AAGAGTGGCCGCCCTGCTTACCAGGTGCGACCACCCAGACTGTGTCCACTGCCATGGACTCCGTGGACACGT |
| | | TAATGTATTTCGTGGGTACTGTTCTGCGCAGTCGCCGGGTCTATCTAACATCTGTCCCTGTATCAAATCATG |
| | | TGGGACCGGGAATGGAGTGACTAGGGTCACTGGAAACAGAAATTTTCTGGGTCTTCTGTTCGATCCCATTGT |
| | | CCAGAGCAGGGTAACAGCTCTGAAGATAACTAGCCACCCAACCCCCACGCACGTCGAGAATGTGCTAACAGG |
| | | AGTGCTCGACGACGGCACCTTGGTGCCGTCCGTCCAAGGCACCCTGGGTCCTCTTACGAATGTCTGACTACT |
| | | TCAGCCGCTTGCTGATATATGAGTGTAAAAAACTTAAGGCCCTGGGCTTACGTTCTTATTGAAGCATGTTGC |
| | | GCACATCAGCGAGCTGGACCGTCCTCCGGGTCGCGTGTAGATTATGGTTCCGTTCTCCTTCTTGATGTTTAA |
| | | ATTTTTGGGGGGGAACCACCGACAAAGCGTCTTTATGATTTCCGCGAACACGGAGTTGGCTACGTGCTTTTG |
| | | GTGGGCTACGTACCCAATGTTAATGTTCTCTACGGATGCCAGTAGCATGCTGATGATCGCCACCACTATCCA |
| | | TGTCTTTCCGTGTCTCCTTGGTATTAGGAATACGCTTGCCTTTTGCTTAAACGTCTGTAAAACACTGTTTGG |
| | | AGTTTCA |
| ORF40 | 192 | AGCGGAGAGGGGGTGGTGCGAGTTGGCAGTTGACGGGTTTGTGATAGCTGGAGTGCTGACCACGGCACAGGA |
| (HHV8 | | CCCATTAACTTTCCTATGTGTTTATTTTTAGCAATGGTCTCCAGAATTCAAGGATCTCAAAAGGGCCTGCCA |
| gp42) | | GATGGCCGGGTTTACTCTGAAGGGGGGGACTTCGGGGGATCTTGTATTCTCATCGCATGCGAACTTGCTCTT |
| | | TTCAACCTCGATGGGATATTTCCTCCATGCAGGCAGTCCAAGGTCGACAGCGGGGACGGGGGGTGAGCCTAA |
| | | CCCACGTCACATCACCGGACCAGACACTGAGGGAAATGGGGAACACAGAAACTCCCCCAACCTCTGCGGCTT |
| | | TGTTACCTGGCTGCAAAGCTTAACCACATGCATTGAACGAGCCCTAAACATGCCTCCCGACACTTCCTGGCT |
| | | GCAGCTGATAGAGGAAGTGATACCCCTGTATTTTCATAGGCGAAGACAAACATCATTCTGGCTCATCCCCCT |
| | | ATCGCACTGTGAAGGGATCCCAGTATGCCCCCCTTTACCATTTGACTGCCTAGCACCAAGGCTGTTTATAGT |
| | | AACAAAGTCCGGACCCATGTGTTACCGGGCAGGCTTTTCGCTTCCTGTGGATGTTAATTACCTGTTCTATTT |
| | | AGAGCAGACTCTGAAAGCTGTCCGGCAAGTTAGCCCACAGGAACACAACCCCCAAGACGCAAAGGAAATGAC |
| | | TCTACAGCTAGAGGCCTGGACCAGGCTTTTATCTTTATTTTGAAAAAAGGGAAACAATGGGGGGTTTGAAAA |
| | | GGGTGCACATTTTCAGATATTTTAAAACTTCATTGTTCTCCAGGTGCTTGGTAAAGATGGTATCAC |
| ORF47 | 193 | GTTCAACATGGACGCATGGTTGCAACAGACGGTCTTTAGGGGCACCCTATCCATCAGTCAGGGGGTGGACGA |
| (HHV8 | | CCGGGATCTGTTACTGGCACCTAAGTGGATTTCCTTTCTGAGCCTCTCATCATTTCTGAAACAGAAACTGCT |
| gp49) | | CTCGCTGCTCAGACAGATTCGGGAACTTAGGCTAACCACCACAGTGTATCCCCCACAGGACAAGCTGATGTG |
| | | GTGGTCCCACTGCTGCGATCCAGAGGATATTAAAGTGGTGATCTTAGGCCAGGACCCGTACCACAAGGGCCA |
| | | AGCTACTGGCCTGGCGTTTAGTGTGGATCCGCAATGTCAGGTTCCACCCAGTTTGAGAAGCATCTTTAGAGA |
| | | GCTAGAGGCTTCCGTCCCCAATTTCAGTACTCCTTCCCACGGGTGCCTCGACAGCTGGGCTCGCCAGGGTGT |
| | | GTTGCTACTAAACACAGTTTTGACGGTGGAGAAGGGGAGGGCCGGCTCACACGAGGGACTTGGCTGGGATTG |
| | | GTTCACGAGTTTCATCATCAGTAGCATATCCTCAAAGTTAGAACATTGCGTTTTTCTCCTGTGGGGGCGCAA |
| | | GGCCATTGACAGAACTCCGCTCATAAACGCACAGAAACACCTGGTGCTTACGGCCCAGCATCCATCTCCGCT |
| | | GGCCTCTCTTGGTGGCCGACACTCGCGATGGCCTCGGTTCCAGGGCTGTAATCACTTTAACCTAGCCAACGA |
| | | CTATTTGACTCGCCACCGGCGTGAGACTGTGGACTGGGGCCTGTTGGAGCAGTAAAGGCAATAACTCGTGTG |
| | | CTTTGTAAATTTCCGCCCCTAGCGGTCAACCCCGTACAAGGCCATGGCGATGTTTGTGAGGACCTCGTCTAG |
| | | CACACACGATGAAGAGAGAATGCTTCCAATTGAAGGAGCGCCTCGCAGACGACCCCCCGTGAAGTTCATATT |
| | | CCCACCTCCACCTCTTTCATCACTTCCAGGATTTGGCAGGCCGCGCGGCTATGCTGGACCCACGGTGATAGA |
| | | TATGTCTGCCCCAGACGACGTCTTCGCCGAGGACACGCCATCGCCGCCAGCAACCCCTCTGGATCTACAGAT |
| | | ATCCCCGGATCAGTCGAGCGGCGAATCTGAATATGACGAGGATGAGGAAGATGAAGATGAAGAAGAAAATGA |
| | | CGATGTTCAGGAGGAAGACGAGCCAGAGGGGTACCCTGCAGACTTTTTTCAACCTTTATCTCACTTGCGCCC |
| | | GAGGCCTCTGGCCAGACGGGCCCATACGCCCAAACCGGTAGCAGTGGTAGCGGGCCGCGTGCGCAGTTCAAC |
| | | GGACACGGCGGAGTCCGAGGCGTCCATGGGATGGGTTAGTCAGGATGACGGATTTTCCCCTGCTGGGCTCTC |
| | | ACCTTCAGACGACGAGGGGGTTGCTATCCTGGAACCGATGGCGGCATACACTGGGACCGGGGCATACGGACT |
| | | TTCACCTGCTTCCAGAAATAGTGTACCTGGAACACAAAGTTCACCATACAGCGACCCTGATGAAGGGCCCTC |
| | | GTGGCGCCCCCTGCGCGCCGCACCCACCGCGATCGTCGACCTGACATCGGACTCTGATAGCGATGACAGTTC |
| | | CAACTCTCCGGACGTGAACAATGAGGCCGCGTTTACCGACGCGCGCCATTTTTCCCACCAGCCACCCTCGTC |
| | | CGAGGAGGACGGAGAAGACCAAGGGGAAGTATTGAGTCAGAGAATCGGGCTCATGGACGTGGGCCAGAAGCG |
| | | CAAAAGGCAGTCTACCGCCTCCTCTGGTAGCGAGGATGTGGTGCGCTGCCAGAGACAACCAAACTTAAGCCG |
| | | CAAAGCAGTGGCGTCCGTGATAATTATATCCTCGGGGAGTGACACAGACGAGGAGCCCTCGTCCGCCGTGAG |
| | | CGTGATCGTGTCTCCGTCGAGCACAAAGGGTCACCTCCCAACCCAATCTCCCAGTACTTCCGCCCACTCGAT |
| | | TTCATCAGGAAGCACAACTACCGCGGGGTCCAGGTGCAGCGACCCAACCCGCATCCTGGCCTCCACGCCACC |
| | | CCTGTGTGGAAACGGTGCATATAACTGGCCGTGGCTGGACTGATA |
| ORF49 | 194 | AAAGGTCGATCTTTACCTTGTCATCTTGCGCCATTTTTGTGGCTGCCTGGACAGTATTCTCACAACAGACTA |
| (HHV8 | | CCCCTTGCGGAGTAAGGTTGACTTTTTAAAGGGGACGTGTCATTGCCACCCAGCTACTGGTTTCTGGGCGGG |
| gp51) | | GCTTAATGAGTCGCCGGTAGCTGCCTGGTATTTAGTGGAGGATAAGCTGTAGCTGGGTCCTATGGGGGTTGG |
| | | GTGGGGAGACCCTAGCGTACATGTGACTGAACATGGAGGTGTGTATCCCAATTCCGGGTATTGGAGATGAAA |
| | | ATTGTGAGAGCTGGAGGGCACAGATTGTGGCATTCGGTACCACATCGGGTTTCGTCAAGACCGAGCGTATTC |
| | | TCAGAGGTCTGTTTCCGGAGCGCGGACACCCGGGGTTCTTAGCGTCCCTGGTGGTCCTGAAGCATACGCTGG |
| | | CTTCCCCGGGGGGGCTCAACACCAGACTGAATCTACTTCCAGTATTACAGATGTTAAAATATGTGGGACAGG |
| | | AAATGTACATGCGGGCAAAATGCCAGGCAACAGCATCTGACATGACTTTGATCTGGGATGACTGCAAAGATA |
| | | GATTTATGCTGATACTGGAACAGGCCTGTGGGTGCCACCAATGTATGACCGTGGTAGAAGAAATCACCCACT |
| | | GTAGCGCCATCTCTGCCCCCCCAAGCTCTTTGTCCCACGGGAGACACATTCTTTCTGCGGGGCTCATCAACT |
| | | TTGCAAGACGCCAGGTTCTCCTTGGTGGGTCAGTGTCTTTTTCTGAGTTTTCTATTCCAGACCTAATACAGA |
| | | CACCGGAGCAATACCCCTTTGTGGATGTGGAGTTCCGGCGGGAGCTTAGCTTGATTTCATCGTGTTTGAACG |
| | | TCTGCTGGCTCTACCACATCTTCATAGAGCACATTACCTCGGACGTGAGACGGTTGGAGTCATGCATGGCCA |
| | | GTGTCCTGGAAGAGTATGGCGGACTGTCACCCACCCGCCCATGGGCAGAGGCAGTGACCTTTTTGAGTCAGC |
| | | TGCCGCGCCCCACCAGGAAACCCTGGAAAGAACTGTCGGTAAGCCGGATCAACGTGGAAGCCCGGCTTTTGG |
| | | ATACCCTGGTGATGCAATTAGAGAAACCGGTTCCTGTGGAAAT |
| ORF50 | 195 | AGTGTTCGCAAGGGCGTCTGTGCCTGCGTTAACTTCCCAGGCAGTTTATTTTTAACAGTTTGGTGCAAAGTG |
| (Rta) | | GAGTTAACCTACAGATTCTACTTAAAATAGCTCATTTTCTCACGAATCTGGTTGATTGTGACTATTTGTGAA |
| (HHV8 | | ACAATAATGATTAAAGGGGGTGGTATTTCCTCCGTTGTCGACTATAACCTGGCGTGTAAACGTGTAACCCTG |
| gp52) | | CCAAATGCCCAGAATGAAGGACATACCTACTAAGAGTTCCCCGGGAACGGACAATTCTGAGAAAGATGAAGC |
| | | TGTCATTGAGGAAGATCTAAGCCTCAACGGGCAACCATTTTTTACGGACAATACTGACGGTGGGGAAAACGA |
| | | AGTCTCTTGGACAAGCTCGCTGTTGTCAACCTACGTAGGTTGCCAGCCCCCGGCCATACCGGTCTGTGAAAC |
| | | GGTCATTGACCTTACAGCGCCTTCCCAAAGTGGCGCGCCCGGTGACGAACATCTGCCATGCTCACTGAATGC |
| | | AGAAACTAAATTCCACATCCCCGATCCTTCCTGGACGCTCTCTCACACACCACCAAGAGGACCACACATTTC |
| | | GCAACAGCTTCCAACTCGCAGATCCAAGAGGCGACTACATAGAAAGTTTGAAGAGGAACGCTTATGCACTAA |
| | | GGCCAAACAGGGCGCAGGTCGCCCCGTGCCTGCGTCTGTAGTTAAGGTAGGGAACATCACCCCCCATTATGG |
| | | GGAAGAACTGACAAGGGGTGACGCCGTCCCAGCCGCCCCTATAACACCCCCCTCCCCGCGCGTTCAACGCCC |
| | | AGCACAGCCCACACATGTCCTGTTTTCTCCTGTTTTTGTCTCTTTAAAGGCCGAAGTATGTGATCAGTCACA |
| | | TTCTCCCACGCGAAAGCAAGGCAGATACGGCCGCGTGTCATCGAAAGCATACACAAGACAGCTGCAGCAGGT |
| | | ATAGACGGGAAACAGGTGTCTATCTTGGCCGGCTGGTTACTCAAATGGGAACAATGGCGCCACCTTGCTGTC |
| | | TTTGTAGGCATTAGAAGAAAAGGATGCACAACTATGTTTCCTAGCGGCGAGATTGGAGGCACATAAGGAACA |
| | | GATTATTTTCCTTCGCGACATGCTGATGCGAATGTGCCAGCAGCCAGCGTCGCCAACGGACGCGCCACTCCC |
| | | ACCATGTTGAAGCTTGGTTGTGCCGTCGTCCGGGAGAACCATGCCAGACTTTGTGTGGTAAGAAGGAATTGT |
| | | TATCCGGCAGCAATATTAAAGGGACCCAAGTTAATCCCTTAATCCTCTGGGATTAATAACCATGAGTTCCAC |
| | | ACAGATTCGCACAGAAATCCCTGTGGCGCTCCTAATCCTATGCCTTTGTCTGGTGGCGTGCCATGCCAATTG |
| | | TCCCACGTATCGTTCGCATTTGGGATTCTGGCAAGAGGGTTGGAGTGGACAGGTTTATCAGGACTGGCTAGG |
| | | CAGGATGAACTGTTCCTACGAGAATATGACGGCCCTAGAGGCCGTCTCCCTAAACGGGACCAGACTAGCAGC |
| | | TGGATCTCCGTCGAGTGAGTATCCAAATGTCTCCGTATCTGTTGAAGATACGTCTGCCTCTGGGTCTGGAGA |
| | | AGATGCAATAGATGAATCGGGGTCGGGGGAGGAAGAGCGTCCCGTGACCTCCCACGTGACTTTTATGACACA |
| | | AAGCGTCCAGGCCACCACAGAACTGACCGATGCCTTAATATCAGCCTTTTCAGGTGTATTACACGTTTCAAC |
| | | TGTAATCCCTCGCAATTGGGTAAACCGTCGGTGTGTAGGGATAAAGCGTAACCTTACGTTCTGTCTCATCTA |
| | | CAGGATCATATTCATCTGGGGAACCATCCAGGACCACGCGAATTCGCGTATCACCGGTCGCAGAAAACGGCA |
| | | GAAATAGTGGTGCTAGTAACCGTGTGCCATTTTCTGCCACCACTACAACGACTAGAGGAAGAGACGCGCACT |
| | | ACAATGCAGAAATACGGACCCATCTTTACATACTATGGGCTGTGGGTTTATTGCTGGGACTTGTCCTTATAC |
| | | TTTACCTGTGCGTTCCACGATGCCGGCGTAAGAAACCCTACATAGTGTAACACAAAACCATAAAAGTA |
| ORF56 | 196 | TCCCACTATATAACCTGGCTGCCAGGTTCCCAAAATAGCCCGCGGCATACGGCTCACTTCCCCCCACATTCC |
| (HHV8 | | CCCCGTGCACAATATAAGAACCAAAGGACATGGTACAAGCAATGATAGACATGGACATTATGAAGGGCATCC |
| gp58) | | TAGAGGGTAAGTCCTCGTCTACAACAGACTTTTCCCATTTCTAACGTATCGTGCTATCTTCGTCGCCCGGCG |
| | | GACCATCCCCCCACCCCTCATTTATCGCGTTTGATATTACAGACTCTGTGTCCTCCTCTGAGTTTGACGAAT |
| | | CGAGGGACGACGAGACGGACGCACCGACACTGGAAGACGAGCAATTGTCCGAACCCGCCGAGCCTCCGGCAG |
| | | ACGAGCGCATCCGTGGTACCCAGTCGGCCCAGGGAATCCCACCCCCCCTGGGCCGCATCCCAAAAAAATCTC |
| | | AAGGTCGTTCTCAACTGCGCAGTGAGATCCAGTTTTGCTCCCCACTGTCTCGACCCAGGTCCCCCTCACCAG |
| | | TAAACAGGTACGGTAAAAAAATCAAGTTTGGAACCGCCGGTCAAAACACACGTCCTCCCCCTGAAAAGCGTC |
| | | CTCGGCGCAGACCACGCGACCGCCTACAATACGGCAGAACAACACGGGGCGGACAGTGTCGCGCTGCACCGA |
| | | AGCGAGCGACCCGCCGTCCGCAGGTCAATTGCCAGCGGCAGGATGACGACGTCAGACAGGGTGTGTCTGACG |
| | | CCGTAAAGAAACTCAGACTCCCTGCGAGCATGATAATTGACGGTGAGAGCCCCCGCTTCGACGACTCGATCA |
| | | TCCCCCGCCACCATGGCGCATGTTTCAATGTCTTCATTCCCGCCCCACCATCCCACGTCCCGGAGGTGTTTA |
| | | CGGACAGGGATATCACCGCTCTCATAAGAGCAGGGGGCAAAGACGACGAACTCATAAACAAAAAAATCAGCG |
| | | CAAAAAAGATTGACCACCTCCACAGACAGATGCTGTCTTTTGTGACCAGCCGCCATAATCAAGCGTACTGGG |
| | | TGAGTTGCCGTCGAGAAACCGCAGCCGCCGGAGGCCTGCAAACGCTTGGGGCTTTCGTGGAGGAACAAATGA |
| | | CGTGGGCCCAGACGGTTGTGCGCCACGGGGGGTGGTTTGATGAGAAGGACATAGATATAATTTTGGACACCG |
| | | CAATATTTGTCTGCAATGCGTTTGTTACCAGATTTAGATTACTTCATCTTTCCTGCGTTTTTGACAAGCAGA |
| | | GCGAGCTAGCACTGATCAAACAGGTGGCATATTTGGTAGCGATGGGAAACCGCTTAGTAGAGGCATGTAACC |
| | | TTCTTGGCGAGGTCAAGCTTAACTTCAGGGGAGGGCTGCTCTTGGCCTTTGTCCTAACTATCCCAGGCATGC |
| | | AGAGTCGCAGAAGTATTTCTGCGCGCGGACAGGAGCTGTTTAGAACACTTCTGGAATACTACAGGCCAGGGG |
| | | ATGTGATGGGGCTACTAAACGTGATAGTAATGGAACATCACAGCTTGTGCAGAAACAGTGAATGTGCAGCGG |
| | | CAACCCGGGCCGCAATGGGGTCGGCCAAATTTAACAAGGGTTTATTCTTTTATCCACTTTCTTAAGGATTGC |
| | | CAAACCCCATGGCAGAGTGTCTCCCGTATTCCATGTAACTCACGTAGCCTTTCTCT |
| ORF57 | 197 | GGATTGCCAAACCCCATGGCAGAGTGTCTCCCGTATTCCATGTAACTCACGTAGCCTTTCTCT |
| (HHV8 | | |
| gp59) | | |
| ORF58 | 198 | TTGAATAATACATGTGTTTTTCTTGGTTTGTTGACCATGACACCCCTCCCTCGCGTCCAAAGGCCGCTTGTA |
| (HHV8 | | TTAGAGGGTGGACAGTGCCTGGGTGCTGTCCCGGGTTATGGGTGTGTGCCAGTAGTTCAACTGCATTGGTTC |
| gp63) | | CCTTTTCCGTAGTGAGTTCTAACCACAAGTTTCCGCAGCCCGACAACCGGCTGGGGGGGGCGGTGTTGAGCT |
| | | GCATATATTGAGTTTTGTTGTTAGATGGCACAGAGTCTACGTGCCAGTGGGGTTGGGGTCCAGCTAGTTGTG |
| | | GCGAGAAAGTCGCCCACGGAAAAGGTGTTTTGTGTCGTGGCTTTTGCCTAAAAAGATGCCTCGCTACACGGA |
| | | GTCGGAATGGCTCACGGACTTTATTATAGATGCTTTAGACAGTGGACGCTTCTGGGGGGTAGGGTGGTTGGA |
| | | TGAACAAAAGAGAATATTCACCGTGCCGGGTCGAAACCGGCGGGAGAGAATGCCAGAAGGCTTCGATGACTT |
| | | CTATGAGGCATTTTTGGAGGAGCGACGTAGGCACGGGCTGCCAGAAATCCCGGAGACTGAGACTGGCCTGGG |
| | | CTGCTTTGGACGGCTATTAAGGACCGCCAATCGAGCCAGACAGGAGAGGCCCTTTACCATCTATAAGGGAAA |
| | | AATGAAACTCAACCGCTGGATTATGACACCTAGGCCATACAAGGGATGTGAAGGATGTCTTGTGTACTTGAC |
| | | GCAGGAACCAGCCATGAAAAACATGCTAAAAGCATTGTTTGGGATCTATCCCCATGATGACAAACACAGAGA |
| | | AAAGGCACTTAGAAGGAGCCTTAGAAAAAAAGCCCAGAGGTAGGATGGTTGATGTACTGGGCGGTGGGTTGT |
| | | GTGGGCGGCGGGATGTACGTGCAGCGGGCATCACGGGAAATTGGAGATGTCACTCAGACTTACCTTTGTGTA |
| | | ATTAACTTTTGTTTAGGGAGGCCGCCAGGAAACAGGCGGCGGCAGTCGCCACGCCCACAACATCCTCCGCAG |
| | | CTGAAGTTTCATCACGGTCACAGAGCGAAGATACGGAATCGAGTGACAGCGAAAACGAACTTTGGGTGGGGG |
| | | CTCAGGGTTTTGTAGGGAGGGATATGCACAGTTTGTTTTTTGAAGAGCCAGAACCGTCGGGGTTTGGGTCAT |
| | | CTGGTCAGTCATCGAGCTTATTAGCTCCGGATTCCCCGCGTCCCTCCACGAGCCAGGTGCAGGGCCCATTAC |
| | | ACGTGCACACCCCGACGGATCTATGTTTGCCAACGGGGGGTTTACCTTCTCCTGTTATTTTTCCACATGAGA |
| | | CACAAGGCTTATTAGCGCCGCCTGCTGGACAGTCGCAAACCCCATTTTCCCCAGAAGGCCCCGTCCCCAGTC |
| | | ATGTCAGTGGGCTGGATGATTGCCTACCGATGGTGGATCACATTGAGGGGTGTTTGTTAGATCTCTTGTCAG |
| | | ATGTTGGCCAGGAGCTTCCTGACTTAGGCGACCTGGGTGAACTTCTGTGTGAAACTGCGAGCCCTCAGGGCC |
| | | CGATGCAGTCGGAGGGAGGTGAGGAGGGGTCCACGGAGAGTGTCTCAGTACTTCCCGCCACGCATCCCCTTG |
| | | AGAGTTCGGCACCTGGGGCCTCTGTCATGGGTTCAGGCCAGGAGCTTCCTGACTTAGGCGACCTGAGTGAAC |
| | | TTCTGTGTGAAACTGCGAGCCCTCAGGGCCCGATGCAGTCGGAGGGAGGTGAGGAGGGGTCCACGGAGAGTG |
| | | TCTCAGTACTTCCCGCCACGCATCCCCTTGAGAGTTCGGCACCTGGGGCCTCTGTCATGGGTTCATCTTTCC |
| | | AAGCTTCCGACAATGTGGATGATTTTATTGATTGTATTCCACCGTTGTGTCGTGATGACCGGGACGTCGAGG |
| | | ACCAAGAGAAAGCTGACCAGACATTTTACTGGTATGGAAGCGACATGAGGCCCAAGGTCTTAACCGCCACCC |
| | | AATCCGTGGCAGCATACCTGAGTAAGAAACAGGCTATTTACAAAGTGGGTGACAAGCTTGTGCCCCTAGTGG |
| | | TGGAAGTGTATTATTTCGGAGAAAAGGTGAAGACCCACTTTGATTTAACGGGGGGCATCGTTATTTGCTCCC |
| | | AAGTCCCAGAGGCCTCCCCTGAACACATATGTCAGACGGTACCCCCGTATAAATGCTTACTTCCCAGAACGG |
| | | CCCACTGTAGTGTGGACGCAAACCGAACTTTGGAACAGACGCTGGACAGGTTTTCCATGGGAGTTGTGGCCA |
| | | TCGGTACAAACATGGGCATTTTTCTGAAGGGATTATTGGAATACCCAGCATACTTTGTTGGAAATGCATCGC |
| | | GAAGAAGAATAGGCAAATGTAGGCCCCTGTCCCACCGCCACGAGATCCAACAAGCTTTTGACGTGGAGCGAC |
| | | ATAATCGAGAACCTGAAGGGTCCCGGTACGCGTCCCTGTTTCTGGGCCGCCGGCCGTCGCCTGAATATGACT |
| | | CGGATCACTATCCAGTCATTTTGCACATTTACCTTGCCCCATTTTACCACAGAGACTAAAATTTTGACAAGT |
| | | CTTCTTGTCACTCTGTCCGGGTACCTCCCTTTGTCTTACCGCCCTCCGTTTTGCACTATAAATATCATTGCC |
| | | GTTAGAAACCAGGCTCTATCCGCAACTTCTATGTTTCCTGTTATAGTAGGCCCATGTGGGCTTGGGAGTGGC |
| | | CAAACTCACTGAGTGGGACATCATTAAAGGTTAGCGCCACCGTGTGGCTGCAA |
| ORF59 | 199 | CACCATGTGCCGCCTGGACAGTGAGCGCGCTCTGTCGCTCTTCAGTTATCTGAGCGGGACGTTGGCGGCGAC |
| (HHV8 | | CCCCTTTCTGTGGTGTTTTATCTTCAAGGCCCTGTACTCGTTCACACTCTTTACCACAGAGATCACGGCCGT |
| gp64) | | GTTTTTCTGGTCGCTGCCAGTCACGCACTTGGCCCTGATATGCATGTGTCTGTGCCCTGCGGCGCAAAAACA |
| | | GCTGGACCGGAGGCTGGAATGGATCTGCGCGTCAGCAGTGTTTGCTGCTGTAGTTTGCGCGGCCTTTTCTGG |
| | | GTTTACATTTTCTCGTGTGCCCTTCATACCGGGTCTGTGCGTACTTAACTGTTTACTGCTGTTACCTTATCC |
| | | GCTAGCCACCGCAACGGCGGTGTATCAGGCGCCGCCAATAGTACACAGGTACTATGAGCTGGGCTTCTGCGG |
| | | AGCATTTATGGTGTACTACCTTCTGTTGTTTAAGAAGGTCTTTGTGTCCGGCGTTTTCTGGCTGCCCTTCAT |
| | | TGTCTTCTTGGTCGGGGGACTTTTGGCATTTAGGCACCTGGAACAGCATGTGTACATCAGGGCCGGAATGCA |
| | | AAGGAGGAGGGCCATATTCATCATGCCCGGGAAGTACATCACCTATTCAGTGTTCCAGGCCTGGGCCTACTG |
| | | TAGGCGCGAGGTTGTCGTGTTTGTGACCTTACTGCTGGCCACCCTGATATCGACGGCCTCGATCGGCCTGCT |
| | | GACTCCGGTCCTGATTGGCCTGGATAAGTATATGACGCTATTTTATGTTGGGTTACTGTCATGCGTGGGCGT |
| | | ATCCGTCGCCTCCCGACGAGCGCTATTTGTTCTCCTGCCTTTGGCGGCAGTGTTGCTCACCTTGGTGCACAT |
| | | ACTTGGATCAGGTCCGGATATGCTCCTAGTTAGGTCCTGCCTCTGCTGCCTATTCCTCGTGAGCATGCTGGC |
| | | CGCAATGGGGGTCGAGATTCAGCTAATTAGGCGAAAACTCCACAGGGCACTTAACGCTCCACAGATGGTATT |
| | | GGCCCTATGCACGGTTGGAAATTTATGTATCTCATGTCTCCTGTCGGT |
| ORF63 | 200 | AGGCCATGGCAGCCCAGCCTCTGTACATGGAGGGAATGGCCTCCACCCACCAAGCTAACTGTATATTCGGAG |
| (HHV8 | | AACATGCTGGATCCCAGTGCCTCAGCAACTGCGTCATGTACCTGGCGTCCAGCTATTATAACAGCGAAACCC |
| gp68) | | CCCTCGTCGACAGAGCCAGCCTGGACGATGTACTTGAACAGGGCATGAGGCTGGACCTCCTCCTACGAAAAT |
| | | CTGGCATGCTGGGATTTAGACAATATGCCCAACTTCATCACATCCCCGGATTCCTCCGCACAGACGACTGGG |
| | | CCACCAAGATCTTCCAGTCTCCAGAGTTTTATGGGCTCATCGGACAGGACGCGGCCATCCGCGAGCCATTCA |
| | | TCGAGTCCTTGAGGTCGGTTTTGAGTCGAAACTACGCGGGCACGGTACAGTACCTGATCATTATCTGCCAGT |
| | | CCAAAGCCGGAGCAATCGTCGTCAAGGACAAAACGTATTACATGTTTGACCCCCACTGCATACCAAACATCC |
| | | CCAACAGTCCTGCACACGTCATAAAGACTAACGACGTTGGCGTTTTATTACCGTACATAGCCACACATGACA |
| | | CTGAATACACCGGGTGCTTCCTTTACTTTATCCCACATGACTACATCAGCCCAGAGCACTACATCGCAAACC |
| | | ACTACCGCACCATTGTGTTCGAAGAACTCCACGGGCCCAGAATGGATATCTCCCGCGGGGTGGAATCATGCT |
| | | CCATCACCGAAATCACGTCCCCTTCTGTATCCCCCGCGCCTAGTGAGGCACCATTGCGCAGGGACTCCACCC |
| | | AATCACAAGACGAAACGCGCCCGCGCAGACCTCGCGTCGTCATTCCTCCTTACGATCCGACAGACCGCCCAC |
| | | GACCGCCTCACCAAGACCGCCCGCCAGAGCAGGCAGCGGGATACGGTGGAAACAAAGGACGCGGCGGTAACA |
| | | AAGGACGCGGCGGAAAGACGGGACGTGGCGGAAATGAAGGACGCGGTGGCCACCAGCCACCAGACGAGCACC |
| | | AGCCCCCACACATCACCGCGGAACACATGGACCAGTCCGACGGACAAGGCGCCGATGGAGACATGGATAGTA |
| | | CACCCGCAAATGGTGAGACATCCGTTACGGAAACCCCGGGCCCCGAACCCAATCCCCCAGCACGGCCTGACA |
| | | GAGAGCCACCGCCCACTCCCCCGGCGACCCCAGGCGCCACAGCGCTGCTCTCTGACCTAACTGCCACAAGAG |
| | | GGCAGAAACGCAAATTTTCCTCGCTTAAAGAATCTTATCCCATCGACAGCCCACCCTCTGACGACGATGATG |
| | | TGTCCCAGCCCTCCCAACAAACGGCTCCGGATACTGAAGATATTTGGATTGACGACCCACTCACACCCTTGT |
| | | ACCCACTAACGGATACACCATCTTTCGACATAACGGCGGACGTCACACCCGACAACACCCACCCCGAGAAAG |
| | | CAGCGGACGGGGACTTTACCAACAAGACCACAAGCACGGATGCGGACAGGTATGCCAGCGCCAGTCAGGAAT |
| | | CGCTGGGCACCCTGGTCTCGCCATACGATTTTACAAACTTGGATACACTGCTGGCAGAGCTGGGCCGGTTGG |
| | | GAACGGCACAGCCTATCCCTGTAATCGTGGACAGACTAACATCGCGACCTTTTCGAGAAGCCAGCGCTCTAC |
| | | AGGCTATGGATAGGATACTAACACACGTGGTCCTAGAATACGGTCTGGTTTCGGGTTACAGCACAGCTGCCC |
| | | CATCCAAATGCACCCACGTCCTCCAGTTTTTCATTTTGTGGGGCGAAAAACTCGGCATACCAACGGAGGACG |
| | | CAAAGACGCTCCTGGAAAGCGCACTGGAGATCCCCGCAATGTGCGAGATCGTCCAACAGGGCCGGTTGAAGG |
| | | AGCCCACGTTCTCCCGCCACATTATAAGCAAGCTAAACCCCTGCTTGGAATCCCTACACGCCACTAGTCGTC |
| | | AGGACTTCAAGTCCCTGATACAGGCATTCAACGCCGAAGGGATTAGGATCGCCTCGCGTGAGAGGGAGACGT |
| | | CCATGGCCGAACTGATAGAAACGATAACCGCCCGCCTTAAACCAAATTTTAACATTGTCTGTGCCCGCCAGG |
| | | ACGCACAAACCATTCAAGACGGCGTCGGTCTCCTCAGGGCCGAGGTTAACAAGAGAAACGCACAGATAGCCC |
| | | AGGAGGCTGCGTATTTTGAGAATATAATCACGGCCCTCTCCACATTCCAACCACCTCCCCAATCGCAACAGA |
| | | CGTTCGAAGTGCTGCCGGACCTCAAACTGCGCACGCTCGTGGAGCACCTGACCCTGGTTGAGGCGCAGGTGA |
| | | CAACGCAAACGGTGGAAAGTCTACAGGCATACCTACAGAGCGCTGCCACTGCTGAGCATCACCTTACCAACG |
| | | TGCCCAACGTCCACAGTATACTGTCTAACATATCCAACACTCTAAAAGTTATAGATTATGTAATTCCAAAAT |
| | | TTAT |
| ORF72 | 201 | GCTTGTGATTTTGTTTAGGGCGGAAA |
| (HHV8 | | |
| gp77) | | |
| ORF73 | 202 | AAGCCACACCTCTCCCCCTTTTTCCTCCCTAGAAGCCACCGTCGCCGCTCCGCACTTGCATTTGGCGCCATG |
| (LANA) | | GGTGCTGGTGTGTGTGGGGGGCAGTGTTCTCACGACCCATCTACCTCAACTGAACACACGGACAACGGCTAG |
| (HHV8 | | CGTACTCTCGCGGCCCAGCGTCGTCGATGGGAGAACCTGACAGAGCACCCTGAAACTCCAGGCTCTACAGGT |
| gp78) | | AGGCCACATACGCTCGCCACTCTATATGGCAACTGCCAATAACCCGCCCTCGGGACTTCTGGATCCCACGCT |
| | | ATGTGAGGATCGGATCTTTTACAATATTCTTGAAATTGAGCCGCGCTTTTTAACTTCTGACTCTGTATTTGG |
| | | GACCTTTCAACAATCTCTTACTTCGCATATGCGTAAGTTACTGGGCACATGGATGTTTTCAGTTTGCCAGGA |
| | | ATACAACCTAGAACCTAACGTGGTCGCGTTGGCCCTTAATCTTTTGGACAGACTCCTACTTATAAAGCAGGT |
| | | GTCCAAAGAACACTTTCAAAAGACAGGGAGCGCCTGCCTGTTAGTGGCCAGTAAGCTCAGAAGCCTCACGCC |
| | | TATTTCTACCAGTTCACTTTGCTATGCCGCGGCAGACTCCTTTTCCCGCCAAGAACTTATAGACCAGGAGAA |
| | | AGAACTCCTTGAGAAGTTGGCGTGGCGAACAGAGGCAGTCTTAGCGACGGACGTCACTTCCTTCTTGTTACT |
| | | TAAATTGCTGGGGGGCTCCCAACACCTGGACTTTTGGCACCACGAGGTCAACACCCTGATTACAAAAGCCTT |
| | | AGTTGACCCAAAGACTGGCTCATTGCCCGCCTCTATTATCAGCGCTGCAGGCTGTGCGCTGTTGGTTCCTGC |
| | | CAACGTCATTCCGCAGGATACCCACTCGGGTGGGGTAGTTCCTCAGCTGGCAAGCATATTGGGATGCGATGT |
| | | TTCCGTTCTACAGGCGGCAGTGGAACAGATCCTAACATCTGTTTCGGACTTTGATCTGCGCATTCTGGACAG |
| | | CTATTAAGCTTGTGATTTTGTTTAGGGCGGAAA |
| ORF74 | 203 | CCCGCGGATGTCTACGTGCCCTTCCCCCTTAATTTAATCTAGCCTCCCGTTCCCATGATGCAGAGAGGCGAA |
| (HHV8 | | TTTGGTTTGTACACAGATGTGACTATGTATTTGTTTTATTATGCGATTAAATGAGGGGTCTGATCCCAAAAG |
| gp80) | | CAATGTTTAGTGGTGGTCGTTGATCTTCTTGACGCTCCATAGGTAGATTGACTGGAACGCCATGGCCCACGG |
| | | GGACATGGACAGGGGTGTTAGGTCTGGTGGAACATGCTGCCACTGCCACGGATGGAACATCAGAGATGGGTC |
| | | TATGATCAGGGCAGCGTGTCGCCCGTCACTGGATGTAAGTCCGGCCACCGTGGAGTTGCCTGTGGGGTTTCT |
| | | GGGATAGTGTCTGGCTGGCAGGGTCTCATCCGCGGCATTTCCATGGTAGGTGAGGGTTATCTCGCCTCGCTG |
| | | TCTCAGTATGTACTCGAGGGCGTCCTGCTCGTACCGGACCCCCAGGTACTCTCCCTGGGCCCAGCTGGGCAG |
| | | CACCGTCCCCCGCAACACTCGGAGGAAAACGCTCTTAGTGTTCTGAGGGATCTGTATGTTTAGCCAGTGGCT |
| | | GTCATACAGCTTGGACACGTTGGTCTCCAGGTTTACCGCCCAGCGCTGGGGTGGTGTGGGTCCGTACGTGTA |
| | | TGGTGAGGATTCCGACCGGCCCACTACACCCAGGGCCACCAGCAGCTGGAAGCCCACCTCGCCACAGCAGAT |
| | | GGAGAATGTGTCGGGTCTGTTTAGAAACTCTGTCAGGGTGGAGGCACAGGTAGGGTCGTTACACAGCGCCAG |
| | | GACCCATCCCCTGGCGCTGGCGTAGCTGGCCTGGCAGCCTGTTCTGAGACATGTAATCAGACCAGAGAACCC |
| | | CGACAAGGACTGTCCTCGTTTAAGCTCTTCCACAGTCACCGTGGCCACCTCAAAGCCCGTGTTCTGCAACGC |
| | | GGCCATGAGCGCGTACGGGGCACTGCTCCCAGGCAGCACCAACGCGGCCACACGGCGCGGGGAGGTGGGGCA |
| | | CGAAAACAGGCGCAGCTGACTCCCAAGGCACATGGCCCTTAGGCTGCCCAGGTGATGCTCCAGACGACCCAG |
| | | GTCCTTCCTGTGCATGTCCTCCAGTGGGTGCAGGGGAGGCGTCACCAGGTTCCACATTTCGTCAGAAAAGGA |
| | | GGTCCATGAGACTTGCAAGGAAGTCAGGGTCTCTTGAAACACAACTGTCTCGTTCTGCAAAACCGTGACGTT |
| | | GTTGCCTTGTCCCTCGGGGCCAACGGTGCCCAGTGGGTGTGCCACGCAGCGGTAGTCCCTGGCCGCCCGCAG |
| | | CACCTCTGACAAGTGTACCTGGGGCACCTCAACCAGTGCCCCAGGGGTCTCTGAAACCATAAGTTCGAGCGG |
| | | GTTAGGGTGGGCGGGTAGTGAGAGCTGCAGTCCCCTGCAGCCGGCCAGGGCCATCTCGATTGCAGATGGGAG |
| | | AAGCCCTCCGTCCCCTATGTCGTGCCCAGATACAATGAGCCTCTTGGACATCAGGTACTTAACAAGCATGAA |
| | | CAGGCTGGCGACCGTGGACGGGTTCAGAGGGGGTATTGGGTGCCTGGATGCCAGGAAGTTGTGCTCGAAGGT |
| | | GGACCCGGCTATGAGACAGCTCTGATTCACGGCCAGGTATACCAGGGCGTTGCCTTCGACCTTTACGTCCGG |
| | | GGTGACCCTGTATCTGGATCCCTTGACCTCGGCCCAGCTGGTAAACACCACCGAGTTGAAGGGAAGGACCTC |
| | | CACCGTTTCTTGCTGTTGTGTGATGCGCACATGGCGCTCCGAAAGCGTCGGAGAGCTGGCAGCCGAGGAGAT |
| | | GGACAGTGCCACTCCCAGCTCCCGGCAGAATTCCTTGCAGGCGAAGAGGCACTCCTGTAGGAGGCCGGCTTG |
| | | GTGGTCCTCTGGACTCCACGCCACGGCGCCAGTTAGCACTACGTCCTGGAGCTTGGACACGGGACTGAACAT |
| | | GAGGTTGGTGAGAGCCTCGGTGATGGCATAGGTGGCCCCGGTGGATACATTAGTAGCCATCTTGTAGGCCTG |
| | | CTCCCCCATGGCCATTGCCTGACCCCTCCACGCTGGCACTGGAAGCAGCTCCTGGGGCAGGGCCTTCACCCA |
| | | GGTCTCGAAGTCCTTGTGTAGGAGGTTGGCCATGGACGGAGTGATGGCCTCCACCGTGTCGGGCACTCTGGG |
| | | CGCCACCCTCTCGGCCAGCATGGACGAGTGCAGCACCAGGTGGTAGTCTGAAACCGGTATGTCCAGGGGTCC |
| | | CACGCCAGCCTGTTGGGCGATGAGGCCGTTGGAGCATCGGTCCATGTGTCGCGTAAAGAACTCCTTGCTGCC |
| | | AACCGTCGAGTGGCGAAGTAACTGGTGGATTGTGGAGCCGGTGGCAAAAAGGCCCCAGTCAACATCCTCGGG |
| | | GTGCCCCGAGACGCGGACACCATCGGACAGCGCCAGCCAGGGGGACGGGGGGGTGGACGACGGCTGGTCTAC |
| | | AGAGAAGACCCTCGTGGTCTCCCCGGTCAGGTCGTCTACTATTCTGATGCCTGGGTGCTCCGAGGTCCTCCC |
| | | GAGGACCGTTACCTGGCACGCGCACAGGCGCGCGGCGCGCTGCAGTACCTCCAACGGGGTCTCGCCCAGATC |
| | | CCCAGGCACCGCGCCCGACTCTGCCACCACCGCAAACACCAGGGAGCAATACACGTTGAGAAAGTGCTCTGC |
| | | CACCGCCGCCTTCACGGCATCCGGACCGGCCGCGGGATCCGCAGGCAGGTGGGTGCGCACCTCGTCGGGTAG |
| | | CTTGGAGACAAACAGCTCCAGGCCGGTCCGCGGCGCCAGCGCCTGCAGGTGCCTCACCACCGGGGCCGGGTC |
| | | ATGCGATCTGTTTAGTCCGGAGAAGATAGGGCCCTTGGCAAGCCGCTGGACCAGCTTCAGGGTCTCCAAGAT |
| | | GCGCACCGCATTGTCGGAGCTGTCGCGATAGAGGTTAGGGTAGGTGTCCGGTCCATCCGTGGGCTCAAACCT |
| | | GCCCAGACACACCACTGTCTGCTGGGGGATCATCCTTCTCAGGGAGATGCATTCTTTGGAAGTAGTGGTAGA |
| | | GATGGAGCAGACTGCCAGGGCGTTGCCAGGAGTGGTGGCGATGGTGCGCACCGTTTTTAAGAAACCCCCCAG |
| | | GGTGGGGACTCCCGCTCCCTGCAGCATCTCGGCCTGCTGTACGCCCTTGGCGAATATGCGACGGAATCGGCT |
| | | GTGCGCACGGGGTCCCAGGGCCGGTTCGGTGGCATACAGGCCGGTGAGGGCCCCCTGTGTCTGTCCGCCTGG |
| | | AAACAGGGTGCTGTGAAACAGCAGGTTGCCAAGGCCGCGAATACCCCTCTGCACGCTGCTGTGGACGTGGGT |
| | | GTACGCTCCGTGGATCCCGAACGCCTGTCTGGCACAGTTCCAGGGCCACCGTTCCATGGTGCATCTTCCCGG |
| | | TATCACAAAGTACCTGGCCACGTTATAATTGTCCCCGGTTGAAGCCTGCACCGCCAGCGGTAGCAGGTCTGC |
| | | CCCCAGGGATATCATAACAGCCTGCATAATGACATCATCTTCAATGTGTGGCCTAGCCACGGGCTGGGGACC |
| | | CTCGGGCACTTCCAACCCCTCGTACGGTACCAGGTCGGTATTTTGTGTAAATGCCCTGATAAACTGAGGTGG |
| | | GTGTGGTTCTAGCAGGGTCTGTGTGATTTTGGACACCAGGTGCCTGCCCACTTCCACTCTAGCCCACTCCTG |
| | | CAATCCTAGCTCTTGCAGCAGAACTGCAAGCTCTGTTGACAATGTTGTGGGCCGGTGGTGCATGTTTGGCCC |
| | | GTAGCCAAAGGATACAACACGCTCGCTCCCCCGTGGCACAGACCGCCTGATGACATGGGGATATCCAAGGAG |
| | | CGGTGACAGCACAGCGAGCACCGTCTGTATTTCCACATCCCGTCTCTCTCGCTCCTCCCTCGAAGTGGGAGG |
| | | TCTTCGGAAAGTTATCCATAGCAGATAGTAGCCTCCGGTGCCACCGGGTACGAGAGTGAGTGTGCCCGTACG |
| | | GCTTGTATAAAAGTTCACAAAAGCTTCCTCATCCGCGGTGAGATCACTCTCCAACCACAGCCCAGTGACGTC |
| | | GTAGGCCATGCCTAGAGGGCGCACCGCCCCCGGGGACACCCTCTGTAGTCAGGCTGCCGAGAAACCCGCGAG |
| | | ATCTCTGGGGAGTAGGAAGAAACTTAGAATCCCCAAATATGTCGCAGTCACAGGTTGTCGGGCAGAGTCTGT |
| | | TTCCGCTTTCATGGGATCCACAGTTACTTGTAGCCATGTCACTAACCTCAAATACTCAAAAAAAGCTATCGA |
| | | TGGAAAAATGCTGTGGTCCTAGGTTAGTCCGTGGGAAACAAAACTTCCTCATACACTTCATCTGCAGGCTGA |
| | | AATGGTGGCGGATCCAGACTCCTTACACCACAGTTGCTCACATTAGAGATACCTGATTGGTTAATACAAGCG |
| | | GACGCACGCGTTGGTGGAGGCGTGTTGTCGCCCAAGATACTAGCATAGGTGACTGTGCGTTCGCTATGTAGT |
| | | TGCTGCATTTCAAGTTGGGTCGTTACTTCTGTGTTGCAAACCCTTACTGGAGATAATGCCATGTCTGTTGTG |
| | | GAACTTAAAATACGCGAGTGTATAACATTTCTAGATGGTAGAGGTGGTAAACGGCGAGCTAAATGATTAACA |
| | | TCGGGACATATCCTGCCTGCATGAGCATGTGGTGTGTCGTGTGGTGTATATATTGGTAATCTTGTTGTTACA |
| | | TTGTTGAACGACACAAGTCTGCTCTCTCGGTAGAGATAACCCACCAGTACGGCTTGGCCAGTACCTAATAAG |
| | | AAAA |
| ORF75 | 204 | ACATTGCTTTTGGGATCAGACCCCTCATTTAATCGCAT |
| (HHV8 | | |
| gp81) | | |
| ORFK4 | 205 | AGAATGCTTTGCCAGCTGCGCATTTACGCGACGGATCTCTAACGATACCCATGTTGGGTCCACAAGTCTAAG |
| (HHV8 | | GCCAGCGAGACAAGAGCGTTTCGTGAAACGTGCCTGCCAAGGAGTGGGATCTCCCAATTACAGGAGAACAGC |
| gp13) | | GAACGGCGCGGGGTGTCGGAAGGCACAACTCTACTGCACAAAATTGTCTTGTAAA |
| ORFK8 | 206 | ACGGGAAACAGGTGTCTATCTTGGCCGGCTGGTTACTCAAATGGGAACAATGGCGCCACCTTGCTGTCTTTG |
| (Zta) | | TAGGCATTAGAAGAAAAGGATGCACAACTATGTTTCCTAGCGGCGAGATTGGAGGCACATAAGGAACAGATT |
| (HHV8) | | ATTTTCCTTCGCGACATGCTGATGCGAATGTGCCAGCAGCCAGCGTCGCCAACGGACGCGCCACTCCCACCA |
| gp53) | | TGTTGAAGCTTGGTTGTGCCGTCGTCCGGGAGAACCATGCCAGACTTTGTGTGGTAAGAAGGAATTGTTATC |
| | | CGGCAGCAATATTAAAGGGACCCAAGTTAATCCCTTAATCCTCTGGGATTAATAACCATGAGTTCCACACAG |
| | | ATTCGCACAGAAATCCCTGTGGCGCTCCTAATCCTATGCCTTTGTCTGGTGGCGTGCCATGCCAATTGTCCC |
| | | ACGTATCGTTCGCATTTGGGATTCTGGCAAGAGGGTTGGAGTGGACAGGTTTATCAGGACTGGCTAGGCAGG |
| | | ATGAACTGTTCCTACGAGAATATGACGGCCCTAGAGGCCGTCTCCCTAAACGGGACCAGACTAGCAGCTGGA |
| | | TCTCCGTCGAGTGAGTATCCAAATGTCTCCGTATCTGTTGAAGATACGTCTGCCTCTGGGTCTGGAGAAGAT |
| | | GCAATAGATGAATCGGGGTCGGGGGAGGAAGAGCGTCCCGTGACCTCCCACGTGACTTTTATGACACAAAGC |
| | | GTCCAGGCCACCACAGAACTGACCGATGCCTTAATATCAGCCTTTTCAGGTGTATTACACGTTTCAACTGTA |
| | | ATCCCTCGCAATTGGGTAAACCGTCGGTGTGTAGGGATAAAGCGTAACCTTACGTTCTGTCTCATCTACAGG |
| | | ATCATATTCATCTGGGGAACCATCCAGGACCACGCGAATTCGCGTATCACCGGTCGCAGAAAACGGCAGAAA |
| | | TAGTGGTGCTAGTAACCGTGTGCCATTTTCTGCCACCACTACAACGACTAGAGGAAGAGACGCGCACTACAA |
| | | TGCAGAAATACGGACCCATCTTTACATACTATGGGCTGTGGGTTTATTGCTGGGACTTGTCCTTATACTTTA |
| | | CCTGTGCGTTCCACGATGCCGGCGTAAGAAACCCTACATAGTGTAACACAAAACCATAAAAGTA |
| ORFK13 | 207 | ATAACAAGCTGTTGCTAATTTTTGGTCCGTAGAATGTATGTATCTGATTT |
| (HHV8 | | |
| gp76) | | |
| ORFK14 | 208 | CTAGATGGACACCCCGTGAACCGTCGTGCTTACCCACCCCCTTCTGATTCTGACAGACAACACTACTATGTC |
| (HHV8 | | CCAAAGACTGTTTTTTACAGCCCGATGGCCCTTCAGGCCTCCTTGAGTGTCTAGCTGGTCCCGTGGTCATTG |
| gp79) | | TGTGGTTTGGCAGTCACTTCCCCATTTTGGTGTCGCGTTTTGGGTTTTGCCCTGCCCCCAGCCAACGTGGAT |
| | | CATATTCTTTCCCGTCAGGGGAGTGACAAGCTATAGGACAGAAAGGTCACCTGGCCCAAACGGAGGATCCTA |
| | | GGTGGGTGTGCATTTATTAGACGTTGGTGTGTTGAAGGACGGATCAGGCGGGGAGGAGGGGGTGGGGGAGAC |
| | | TTACTGCAGCACTAGGTTAGGTTGAAAGCCGGGGTAAAAGGCGTGGCTAAACAACACCTATACTACTTGTTA |
| | | TTGTAGGCCATGGCGGCCGAGGATTTCCTAACCATCTTCTTAGATGATGATGAATCCTGGAATGAAACTCTA |
| | | AATATGAGCGGATATGACTACTCTGGAAACTTCAGCCTAGAAGTGAGCGTGTGTGAGATGACCACCGTGGTG |
| | | CCTTACACGTGGAACGTTGGAATACTCTCTCTGATTTTCCTCATAAATGTTCTTGGAAATGGATTGGTCACC |
| | | TACATTTTTTGCAAGCACCGATCGCGGGCAGGAGCGATAGATATACTGCTCCTGGGTATCTGCCTAAACTCG |
| | | CTGTGTCTTAGCATATCTCTATTGGCAGAAGTGTTGATGTTTTTGTTTCCCAATATCATCTCCACAGGCTTG |
| | | TGCAGACTTGAAATTTTTTTTTACTATTTATATGTCTACTTGGATATCTTCAGTGTTGTGTGCGTCAGTCTA |
| | | GTGAGGTACCTCCTGGTGGCATATTCTACGCGTTCCTGGCCCAAGAAGCAGTCCCTCGGATGGGTACTGACA |
| | | TCCGCTGCACTGTTAATTGCATTGGTGCTGTCGGGGGATGCCTGTCGACACAGGAGCAGGGTGGTCGACCCG |
| | | GTCAGCAAGCAGGCCATGTGTTATGAGAACGCGGGAAACATGACTGCAGACTGGCGACTGCATGTCAGAACC |
| | | GTGTCAGTTACTGCAGGTTTCCTGTTACCCCTGGCCCTCCTTATTCTGTTTTATGCTCTCACCTGGTGTGTG |
| | | GTGAGGAGGACAAAGCTGCAAGCCAGGCGGAAGGTAAGGGGGGTGATTGTTGCTGTGGTGCTGCTGTTTTTT |
| | | GTGTTTTGCTTCCCTTACCACGTACTAAATCTACTGGACACTCTGCTAAGGCGACGCTGGATCCGGGACAGC |
| | | TGCTATACGCGGGGGTTGATAAACGTGGGTCTGGCAGTAACCTCGTTACTGCAGGCACTGTACAGCGCCGTG |
| | | GTTCCCCTGATATACTCCTGCCTGGGATCCCTCTTTAGGCAGAGGATGTACGGTCTCTTCCAAAGCCTCAGG |
| | | CAGTCTTTCATGTCCGGCGCCACCACGTAGCCCGCGGATGTCTACGTGCCCTTCCCCCTTAATTTAATCTAG |
| | | CCTCCCGTTCCCATGATGCAGAGAGGCGAATTTGGTTTGTACACAGATGTGACTATGTATTTGTTTTATTAT |
| | | GCGATTAAATGAGGGGTCTGATCCCAAAAGCAATGTTTAGTGGTGGTCGTTGATCTTCTTGACGCTCCATAG |
| | | GTAGATTGACTGGAACGCCATGGCCCACGGGGACATGGACAGGGGTGTTAGGTCTGGTGGAACATGCTGCCA |
| | | CTGCCACGGATGGAACATCAGAGATGGGTCTATGATCAGGGCAGCGTGTCGCCCGTCACTGGATGTAAGTCC |
| | | GGCCACCGTGGAGTTGCCTGTGGGGTTTCTGGGATAGTGTCTGGCTGGCAGGGTCTCATCCGCGGCATTTCC |
| | | ATGGTAGGTGAGGGTTATCTCGCCTCGCTGTCTCAGTATGTACTCGAGGGCGTCCTGCTCGTACCGGACCCC |
| | | CAGGTACTCTCCCTGGGCCCAGCTGGGCAGCACCGTCCCCCGCAACACTCGGAGGAAAACGCTCTTAGTGTT |
| | | CTGAGGGATCTGTATGTTTAGCCAGTGGCTGTCATACAGCTTGGACACGTTGGTCTCCAGGTTTACCGCCCA |
| | | GCGCTGGGGTGGTGTGGGTCCGTACGTGTATGGTGAGGATTCCGACCGGCCCACTACACCCAGGGCCACCAG |
| | | CAGCTGGAAGCCCACCTCGCCACAGCAGATGGAGAATGTGTCGGGTCTGTTTAGAAACTCTGTCAGGGTGGA |
| | | GGCACAGGTAGGGTCGTTACACAGCGCCAGGACCCATCCCCTGGCGCTGGCGTAGCTGGCCTGGCAGCCTGT |
| | | TCTGAGACATGTAATCAGACCAGAGAACCCCGACAAGGACTGTCCTCGTTTAAGCTCTTCCACAGTCACCGT |
| | | GGCCACCTCAAAGCCCGTGTTCTGCAACGCGGCCATGAGCGCGTACGGGGCACTGCTCCCAGGCAGCACCAA |
| | | CGCGGCCACACGGCGCGGGGAGGTGGGGCACGAAAACAGGCGCAGCTGACTCCCAAGGCACATGGCCCTTAG |
| | | GCTGCCCAGGTGATGCTCCAGACGACCCAGGTCCTTCCTGTGCATGTCCTCCAGTGGGTGCAGGGGAGGCGT |
| | | CACCAGGTTCCACATTTCGTCAGAAAAGGAGGTCCATGAGACTTGCAAGGAAGTCAGGGTCTCTTGAAACAC |
| | | AACTGTCTCGTTCTGCAAAACCGTGACGTTGTTGCCTTGTCCCTCGGGGCCAACGGTGCCCAGTGGGTGTGC |
| | | CACGCAGCGGTAGTCCCTGGCCGCCCGCAGCACCTCTGACAAGTGTACCTGGGGCACCTCAACCAGTGCCCC |
| | | AGGGGTCTCTGAAACCATAAGTTCGAGCGGGTTAGGGTGGGCGGGTAGTGAGAGCTGCAGTCCCCTGCAGCC |
| | | GGCCAGGGCCATCTCGATTGCAGATGGGAGAAGCCCTCCGTCCCCTATGTCGTGCCCAGATACAATGAGCCT |
| | | CTTGGACATCAGGTACTTAACAAGCATGAACAGGCTGGCGACCGTGGACGGGTTCAGAGGGGGTATTGGGTG |
| | | CCTGGATGCCAGGAAGTTGTGCTCGAAGGTGGACCCGGCTATGAGACAGCTCTGATTCACGGCCAGGTATAC |
| | | CAGGGCGTTGCCTTCGACCTTTACGTCCGGGGTGACCCTGTATCTGGATCCCTTGACCTCGGCCCAGCTGGT |
| | | AAACACCACCGAGTTGAAGGGAAGGACCTCCACCGTTTCTTGCTGTTGTGTGATGCGCACATGGCGCTCCGA |
| | | AAGCGTCGGAGAGCTGGCAGCCGAGGAGATGGACAGTGCCACTCCCAGCTCCCGGCAGAATTCCTTGCAGGC |
| | | GAAGAGGCACTCCTGTAGGAGGCCGGCTTGGTGGTCCTCTGGACTCCACGCCACGGCGCCAGTTAGCACTAC |
| | | GTCCTGGAGCTTGGACACGGGACTGAACATGAGGTTGGTGAGAGCCTCGGTGATGGCATAGGTGGCCCCGGT |
| | | GGATACATTAGTAGCCATCTTGTAGGCCTGCTCCCCCATGGCCATTGCCTGACCCCTCCACGCTGGCACTGG |
| | | AAGCAGCTCCTGGGGCAGGGCCTTCACCCAGGTCTCGAAGTCCTTGTGTAGGAGGTTGGCCATGGACGGAGT |
| | | GATGGCCTCCACCGTGTCGGGCACTCTGGGCGCCACCCTCTCGGCCAGCATGGACGAGTGCAGCACCAGGTG |
| | | GTAGTCTGAAACCGGTATGTCCAGGGGTCCCACGCCAGCCTGTTGGGCGATGAGGCCGTTGGAGCATCGGTC |
| | | CATGTGTCGCGTAAAGAACTCCTTGCTGCCAACCGTCGAGTGGCGAAGTAACTGGTGGATTGTGGAGCCGGT |
| | | GGCAAAAAGGCCCCAGTCAACATCCTCGGGGTGCCCCGAGACGCGGACACCATCGGACAGCGCCAGCCAGGG |
| | | GGACGGGGGGGTGGACGACGGCTGGTCTACAGAGAAGACCCTCGTGGTCTCCCCGGTCAGGTCGTCTACTAT |
| | | TCTGATGCCTGGGTGCTCCGAGGTCCTCCCGAGGACCGTTACCTGGCACGCGCACAGGCGCGCGGCGCGCTG |
| | | CAGTACCTCCAACGGGGTCTCGCCCAGATCCCCAGGCACCGCGCCCGACTCTGCCACCACCGCAAACACCAG |
| | | GGAGCAATACACGTTGAGAAAGTGCTCTGCCACCGCCGCCTTCACGGCATCCGGACCGGCCGCGGGATCCGC |
| | | AGGCAGGTGGGTGCGCACCTCGTCGGGTAGCTTGGAGACAAACAGCTCCAGGCCGGTCCGCGGCGCCAGCGC |
| | | CTGCAGGTGCCTCACCACCGGGGCCGGGTCATGCGATCTGTTTAGTCCGGAGAAGATAGGGCCCTTGGCAAG |
| | | CCGCTGGACCAGCTTCAGGGTCTCCAAGATGCGCACCGCATTGTCGGAGCTGTCGCGATAGAGGTTAGGGTA |
| | | GGTGTCCGGTCCATCCGTGGGCTCAAACCTGCCCAGACACACCACTGTCTGCTGGGGGATCATCCTTCTCAG |
| | | GGAGATGCATTCTTTGGAAGTAGTGGTAGAGATGGAGCAGACTGCCAGGGCGTTGCCAGGAGTGGTGGCGAT |
| | | GGTGCGCACCGTTTTTAAGAAACCCCCCAGGGTGGGGACTCCCGCTCCCTGCAGCATCTCGGCCTGCTGTAC |
| | | GCCCTTGGCGAATATGCGACGGAATCGGCTGTGCGCACGGGGTCCCAGGGCCGGTTCGGTGGCATACAGGCC |
| | | GGTGAGGGCCCCCTGTGTCTGTCCGCCTGGAAACAGGGTGCTGTGAAACAGCAGGTTGCCAAGGCCGCGAAT |
| | | ACCCCTCTGCACGCTGCTGTGGACGTGGGTGTACGCTCCGTGGATCCCGAACGCCTGTCTGGCACAGTTCCA |
| | | GGGCCACCGTTCCATGGTGCATCTTCCCGGTATCACAAAGTACCTGGCCACGTTATAATTGTCCCCGGTTGA |
| | | AGCCTGCACCGCCAGCGGTAGCAGGTCTGCCCCCAGGGATATCATAACAGCCTGCATAATGACATCATCTTC |
| | | AATGTGTGGCCTAGCCACGGGCTGGGGACCCTCGGGCACTTCCAACCCCTCGTACGGTACCAGGTCGGTATT |
| | | TTGTGTAAATGCCCTGATAAACTGAGGTGGGTGTGGTTCTAGCAGGGTCTGTGTGATTTTGGACACCAGGTG |
| | | CCTGCCCACTTCCACTCTAGCCCACTCCTGCAATCCTAGCTCTTGCAGCAGAACTGCAAGCTCTGTTGACAA |
| | | TGTTGTGGGCCGGTGGTGCATGTTTGGCCCGTAGCCAAAGGATACAACACGCTCGCTCCCCCGTGGCACAGA |
| | | CCGCCTGATGACATGGGGATATCCAAGGAGCGGTGACAGCACAGCGAGCACCGTCTGTATTTCCACATCCCG |
| | | TCTCTCTCGCTCCTCCCTCGAAGTGGGAGGTCTTCGGAAAGTTATCCATAGCAGATAGTAGCCTCCGGTGCC |
| | | ACCGGGTACGAGAGTGAGTGTGCCCGTACGGCTTGTATAAAAGTTCACAAAAGCTTCCTCATCCGCGGTGAG |
| | | ATCACTCTCCAACCACAGCCCAGTGACGTCGTAGGCCATGCCTAGAGGGCGCACCGCCCCCGGGGACACCCT |
| | | CTGTAGTCAGGCTGCCGAGAAACCCGCGAGATCTCTGGGGAGTAGGAAGAAACTTAGAATCCCCAAATATGT |
| | | CGCAGTCACAGGTTGTCGGGCAGAGTCTGTTTCCGCTTTCATGGGATCCACAGTTACTTGTAGCCATGTCAC |
| | | TAACCTCAAATACTCAAAAAAAGCTATCGATGGAAAAATGCTGTGGTCCTAGGTTAGTCCGTGGGAAACAAA |
| | | ACTTCCTCATACACTTCATCTGCAGGCTGAAATGGTGGCGGATCCAGACTCCTTACACCACAGTTGCTCACA |
| | | TTAGAGATACCTGATTGGTTAATACAAGCGGACGCACGCGTTGGTGGAGGCGTGTTGTCGCCCAAGATACTA |
| | | GCATAGGTGACTGTGCGTTCGCTATGTAGTTGCTGCATTTCAAGTTGGGTCGTTACTTCTGTGTTGCAAACC |
| | | CTTACTGGAGATAATGCCATGTCTGTTGTGGAACTTAAAATACGCGAGTGTATAACATTTCTAGATGGTAGA |
| | | GGTGGTAAACGGCGAGCTAAATGATTAACATCGGGACATATCCTGCCTGCATGAGCATGTGGTGTGTCGTGT |
| | | GGTGTATATATTGGTAATCTTGTTGTTACATTGTTGAACGACACAAGTCTGCTCTCTCGGTAGAGATAACCC |
| | | ACCAGTACGGCTTGGCCAGTACCTAATAAGAAAA |

Varicella zoster virus

| | | |
|---|---|---|
| ORF16 | 209 | GTGCAACTTTTGCTTATATTTTACATACAAACTTGTGTGTACCATAGATGAACACATTTTTATTTGTTTTGAA |
| | | TTATTAAACTTAAGACATGGCCGTGAATGGTGAAAGAGCTGTCCATGATGAAAACCTGGGTGTGTTAGACAGA |
| | | GAATTAATCCGCGCTCAATCAATCCAAGGATGTGTCGGAAACCCTCAAGAATGTAATTCGTGTGCAATAACCT |
| | | CAGCATCGCGGTTGTTTCTCGTGGGACTACAAGCAAGCGTTATCACGTCCGGGTTAATTTTACAATATCACGT |
| | | CTGCGAAGCTGCCGTCAATGCAACTATTATGGGGTTGATCGTCGTTTCGGGGTTATGGCCAACATCCGTGAAA |
| | | TTTCTACGCACATTAGCAAAATTGGGACGATGTTTGCAGACGGTGGTCGTGTTGGGTTTTGCTGTGTTATGGG |
| | | CGGTTGGTTGCCCAATATCCCGGGATCTTCCATTTGTAGAATTACTGGGAATTTCCATATCC |
| ORF47 | 210 | GCCCCCAGCCAGCCAAAAAAATTGCCCGTGTGGGAGGTCTACAGCACCCTTTTGTAAAAACGGATATTAACAC |
| | | GATTAACGTTGAACACCATTTTATAGACACGCTACAGAAGACATCACCGAACATGGACTGTCGCGGGATGACA |
| | | GCGGGTATTTTTATTCGTTTATCCCACATGTATAAAATTCTAACAACTCTGGAGTCTCCAAATGATGTAACCT |
| | | ACACAACACCCGGTTCTACCAACGCACTGTTCTTTAAGACGTCCACACAGCCTCAGGAGCCGCGTCCGGAAGA |
| | | GTTAGCATCCAAATTAACCCAAGACGACATTAAACGTATTCTATTAACAATAGAATCGGAGACTCGTGGTCAG |
| | | GGCGACAATGCCATTTGGACACTACTCAGACGAAATTTAATCACCGCATCAACTCTTAAATGGAGTGTATCTG |
| | | GACCCGTCATTCCACCTCAGTGGTTTTACCACCATAACACTACAGACACATACGGTGATGCG |
| ORF52 | 211 | CAAAAAAACACGCCGCAACAACCCATCCTTAAAATAAAAGGTTTATTTACTTTACAACCCGTGGTGA |
| ORF55 | 212 | AGCATTGTATAAAAACACGCATGCGGGCTTGCTGTTCTCATTTCTAGGTTTTGTCTTAAATACACCCGCCATG |
| | | AGCATCTCTGGACCCCCAACGACGTTTATTTTATATAGGTTACATGGGGTTAGGCGGGTTCTTCACTGGACTT |
| | | TACCGGATCATGAACAAACACTCTACGCATTTACGGGTGGGTCAAGATCAATGGCGGTGAAGACGGACGCTCG |
| | | ATGTGATACAATGAGCGGTGGTATGATCGTCCTTCAACACACCCATACAGTGACCCTGCTAACCATAGACTGT |
| | | TCTACTGACTTTTCATCATACGCATTTACGCACCGGGATTTCCACTTACAGGACAAACCCCACGCAACATTTG |
| | | CGATGCCGTTTATGTCCTGGGTCGGTTCTGACCCAACATCTCAGCTGTACAGTAATGTGGGGGGGGTACTATC |
| | | CGTAATAACGGAAGATGACCTATCCATGTGTATCTCAATTGTTATATACGGTTTACGGGTAA |
| ORF59 | 213 | CACTCCAATCGACCCTCTTGCGTACCATAATGTTTTCGGAGTTGCCTCCTTCCGTACCGACGGCATTGCTTCA |
| | | ATGGGGTTGGGGATTGCATCGTGGACCGTGTTCGATCCCAAATTTTAAACAGGTAGCCAGCCAACACAGTGTT |
| | | CAGAACGATTTTACAGAAAATAGCGTTGATGCAAATGAAAAATTTCCGATTGGGCACGCGGGCTGTATTGAGA |
| | | AAACCAAAGACGACTATGTACCATTTGATACGTTGTTCATGGTATCATCTATTGACGAACTTGGGCGGAGACA |
| | | ATTAACCGACACCATCCGCCGCAGCTTGGTTATGAACGCCTGTGAAATAACGGTCGCGTGTACGAAAACCGCA |
| | | GCCTTTTCTGGTCGAGGCGTGTCACGACAAAACACGTGACCCTATCTAAAAATAAATTCAATCCATCCAGTC |
| | | ATAAGAGCCTGCAAATGTTTGTGTTGTGTCAAAAAACCCATGCACCCCGTGTCAGAAACCTA |
| ORF61 | 214 | TTTGTTGGGAGGGGGAAGGAAATGCCTTAAACATCCACAGTCTGCTTTATTACCAACTGTATGTAAATTATGA |
| | | TCATTAAACGTGCATTTTAAAAATACCTGAGTGTTGC |
| ORF62 | 215 | CGGAGTCCCCTCCTTTTCTCGTGAGCGCCACTGGCGCGCGGACTGTTTGTTGTTAATAAAAGCGGAACGGTTT |
| | | TTATGAAAAAAGTGT |

| | | |
|---|---|---|
| | SID | |
| miRNA | NO | Representative sequence |

miRNAs:
Herpes simplex virus

| | | |
|---|---|---|
| hsv1-miR-H1 | 216 | UGGAAGGACGGGAAGUGGAAG |
| hsv1-miR-LAT | 217 | UGGCGGCCCGGCCCGGGGCC |
| Epstein Barr virus | | |
| ebv-miR-BART1-3p | 218 | UAGCACCGCUAUCCACUAUGUCU |
| ebv-miR-BART1-5p | 219 | UCUUAGUGGAAGUGACGUGCUGU |
| ebv-miR-BART2 | 220 | UAUUUUCUGCAUUCGCCCUUGC |
| ebv-miR-BART3-3p | 221 | CGCACCACUAGUCACCAGGUGU |
| ebv-miR-BART3-5p | 222 | AACCUAGUGUUAGUGUUGUGCU |
| ebv-miR-BART4 | 223 | GACCUGAUGCUGCUGGUGUGCU |
| ebv-miR-BART5 | 224 | CAAGGUGAAUAUAGCUGCCCAUCG |
| ebv-miR-BART6-3p | 225 | CGGGGAUCGGACUAGCCUUAGA |
| ebv-miR-BART6-5p | 226 | GGUUGGUCCAAUCCAUAGGCUU |
| ebv-miR-BART7 | 227 | CAUCAUAGUCCAGUGUCCAGGG |
| ebv-miR-BART8-3p | 228 | GUCACAAUCUAUGGGGUCGUAG |
| ebv-miR-BARTS-5p | 229 | UACGGUUUCCUAGAUUGUACAG |
| ebv-miR-BART9 | 230 | UAACACUUCAUGGGUCCCGUAG |
| ebv-miR-BART10 | 231 | ACAUAACCAUGGAGUUGGCUGU |
| ebv-miR-BART11-3p | 232 | ACGCACACCAGGCUGACUGCC |
| ebv-miR-BART11-5p | 233 | GACAGUUUGGUGCGCUAGUUGU |
| ebv-miR-BART12 | 234 | UCCUGUGGUGUUUGGUGUGGUUU |
| ebv-miR-BART13 | 235 | UGUAACUUGCCAGGGACGGCUGA |
| ebv-miR-BART14-3p | 236 | UAAAUGCUGCAGUAGUAGGGAU |
| ebv-miR-BART14-5p | 237 | UACCCUACGCUGCCGAUUUACA |
| ebv-miR-BART15 | 238 | AGUGGUUUUGUUUCCUUGAUAG |
| ebv-miR-BART16 | 239 | AUAGAGUGGGUGUGUGCUCUUG |
| ebv-miR-BART17-3p | 240 | UUGUAUGCCUGGUGUCCCCUUA |
| ebv-miR-BART17-5p | 241 | AAGAGGACGCAGGCAUACAAGG |
| ebv-miR-BART18 | 242 | CAAGUUCGCACUUCCUAUACAG |
| ebv-miR-BART19 | 243 | UGUUUUGUUUGCUUGGGAAUGC |
| ebv-miR-BART20-3p | 244 | CAUGAAGGCACAGCCUGUUACC |
| ebv-miR-BART20-5p | 245 | GUAGCAGGCAUGUCUUCAUUCC |
| ebv-miR-BHRF1-1 | 246 | UAACCUGAUCAGCCCCGGAGUU |
| ebv-miR-BHRF1-2* | 247 | AAAUUCUGUUGCAGCAGAUAGC |
| ebv-miR-BHRF1-3 | 248 | UAACGGGAAGUGUGUAAGCACAC |

Human cytomegalovirus

| | | | |
|---|---|---|---|
| hcmv-miR-UL22-1 | 249 | UCACGGGAAGGCUAGUUAGAC | / |
| hcmv-miR-UL22A-1* | 250 | UAACUAGCCUUCCCGUGAGA | |
| hcmv-miR-UL31-1 | 251 | CGGCAUGUUGCGCGCCGUGAU | |
| hcmv-miR-UL36-1 | 252 | UCGUUGAAGACACCUGGAAAGA | |
| hcmv-miR-UL36-1-N | 253 | AGACACCUGGAAAGAGGACGU | |
| hcmv-miR-UL53-1 | 254 | UGCGCGAGACCUGCUCGUUGC | |
| hcmv-miR-UL54-1 | 255 | UGCGCGUCUCGGUGCUCUCGG | |
| hcmv-miR-UL70-3p | 256 | GGGGAUGGGCUGGCGCGCGG | |
| hcmv-miR-UL70-5 | 257 | UGCGUCUCGGCCUCGUCCAGA | |
| hcmv-miR-UL102-1 | 258 | UGGCCAUGUCGUUUCGCGUCG | |
| hcmv-miR-UL102-2 | 259 | UGGCGUCGUCGCUCGGCGGGU | |
| hcmv-miR-UL111a-1 | 260 | UGACGUUGUUUGUGGGUGUUG | |
| hcmv-miR-UL112-1 | 261 | AAGUGACGGUGAGAUCCAGGCU | |
| hcmv-miR-UL148D-1 | 262 | UCGUCCUCCCCUUCUUCACCG | |
| hcmv-miR-US4 | 263 | CGACAUGGACGUGCAGGGGGAU | |
| hcmv-miR-US5-1 | 264 | UGACAAGCCUGACGAGAGCGU | |
| hcmv-miR-US5-2 | 265 | UUAUGAUAGGUGUGACGAUGUC | |
| hcmv-miR-US5-2-N | 266 | UGAUAGGUGUGACGAUGUCUU | |
| hcmv-miR-US25-1 | 267 | AACCGCUCAGUGGCUCGGACC | |
| hcmv-miR-US25-2-5p | 268 | AGCGGUCUGUUCAGGUGGAUGA | |
| hcmv-miR-US25-2-3p | 269 | AUCCACUUGGAGAGCUCCCGCGG | |
| hcmv-miR-US29-1 | 270 | UUGGAUGUGCUCGGACCGUGA | |
| hcmv-miR-US33-1 | 271 | GAUUGUGCCCGGACCGUGGGCG | |

Kaposi's sarcoma-associated hemesvirus

| | | |
|---|---|---|
| kshv-miR-K12-1 | 272 | AUUACAGGAAACUGGGUGUAAGC |
| kshv-miR-K12-2 | 273 | AACUGUAGUCCGGGUCGAUCUG |
| kshv-miR-K12-3 | 274 | UCACAUUCUGAGGACGGCAGCG |
| kshv-miR-K12-3* | 275 | UCGCGGUCACAGAAUGUGACA |
| kshv-miR-K12-4-5 | 276 | AGCUAAACCGCAGUACUCUAGG |
| kshv-miR-K12-4-3p | 277 | UAGAAUACUGAGGCCUAGCUGA |
| kshv-miR-K12-5 | 278 | UAGGAUGCCUGGAACUUGCCGG |
| kshv-miR-K12-6-5p | 279 | CCAGCAGCACCUAAUCCAUCGG |
| kshv-miR-K12-6-3 | 280 | UGAUGGUUUUCGGGCUGUUGAG |
| kshv-miR-K12-7 | 281 | UGAUCCCAUGUUGCUGGCGCU |
| kshv-miR-K12-8 | 282 | UAGGCGCGACUGAGAGAGCACG |
| kshv-miR-K12-9* | 283 | ACCCAGCUGCGUAAACCCCGCU |
| kshv-miR-K12-9 | 284 | CUGGGUAUACGCAGCUGCGUAA |
| kshv-miR-K12-10a | 285 | UAGUGUUGUCCCCCCGAGUGGC |
| kshv-miR-K12-10b | 286 | UGGUGUUGUCCCCCCGAGUGGC |
| kshv-miR-K12-11 | 287 | UUAAUGCUUAGCCUGUGUCCGA |
| kshv-miR-K12-12 | 288 | ACCAGGCCACCAUUCCUCUCCG |

Human (homo sapiens)

| | | |
|---|---|---|
| hsa-let-7a | 289 | UGAGGUAGUAGGUUGUAUAGUU |
| hsa-let-7b | 290 | CUAUACAACCUACUGCCUUCCC |
| hsa-let-7c | 291 | UGAGGUAGUAGGUUGUAUGGUU |
| hsa-let-7d | 292 | AGAGGUAGUAGGUUGCAUAGUU |
| hsa-let-7e | 293 | UGAGGUAGGAGGUUGUAUAGUU |
| hsa-let-7f | 294 | UGAGGUAGUAGAUUGUAUAGUU |
| hsa-let-7g | 295 | UGAGGUAGUAGUUUGUACAGUU |
| hsa-let-7i | 296 | UGAGGUAGUAGUUUGUGCUGUU |
| hsa-miR-1 | 297 | UGGAAUGUAAAGAAGUAUGUAU |
| hsa-miR-9 | 298 | UCUUUGGUUAUCUAGCUGUAUGA |
| hsa-miR-15a | 299 | CAGGCCAUAUUGUGCUGCCUCA |
| hsa-miR-15b | 300 | CGAAUCAUUAUUUGCUGCUCUA |
| hsa-miR-16 | 301 | UAGCAGCACGUAAAUAUUGGCG |
| hsa-miR-17 | 302 | CAAAGUGCUUACAGUGCAGGUAG |
| hsa-miR-17-5p | 303 | CAAAGUGCUUACAGUGCAGGUAGU |
| hsa-miR-18a | 304 | UAAGGUGCAUCUAGUGCAGAUAG |
| hsa-miR-18b | 305 | UAAGGUGCAUCUAGUGCAGAUAG |
| hsa-miR-20a | 306 | ACUGCAUUAUGAGCACUUAAAG |
| hsa-miR-20b | 307 | CAAAGUGCUCAUAGUGCAGGUAG |
| hsa-miR-23a | 308 | AUCACAUUGCCAGGGAUUUCC |
| hsa-miR-23b | 309 | AUCACAUUGCCAGGGAUUACC |
| hsa-miR-24 | 310 | UGGCUCAGUUCAGCAGGAACAG |
| hsa-miR-30a-5p | 311 | UGUAAACAUCCUCGACUGGAAG |
| hsa-miR-30a-3 | 312 | CUUUCAGUCGGAUGUUUGCAGC |
| hsa-miR-30b | 313 | CUGGGAGGUGGAUGUUUACUUC |
| hsa-miR-30c | 314 | UGUAAACAUCCUACACUCUCAGC |
| hsa-miR-30e-5p | 315 | UGUAAACAUCCUUGACUGGA |
| hsa-miR-30e-3p | 316 | CUUUCAGUCGGAUGUUUACAGC |
| hsa-miR-93 | 317 | CAAAGUGCUGUUCGUGCAGGUAG |
| hsa-miR-98 | 318 | UGAGGUAGUAAGUUGUAUUGUU |
| hsa-miR-99a | 319 | AACCCGUAGAUCCGAUCUUGUG |
| hsa-miR-99b | 320 | CACCCGUAGAACCGACCUUGCG |
| hsa-miR-100 | 321 | AACCCGUAGAUCCGAACUUGUG |
| hsa-miR-103 | 322 | AGCAGCAUUGUACAGGGCUAUGA |
| hsa-miR-105 | 323 | UCAAAUGCUCAGACUCCUGUGGU |
| hsa-miR-106a | 324 | AAAAGUGCUUACAGUGCAGGUAG |
| hsa-miR-106b | 325 | UAAAGUGCUGACAGUGCAGAU |
| hsa-miR-107 | 326 | AGCAGCAUUGUACAGGGCUAUCA |
| hsa-miR-124a | 327 | UUAAGGCACGCGGUGAAUGCCA |
| hsa-miR-125a | 328 | ACAGGUGAGGUUCUUGGGAGCC |
| hsa-miR-125b | 329 | UCCCUGAGACCCUAACUUGUGA |
| hsa-miR-126 | 330 | UCGUACCGUGAGUAAUAAUGCG |
| hsa-miR-129 | 331 | CUUUUUGCGGUCUGGGCUUGC |
| hsa-miR-132 | 332 | UAACAGUCUACAGCCAUGGUCG |
| hsa-miR-134 | 333 | UGUGACUGGUUGACCAGAGGGG |
| hsa-miR-137 | 334 | UUAUUGCUUAAGAAUACGCGUAG |
| hsa-miR-138 | 335 | AGCUGGUGUUGUGAAUCAGGCCG |
| hsa-miR-141 | 336 | UAACACUGUCUGGUAAAGAUGG |
| hsa-miR-142-3p | 337 | UGUAGUGUUUCCUACUUUAUGGA |
| hsa-miR-142-5p | 338 | CAUAAAGUAGAAAGCACUACU |
| hsa-miR-145 | 339 | GUCCAGUUUUCCCAGGAAUCCCU |
| hsa-miR-150 | 340 | UCUCCCAACCCUUGUACCAGUG |
| hsa-miR-154 | 341 | UAGGUUAUCCGUGUUGCCUUCG |
| hsa-miR-181a | 342 | AACAUUCAACGCUGUCGGUGAGU |
| hsa-miR-181b | 343 | AACAUUCAUUGCUGUCGGUGGGU |
| hsa-miR-181c | 344 | AACAUUCAACCUGUCGGUGAGU |
| hsa-miR-181d | 345 | AACAUUCAUUGUUGUCGGUGGGU |
| hsa-miR-182* | 346 | UGGUUCUAGACUUGCCAACUA |
| hsa-miR-184 | 347 | UGGACGGAGAACUGAUAAGGGU |
| hsa-miR-194 | 348 | UGUAACAGCAACUCCAUGUGGA |
| hsa-miR-195 | 349 | UAGCAGCACAGAAAUAUUGGC |
| hsa-miR-196a | 350 | UAGGUAGUUUCAUGUUGUUGGG |
| hsa-miR-196b | 351 | UAGGUAGUUUCCUGUUGUUGGG |
| hsa-miR-197 | 352 | UUCACCACCUUCUCCACCCAGC |
| hsa-miR-199a | 353 | CCCAGUGUUCAGACUACCUGUUC |
| hsa-miR-199b | 354 | CCCAGUGUUUAGACUAUCUGUUC |
| hsa-miR-200a | 355 | UAACACUGUCUGGUAACGAUGU |
| hsa-miR-200b | 356 | UAAUACUGCCUGGUAAUGAUGA |
| hsa-miR-200c | 357 | UAAUACUGCCGGGUAAUGAUGGA |
| hsa-miR-202 | 358 | GUGCCAGCUGCAGUGGGGGAG |
| hsa-miR-205 | 359 | UCCUUCAUUCCACCGGAGUCUG |
| hsa-miR-206 | 360 | UGGAAUGUAAGGAAGUGUGUGG |
| hsa-miR-210 | 361 | CUGUGCGUGUGACAGCGGCUGA |
| hsa-miR-212 | 362 | UAACAGUCUCCAGUCACGGCC |
| hsa-miR-213 | 363 | ACCAUCGACCGUUGAUUGUACC |
| hsa-miR-214 | 364 | ACAGCAGGCACAGACAGGCAGU |
| hsa-miR-219 | 365 | AGGGUAAGCUGAACCUCUGAU |
| hsa-miR-296 | 366 | AGGGCCCCCCCUCAAUCCUGU |
| hsa-miR-299-3p | 367 | UAUGUGGGAUGGUAAACCGCUU |
| hsa-miR-302a | 368 | UAAGUGCUUCCAUGUUUUGGUGA |
| hsa-miR-302b | 369 | UAAGUGCUUCCAUGUUUUAGUAG |
| hsa-miR-302c | 370 | UAAGUGCUUCCAUGUUUCAGUGG |
| hsa-miR-302d | 371 | UAAGUGCUUCCAUGUUUGAGUGU |
| hsa-miR-324-3p | 372 | ACUGCCCCAGGUGCUGCUGG |
| hsa-miR-326 | 373 | CCUCUGGGCCCUUCCUCCAG |
| hsa-miR-328 | 374 | CUGGCCCUCUCUGCCCUUCCGU |
| hsa-miR-329 | 375 | AACACACCUGGUUAACCUCUUU |
| hsa-miR-330-5p | 376 | UCUCUGGGCCUGUGUCUUAGGC |
| hsa-miR-330 (-3p) | 377 | GCAAAGCACACGGCCUGCAGAGA |
| hsa-miR-337 (-3p) | 378 | UCCAGCUCCUAUAUGAUGCCUUU |
| hsa-miR-338 (-3p) | 379 | UCCAGCAUCAGUGAUUUUGUUGA |
| hsa-miR-339 (-5p) | 380 | UCCCUGUCCUCCAGGAGCUCA |
| hsa-miR-340 | 381 | UUAUAAAGCAAUGAGACUGAUU |
| hsa-miR-346 | 382 | UGUCUGCCCGCAUGCCUGCCUCU |
| hsa-miR-367 | 383 | AAUUGCACUUUAGCAAUGGUGA |
| hsa-miR-371 (-3p) | 384 | GUGCCGCCAUCUUUUGAGUGU |
| hsa-miR-372 | 385 | AAAGUGCUGCGACAUUUGAGCGU |
| hsa-miR-373 | 386 | GAAGUGCUUCGAUUUUGGGGUGU |
| hsa-miR-374 | 387 | UUAUAAUACAACCUGAUAAGUG |
| (same as 374a) | | |
| hsa-miR-381 | 388 | UAUACAAGGGCAAGCUCUCUGU |
| hsa-miR-424 | 389 | CAGCAGCAAUUCAUGUUUUGAA |
| hsa-miR-425 | 390 | AAUGACACGAUCACUCCCGUUGA |
| hsa-miR-429 | 391 | UAAUACUGUCUGGUAAAACCGU |
| hsa-miR-448 | 392 | UUGCAUAUGUAGGAUGUCCCAU |
| hsa-miR-450 | 393 | UUUUGCAAUAUGUUCCUGAAUA |
| (same as 450b-5p) | | |
| hsa-miR-450b-3p | 394 | UUGGGAUCAUUUUGCAUCCAUA |
| hsa-miR-451 | 395 | AAACCGUUACCAUUACUGAGUU |
| hsa-miR-453 | 396 | AGGUUGUCCGUGGUGAGUUCGCA |
| hsa-miR-455 (-5p) | 397 | UAUGUGCCUUUGGACUACAUCG |
| hsa-miR-490 (-3p) | 398 | CAACCUGGAGGACUCCAUGCUG |
| hsa-miR-491 (-5p) | 399 | AGUGGGGAACCCUUCCAUGAGGA |
| hsa-miR-492 | 400 | AGGACCUGCGGGACAAGAUUCUU |
| hsa-miR-495 | 401 | AAACAAACAUGGUGCACUUCUU |
| hsa-miR-497 | 402 | CAGCAGCACACUGUGGUUUGU |
| hsa-miR-502 (-5p) | 403 | AUCCUUGCUAUCUGGGUGCUA |
| hsa-miR-503 | 404 | UAGCAGCGGGAACAGUUCUGCAG |
| hsa-miR-510 | 405 | UACUCAGGAGAGUGGCAAUCAC |
| hsa-miR-518b | 406 | CAAAGCGCUCCCCUUUAGAGGU |
| hsa-miR-518c | 407 | CAAAGCGCUUCUCUUUAGAGUGU |
| hsa-miR-518d | 408 | CAAAGCGCUUCCCUUUGGAGC |
| hsa-miR-519d | 409 | CAAAGUGCCUCCCUUUAGAGUG |
| hsa-miR-520a* | 410 | CUCCAGAGGGAAGUACUUUCU |
| (same as 520a-5p) | | |
| hsa-miR-520b | 411 | AAAGUGCUUCCUUUUAGAGGG |
| hsa-miR-520c | 412 | AAAGUGCUUCCUUUUAGAGGGU |
| (same as 520c-3p) | | |
| hsa-miR-520d | 413 | AAAGUGCUUCUCUUUGGUGGGUU |
| (same as 520d-3p) | | |
| hsa-miR-520g | 414 | ACAAAGUGCUUCCCUUUAGAGUGU |
| hsa-miR-520h | 415 | ACAAAGUGCUUCCCUUUAGAGU |
| hsa-miR-522 | 416 | AAAAUGGUUCCCUUUAGAGUGU |
| hsa-miR-525 (-5p) | 417 | CUCCAGAGGGAUGCACUUUCU |
| hsa-miR-526b | 418 | CUCUUGAGGGAAGCACUUUCUGU |
| hsa-548d-3p | 419 | CAAAAACCACAGUUUCUUUUGC |
| hsa-miR-548k | 420 | AAAAGUACUUGCGGAUUUUGCU |
| hsa-miR-551a | 421 | GCGACCCACUCUUGGUUUCCA |
| hsa-miR-551b | 422 | GCGACCCAUACUUGGUUUCAG |
| hsa-miR-552 | 423 | AACAGGUGACUGGUUAGACAA |
| hsa-miR-592 | 424 | UUGUGUCAAUAUGCGAUGAUGU |
| hsa-miR-598 | 425 | UACGUCAUCGUUGUCAUCGUCA |
| hsa-miR-652 | 426 | AAUGGCGCCACUAGGGUUGUG |
| hsa-miR-769-3p | 427 | CUGGGAUCUCCGGGGUCUUGGUU |
| hsa-miR-1226 | 428 | UCACCAGCCCUGUGUUCCCUAG |

### Example 2

### Suppression of Immediate-Early Viral Gene Expression by Herpesvirus-Coded MicroRNAs

As described above, a quantitative algorithm was developed and applied to predict target genes of microRNAs encoded by herpesviruses. While there is almost no conservation among microRNAs of different herpesvirus subfamilies, a common pattern of regulation emerged. The algorithm predicts that herpes simplex virus, human cytomegalovirus, Epstein-Barr virus, Kaposi's sarcoma-associated herpesvirus and varicella zoster virus all employ microRNAs to suppress expression of their own genes, including their immediate-early genes.

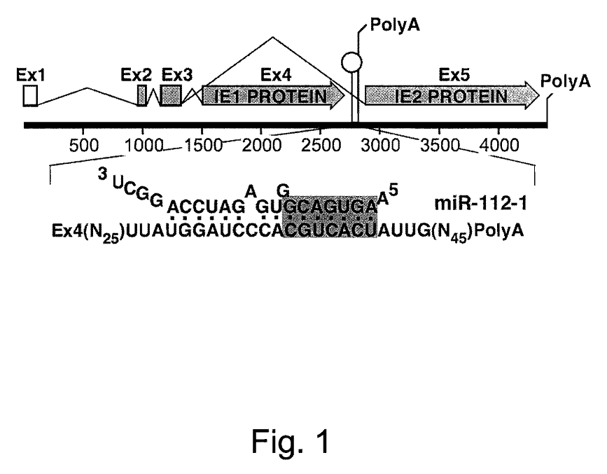

In the case of human cytomegalovirus, a virus-coded microRNA, (miR-UL112-1) that is predicted by the algorithm described herein was predicted to target the viral immediate-early protein 1 (IE1) mRNA within its 3′UTR (FIG. 1). The HCMV IE1 mRNA is an immediate-early product that is expressed from the major immediate-early locus at the very start of infection. The IE1 protein is multifunctional and is involved in transcriptional activation of the viral genome, in part by influencing cellular histone deacetylase activity. It is not essential for lytic virus growth, but mutations within this open reading frame significantly delay virus replication and reduce virus yield.

This example describes experiments designed to test that prediction. Mutant viruses were generated that were unable to express the microRNA, or encoded an immediate-early 1 mRNA lacking its target site. Analysis of RNA and protein within infected cells demonstrated that miR-UL112-1 inhibits expression of the major immediate-early protein.

Materials and Methods:

Cells, viruses and Plasmids. MRC5 and HEK293T cells were propagated in medium with 10% fetal bovine serum or 10% newborn calf serum, respectively.

The wild-type virus used in these studies is BFXwt-GFP. It is a derivative of a bacterial artificial chromosome (BAC) clone of the HCMV VR1814 clinical isolate in which a green fluorescent protein (GFP) expression cassette has been inserted upstream of the US7 ORF. Three derivatives of BFXwt-GFP were produced by using galK selection and counter selection to modify BAC DNAs. BFXdlIE1cis⁻ lacks the 7-nucleotide seed sequence for miR-112-1 within the IE1 3′UTR, BFXsub112-1⁻ contains 12 single base-pair substitutions that block expression of miR-112-1, BFXsub112-1r is a repaired derivative of BFXsub12-1⁻. Virus was generated by electroporation of MRC5 cells with BAC DNA (20 μg) plus an HCMV pp71-expressing plasmid (pCGNpp71). Virions were purified by centrifugation through a 20% sorbitol cushion. Virus titers were calculated by infecting fibroblasts and counting IE2-positive foci at 24 hours post-inoculation (hpi).

mRNA and miRNA quantification. Real-time RT-PCR was performed on total RNA isolated from the cells using the mirVana miRNA isolation kit (Ambion Inc, Austin, Tex.), which isolates total RNA while preserving the miRNA population. DNA was removed by using the DNA-free reagent kit (Ambion Inc). Equal aliquots of total RNA were reverse transcribed using the Taqman Reverse Transcription kit with random hexamers according to the manufacture's protocol (Applied Biosystems, Foster City, Calif.). To measure mRNA levels, real-time PCR was performed with SYBR green PCR master mix (Applied Biosystems) and primers specific to exon 4 of IE1.

To measure levels of miR-UL112-1, a modified TaqMan-based stem loop RT-PCR reaction was performed. TaqMan MicroRNA Reverse Transcription Kit (Applied Biosystems) was used according to the manufacturer's protocol with stem-loop oligonucleotide: 5′GTCGTATCCAGTGCAGGGTCCGAGGTATTCGCACTGGATACGACAGCCTG-3′ (SEQ ID NO: 429). A 1:15 dilution of the product from the reverse transcriptase reaction was used in a TaqMan quantitative PCR reaction along with 1.5 mM of forward primer, 0.7 mM of reverse primer, 0.2 mM of TaqMan probe, and 1× Universal TaqMan PCR Master mix (Applied Biosystems). The results were normalized by quantifying the levels of human U6B small nuclear RNA using the RNU6B Taqman control assay (Applied Biosystems).

Protein quantification. MRC5 cells were infected at a multiplicity of 3 pfu/cell. Cells were starved for methionine and cystine prior to labeling by incubating for 1 h in medium with 10% dialyzed fetal bovine serum. EasyTag Express Protein Labeling Mix (100 μCi; Perkin Elmer, Waltham, Mass.) was added to the cells for 1 h after which the labeling medium was replaced with medium containing 10% fetal calf serum for 10 min to allow stalled translation to complete. Cells were washed in PBS and then lysed in buffer containing 20 mM Tris Acetate pH 7.5, 0.27 M sucrose, 1 mM EDTA, 1 mM EGTA, 1 mM sodium orthovanadate, 10 mM sodium β-glycerophosphate, 50 mM sodium fluoride, 5 mM sodium pyrophosphate and 1% Triton X-100. One tablet of Complete Mini Protease inhibitor (Roche Applied Science) was added per 10 ml lysis buffer. Protein concentration was calculated by Bradford assay.

Aliquots (10 μg) were subjected to western blot assay using monoclonal antibodies specific for HCMV IE1 (1B12), HCMV UL99 (10B4) and monoclonal anti-tubulin antibody (Sigma-Aldrich St. Louis, Mo.). An anti-mouse HRP conjugated antibody was used along with the ECL plus detection kit (Amersham) to detect specific bands. Chemiluminescence was analyzed using a phosphorimager and ImageQuant TL software (GE Healthcare Life Sciences, Piscataway, N.J.).

For immunoprecipitation assays, aliquots of lysate (5 or 10 μg protein) were pre-cleared with Protein A/G Plus Agarose beads (Santa Cruz Biotechnology, Santa Cruz, Calif.) for 4 h at 4° C. Anti-IE1 monoclonal antibody (1B12) and Protein A/G Plus Agarose were added to the supernatant which was incubated overnight at 4° C. with shaking. Immunopreciptated complexes were washed three times with RIPA buffer (50 mM Tris-HCl pH7.4, 1% NP-40, 0.25% Na-deoxycholate, 150 mM NaCl, 1 mM EDTA) supplemented with Complete Mini Protease inhibitor (Roche). Beads were boiled in 2×SDS loading buffer and run on an 8% SDS-PAGE gel to separate the immunoprecipated complexes. Gels were dried and exposed to a phosphor screen, which was analyzed using a phosphorimager and ImageQuant TL software.

Results:

HCMV IE1 protein synthesis is suppressed by miR-UL-112-1. Inhibition of any of the genes in Table 7 of Example 1 could potentially favor latency, but we considered IE1 to be a prime target, given its central role at the start of the HCMV transcriptional cascade. IE1 is one of two main products of the HCMV major IE locus, the other being IE2. IE1 and IE2 are required to execute the transcriptional program of the virus, and they almost certainly influence the choice between latency and lytic replication. A mutant virus unable to produce a functional IE1 protein replicates efficiently only after infection at a high input multiplicity; at lower multiplicities it fails to accumulate normal levels of early mRNAs. It activates transcription at least in part by controlling histone modifications.

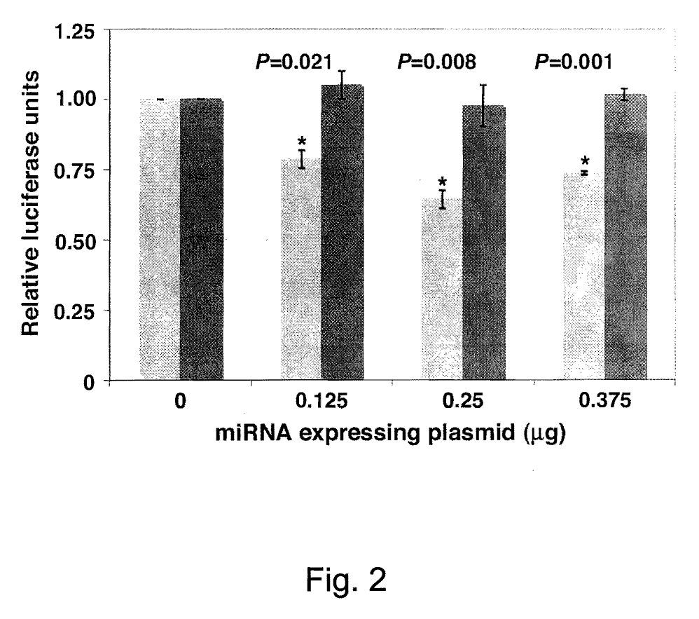

The algorithm predicted a single binding site for miR-UL112-1 within the 99 nucleotide 3′UTR of the IE1 mRNA. To test the prediction that miR-UL12-1 inhibits translation of IE1 protein, we prepared two reporter constructs. The first contained the wild-type IE1 3′UTR downstream of the luciferase coding region and the second contained a derivative of the 3′UTR lacking the 7-nucleotide seed sequence predicted to be the target of the miRNA (FIG. 1, shaded sequence). HEK293T cells were cotransfected with set amounts of the reporter plasmids and increasing amounts of an effector plasmid expressing the miR-UL112-1 precursor hairpin sequence. The miRNA induced a statistically significant reduction in luciferase expression from the reporter with a wild-type IE1 3′UTR (maximum repression=60%) but not from the modified 3′UTR lacking the seed sequence (FIG. 2), arguing that miR-UL112-1 targets the seed sequence within the IE1 3′UTR to reduce translation or degrade the RNA.

Next, three viruses were generated to test whether miR-UL112-1 targets IE1 expression within an HCMV-infected cell. The first, BFXdlIE1cis⁻, lacks the 7-nucleotide seed sequence within the IE1 3′UTR that is targeted by the miRNA. The second, BFXsub112-1⁻, is unable to express the miRNA. The miR-UL112-1 precursor is encoded on the DNA strand opposite UL114, and disruption of this ORF inhibits virus replication. Consequently, we substituted 12 nucleotides within the miR-UL112-1 precursor sequence while maintaining the coding sequence of the UL114 ORF. The miR-UL112-1 mutation was repaired in the final virus, BFXsub112-1r, to control for potential off-target mutations. The viruses grew normally in fibroblasts. We also monitored accumulation of miR-UL112-1 by quantitative RT-PCR. The miRNA accumulated to a detectable level between 8-12 h after infection with wild-type virus and then increased as the infection progressed. No miR-UL112-1 was detected at 48 h after infection with BFXsub12-1⁻, a time at which the miRNA was readily detected in cells infected with the other viruses.

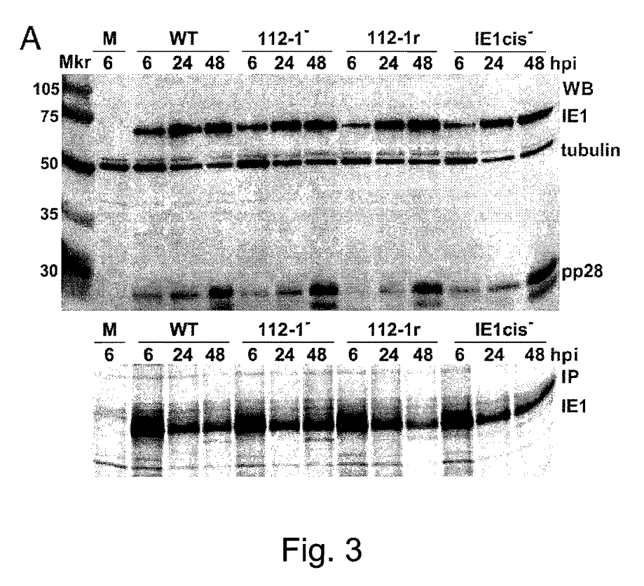

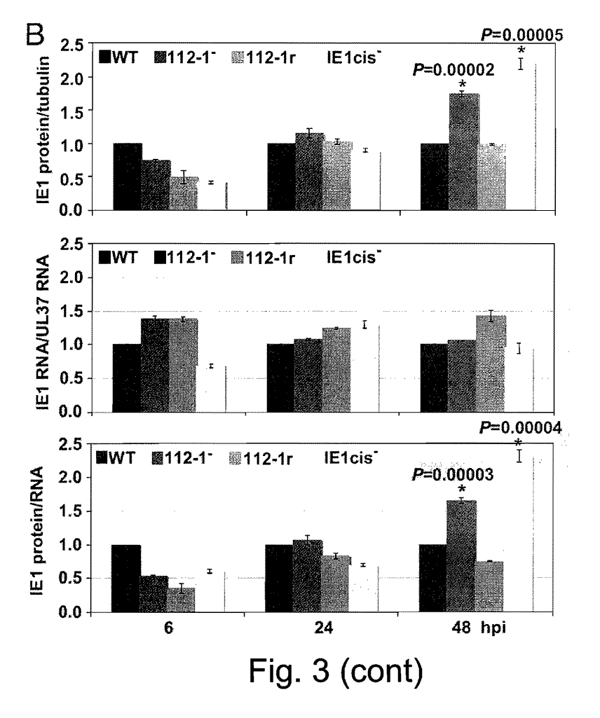

To determine if IE1 protein levels were affected by the expression of miR-UL112-1, we prepared extracts from infected cells after a 1 h ³⁵S-labeling period at 6, 24 and 48 hpi with wild-type or mutant viruses. We did not monitor cells later than 48 hpi, even though the miRNA accumulated to higher levels at 72 hpi, because infected cells show severe cytopathic effect at the later time. We first examined the steady state levels of several proteins by western blot assay (FIG. 3A, top panel). Tubulin levels, which are not altered by infection, provided a precise measure of the amount of cellular protein analyzed in each sample; and the accumulation of the late HCMV protein, pp28, confirmed that all infections progressed normally. We monitored IE1 steady state levels, but little difference was evident after infection with wild-type and mutant viruses. This was presumably because IE1 protein has a>20 h half life, and it accumulates to a high level before the miRNA is available.

Next, IE1 was immunoprecipitated from extracts and subjected to electrophoresis to identify protein synthesized during each 1 h labeling period (FIG. 3A, bottom panel). The rate of IE1 synthesis was substantially greater at 6 hpi than at later times for all viruses, probably because the promoter responsible for the production of IE1 mRNA is repressed late after infection. Radioactivity in the IE1-specific band was quantified relative to the level of tubulin, and FIG. 3B (top panel) presents the results of two independent experiments, each analyzed by performing three independent immunoprecipitations. At 6 and 24 hpi, we did not observe an effect attributable to miR-UL112-1 activity, consistent with the observation that the miRNA is not detected at 6 hpi and relatively little is present at 24 hpi. In contrast, at 48 hpi when the miRNA has accumulated to higher levels, the miR-UL112-1-deficient and the IE1 target site-deficient mutants exhibited statistically significant increases (˜2-fold) in IE1 protein synthesis relative to the wild-type and revertant viruses.

At each time protein extracts were prepared, total RNA was isolated from a duplicate sample, and the amount of IE1 RNA was determined relative to the level of an independent IE RNA (UL37) by quantitative RT-PCR. IE1 RNA levels varied little among the viruses (FIG. 3B, middle panel), indicating that the miRNA does not significantly alter the stability of IE1 mRNA and supporting the conclusion that the changes in IE1 protein levels result from the inhibition of translation. The ratio of IE1 protein to RNA was calculated (FIG. 3B, bottom panel), confirming a significant increase in protein synthesis when either the miRNA or its target site is disrupted.

Summary:

The experiments described above confirmed the predicted inhibition of HCMV IE1 translation by miR-UL112-1 within transfected cells by using reporter constructs (FIG. 2) and within virus-infected fibroblasts by analyzing mutant viruses (FIG. 3). Given the broad range of predicted targets (see Example 1), it is believed that herpesvirus-coded miRNAs exert regulatory effects directly on viral gene expression during replication and spread within infected hosts. This regulation could have many consequences, e.g., downregulating viral genes as the infectious cycle progresses to avoid toxicity and helping to modulate viral gene expression to optimize replication in a variety of different cell types. The results also suggest that virus-coded miRNAs could play a central role in the establishment and maintenance of latency. Because they target E products that act at the top of the lytic cascade, miRNAs expressed in cells destined for a latent infection can potentially antagonize the cascade and thereby favor entry into latency. Further, miRNAs expressed during latency could help to prevent reactivation by inhibiting translation of IE transactivators.

### Example 3

### HCMV IE2 mRNA is Targeted by a Cell-Coded miRNA

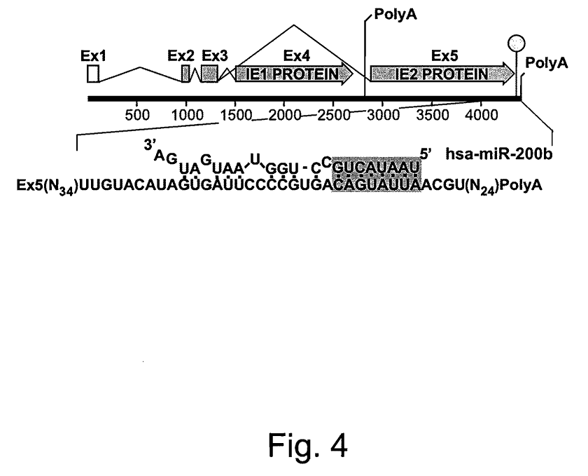

The HCMV genome encodes a second protein, the UL122-coded IE2 protein, whose mRNA is generated by an alternative splicing event within the major immediate-early locus (FIG. 4). The IE2 mRNA lacks the fourth exon that is present in the IE1 mRNA and incorporates an alternative fifth exon. The IE2 protein is multifunctional and is believed to be involved in transcriptional activation of both viral and cellular genes. It has been reported to be an essential protein, as mutations within this open reading frame render the virus defective for growth. It is believed that the expression of the IE2 protein is very important for reactivation of viral transcription from latency.

The algorithm described above predicted that the 3′UTR of the IE2 mRNA contains a site that would be a target of three related but different human-encoded miRNAs: hsa-miR-200b, hsa-miR-200c and hsa-miR-429. The algorithm predicted that any one of these three miRNAs would bind to the 3′UTR of the IE2 mRNA and inhibit its translation. As hsa-miR-200b, hsa-miR-200c and hsa-miR-429 all share a common seed sequence, the binding of has-200b is shown as a representative sample of the interaction between the miRNA and the 3′UTR if IE2 (FIG. 4). According to the algorithm's prediction, the presence of these miRNAs should inhibit viral replication, and, as a result, these miRNAs might be present at reduced levels or not at all in cells where HCMV replicates most efficiently, e.g., fibroblasts.

This example describes experiments which are designed to test the prediction that human encoded miRNAs are able to target viral encoded mRNAs and that this targeting results in the reduced expression level of the subsequent gene product. Assays were performed which allow for the quantification of gene expression in the presence of targeting miRNAs. Additionally, mutants were generated which tests the hypothesis that the miRNAs are targeting through sequences directly predicted by the algorithm.

Materials and Methods:

Cells and Plasmids. 4T07 cells were propagated in DMEM medium with 10% fetal bovine serum. miRNA expressing retroviruses were constructed by cloning cluster 1 into pMSCV/puro (Clontech; Mountain View, Calif.). Cluster 1 contains hsa-miR-200b. Cluster 2 which contains hsa-miR-200c was PCR amplified and cloned into pMSCV/hygro (Clontech). Retroviruses were generated by transiently transfecting 10 ug of the above retrovirus plasmids into the Phoenix Retrovirus Expression System cells (Orbigen; San Diego, Calif.) for 48 hours. Supernatants from transfected cells were filtered through a 0.45μ filter and used to infect 4T07 cells. As a control, 4T07 cells were also transduced with the empty parental retroviruses that lack either cluster 1 or cluster 2. Transduced cells were selected with Hygromycin (300 ug/ml) and Puromycin (4 ug/ml) for three rounds of selection.

The pMIR-Report plasmid was digested with SpeI and HindIII to allow for the insertion of both wild type and mutant IE2 3′UTR sequence. The mutant IE2 3′UTR was generated by GalK recombination utilizing galK insertion primers. Removal of the galK gene from the 3′UTR of IE2 by homologous recombination to introduce a mutant miRNA binding site was directed using a double stranded DNA oligonucleotide. The he 3′UTRs were amplified for cloning into the pMIR-Report vectors. All constructs were confirmed by sequencing.

miRNA quantification: The levels of miRNA expression were measured using the TaqMan microRNA assay stem (applied Biosystems) from total RNA isolated from 10e6 cells using the mirVana miRNA isolation kit (Ambion). Normalization for the hsa-miR-200b and hsa-miR200c was performed by normalization to the endogenous small nucleolar RNA RNU44.

Transfection assays. 4T07 or 4T07/C1C2 cells were transfected with 250 ng of either pMIR-Report (empty vector), pMIR-Report with a wild type IE2 3′UTR (IE2 3′UTR), pMIR-Report with a mutant IE2 3′UTR (Mutant IE2 3′UTR), or pMIR-Report with an anti-sense miR-200b binding site (mir-200b pos control). Cells were also transfected with a Renilla luciferase containing plasmid (pCMV-Ren) as a transfection efficiency control and a protein isolation control. Transfections were performed using the Fugene 6 transfection reagent (Roche) and transfected cells were incubated at 37° C. for 48 hours. Both Firefly and Renilla luciferase quantities were measured utilizing the Dual Luciferase Reporter Assay System (Promega).

Results:

The 3′UTR of IE2 is targeted by hsa-miR200b and hsa-mir200c. To investigate if the miRNAs are present in cells that are permissive for efficient HCMV replication, a miRNA microarray assay was performed. Total RNA was isolated from MRC5 cells (highly permissive embryonic lung fibroblasts) that were either mock-infected or infected with a multiplicity of infection of 3 viruses per cell with HCMV for 24 hours. The RNA was fluorescently labeled utilizing a commercially available end labeling ligation reaction kit (Ambion; Santa Clara, Calif.). Human miRNA Oligo microarrays which contain all the 723 human and the 76 viral miRNAs within the Sanger miRNA database release 10.1 (Ambion) were utilized to screen for miRNA expression within the permissive MRC5 cells. Hybridization and subsequent scanning were performed using standard techniques. The three miRNAs that target the 3′UTR of IE2 are not expressed in the permissive MRC5 cells at a detectable level, as predicted.

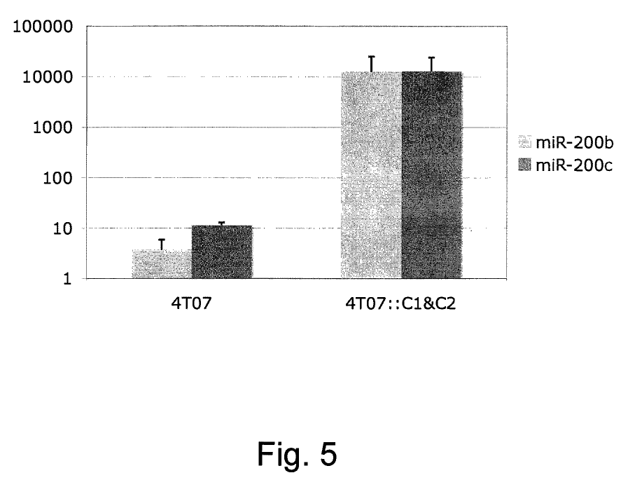

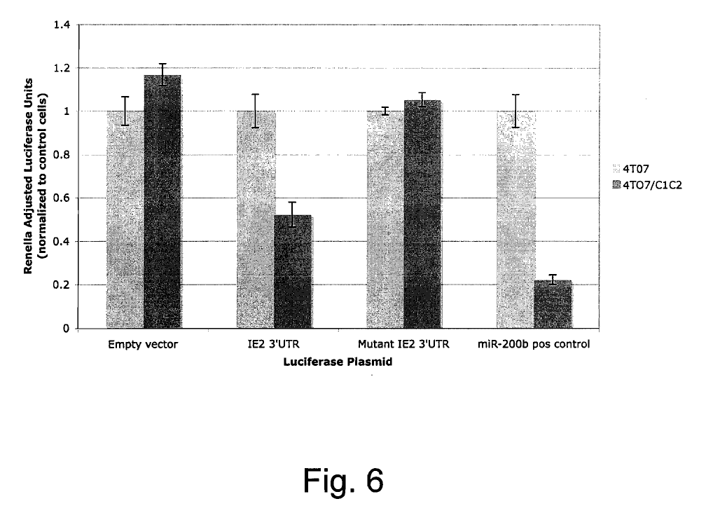

To determine if the human cell-coded miRNAs can repress expression of a transcript containing the HCMV IE2 3′UTR, a firefly luciferase reporter system was utilized. The 3′UTR of IE2 was cloned downstream from a reporter plasmid (pMIR-Report) where the HCMV major immediate-early promoter controls the firefly luciferase open reading frame expression. Additionally, a mutated 3′UTR of IE2 where four nucleotides within the predicted seed sequence are changed to four cistines was cloned into the same reporter vector. As a positive control, a 3′UTR containing a sequence complementary to hsa-miR-200b was utilized in the transfections. Transient transfection assays were performed using a mouse carcinoma cell line (4T07) that has been reported to express hsa-miR-200b, hsa-miR-200c and hsa-miR-429 to low levels. Transduction of 4T07 cells with retroviruses which express hsa-miR-200b and hsa-miR-200c (4T07/C1C2) significantly increases the expression of the miRNAs>1000 fold (FIG. 5) as determined by real time PCR. These cells were transiently transfected with the above-mentioned plasmids to assay miRNA-mediated repression of the reporter genes. After 48 hours, lysates were collected and assayed for luciferase activity (as well as Renilla luciferase activity as a transfection control). Transient transfections of these cells with either an empty reporter or with the mutated 3′UTR of IE2 in the presence of high hsa-miR200b and hsa-miR200c showed no repression in the reporter gene when compared to the control cells (FIG. 6). However, the wild type 3′UTR of IE2 demonstrated a 50% repression compared to the control cells. The positive control plasmid demonstrated nearly a 5-fold reduction in the levels of the reporter gene confirming the ability of the miRNAs to repress a known target (FIG. 6). The level of repression with the wild type IE2 3′UTR is similar to that which has been previously reported for luciferase-based miRNA assay systems, thereby demonstrating that the human miRNAs target the 3′ UTR of the IE2 mRNA. Additionally, the loss of repression with the four nucleotide substitution demonstrates that the repression is mediated through the sequence predicted by the above-mentioned algorithm.

Summary:

The experiments described above confirmed the prediction that human encoded miRNAs can target the 3′UTR of viral transcripts. Specifically, the algorithm predicted that several cellular miRNAs target the 3′UTR of HCMV IE2. Cells that express the miRNAs to high levels (FIG. 5) can repress by 2 fold the levels of reporter gene when the wild type sequence is present but not when the mutated 3′UTR is used (FIG. 6). These results confirm that the above-mentioned algorithm can predict cellular miRNA targeting of viral transcripts.

The algorithm predicts that there are several miRNAs encoded by human cells that can target specific viral targets thereby modulating viral gene expression. The consequences of these interactions can lead to several different potential outcomes, including but not limited to inhibition of viral replication, reduced cytopathic effect of infected cells, reduced toxicity of infected cells, the establishment of viral latency, restriction of cell types upon infection and the potential identification of potent anti-viral agents.

The present invention is not limited to the embodiments described and exemplified above, but is capable of variation and modification within the scope of the appended claims.

What is claimed:

1. A method of identifying miRNA hybridization targets in a population of mRNA molecules, wherein the population of mRNA molecules corresponds to mRNAs encoded by one or more selected genomes, the method comprising the steps of:
a) providing one or more databases comprising selected miRNA sequences and sequences representing 3′ untranslated regions (3′UTRs) of the population of mRNA molecules;
b) determining one or more seed oligomers for each of the selected miRNA molecules;
c) computing the probability (p) of finding an oligomer complementary to a seed oligomer at any position of a random background sequence generated using a kth order Markov model based on the sequence composition of the 3′ UTRs;
d) counting the number (c) of occurrences of an oligomer in each 3′UTR that is complementary to a seed oligomer, thereby creating a collection of miRNA-3′UTR pairs;
e) providing a score for each miRNA-3′UTR pair, wherein the score is determined by a single hypothesis p-value PV_{SH} of a binomial distribution, computed by
$${PV}_{SH} ( l , c , p ) = \frac{B ( p , c , l - c + 1 )}{B ( c , l - c + l )} ;$$
wherein 1 is the length of the 3′ UTR, B(x,a,b) is the incomplete beta function and B(a,b) is the usual beta function, defined by
$$B ( x , a , b ) = {\int }_{0}^{x} {{u}^{a - 1} ( 1 - u )}^{b - 1} d u , \\ B ( a , b ) = B ( 1 , a , b ) ;$$
f) ranking the miRNA-3′UTR pairs according to their score PV_{SH}, wherein the highest rank corresponds to the smallest PV_{SH};
g) evaluating the statistical significance of the t highest-ranking microRNA-target pairs, wherein t is an integer number between 1 and the total number of pairs tested, by generating N random genomes analogous to the selected genome, wherein each random genome comprises the same number of 3′UTRs as the selected genome, and each corresponding 3′UTR is of the same length and is based on the same kth Markov model as the corresponding 3′UTR in the selected genome;
h) repeating steps c) through f) for each of the N random genomes;
i) evaluating the statistical significance of the t highest-ranking miRNA-3′UTR pairs from step f) for the selected genome by (1) counting the number Nₜ of the randomly generated genomes in which the tth pair exhibits PV_{SH} smaller than the tth pair in the selected genome and (2) computing the p-value PV_{MH}(t) corrected for Multiple Hypothesis Testing from the formula
$${PV}_{MH} ( t ) = \frac{{N}_{t}}{N} ;$$
wherein PV_{MH}(t) is the probability of finding higher scores for the t highest-ranking miRNA-3′UTR pairs in the random genome as compared with the selected genome; and
j) identifying the miRNA hybridization targets by assessing each PV_{MH}(t), wherein a smaller PV_{MH}(t), correlates with a higher probability that the predicted targets are miRNA hybridization targets.

2. The method of claim 1, wherein the seed oligomers are heptamers or hexamers.

3. The method of claim 2, wherein the hexamers are determined from positions 2-7 or 3-8 from the 5′ end of the miRNA sequences and the heptamers are determined from positions 2-8 from the 5′ end of the miRNA sequences.

4. The method of claim 1, wherein the 3′UTRs are determined experimentally or computationally.

5. The method of claim 1, wherein the miRNA sequences are human or viral and the one or more selected genomes is a virus genome.

6. The method of claim 5, wherein the viral miRNA sequences and the one or more selected genomes are from herpes viruses.

7. A system for identifying miRNA hybridization targets comprising: an input interface for inputting mRNA sequences, a database of mRNA sequences or a link for connecting to a remote data input interface, data or a database of mRNA sequences; an input interface for inputting miRNA sequences, a database of miRNA sequences or a link for connecting to a remote data input interface, data or a database of miRNA sequences; a processor with instructions for comparing mRNA sequences to miRNA sequences to identify miRNA hybridization targets according to the method of claim 1.

8. The system of claim 7, comprising a link for connecting to a database of mRNA sequences.

9. The system of claim 7, comprising an input interface for inputting miRNA sequences.

10. A computer program comprised in a computer readable medium for implementation on a computer system for identifying miRNA hybridization targets, the program comprising instructions for performing the steps of the method of claim 1.

11. A complex comprising an mRNA hybridization target to which is hybridized a miRNA or siRNA derivative thereof, wherein the hybridization of the miRNA or siRNA derivative thereof to the mRNA hybridization target is predicted by a method comprising the steps of:
a) providing one or more databases comprising selected miRNA sequences and sequences representing 3′ untranslated regions (3′UTRs) of the population of mRNA molecules;
b) determining one or more seed oligomers for each of the selected miRNA molecules;
c) computing the probability (p) of finding an oligomer complementary to a seed oligomer at any position of a random background sequence generated using a kth order Markov model based on the sequence composition of the 3′ UTRs;
d) counting the number (c) of occurrences of an oligomer in each 3′UTR that is complementary to a seed oligomer, thereby creating a collection of miRNA-3′UTR pairs;
e) providing a score for each miRNA-3′UTR pair, wherein the score is determined by a single hypothesis p-value PV_{SH} of a binomial distribution, computed by
$${PV}_{SH} ( l , c , p ) = \frac{B ( p , c , l - c + 1 )}{B ( c , l - c + l )} ;$$
wherein l is the length of the 3′ UTR, B(x,a,b) is the incomplete beta function and B(a,b) is the usual beta function, defined by
$$B ( x , a , b ) = {\int }_{0}^{x} {{u}^{a - 1} ( 1 - u )}^{b - 1} d u , \\ B ( a , b ) = B ( 1 , a , b ) ;$$
f) ranking the miRNA-3′UTR pairs according to their score PV_{SH}, wherein the highest rank corresponds to the smallest PV_{SH};
g) evaluating the statistical significance of the t highest-ranking microRNA-target pairs, wherein t is an integer number between 1 and the total number of pairs tested, by generating N random genomes analogous to the selected genome, wherein each random genome comprises the same number of 3′UTRs as the selected genome, and each corresponding 3′UTR is of the same length and is based on the same kth Markov model as the corresponding 3′UTR in the selected genome;
h) repeating steps c) through f) for each of the N random genomes;
i) evaluating the statistical significance of the t highest-ranking miRNA-3′UTR pairs from step f) for the selected genome by (1) counting the number Nₜ of the randomly generated genomes in which the tth pair exhibits PV_{SH} smaller than the tth pair in the selected genome and (2) computing the p-value PV_{MH}(t) corrected for Multiple Hypothesis Testing from the formula
$${PV}_{MH} ( t ) = \frac{{N}_{t}}{N} ;$$
wherein PV_{MH}(t) is the probability of finding higher scores for the t highest-ranking miRNA-3′UTR pairs in the random genome as compared with the selected genome; and
j) identifying the miRNA hybridization targets by assessing each PV_{MH}(t), wherein a smaller PV_{MH}(t), correlates with a higher probability that the predicted targets are miRNA hybridization targets.

12. The complex of claim 11, wherein the mRNA hybridization targets are viral 3′ untranslated regions (3′UTRs) from herpes simplex virus 1 or 2 (HSV), Epstein-Barr virus (EBV), human cytomegalovirus (HCMV), Kaposi's sarcoma-related herpesvirus (KSHV) or varicella zoster virus (VZV).

13. The complex of claim 12, wherein the viral 3′UTRs are
a) HSV 3′UTRs RL1 (ICP 34.5), RL2 (ICP0), UL1, UL2, UL5, UL9, UL11, UL13, UL14, UL16, UL20, UL24, UL34, UL35, UL37, UL39, UL42, UL47, UL49A, UL51, UL52, US1 (US1.5, ICP22), US8, US8A, US9, US11, or US12 (ICP47);
b) EBV 3′UTRs BALF2, BALF3, BALF5, BARF0, BaRF1, BARF1, BBLF4, BDLF 3.5, BDLF4, BFRF2, BGLF1, BGLF2, BGLF3, BGLF 3.5, BHLF1, BHRF1, BLLF3, BMRF1, BNRF1, BOLF1, BRLF1, BSLF2/BMLF1, BVLF1, BXLF1, BXRF1, BZLF1, BZLF2, LF3, LMP-1, LMP-2A, or LMP-2B;
c) HCMV 3′UTRs IE1 (UL123), IE2 (UL122), RL1, RL10, UL3, UL16, UL17, UL20, UL26, UL29, UL31, UL32, UL33, UL34, UL37, UL38, UL40, UL43, UL44, UL45, UL50, UL51, UL52, UL54, UL57, UL60, UL61, UL67, UL69, UL78, UL79, UL80, UL86, UL87, UL91, UL92, UL95, UL97, UL98, UL10, UL103, UL105, UL107, UL112-113, UL117, UL120, UL137, UL141a, UL151, UL151a, UL153, US7, US10, US12, US14, US24, US26, US27, US28, New ORF1, or New ORF3;
d) KSHV 3′UTRs ORF6, ORF7, ORF8, ORF9, ORF16, ORF18, ORF21, ORF25, ORF26, ORF28, ORF32, ORF40, ORF47, ORF49, ORF 50 (Rta), ORF56, ORF57, ORF58, ORF59, ORF63, ORF72, ORF73 (LANA), ORF74, ORF75, ORFK4, ORFK8 (Zta), ORFK13, and ORFK14; or
e) VZV 3′UTRs ORF16, ORF47, ORF52, ORF55, ORF59, ORF61, or ORF62.

14. The complex of claim 13, wherein the miRNAs are:
a) HSV miRNAs hsv1-miR-H1, or hsv1-miR-LAT;
b) EBV miRNAs ebv-miR-BART1-3p, ebv-miR-BART1-5p, ebv-miR-BART2, ebv-miR-BART3-3p, ebv-miR-BART3-5p, ebv-miR-BART4, ebv-miR-BART5, ebv-miR-BART6-3p, ebv-miR-BART6-5p, ebv-miR-BART7, ebv-miR-BART8-3p, ebv-miR-BART8-5p, ebv-miR-BART9, ebv-miR-BART10, ebv-miR-BART11-3p, ebv-miR-BART11-5p, ebv-miR-BART12, ebv-miR-BART13, ebv-miR-BART14-3p, ebv-miR-BART14-5p, ebv-miR-BART15, ebv-miR-BART16, ebv-miR-BART17-3p, ebv-miR-BART17-5p, ebv-miR-BART18, ebv-miR-BART19, ebv-miR-BART20-3p, ebv-miR-BART20-5p, ebv-miR-BHRF1-1, ebv-miR-BHRF1-2*, or ebv-miR-BHRF1-3;
c) HCMV miRNAs hcmv-miR-UL22-1, hcmv-miR-UL22A-1*, hcmv-miR-UL31-1, hcmv-miR-UL36-1, hcmv-miR-UL36-1-N, hcmv-miR-UL53-1, hcmv-miR-UL54-1, hcmv-miR-UL70-3p, hcmv-miR-UL70-5p, hcmv-miR-UL102-1, hcmv-miR-UL102-2, hcmv-miR-UL111a-1, hcmv-miR-UL112-1, hcmv-miR-UL148D-1, hcmv-miR-US4, hcmv-miR-US5-1, hcmv-miR-US5-2, hcmv-miR-US5-2-N, hcmv-miR-US25-1, hcmv-miR-US25-2-5p, hcmv-miR-US25-2-3p, hcmv-miR-US29-1, or hcmv-miR-US33-1;
d) KSHV miRNAs kshv-miR-K12-1, kshv-miR-K12-2, kshv-miR-K12-3, kshv-miR-K12-3*, kshv-miR-K12-4-5p, kshv-miR-K112-4-3p, kshv-miR-K112-5, kshv-miR-K12-6-5p, kshv-miR-K12-6-3p, kshv-miR-K12-7, kshv-miR-K12-8, kshv-miR-K12-9*, kshv-miR-K12-9, kshv-miR-K12-10a, kshv-miR-K12-10b, kshv-miR-K12-11, or kshv-miR-K12-12; or
e) human miRNAs
(i) targeting HSV: hsa-miR-138, hsa-miR-205, hsa-miR-326, hsa-miR-381, hsa-miR-425, hsa-miR-492, or hsa-miR-522;
(ii) targeting EBV: hsa-miR-24, hsa-miR-214, hsa-miR-296, hsa-miR-328, hsa-miR-346, or hsa-miR-502;
(iii) targeting HCMV: hsa-miR-15a, hsa-miR-15b, hsa-miR-16, hsa-miR-103, hsa-miR-107, hsa-miR-126, hsa-miR-142-5p, hsa-miR-184, hsa-miR-194, hsa-miR-195, hsa-miR-200b, hsa-miR-200c, hsa-miR-202, hsa-miR-326, hsa-miR-330-5p, hsa-miR-367, hsa-miR-424, hsa-miR-429, hsa-miR-450-b-3p, hsa-miR-497, hsa-miR-503, hsa-miR-548d-3p, hsa-miR-548k, hsa-miR-551a, hsa-miR-551b, hsa-miR-552, hsa-miR-592, hsa-miR-598, hsa-miR-652, hsa-miR-769-3-p, or hsa-miR-1226;
(iv) targeting KSHV: hsa-let-7a, hsa-let-7b, hsa-let-7c, hsa-let-7d, hsa-let-7e, hsa-let-7f, hsa-let-7g, hsa-let-7i, hsa-miR-1, hsa-miR-9, hsa-miR-15a, hsa-miR-15b, hsa-miR-16, hsa-miR-17-5p, hsa-miR-18a, hsa-miR-18b, hsa-miR-20a, hsa-miR-20b, hsa-miR-23a, hsa-miR-23b, hsa-miR-30a-5p, hsa-miR-30a-3p, hsa-miR-30b, hsa-miR-30c, hsa-miR-30e-5p, hsa-miR-30e-3p, hsa-miR-93, hsa-miR-98, hsa-miR-105, hsa-miR-106a, hsa-miR-106b, hsa-miR-125a, hsa-miR-125b, hsa-miR-129, hsa-miR-134, hsa-miR-137, hsa-miR-141, hsa-miR-142-3p, hsa-miR-145, hsa-miR-150, hsa-miR-154, hsa-miR-181a, hsa-miR-181b, hsa-miR-181c, hsa-miR-181d, hsa-miR-182*, hsa-miR-194, hsa-miR-195, hsa-miR-196a, hsa-miR-196b, hsa-miR-199a, hsa-miR-199b, hsa-miR-200a, hsa-miR-205, hsa-miR-206, hsa-miR-210, hsa-miR-213, hsa-miR-299-3p, hsa-miR-302a, hsa-miR-302b, hsa-miR-302c, hsa-miR-302d, hsa-miR-324-3p, hsa-miR-326, hsa-miR-329, hsa-miR-337, hsa-miR-338, hsa-miR-340, hsa-miR-346, hsa-miR-372, hsa-miR-373, hsa-miR-424, hsa-miR-448, hsa-miR-450, hsa-miR-453, hsa-miR-455, hsa-miR-490, hsa-miR-491, hsa-miR-492, hsa-miR-497, hsa-miR-518b, hsa-miR-518c, hsa-miR-518d, hsa-miR-519d, hsa-miR-520a, hsa-miR-520b, hsa-miR-520c, hsa-miR-520d, hsa-miR-520g, hsa-miR-520h, hsa-miR-525, or hsa-miR-526b; or
(v) targeting VZV: hsa-miR-99a, hsa-miR-99b, hsa-miR-100, hsa-miR-124a, hsa-miR-132, hsa-miR-141, hsa-miR-150, hsa-miR-197, hsa-miR-200a, hsa-miR-212, hsa-miR-219, hsa-miR-330, hsa-miR-374, hsa-miR-371, hsa-miR-339, hsa-miR-451, hsa-miR-495, and hsa-miR-510.

15. The complex of claim 14, comprising miRNA-3′UTR pairs wherein:
a) the 3′UTRs are from HSV and the pairs are: hsv1-miR-LAT targeting ICP0 (RL2); hsv1-miR-LAT targeting UL9; hsv1-miR-LAT targeting UL42; hsv1-miR-LAT targeting ICP34.5 (RL1); hsa-miR-138 targeting ICP0 (RL2); hsa-miR-425 targeting UL47; hsa-miR-381 targeting ICP22 (US1); hsa-miR-522 targeting UL5; hsa-miR-326 targeting ICP47 (US12); hsa-miR-205 targeting UL2; or hsa-miR-492 targeting UL52;
b) the 3′UTRs are from EBV and the pairs are: ebv-miR-BHRF1-3 or ebv-miR-BART15 targeting BZLF1 or BRLF1; ebv-miR-BART2 or ebv-miR-BART6-3p targeting BALF5; ebv-miR-BART-1-3p targeting BHRF1; ebv-miR-BART10 targeting BBLF4; ebv-miR-BHRF1-3 targeting BSLF2/BMLF1 (Mta); ebv-miR-BART17-5p targeting BMRF1; ebv-miR-BART6-3p targeting LF3; hsa-miR-24 targeting BHRF1; hsa-miR-214 targeting BXLF1; hsa-miR-296 targeting BALF5; hsa-miR-296 or hsa-miR-328 targeting LMP-2A or LMP-2B; or hsa-miR-346 or hsa-miR-502 targeting LMP-1;
c) the 3′UTRs are from HCMV and the pairs are: hcmv-miR-UL112-1 targeting IE1 (UL123); hcmv-miR-UL36-1 targeting UL37; hcmv-miR-UL53-1 targeting UL52; hcmv-miR-UL54-1 targeting UL112-113 or UL45; hcmv-miR-US25-2-5p targeting UL57; hcmv-miR-UL148D-1 targeting UL26, UL98, UL103 or UL151a; hcmv-miR-US5-1 or US5-2 targeting US7; hcmv-miR-US25-2-3p targeting UL32; hcmv-miR-US33-1 targeting US28; hsa-miR-200b, 200c or 429 targeting IE2 (UL122); hsa-miR-769-3-p or 450-b-3p targeting IE1 (UL123); hsa-miR-503 targeting UL44 or UL37; hsa-miR-503 or 592 targeting UL54; hsa-miR-142-5p targeting UL97, UL33 or US 27; hsa-miR-103, 107, 202, 15a, 15b, 16, 195, 424 or 497 targeting UL38; hsa-miR-367 targeting UL57; hsa-miR-1226 targeting UL50; hsa-miR-184 targeting UL31; hsa-miR-16, 15b, 195, 424, 15a or 497 targeting UL78; hsa-miR-652 targeting New ORF3; hsa-miR-552 targeting UL91; hsa-miR-548k targeting UL29; hsa-miR-330-5p or 326 targeting New ORF1; hsa-miR-548d-3p targeting UL107; hsa-miR-598 targeting UL60; hsa-miR-126 targeting UL20; hsa-miR-194 targeting UL17; hsa-miR-551a or 551b targeting UL100; or hsa-miR-503 targeting RL1;
d) the 3′UTRs are from KSHV and the pairs are: kshv-miR-K12-6-3p targeting Zta (ORF K8) or Rta (ORF 50); kshv-miR-K12-8 targeting ORF9; kshv-miR-K12-10b targeting LANA (ORF73); hsa-miR-302b*, 105, 150, 210, 142-3p, 302a-d, 372, 373, 520a-e, 526b*, 93, 17-5p, 519d, 20a-b, 106a-b, 199a-b, or 520g-h targeting ORF6; hsa-miR-329, 141, 200a, 324-3p, 213, 182*, 105, 455, 518b-d, 453 or 98, or hsa-let-7a-g or i, targeting LANA (ORF73); hsa-miR-199a-b, 137, 205, 154, 346, 340, 490, 9, 1, 206, 492, 299-3p, or 491 targeting ORF56; hsa-miR-129, 450, 448, 134, 196a-b, 337, 141, 200a, 194, 30a-5p, 30a-3p, 30b-d, 30e-5p, 30e-3p, 195, 15a-b, 16, 424, or 497 targeting ORF58; or hsa-miR-326, 181a-d, 181a, 23a-b, 125a-b, 340, 18a-b, 520a*, 525, 145, or 338 targeting ORF21; or
e) the 3′UTRs are from VZV and the pairs are: hsa-miR-132, 212, 451, or 495 targeting ORF62; hsa-miR-510, 150, 124a, or 330 targeting ORF61; hsa-miR-197 targeting ORF52; hsa-miR-374 targeting ORF16; hsa-miR-371, 219, or 339 targeting ORF47; hsa-miR-141 or 200a targeting ORF59; or hsa-miR-99a, 99b, or 100 targeting ORF55.

16. The complex of claim 14, comprising miRNA-3′UTR pairs wherein:
a) the 3′UTRs are from HSV and the pairs are: hsv1-miR-H1, targeting UL35, US9, UL24, UL34 or US8A; or hsv1-mir-LAT, targeting RL1, RL2, UL20, UL42, UL1, UL49A, UL52, UL9, UL11, UL51, UL39, UL47, US8A, UL16, UL13, UL37, UL14 or US11;
b) the 3′UTRs are from EBV and the pairs are: ebv-miR-BART1-3p, targeting BRLF1, BHRF1 or BGLF2; ebv-miR-BART2 targeting BKRF2; ebv-miR-BART5 targeting BNRF1 or BARF1; ebv-miR-BART6-3p targeting LF3; ebv-miR-BART6-5p targeting BALF3; ebv-miR-BART10 targeting BHLF1; 18 targeting BFRF2, BLRF2 or LF1; ebv-miR-BART13 targeting BSLF1; ebv-miR-BART15 targeting BZLF1 or BaRF1; ebv-miR-BART16 targeting BHLF1; ebv-miR-BART17-3p targeting BNRF1; ebv-miR-BART20-3p targeting BLLF3; ebv-miR-BHRF1-1 targeting BaRF1; ebv-miR-BHRF1-2 targeting BALF3; ebv-miR-BHRF1-2* targeting BGRF1/BDRF1 or BZLF2; or ebv-miR-BHRF1-3 targeting BZLF1, BSLF2/BMLF1 or BDLF3.5;
c) the 3′UTRs are from HCMV and the pairs are: hcmv-miR-UL22-1 targeting RL4; hcmv-miR-UL36-1 targeting UL138; hcmv-miR-UL36-1-N targeting UL16 or UL98; hcmv-miR-UL53-1 targeting UL61 or UL67; hcmv-miR-UL54-1 targeting UL112-113 or UL86; hcmv-miR-UL70-5p targeting UL141a, UL80, US14 or UL3; hcmv-miR-UL102-1 targeting UL104; hcmv-miR-UL102-2 targeting UL87; hcmv-miR-UL112-1 targeting UL34, UL123 or UL31; hcmv-miR-UL148D-1 targeting US9, UL103, UL92 or UL93; hcmv-miR-US4 targeting UL10 or UL16; hcmv-miR-US5-1 targeting UL60 or RL10; hcmv-miR-US5-2 targeting UL103; hcmv-miR-US5-2-N targeting US7, US23 or UL60; hcmv-miR-US25-1 targeting UL61; hcmv-miR-US25-2-5p targeting UL153, UL57 or UL7; hcmv-miR-US25-2-3p targeting UL18; hcmv-miR-US29-1 targeting UL153; or hcmv-miR-US33-1 targeting UL69, UL102 or US28; or
d) the 3′UTRs are from KSHV and the pairs are: kshv-miR-K12-2 targeting ORF63; kshv-miR-K12-3 targeting ORF31 or ORF32; kshv-miR-K12-3* targeting ORF16; kshv-miR-K12-4-5p targeting ORF74, ORFK14 or ORF72; kshv-miR-K12-4-3p targeting ORF49, ORF57 or ORF64; kshv-miR-K12-5 targeting ORF56; kshv-miR-K12-6-5p targeting ORF28, ORF16, ORF8 or ORF27; kshv-miR-K12-6-3p targeting ORFK8 or ORF50; kshv-miR-K12-7 targeting ORFK4; kshv-miR-K12-8 targeting ORF18; kshv-miR-K12-9 targeting ORF K4 or ORF67; kshv-miR-K12-10a or kshv-miR-K12-10b targeting ORF25; or kshv-miR-K12-12 targeting ORF67.

17. The complex of claim 16, wherein the 3′UTRs are from HCMV and the pairs are: hcmv-miR-US5-2 targeting UL103; hcmv-miR-UL54-1 targeting UL112-113; hcmv-miR-US5-1 targeting RL10; hcmv-miR-UL112-1 targeting UL31; hcmv-miR-UL70-5p targeting UL80;
hcmv-miR-UL112-1 targeting UL34; hcmv-miR-UL70-5p targeting UL3; hcmv-miR-US33-1 targeting UL69; hcmv-miR-US25-2-5p targeting UL57; or hcmv-miR-UL112-1 targeting UL123(IE1).

18. A siRNA or a chemically modified analog of a miRNA, which hybridizes with one or more mRNA targets selected from:
a) HSV 3′UTRs RL1 (ICP 34.5), RL2 (ICP0), UL1, UL2, UL5, UL9, UL11, UL13, UL14, UL16, UL20, UL24, UL34, UL35, UL37, UL39, UL42, UL47, UL49A, UL51, UL52, US1 (US1.5, ICP22), US8, US8A, US9, US11, or US12 (ICP47);
b) EBV 3′UTRs BALF2, BALF3, BALF5, BARF0, BaRF1, BARF1, BBLF4, BDLF 3.5, BDLF4, BFRF2, BGLF1, BGLF2, BGLF3, BGLF 3.5, BHLF1, BHRF1, BLLF3, BMRF1, BNRF1, BOLF1, BRLF1, BSLF2/BMLF1, BVLF1, BXLF1, BXRF1, BZLF1, BZLF2, LF3, LMP-1, LMP-2A, or LMP-2B;
c) HCMV 3′UTRs IE1 (UL123), IE2 (UL122), RL1, RL10, UL3, UL16, UL17, UL20, UL26, UL29, UL31, UL32, UL33, UL34, UL37, UL38, UL40, UL43, UL44, UL45, UL50, UL51, UL52, UL54, UL57, UL60, UL61, UL67, UL69, UL78, UL79, UL80, UL86, UL87, UL91, UL92, UL95, UL97, UL98, UL10, UL103, UL105, UL107, UL112-113, UL117, UL120, UL137, UL141a, UL151, UL151a, UL153, US7, US10, US12, US14, US24, US26, US27, US28, New ORF1, or New ORF3;
d) KSHV 3′UTRs ORF6, ORF7, ORF8, ORF9, ORF16, ORF18, ORF21, ORF25, ORF26, ORF28, ORF32, ORF40, ORF47, ORF49, ORF 50 (Rta), ORF56, ORF57, ORF58, ORF59, ORF63, ORF72, ORF73 (LANA), ORF74, ORF75, ORFK4, ORFK8 (Zta), ORFK13, and ORFK14; or
e) VZV 3′UTRs ORF16, ORF47, ORF52, ORF55, ORF59, ORF61, or ORF62.

19. The siRNA or chemically modified miRNA of claim 18, comprising a seed sequence of a miRNA selected from:
a) HSV miRNAs hsv1-miR-H1, or hsv1-miR-LAT;
b) EBV miRNAs ebv-miR-BART1-3p, ebv-miR-BART1-5p, ebv-miR-BART2, ebv-miR-BART3-3p, ebv-miR-BART3-5p, ebv-miR-BART4, ebv-miR-BART5, ebv-miR-BART6-3p, ebv-miR-BART6-5p, ebv-miR-BART7, ebv-miR-BART8-3p, ebv-miR-BART8-5p, ebv-miR-BART9, ebv-miR-BART10, ebv-miR-BART11-3p, ebv-miR-BART11-5p, ebv-miR-BART12, ebv-miR-BART13, ebv-miR-BART14-3p, ebv-miR-BART14-5p, ebv-miR-BART15, ebv-miR-BART16, ebv-miR-BART17-3p, ebv-miR-BART17-5p, ebv-miR-BART18, ebv-miR-BART19, ebv-miR-BART20-3p, ebv-miR-BART20-5p, ebv-miR-BHRF1-1, ebv-miR-BHRF1-2*, or ebv-miR-BHRF1-3;
c) HCMV miRNAs hcmv-miR-UL22-1, hcmv-miR-UL22A-1*, hcmv-miR-UL31-1, hcmv-miR-UL36-1, hcmv-miR-UL36-1-N, hcmv-miR-UL53-1, hcmv-miR-UL54-1, hcmv-miR-UL70-3p, hcmv-miR-UL70-5p, hcmv-miR-UL102-1, hcmv-miR-UL102-2, hcmv-miR-UL111a-1, hcmv-miR-UL112-1, hcmv-miR-UL148D-1, hcmv-miR-US4, hcmv-miR-US5-1, hcmv-miR-US5-2, hcmv-miR-US5-2-N, hcmv-miR-US25-1, hcmv-miR-US25-2-5p, hcmv-miR-US25-2-3p, hcmv-miR-US29-1, or hcmv-miR-US33-1;
d) KSHV miRNAs kshv-miR-K12-1, kshv-miR-K12-2, kshv-miR-K12-3, kshv-miR-K12-3*, kshv-miR-K112-4-5p, kshv-miR-K112-4-3p, kshv-miR-K12-5, kshv-miR-K12-6-5p, kshv-miR-K12-6-3p, kshv-miR-K12-7, kshv-miR-K12-8, kshv-miR-K12-9*, kshv-miR-K12-9, kshv-miR-K12-10a, kshv-miR-K12-10b, kshv-miR-K12-11, or kshv-miR-K12-12; or
e) human miRNAs
(i) targeting HSV: hsa-miR-138, hsa-miR-205, hsa-miR-326, hsa-miR-381, hsa-miR-425, hsa-miR-492, or hsa-miR-522;
(ii) targeting EBV: hsa-miR-24, hsa-miR-214, hsa-miR-296, hsa-miR-328, hsa-miR-346, or hsa-miR-502;
(iii) targeting HCMV: hsa-miR-15a, hsa-miR-15b, hsa-miR-16, hsa-miR-103, hsa-miR-107, hsa-miR-126, hsa-miR-142-5p, hsa-miR-184, hsa-miR-194, hsa-miR-195, hsa-miR-200b, hsa-miR-200c, hsa-miR-202, hsa-miR-326, hsa-miR-330-5p, hsa-miR-367, hsa-miR-424, hsa-miR-429, hsa-miR-450-b-3p, hsa-miR-497, hsa-miR-503, hsa-miR-548d-3p, hsa-miR-548k, hsa-miR-551a, hsa-miR-551b, hsa-miR-552, hsa-miR-592, hsa-miR-598, hsa-miR-652, hsa-miR-769-3-p, or hsa-miR-1226;
(iv) targeting KSHV: hsa-let-7a, hsa-let-7b, hsa-let-7c, hsa-let-7d, hsa-let-7e, hsa-let-7f, hsa-let-7g, hsa-let-7i, hsa-miR-1, hsa-miR-9, hsa-miR-15a, hsa-miR-15b, hsa-miR-16, hsa-miR-17-5p, hsa-miR-18a, hsa-miR-18b, hsa-miR-20a, hsa-miR-20b, hsa-miR-23a, hsa-miR-23b, hsa-miR-30a-5p, hsa-miR-30a-3p, hsa-miR-30b, hsa-miR-30c, hsa-miR-30e-5p, hsa-miR-30e-3p, hsa-miR-93, hsa-miR-98, hsa-miR-105, hsa-miR-106a, hsa-miR-106b, hsa-miR-125a, hsa-miR-125b, hsa-miR-129, hsa-miR-134, hsa-miR-137, hsa-miR-141, hsa-miR-142-3p, hsa-miR-145, hsa-miR-150, hsa-miR-154, hsa-miR-181a, hsa-miR-181b, hsa-miR-181c, hsa-miR-181d, hsa-miR-182*, hsa-miR-194, hsa-miR-195, hsa-miR-196a, hsa-miR-196b, hsa-miR-199a, hsa-miR-199b, hsa-miR-200a, hsa-miR-205, hsa-miR-206, hsa-miR-210, hsa-miR-213, hsa-miR-299-3p, hsa-miR-302a, hsa-miR-302b, hsa-miR-302c, hsa-miR-302d, hsa-miR-324-3p, hsa-miR-326, hsa-miR-329, hsa-miR-337, hsa-miR-338, hsa-miR-340, hsa-miR-346, hsa-miR-372, hsa-miR-373, hsa-miR-424, hsa-miR-448, hsa-miR-450, hsa-miR-453, hsa-miR-455, hsa-miR-490, hsa-miR-491, hsa-miR-492, hsa-miR-497, hsa-miR-518b, hsa-miR-518c, hsa-miR-518d, hsa-miR-519d, hsa-miR-520a, hsa-miR-520b, hsa-miR-520c, hsa-miR-520d, hsa-miR-520g, hsa-miR-520h, hsa-miR-525, or hsa-miR-526b; or
(v) targeting VZV: hsa-miR-99a, hsa-miR-99b, hsa-miR-100, hsa-miR-124a, hsa-miR-132, hsa-miR-141, hsa-miR-150, hsa-miR-197, hsa-miR-200a, hsa-miR-212, hsa-miR-219, hsa-miR-330, hsa-miR-374, hsa-miR-371, hsa-miR-339, hsa-miR-451, hsa-miR-495, and hsa-miR-510.

20. The siRNA or chemically modified miRNA of claim 19, wherein the seed sequence comprises, as at least a portion thereof, one of the following sequences or its complement:
a) from HSV, TCCTTC or GGCCGC;
b) from EBV, CGGTGCT, CACTAAG, AGAAAAT, GTGGTGC, ACTAGGT, ATCAGGT, TCACCTT, GATCCCC, GACCAAC, CTATGAT, ATTGTGA, AAACCGT, AAGTGTT, GGTTATG, GTGTGCG, AAACTGT, CCACAGG, AAGTTAC, AGCATTT, GTAGGGT, AAACCAC, CACTCTA, GCATACA, GTCCTCT, CGAACTT, ACAAAAC, CCTTCAT, CCTGCTA, TCAGGTT, AAAAGAT, CAGAATT, or TCCCGTT;
c) from HCMV, TCCCGTG, GCTAGTT, TCTGGTG, ACATGCC, TTCAACG, AGGTGTC, CTCGCGC, GACGCGC, CCATCCC, GAGACGC, CATGGCC, CGACGCC, CAACGTC, CGTCACT, GAGGACG, CCATGTC, GCTTGTC, TATCATA, ACCTATC, GAGCGGT, AGACCGC, AAGTGGA, ACATCCA, or GCACAAT;
d) from KSHV, CCTGTA, CTACAG, GAATGT, GACCGC, GTTTAG, GTATTC, GCATCC, GCTGCT, AACCAT, TGGGAT, CGCGCC, AGCTGG, ATACCC, CAACAC, CAACAC, AGCATT, or GGCCTG.

21. A vector comprising a polynucleotide which, when expressed in a mammalian cell, produces a transcript that is processed within the cell to form a miRNA or a siRNA derivative thereof, which is capable of binding to a viral 3′UTR selected from:
a) HSV 3′UTRs RL1 (ICP 34.5), RL2 (ICP0), UL1, UL2, UL5, UL9, UL11, UL13, UL14, UL16, UL20, UL24, UL34, UL35, UL37, UL39, UL42, UL47, UL49A, UL51, UL52, US1 (US1.5, ICP22), US8, US8A, US9, US11, or US12 (ICP47);
b) EBV 3′UTRs BALF2, BALF3, BALF5, BARF0, BaRF1, BARF1, BBLF4, BDLF 3.5, BDLF4, BFRF2, BGLF1, BGLF2, BGLF3, BGLF 3.5, BHLF1, BHRF1, BLLF3, BMRF1, BNRF1, BOLF1, BRLF1, BSLF2/BMLF1, BVLF1, BXLF1, BXRF1, BZLF1, BZLF2, LF3, LMP-1, LMP-2A, or LMP-2B;
c) HCMV 3′UTRs IE1 (UL123), IE2 (UL122), RL1, RL10, UL3, UL16, UL17, UL20, UL26, UL29, UL31, UL32, UL33, UL34, UL37, UL38, UL40, UL43, UL44, UL45, UL50, UL51, UL52, UL54, UL57, UL60, UL61, UL67, UL69, UL78, UL79, UL80, UL86, UL87, UL91, UL92, UL95, UL97, UL98, UL100, UL103, UL105, UL107, UL112-113, UL117, UL120, UL137, UL141a, UL151, UL151a, UL153, US7, US10, US12, US14, US24, US26, US27, US28, New ORF1, or New ORF3;
d) KSHV 3′UTRs ORF6, ORF7, ORF8, ORF9, ORF16, ORF18, ORF21, ORF25, ORF26, ORF28, ORF32, ORF40, ORF47, ORF49, ORF 50 (Rta), ORF56, ORF57, ORF58, ORF59, ORF63, ORF72, ORF73 (LANA), ORF74, ORF75, ORFK4, ORFK8 (Zta), ORFK13, and ORFK14; or
e) VZV 3′UTRs ORF16, ORF47, ORF52, ORF55, ORF59, ORF61, or ORF62.

22. The vector of claim 21, comprising a polynucleotide which, when expressed in a mammalian cell, produces a transcript that is processed within the cell to form a miRNA or an siRNA derivative of a miRNA comprising one or more of:
a) HSV miRNAs hsv1-miR-H1, or hsv1-miR-LAT;
b) EBV miRNAs ebv-miR-BART1-3p, ebv-miR-BART1-5p, ebv-miR-BART2, ebv-miR-BART3-3p, ebv-miR-BART3-5p, ebv-miR-BART4, ebv-miR-BART5, ebv-miR-BART6-3p, ebv-miR-BART6-5p, ebv-miR-BART7, ebv-miR-BART8-3p, ebv-miR-BART8-5p, ebv-miR-BART9, ebv-miR-BART10, ebv-miR-BART11-3p, ebv-miR-BART11-5p, ebv-miR-BART12, ebv-miR-BART13, ebv-miR-BART14-3p, ebv-miR-BART14-5p, ebv-miR-BART15, ebv-miR-BART16, ebv-miR-BART17-3p, ebv-miR-BART17-5p, ebv-miR-BART18, ebv-miR-BART19, ebv-miR-BART20-3p, ebv-miR-BART20-5p, ebv-miR-BHRF1-1, ebv-miR-BHRF1-2*, or ebv-miR-BHRF1-3;
c) HCMV miRNAs hcmv-miR-UL22-1, hcmv-miR-UL22A-1*, hcmv-miR-UL31-1, hcmv-miR-UL36-1, hcmv-miR-UL36-1-N, hcmv-miR-UL53-1, hcmv-miR-UL54-1, hcmv-miR-UL70-3p, hcmv-miR-UL70-5p, hcmv-miR-UL102-1, hcmv-miR-UL102-2, hcmv-miR-UL111a-1, hcmv-miR-UL112-1, hcmv-miR-UL148D-1, hcmv-miR-US4, hcmv-miR-US5-1, hcmv-miR-US5-2, hcmv-miR-US5-2-N, hcmv-miR-US25-1, hcmv-miR-US25-2-5p, hcmv-miR-US25-2-3p, hcmv-miR-US29-1, or hcmv-miR-US33-1;
d) KSHV miRNAs kshv-miR-K12-1, kshv-miR-K12-2, kshv-miR-K12-3, kshv-miR-K12-3*, kshv-miR-K12-4-5p, kshv-miR-K12-4-3p, kshv-miR-K12-5, kshv-miR-K12-6-5p, kshv-miR-K12-6-3p, kshv-miR-K12-7, kshv-miR-K12-8, kshv-miR-K12-9*, kshv-miR-K12-9, kshv-miR-K12-10a, kshv-miR-K12-10b, kshv-miR-K12-11, or kshv-miR-K12-12; or
e) human miRNAs
(i) targeting HSV: hsa-miR-138, hsa-miR-205, hsa-miR-326, hsa-miR-381, hsa-miR-425, hsa-miR-492, or hsa-miR-522;
(ii) targeting EBV: hsa-miR-24, hsa-miR-214, hsa-miR-296, hsa-miR-328, hsa-miR-346, or hsa-miR-502;
(iii) targeting HCMV: hsa-miR-15a, hsa-miR-15b, hsa-miR-16, hsa-miR-103, hsa-miR-107, hsa-miR-126, hsa-miR-142-5p, hsa-miR-184, hsa-miR-194, hsa-miR-195, hsa-miR-200b, hsa-miR-200c, hsa-miR-202, hsa-miR-326, hsa-miR-330-5p, hsa-miR-367, hsa-miR-424, hsa-miR-429, hsa-miR-450-b-3p, hsa-miR-497, hsa-miR-503, hsa-miR-548d-3p, hsa-miR-548k, hsa-miR-551a, hsa-miR-551b, hsa-miR-552, hsa-miR-592, hsa-miR-598, hsa-miR-652, hsa-miR-769-3-p, or hsa-miR-1226;
(iv) targeting KSHV: hsa-let-7a, hsa-let-7b, hsa-let-7c, hsa-let-7d, hsa-let-7e, hsa-let-7f, hsa-let-7g, hsa-let-7i, hsa-miR-1, hsa-miR-9, hsa-miR-15a, hsa-miR-15b, hsa-miR-16, hsa-miR-17-5p, hsa-miR-18a, hsa-miR-18b, hsa-miR-20a, hsa-miR-20b, hsa-miR-23a, hsa-miR-23b, hsa-miR-30a-5p, hsa-miR-30a-3p, hsa-miR-30b, hsa-miR-30c, hsa-miR-30e-5p, hsa-miR-30e-3p, hsa-miR-93, hsa-miR-98, hsa-miR-105, hsa-miR-106a, hsa-miR-106b, hsa-miR-125a, hsa-miR-125b, hsa-miR-129, hsa-miR-134, hsa-miR-137, hsa-miR-141, hsa-miR-142-3p, hsa-miR-145, hsa-miR-150, hsa-miR-154, hsa-miR-181a, hsa-miR-181b, hsa-miR-181c, hsa-miR-181d, hsa-miR-182*, hsa-miR-194, hsa-miR-195, hsa-miR-196a, hsa-miR-196b, hsa-miR-199a, hsa-miR-199b, hsa-miR-200a, hsa-miR-205, hsa-miR-206, hsa-miR-210, hsa-miR-213, hsa-miR-299-3p, hsa-miR-302a, hsa-miR-302b, hsa-miR-302c, hsa-miR-302d, hsa-miR-324-3p, hsa-miR-326, hsa-miR-329, hsa-miR-337, hsa-miR-338, hsa-miR-340, hsa-miR-346, hsa-miR-372, hsa-miR-373, hsa-miR-424, hsa-miR-448, hsa-miR-450, hsa-miR-453, hsa-miR-455, hsa-miR-490, hsa-miR-491, hsa-miR-492, hsa-miR-497, hsa-miR-518b, hsa-miR-518c, hsa-miR-518d, hsa-miR-519d, hsa-miR-520a, hsa-miR-520b, hsa-miR-520c, hsa-miR-520d, hsa-miR-520g, hsa-miR-520h, hsa-miR-525, or hsa-miR-526b; or
(v) targeting VZV: hsa-miR-99a, hsa-miR-99b, hsa-miR-100, hsa-miR-124a, hsa-miR-132, hsa-miR-141, hsa-miR-150, hsa-miR-197, hsa-miR-200a, hsa-miR-212, hsa-miR-219, hsa-miR-330, hsa-miR-374, hsa-miR-371, hsa-miR-339, hsa-miR-451, hsa-miR-495, and hsa-miR-510.

23. A pharmaceutical composition for treatment of herpes virus infection caused by HSV, EBV, HCMV, KSHV or VSV, comprising a pharmaceutical carrier and miRNA comprising one or more of:
a) HSV miRNAs hsv1-miR-H1, or hsv1-miR-LAT;
b) EBV miRNAs ebv-miR-BART1-3p, ebv-miR-BART1-5p, ebv-miR-BART2, ebv-miR-BART3-3p, ebv-miR-BART3-5p, ebv-miR-BART4, ebv-miR-BART5, ebv-miR-BART6-3p, ebv-miR-BART6-5p, ebv-miR-BART7, ebv-miR-BART8-3p, ebv-miR-BART8-5p, ebv-miR-BART9, ebv-miR-BART10, ebv-miR-BART11-3p, ebv-miR-BART11-5p, ebv-miR-BART12, ebv-miR-BART13, ebv-miR-BART14-3p, ebv-miR-BART14-5p, ebv-miR-BART15, ebv-miR-BART16, ebv-miR-BART17-3p, ebv-miR-BART17-5p, ebv-miR-BART18, ebv-miR-BART19, ebv-miR-BART20-3p, ebv-miR-BART20-5p, ebv-miR-BHRF1-1, ebv-miR-BHRF1-2*, or ebv-miR-BHRF1-3;
c) HCMV miRNAs hcmv-miR-UL22-1, hcmv-miR-UL22A-1*, hcmv-miR-UL31-1, hcmv-miR-UL36-1, hcmv-miR-UL36-1-N, hcmv-miR-UL53-1, hcmv-miR-UL54-1, hcmv-miR-UL70-3p, hcmv-miR-UL70-5p, hcmv-miR-UL102-1, hcmv-miR-UL102-2, hcmv-miR-UL111a-1, hcmv-miR-UL112-1, hcmv-miR-UL148D-1, hcmv-miR-US4, hcmv-miR-US5-1, hcmv-miR-US5-2, hcmv-miR-US5-2-N, hcmv-miR-US25-1, hcmv-miR-US25-2-5p, hcmv-miR-US25-2-3p, hcmv-miR-US29-1, or hcmv-miR-US33-1;
d) KSHV miRNAs kshv-miR-K12-1, kshv-miR-K12-2, kshv-miR-K12-3, kshv-miR-K12-3*, kshv-miR-K12-4-5p, kshv-miR-K12-4-3p, kshv-miR-K12-5, kshv-miR-K12-6-5p, kshv-miR-K12-6-3p, kshv-miR-K12-7, kshv-miR-K12-8, kshv-miR-K12-9*, kshv-miR-K12-9, kshv-miR-K12-10a, kshv-miR-K12-10b, kshv-miR-K12-11, or kshv-miR-K12-12; or
e) human miRNAs
(i) targeting HSV: hsa-miR-138, hsa-miR-205, hsa-miR-326, hsa-miR-381, hsa-miR-425, hsa-miR-492, or hsa-miR-522;
(ii) targeting EBV: hsa-miR-24, hsa-miR-214, hsa-miR-296, hsa-miR-328, hsa-miR-346, or hsa-miR-502;
(iii) targeting HCMV: hsa-miR-15a, hsa-miR-15b, hsa-miR-16, hsa-miR-103, hsa-miR-107, hsa-miR-126, hsa-miR-142-5p, hsa-miR-184, hsa-miR-194, hsa-miR-195, hsa-miR-200b, hsa-miR-200c, hsa-miR-202, hsa-miR-326, hsa-miR-330-5p, hsa-miR-367, hsa-miR-424, hsa-miR-429, hsa-miR-450-b-3p, hsa-miR-497, hsa-miR-503, hsa-miR-548d-3p, hsa-miR-548k, hsa-miR-551a, hsa-miR-551b, hsa-miR-552, hsa-miR-592, hsa-miR-598, hsa-miR-652, hsa-miR-769-3-p, or hsa-miR-1226;
(iv) targeting KSHV: hsa-let-7a, hsa-let-7b, hsa-let-7c, hsa-let-7d, hsa-let-7e, hsa-let-7f, hsa-let-7g, hsa-let-7i, hsa-miR-1, hsa-miR-9, hsa-miR-15a, hsa-miR-15b, hsa-miR-16, hsa-miR-17-5p, hsa-miR-18a, hsa-miR-18b, hsa-miR-20a, hsa-miR-20b, hsa-miR-23a, hsa-miR-23b, hsa-miR-30a-5p, hsa-miR-30a-3p, hsa-miR-30b, hsa-miR-30c, hsa-miR-30e-5p, hsa-miR-30e-3p, hsa-miR-93, hsa-miR-98, hsa-miR-105, hsa-miR-106a, hsa-miR-106b, hsa-miR-125a, hsa-miR-125b, hsa-miR-129, hsa-miR-134, hsa-miR-137, hsa-miR-141, hsa-miR-142-3p, hsa-miR-145, hsa-miR-150, hsa-miR-154, hsa-miR-181a, hsa-miR-181b, hsa-miR-181c, hsa-miR-181d, hsa-miR-182*, hsa-miR-194, hsa-miR-195, hsa-miR-196a, hsa-miR-196b, hsa-miR-199a, hsa-miR-199b, hsa-miR-200a, hsa-miR-205, hsa-miR-206, hsa-miR-210, hsa-miR-213, hsa-miR-299-3p, hsa-miR-302a, hsa-miR-302b, hsa-miR-302c, hsa-miR-302d, hsa-miR-324-3p, hsa-miR-326, hsa-miR-329, hsa-miR-337, hsa-miR-338, hsa-miR-340, hsa-miR-346, hsa-miR-372, hsa-miR-373, hsa-miR-424, hsa-miR-448, hsa-miR-450, hsa-miR-453, hsa-miR-455, hsa-miR-490, hsa-miR-491, hsa-miR-492, hsa-miR-497, hsa-miR-518b, hsa-miR-518c, hsa-miR-518d, hsa-miR-519d, hsa-miR-520a, hsa-miR-520b, hsa-miR-520c, hsa-miR-520d, hsa-miR-520g, hsa-miR-520h, hsa-miR-525, or hsa-miR-526b; or
(v) targeting VZV: hsa-miR-99a, hsa-miR-99b, hsa-miR-100, hsa-miR-124a, hsa-miR-132, hsa-miR-141, hsa-miR-150, hsa-miR-197, hsa-miR-200a, hsa-miR-212, hsa-miR-219, hsa-miR-330, hsa-miR-374, hsa-miR-371, hsa-miR-339, hsa-miR-451, hsa-miR-495, and hsa-miR-510.

24. The pharmaceutical composition of claim 23, comprising one or more modifications selected from: (1) the miRNA comprising at least one chemical modification; (2) the miRNA being replaced with a siRNA that hybridizes with the herpes virus sequence with which the miRNA hybridizes in situ; (3) the miRNA being provided as a vector with a polynucleotide that, when transcribed and processed in a mammalian cell, produces the one or more miRNAs; or (4) the polynucleotide being customized to produce a siRNA that hybridizes with the herpes virus sequence with which the miRNA hybridizes in situ.


SEQUENCE LISTING (429 sequences)

SEQ ID NO: 1 - DNA, 199 bp, herpes simplex virus
atggcaggagccgcgcatatatacgcttggagccagcccgccctcacagggcgggccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggta

SEQ ID NO: 2 - DNA, 146 bp, herpes simplex virus
gggacgccccccgtgtttgtggggaggggggggtcgggcgctgggtggtctctggccgcgcccactacaccagccaatccgtgtcggggaggggaaaagtgaaagacacgggcaccacacaccagcgggtcttttgtgttggccct

SEQ ID NO: 3 - DNA, 500 bp, herpes simplex virus
cgatgcctcgacggaaacccgtccgggttcggggggcgaaccggccgcctgtcgctcgtcagggccggcggcgctcctcgccgccctagaggctggtcccgctggtgtgacgttttcctcgtccgcgccccccgaccctcccatggatttaacaaacgggggggtgtcgcctgcggcgacctcggcgcctctggactggaccacgtttcggcgtgtgtttctgatcgacgacgcgtggcggcccctgatggagcctgagctggcgaaccccttaaccgcccacctcctggccgaatataatcgtcggtgccagaccgaagaggtgctgccgccgcgggaggatgtgttttcgtggactcgttattgcacccccgacgaggtgcgcgtggttatcatcggccaggacccatatcaccaccccggccaggcgcacggacttgcgtttagcgtgcgcgcgaacgtgccgcctcccccgagtcttcggaatgtcttggcggccg

SEQ ID NO: 4 - DNA, 54 bp, herpes simplex virus
aaggcatcgacgtccggggtttttgtcggtgggggcttttgggtatttccgatg

SEQ ID NO: 5 - DNA, 500 bp, herpes simplex virus
cccgccgtccccttacagttccaccgaacccggcccgggggactcactacccaccgcgagatgtccaatccacagacgaccatcgcgtatagcctatgccacgccagggcctcgctgaccagcgcactgcccgacgccgcgcaggtggtgcatgtttttgagtacggcacccgcgcgatcatggtacggggccgggagcgccaggaccgcctgccgcgcggaggcgttgttatccagcacacccccattgggctgttggtgattatcgactgtcgcgccgaattttgtgcctaccgctttataggccgggacagcaaccagaagctcgaacgcgggtgggacgcccatatgtacgcgtatccgttcgactcctgggtcagctcctcgcgcggcgaaagcgcccggagcgccacggccggcattttgaccgtggtctggaccgcggacaccatttacatcactgcaaccatttacgggtcgcccccagaggagacgccagg

SEQ ID NO: 6 - DNA, 500 bp, herpes simplex virus
gtctcgggaccgcactcgttcggtacgtggtcgtccgcggaccggcggcgctgttgccggaacgcaccgaggggccaagttggcccccggacccgggccgtttcccacccccaccccaaccccaaaaaccgccccccccccgtcaccggtttccgcgacccaccgggcccggccaggcacggcagcatgggacccacagaccgcccgtgatccttaggggccgtgcgatggacaccgcagatatcgtgtgggtggaggagagcgtcagcgccattaccctttacgcggtatggctgcccccccgcgctcgcgagtacttccacgccctggtgtattttgtatgtcgcaacgccgcaggggagggtcgcgcgcgctttgcggaggtctccgtcaccgcgacggagctgcgggatttctacggctccgcggacgtctccgtccaggccgtcgtggcggccgcccgcgccgcgacgacgccggccgcctccccgctggagccc

SEQ ID NO: 7 - DNA, 499 bp, herpes simplex virus
aaaccaaaacaatgttctgtatacggtcgcacgcgtgtcgtttttaaaaaacccacaatcgccggggtgagggggggggggggacggtgatagtaacgggatcggacgccacacaccagacatacaccacggtcgggttaaacacaaacggtttattaaaacggaaccaaacagctaccaacggcggacggtgctgtacacggggtcctcggcgggctcggggtcgtaccccccaacggtgtcatagatgggatcgtcgtcgggcaggtgccgcgggtgttgtatcttggcgtacaatacgtcggtttggtcgtccgccacctcgtcgtaaatcggctccccgtcggaatctccgtaccggtcgagctggccgccgtatgagatcgcgtaggggtcttccgcatattcgggaatcccgggcgggctgccgggtgcgggcctgtggcggccgtctcgcgatccgcgcatggaactgcgtacgcgcttgagggcggaatgt

SEQ ID NO: 8 - DNA, 500 bp, herpes simplex virus
gaatcagcgttcacccggcggcgcgctcaaccaccgctccccccacgtcgtctcggaaatggagtccacggtaggcccagcatgtccgccgggacgcaccgtgactaagcgtccctgggccctggccgaggacacccctcgtggccccgacagcccccccaagcgcccccgccctaacagtcttccgctgacaaccaccttccgtcccctgccccccccaccccagacgacatcagctgtggacccgagctcccattcgcccgttaaccccccacgtgatcagcacgccaccgacaccgcagacgaaaagccccgggccgcgtcgccggcactttctgacgcctcagggcctccgaccccagacattccgctatctcctgggggcacccacgcccgcgacccggacgccgatcccgactccccggaccttgactctatgtggtcggcgtcggtgatccccaacgcgctgccctcccatatactagccgagacgttcgagc

SEQ ID NO: 9 - DNA, 500 bp, herpes simplex virus
gccgctcgtctcatcgccgcgcgtcccccgagacgcccggtacggcggccaaactgaaccgcccgcccctgcgcagatcccaggcggcgttaaccgcacccccctcgtccccctcgcacatcctcaccctcacgcgcatccgcaagctatgcagccccgtgttcgccatcaaccccgccctacactacacgaccctcgagatccccggggcccgaagcttcggggggtctgggggatacggtgacgtccaactgattcgcgaacataagcttgccgttaagaccataaaggaaaaggagtggtttgccgttgagctcatcgcgaccctgttggtcggggagtgcgttctacgcgccggccgcacccacaacatccgcggcttcatcgcgcccctcgggttctcgctgcaacaacgacagatagtgttccccgcgtacgacatggacctcggtaagtatatcggccaactggcgtccctgcgcacaacaaacccctcggtc

SEQ ID NO: 10 - DNA, 500 bp, herpes simplex virus
aaatcagtgcccacggggcagactttcctcccgcgtctggttgtgtgtgtatgtgggtgggtgggtgtgggtcgggtcgacccggggccccttgggagagccatgcgaaagaaaagaggacttacgtttgtgttgtggctggaggcaaacacgatggtactgcgcgacccgtccggaaacgagaaggagatggtttcccctttaacgtggtccactcgggccgaaccgaaccagccccgcaggcaggcgtcgatctcctcaaacaccggctcggtcgccttgcggatgtgcgccgtgtagccgatcttgatcccccgaaaggaggccagcgacagcgcgatgaggggcaccagaaaccaggtcttgccgtggcgccgggggacgagaaacacggtggcgcgctggcggaagtggcgcacggccgcgtcgctaaacagggggatctcaaacacgagacgcaggaacgtgttgacctgctccgcgtggtccccgaggagcac

SEQ ID NO: 11 - DNA, 500 bp, herpes simplex virus
cgggggtggggcgggggggggggtatataaggcctgggatcccacgtccccgggtctgttggggacactgggttctcctggaacgaggccgcagccttctcccggtgcctttcccccccgaccggcacccggcctctcacacagcatcccccgcctttttgggtccgggcccgtcgtgtctttcggtggaccttgggccgtcgggcacgtacacgggtggccgggcgttggggtggatcttagcctccccgggccaatatcgctagagacagccgatctccacgcgaccccatggccgctcccaaccgcgaccctccgggataccggtatgccgcggccatggtgccgaccgggtccctccttagcacgatcgaggtggcgtcgcatcgacgcctgtttgattttttttcccgcgtgcgctccgatgcaaacagcctgtacgacgtcgagttcgacgccctgctggggtcgtattgcaacaccctgtcgctcgtgcgctt

SEQ ID NO: 12 - DNA, 192 bp, herpes simplex virus
gagtgtttcgttccttccccctccccccgcgtcagacaaaccctaaccaccgcttaagcggcccccgcgaggtccgaagactcatttggatccggcgggagccacccgacaacagcccccgggttttcccacgccagacgccggtccgctgtgccatcgcgccccctcatcccaccccccatcttgtcccca

SEQ ID NO: 13 - DNA, 477 bp, herpes simplex virus
aaaaggacgcaccgccgccctaatcgccagtgcgttccggacgccttcgccccacacagccctcccgaccgacacccccatatcgcttcccgacctccggtcccgatggccgtcccgcaatttcaccgccccagcaccgttaccaccgatagcgtccgggcgcttggcatgcgcgggctcgtcttggccaccaataactctcagtttatcatggataacaaccacccgcacccccagggcacccaaggggccgtgcgggagtttctccgcggtcaggcggcggcgctgacggaccttggtctggcccacgcaaacaacacgtttaccccgcagcctatgttcgcgggcgacgccccggccgcctggttgcggcccgcgtttggcctgcggcgcacctattcaccgtttgtcgttcgagaaccttcgacgcccgggaccccgtgaggcccggggagttccttctggggtgttttaatc

SEQ ID NO: 14 - DNA, 33 bp, herpes simplex virus
ggcccggggagttccttctggggtgttttaatc

SEQ ID NO: 15 - DNA, 500 bp, herpes simplex virus
agctttattatgttacgcccacccccgtgtgttgttctcggtgttatggtgtgcgggcgggcgggggggggggtggaagaccaagacagacaaacgcagctcggtttttgggaagcgatcaccgcgactcgtagcctaatcaggggaaccggggccatggtacgggggcatgggtggcggaaacaacactaaccccgggggtccggtccataaacaggccgggtctctggccagcagggcacatatgatcgcgggcaccccaccgcactccacgatggaacgcgggggggatcgcgacatcgtggtcaccggtgctcggaaccagttcgcgcccgacctggagccgggggggtcggtatcgtgcatgcgctcgtcgctgtcctttctcagcctcatatttgatgtgggccctcgcgacgtcctgtccgcggaggccatcgagggatgtttggtcgaggggggcgagtggacgcgcgcgaccgcgggccctgggccgccgc

SEQ ID NO: 16 - DNA, 500 bp, herpes simplex virus
ccgacaaaccccctccgcgccaggcccgccgccactgtcgtcgccgtcccacgctctcccctgctgccatggattccgcggccccagccctctcccccgctctgacggcccttacggaccagagcgcgacggcggacctggcgatccagattccaaagtgccccgaccccgagaggtacttctacacctcccagtgtcccgacattaaccacctgcgctccctcagcatccttaaccgctggctggaaaccgagcttgttttcgtgggggacgaggaggacgtctccaagctttccgagggcgagctcagcttttaccgcttcctcttcgctttcctgtcggccgccgacgacctggttacggaaaacctgggcggcctctccggcctgtttgagcagaaggacattctccactactacgtggagcaggaatgcatcgaagtcgtacactcgcgcgtgtacaacatcatccagctggtgcttttccacaacaacgaccag

SEQ ID NO: 17 - DNA, 53 bp, herpes simplex virus
cggggcggggccttggcggccgcccaactctcgcaccatcccgggttaatgta

SEQ ID NO: 18 - DNA, 500 bp, herpes simplex virus
gctcctcccgataaaaagcgccccgatggccctggacgcggcataactccgaccggcgggtcccgaccgaacgggcgtcaccatgcagcgccggacgcgcggcgcgagctccctgcggctggcgcggtgcctgacgcctgccaacctgatccgcggcgacaacgcgggcgttcccgagcggcgcatcttcggcgggtgtctgctccccaccccggaggggctccttagcgcggccgtgggcgccttgcggcagcgctccgacgacgcgcagccggcgtttctgacctgcaccgatcgcagcgtccggttggccgcgcggcaacacaacacggttcccgagagtttgatcgtggacgggctcgccagcgacccgcactacgagtacatccggcactacgcttcggccgccacccaggcgctgggcgaggtggagctgcccggcggccagttgagccgcgccatcctcacgcagtactggaagtacctgcagacggtggtgc

SEQ ID NO: 19 - DNA, 480 bp, herpes simplex virus
acccgccctgtgtggggtgaggggtgggggtggagggtgtcccaggacttccccttcctcgcggaaaccgagaccgtttggggcgtgtctgtttcttggcccctggggattggttagacccatgggttgtggttatatgcacttcctataagactctcccccaccgcccacagagggccactcacgcatccccagtgggttttgcggaccctctcttctctcccgggccgcccctatcgctcgacctctccacacctgcaccacccccgccgtccgaacccaggcctaattgtccgcgcatccgaccctagcgtgttcgtggaaccatgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccaga

SEQ ID NO: 20 - DNA, 500 bp, herpes simplex virus
atgcgtgttttcatccaacccgtgtgttttgtgtttgtgggatggaggggcgggtgtgatagacccacaggcatccaacataaacaactacacacaggaaagatgcgatacaaacgttttttattgcccggaacgaacccaaagctgtgggctaaataccggtagaaccaaaacccccggtcccgcgctcgctcgggggggcctccgcgtcaaactcgttcgtaaacaccaggagcggcgggttcctgggttcggcggttgagtccggaacacccctggggtagtttcgaagcgctttggtcccgtgaaagttgtccggggggatccaaggaagagcgtccgcccccgcaaccaggagctgggcgaccttggcgccggcctcgagggtcacaggaacccccgtaaggttgtaaacaacaaacgcacatacgtgcccggggagccagcgcgtaggaacgaccaggaggccgcgggcgttgagcgacgaccgccccaacaca

SEQ ID NO: 21 - DNA, 500 bp, herpes simplex virus
taacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacctgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggcgtcgtgagtcccgcccacaagatggtggccccggccacctacctcttgaactacgcaggccgcatcgtatcgagcgtgttcctgcagtacccctacacgaaaatt

SEQ ID NO: 22 - DNA, 34 bp, herpes simplex virus
gtccggtcgccccgacccccttgtatgtccccaa

SEQ ID NO: 23 - DNA, 500 bp, herpes simplex virus
ggcgccccatcccgaggccccacgtcggtcgccgaactgggcgaccgccggcgaggtggacgtcggagacgagctaatcgcgatttccgacgaacgcggacccccccgacatgaccgcccgcccctcgccacgtcgaccgcgccctcgccacacccgcgacccccgggctacacggccgttgtctccccgatggccctccaggctgtcgacgccccctccctgtttgtcgcctggctggccgctcggtggctccggggggcttccggcctgggggccgtcctgtgtgggattgcgtggtatgtgacgtcaattgcccgaggcgcataaagggccggtggtccgcctagccgcagcaaattaaaaatcgtgagtcacagcgaccgcaacttcccacccggagctttcttccggcctcgatgacgtcccggctctccgatcccaactcctcagcgcgatccgacatgtccgtgccgctttatcccacggcctcgccagtttc

SEQ ID NO: 24 - DNA, 444 bp, herpes simplex virus
agggccggtggtccgcctagccgcagcaaattaaaaatcgtgagtcacagcgaccgcaacttcccacccggagctttcttccggcctcgatgacgtcccggctctccgatcccaactcctcagcgcgatccgacatgtccgtgccgctttatcccacggcctcgccagtttcggtcgaagcctactactcggaaagcgaagacgaggcggccaacgacttcctcgtacgcatgggccgccaacagtcggtattaaggcgtcgacgcagacgcacccgctgcgtcggcatggtgatcgcctgtctcctcgtggccgttctgtcgggcggatttggggcgctcctgatgtggctgctccgctaaaagaccgcatcgacacgcgcgtccttcttgtcgtctctcttcccccccatcaccccgcaatttgcacccagcctttaactac

SEQ ID NO: 25 - DNA, 82 bp, herpes simplex virus
aagaccgcatcgacacgcgcgtccttcttgtcgtctctcttcccccccatcaccccgcaatttgcacccagcctttaactac

SEQ ID NO: 26 - DNA, 500 bp, herpes simplex virus
cccgggcaagtatgcccccctggcgagcccagaccccttctccccacaacatggagcatacgctcgggcccgcgtcgggatccacaccgcggttcgcgtcccgcccaccggaagcccaacccacacgcacttgcggcaagacccgggcgatgagccaacctcggatgactcagggctctaccctctggacgcccgggcgcttgcgcacctggtgatgttgcccgcggaccaccgggccttctttcgaaccgtggtcgaggtgtctcgcatgtgcgctgcaaacgtgcgcgatcccccgcccccggctacaggggccatgttgggccgccacgcgcggctggtccacacccagtggctccgggccaaccaagagacgtcgcccctgtggccctggcggacggcggccattaactttatcaccaccatggccccccgcgtccaaacccaccgacacatgcacgacctgttgatggcctgtgctttctggtgctgtctgacac

SEQ ID NO: 27 - DNA, 500 bp, herpes simplex virus
gtcccgggtacgaccatcacccgagtctctgggcggagggtggttcccccccgtggctctcgagatgagccagacccaacccccggccccagttgggccgggcgacccagatgtttacttaaaaggcgtgccgtccgccggcatgcaccccagaggtgttcacgcacctcgaggacacccgcgcatgatctccggacccccgcaacggggtgataatgatcaagcggcggggcaatgtggagattcgggtctactacgagtcggtgcggacactacgatctcgaagccatctgaagccgtccgaccgccaacaatccccaggacaccgcgtgttccccgggagccccgggttccgcgaccaccccgagaacctagggaacccagagtaccgcgagctcccagagaccccagggtaccgcgtgaccccagggatccacgacaaccccggtctcccagggagccccggtctccccgggagccccggtctccccgggagcccc

SEQ ID NO: 28 - DNA, 370 bp, Epstein Barr virus
agacccctggggcggcgatgtcggggctgctggcggcggcgtacagccaggtgtacgccctggcggttgagctgagcgtgtgcacccggctggacccccggagtctggacgtggctgcggtggtgcgcaacgccggcctgctggccgagctggaggccatcctccttccccgtttgagacggcagaatgaccgtgcatgcagcgccctgtccctggagctggtgcacctgctagagaactcgagagaggcctctgccgcgctgctcgcccctggtagaaagggtacccgggtcccgcctctccgtaccccctcagtcgcgtactctgtggagttttacggggggcataaagtcgatgtaagtttgtgcct

SEQ ID NO: 29 - DNA, 500 bp, Epstein Barr virus
ggtgctaagcgtggtcgtgctgctagccgccctggcgtgccgtctcggtgcgcagaccccagagcagcccgcaccccccgccaccacggtgcagcctaccgccacgcgtcagcaaaccagctttcctttccgagtctgcgagctctccagccacggcgacctgttccgcttctcctcggacatccagtgtccctcgtttggcacgcgggagaatcacacggagggcctgttgatggtgtttaaagacaacattattccctactcgtttaaggtccgctcctacaccaagatagtgaccaacattctcatctacaatggctggtacgcggactccgtgaccaaccggcacgaggagaagttctccgttgacagctacgaaactgaccagatggataccatctaccagtgctacaacgcggtcaagatgacaaaagatgggctgacgcgcgtgtatgtagaccgcgacggagttaacatcaccgtcaacctaaagcccaccg

SEQ ID NO: 30 - DNA, 500 bp, Epstein Barr virus
gacccaaagtgagggggcctgagactggaccctactactattctctcgtttaaacgagagaagagagcggcgagagcagactccgaatatccccaaagtcaagggaaaggaagggggcccttagcatgggaggcgcggcgacgagcgggatagcaggacggggggctggcgaagattcccaaccgggggatcgctgaatctagtatgaaggctggcaaagatccccagtggagcgaagctagtgcagggggctcggcattcctaggagaaggagcctcgccttgagggcaaagacccccccaagcctctcatcagaatctcaaccgatttcgtcagccgcttcagacagccgcggttgtcatcatcatcgggaaaggcggtgggatcatgaagcccccaggggagcgtggcccgtggatctgtgaaactcacagtttattttctccaaatcgctccttgcaacaatggacacgcaagggcgaatgcagaaaatagtctgg

SEQ ID NO: 31 - DNA, 500 bp, Epstein Barr virus
aatctctatgtcatttattaggcacaaacttacatcgactttatgccccccgtaaaactccacagagtacgcgactgagggggtacggagaggcgggacccgggtaccctttctaccaggggcgagcagcgcggcagaggcctctctcgagttctctagcaggtgcaccagctccagggacagggcgctgcatgcacggtcattctgccgtctcaaacggggaaggaggatggcctccagctcggccagcaggccggcgttgcgcaccaccgcagccacgtccagactccgggggtccagccgggtgcacacgctcagctcaaccgccagggcgtacacctggctgtacgccgccgccagcagccccgacatcgccgccccaggggtctctagacctcgagtccggggagaacggtggccagacggcgcttgcgtctgcccccggagccctgccctcctccacccagcagcagcccggccgaggcctgcgacgcggtgct

SEQ ID NO: 32 - DNA, 500 bp, Epstein Barr virus
gtcagggtggctacttgctcaggtttctgggcataaattctcctgcctgcctctgctctggtacgttggcttctgctgctgcttgtgatcatggaaaccactcagactctccgctttaagaccaaggccctagccgtcctgtccaagtgctatgaccatgcccagactcatctcaagggaggagtgctgcaggtaaaccttctgtctgtaaactatggaggcccccggctggccgccgtggccaacgcaggcacggccgggctaatcagcttcgaggtctcccctgacgctgtggccgagtggcagaatcaccagagcccagaggaggccccggccgccgtgtcatttagaaaccttgcctacgggcgcacctgtgtcctgggcaaggagctgtttggctcggctgtggagcaggcttccctgcaattttacaagcggccacaagggggttcccggcctgaatttgttaagctcactatggaatatgatgataaggtgtc

SEQ ID NO: 33 - DNA, 500 bp, Epstein Barr virus
acgcacttgcctatttcaccttgttttagtgtggcattgggggggtggcattgcgggtggatagcctcgcgactcgtgggaaaatgggcggaagggcaccgtgggaaaatagttccaggtgacagcagcagtgtgtgaagattgtcacagctgctggtttggagaaaacgggggtgggcggtgatcagggagaacaattccccggggacacctgcacgagacccctgggctctcaggaactccgcccaggtcttgccaattggggtgatcctgtagcgccgcggtttcagcatcacaggttattttgcctgaagcttgctggggcgtaaatccctctcgccttgtttctcagagagcatttcaggccggttttgcagtcgctgctgcagctatggggtccctagaaatggtgccaatgggcgcgggtccccctagccccggcggggatccggatgggtacgatggcggaaacaactcccaatatccatctgcttctggct

SEQ ID NO: 34 - DNA, 500 bp, Epstein Barr virus
ataaaacaacagacatgcagactccaggttatgacattttatttacagccatggccaattgtagttgttattgcccttaatggggggggtggtttccatcatgtgtttattgtatgtattgggacttgaaggtggaggggggcggcgtggagctgggcctctaagtacaggtcgcgtaggtctatggggacccttgtctttggtggattgctgaactggggctggtggcctgggaggtgctgaggcccgtcccctgaccggcgcgggagccggcggcctcggaggtgcccgggtgcgtggtcgggagaacgaaggcgtgggtgtcagacctgaagactgttgggtagatggcgagactcttgaagatcgtgaggcctgagagccgggggttgcttcatcctcgtcgctctcgctgtagtcagactcgtctgaatctgaaggatgccacgaggggtcgctatcactgccctcagatgggtcttcgtcactggggtactctt

SEQ ID NO: 35 - DNA, 500 bp, Epstein Barr virus
gcctcccgcggggggaggggggcacggatgagcccaatcctcgccacctgtgctcgtatagtaagctggagttccatctcccgttacctgagagcatggcctccgtgtttgcctgctggggctgtggcgagtaccacgtatgtgatggatccagcgagtgcaccctgattgagacccatgagggagtggtgtgcgcccttacaggcaactacatggggccgcatttccagccggcgctgaggccctggaccgagatccgacaagacacacaggaccagcgggacaagtgggagcctgaacaagtccagggcctggttaagactgtggtcaatcacctctatcactactttctgaatgagaatgtcatctccggggtcagcgaggccctctttgatcaggagggggcgctgaggcctcacatcccggccctggtttcctttgtgttcccttgctgcctgatgctgtttaggggggcctcctccgagaaggtggtggatgtg

SEQ ID NO: 36 - DNA, 500 bp, Epstein Barr virus
gtggcctcgggacccccctcctcgtgcacctatttgttcccgacacggttatggcagagctttgccccaatcgcgtgccaaactgcgagggggcctggtgccagactctcttcagtgaccggacgggtctcacgagggtctgccgcgtgtttgctgctcggggcatgctgcccggacggcctagccatcggggcacgtttaccagtgtgccagtgtactgcgatgagggccttccagagctctacaaccccttccacgtggccgcccttcgattttacgatgaaggagggctggttggggagctacagatttattacctgtctctctttgagggggccaaaagggctctgaccgacgggcatcttatcagagaggcctctggggtccaggagtctgctgcggctatgcagcccatacctatagatcctgggccccccggaggggcgggtatagagcatatgccggtggccgcggcccaggtcgagcaccctaaaacgtat

SEQ ID NO: 37 - DNA, 485 bp, Epstein Barr virus
atttcaagagctgaaccagaataatctccccaatgatgtttttcgggaggctcaaagaagttacctggtatttctgacatcccagttctgctacgaagagtacgtgcagaggacttttggggtgcctcggcgccaacgcgccatagacaagaggcagagagccagtgtggctggggctggtgctcatgcacaccttggcgggtcatccgccacccccgtccagcaggctcaggccgccgcatccgctgggaccggggccttggcatcatcagcgccgtccacggccgtagcccagtccgcgaccccctctgtttcttcatctattagcagcctccgggccgcgacttcgggggcgactgccgccgcctccgccgccgcagccgtcgataccgggtcaggtggcgggggacaaccccacgacaccgccccacgcggggcacgtaagaaacagtagagggcacgaaacatggtgtatgcactttatt

SEQ ID NO: 38 - DNA, 500 bp, Epstein Barr virus
ccgggaacagcttcgcaagttcctcaacaaggagtgcctctgggtgctgagcgatgcctctacgccccagatgaaagtctatacggccacaaccgccgtgtcagctgtgtacgtgcctcagatagccggacctcctaaaacctacatgaatgttaccctcattgtgctgaagcccaagaagaagcccacctatgtgaccgtctacatcaatggaaccctagccaccgtggccaggcccgaggttctcttcactaaggcagtccaggggccacacagcctgactctcatgtactttggggtattctcagatgcagtgggtgaggcggtgcctgtggagattaggggtaaccctgtagtcacctgcacagatctgaccacggcccacgtctttaccacctcaaccgccgttaaaacagtagaagaactgcaagatatcacaccctcggagatcatcccactgggacggggtggtgcctggtatgcagaaggggccctgtaca

SEQ ID NO: 39 - DNA, 378 bp, Epstein Barr virus
agcaggtggcacacattacggtgctggagattttcccactgtgcctaaacgtgatggtgctggtctccttgttgacctctacacgcttggagtcgaagctcttggtcaaggtgtcaataatttcagtgaaaacggcggacgcgacatgtttctggtgagccacgtagcctatttgcacgttggagagattcgagaggatgaggctgatgatggccacgactatccaggtcttgccgtggcgcctggggataagaaacacgctggctttttgcttaaaaatgtgcagcttctccagcgtcatttcttccaatccgaaagcactttgaaagatgtcaaacatggtgtctgtaatctctaaagatttgattgagatcagaa

SEQ ID NO: 40 - DNA, 500 bp, Epstein Barr virus
ttctaagcgagatctggtggcccagcaactaagagcctcggtagaaaagagagcggctgtgagcgcacgtgacagatttgggagggaccacgctctgtttgaaacacagtttacatctgctcggggtgccttagagtccctgcgccacgcaagggagacgtttgagtccaaacagctaatttctacctatcagagggtggtcaccgcgaccaagactcaatttccaaaaatcaactacaagcagctagagcgggtggaggagctccgtgagcaggagcttgaggccagagacgagctgcgacaggccctcgagccatttgaggaacatggatgtgaatatggctgcggagttgagcccgacgaactcctccagcagtggcgagttgagtgtctccccagaaccccctcgagagacccaggcctttttggggaaggtgactgtcattgattacttcacctttcagcacaaacacctgaaggtgaccaacattgatgacatg

SEQ ID NO: 41 - DNA, 500 bp, Epstein Bar virus
ttacttcacctttcagcacaaacacctgaaggtgaccaacattgatgacatgacggagaccctctatgtaaagctgccggagaacatgacgcgctgtgatcacctccccattacctgcgagtatctgctggggcgggggagctacggggccgtgtatgcacatgcagataatgccacggtcaaactctatgactctgtgacggagctgtatcacgagctcatggtgtgtgacatgattcagattgggaaggccacggccgaggatgggcaggacaaggccctggtggactacctgtcggcctgcacgtcctgccacgccctgtttatgccccagttcagatgcagtctccaggattatggccactggcatgatggtagtattgagcccctggtgcggggctttcagggcctcaaagatgccgtttactttctgaatcggcactgcggcctcttccattcggacattagccccagcaacatcctggtggatttcacagaca

SEQ ID NO: 42 - DNA, 257 bp, Epstein Barr virus
tgcagtgtccctgctgcccatggaatgctcagaccccgggttggtggcactgttgcgcccggccctgtacactacactctaaaagtaacctgtctacttcgccatgcttcttacactactcacctacatgtcaaccgcctctaccctccccatgggatggcggcggttatgttttccccatgttgcgggtgccggcccttacaacaggttttggcaacgagagcaatacacaattaggctaaaagcagccacctatc

SEQ ID NO: 43 - DNA, 500 bp, Epstein Barr virus
tctatacattttctcagcactttatatgaatcagggtcattgggcctgcggggaactgagccagtaggatattaggcaagggtgacacagtgcccatgcattataatttaaccaaacagtggtcgtgagttttaggccggccatgggggcttacaagaataacatgccaatgacccggcccccacttttaaattctgttgcagcagatagctgatacccaatgttatcttttgcggcagaaattgaaagtgctggccatatctacaattgggtgtcctaggtgggatatacgcctgtggtgttctaacgggaagtgtgtaagcacacacgtaatttgcaagcggtgcttcacgctcttcgttaaaataacacaaggacaagatactaaagaaataactgaggtgagtgtgggaagatgggaatactatgtgttatgttaacgggtgagagcctatactgcagcccagactcggggggaggaggaaatggtaagagttata

SEQ ID NO: 44 - DNA, 24 bp, Epstein Barr virus
caccttcatatcccttgttttacc

SEQ ID NO: 45 - DNA, 500 bp, Epstein Barr virus
caccatgttctcgtgcaagcagcacctgtccctgggggcctgtgtcttctgtctcggcctcctggccagcacccccttcatttggtgctttgtctttgccaacctgctctctctggagatcttctcaccgtggcagacacacgtgtacaggcttggattcccgacggcatgcctaatggccgtcctctggacgctggtacccgccaagcacgcggtgagggccgtcactccagccatcatgctgaatattgccagcgccttgatcttcttctccctcagagtctactcgaccagcacgtgggtttctgccccctgtctctttctggccaacctgcctctcttatgcctgtggccccggctggccatcgagattgtttacatctgcccggctatacaccaaaggttctttgaacttgggttgctcttggcctgcaccatctttgccctgtccgtggtctccagggccctggaggtgtcggctgtcttcatgtctccatttt

SEQ ID NO: 46 - DNA, 148 bp, Epstein Barr virus
ccagtcaccttccagactatgcatacactgaatttagcctgatattgtccccctagccccgggcccagccctcctcagaaaactctgcatggagaagctggacgtgaacctcccccccagacctgtgtgctgtatttacaaacactac

SEQ ID NO: 47 - DNA, 500 bp, Epstein Barr virus
cggcgactgggggcaaagccagcgcacccggggaaccggccccgtgcgcggaatcaggaccatggatgtgaatgcccccgggggcgggagtggaggctcggccctccgcatcctaggcacggcctcgtgcaaccaggcccactgcaagtttggccgctttgccggcatccagtgcgtcagcaactgcgtcctctacctggtcaagagcttcctggccggccgccccctgacctcccgccctgagctggacgaggtcctggacgagggggcgcggctggatgccctcatgcgccagagcggcatcctcaaggggcacgagatggcccagttgacggacgtgcccagctccgtggtcctgaggggcggtgggcgcgtgcacatataccgctcggcggagatctttggcctcgtcctattccctgcccagatcgcaaactcggcagttgttcagtccctggccgaggtcctgcacggcagttacaacggggtggcccagttca

SEQ ID NO: 48 - DNA, 480 bp, Epstein Barr virus
acacttctgaaaactgcctcctcctcttttagaaactatgcatgagccacaggcattgctaatgtacctcatagacacacctaaatttagcacgtcccaaaccatgacatcacagaggaggctggtgccttggctttaaaggggagatgttagacaggtaactcactaaacattgcaccttgccggccacctttgctatctttgctgaagatgatggacccaaactcgacttctgaagatgtaaaatttacacctgacccataccaggtgccttttgtacaagcttttgaccaagctaccagagtctatcaggacctgggagggccatcgcaagctcctttgccttgtgtgctgtggccggtgctgccagagcctctgccacaaggccagctaactgcctatcatgtttcaaccgctccgactgggtcgtggttttctgcccctcagcctgctcctgagaatgcttatcaagcttatgca

SEQ ID NO: 49 - DNA, 500 bp, Epstein Barr virus
atggttaaactgaatctccacctgtgtaacctcactgtaattctatgggaataacaagggaagagggaaaagagactgcgaaaattcagtcatatcggatgcctcacgcgaagggaaacgtgggaggcgaatgtagcccctaggcctgccacgtgggtctcatgggggaatgagggaaaaggccctaattcagccacctcccctgtggccgacttctggaacatttgaggaggcacacaaaatgaggaacggtgattaggcactggacacacatggcactcatggtacggtgataactgacagagccgtgtctcctgacgccaatgccaactcccccaaacatgtcctgttagctggtgcggttataactgccagagctgtgtttcccgacgccaatgctaactccccaaacatgtcctgtgagttttgcccataaatgaccccatccactgccacccctgggttcatttcctcccgttagcccaatgtaataagaggaa

SEQ ID NO: 50 - DNA, 171 bp, Epstein Barr virus
cccagcgtcaggaagtacagccggtcgtagtcatccgaggctgagaactgacgctccaggatctcccgcgccgcaagcatgggcgaggggcgccccagggcaacaccgacgccgtcctcgaaggctagacgcagctgtgtgcgcgccgccagcatggcagccgggtcgtga

SEQ ID NO: 51 - DNA, 500 bp, Epstein Barr virus
gatgcagttgctctgtgttttttgcctggtgttgctatgggaggtgggggctgccagcctcagcgaggttaagctgcacctggacatagaggggcatgcttcgcattacaccatcccatggaccgaactgatggcaaaggtcccaggccttagcccagaggcgctgtggagagaggcaaatgtcaccgaagatttggcgtctatgcttaaccgctacaagttaatttacaagacgtctggtacccttggtattgcgctggccgagcctgtcgatatccctgctgtctctgaaggatccatgcaagtggatgcatctaaggtccatcccggagtcattagcggcctgaattcccctgcctgcatgcttagtgccccccttgagaagcagctcttctactatattggcaccatgctgcccaacacgcggccacacagctatgtcttttatcagctgcgctgtcacttgtcttatgtggccctgtccatcaacggggacaagt

SEQ ID NO: 52 - DNA, 500 bp, Epstein Barr virus
gctgctccgcgtggagctggacggcatcatgcgtgaccacctggccagggcggaggagatccgccaggacctggatgctgtagtggccttctctgatggcctggagagcatgcaggtcaggtccccctccacgggagggcgctctgcgccagccccgccctccccatccccagcccagccgttcactcggctcaccgggaacgcccagtatgcagtctcaatctctcccacggacccccctctgatggtggccggcagcctggctcaaacgctgcttggtaatctgtacgggaacatcaaccagtgggtaccgtccttcggaccctggtacaggaccatgtcggctaatgccatgcagcggcgcgtgttccctaagcagctgaggggcaacctgaactttaccaactccgtctccctaaagctgatgacagaagtggtggcggtgcttgagggcaccacccaggactttttctcagacgtcaggcacctgccagacctcc

SEQ ID NO: 53 - DNA, 53 bp, Epstein Barr virus
ctcccgttattgaaaccacgcctgcttcacgcctcgtttactaatggaatatt

SEQ ID NO: 54 - DNA, 500 bp, Epstein Barr virus
caggggtcaccttggatccccttaatctagctcactttcagtggatgcatcgtagtcagtctgcttcgcgtcctttgggaacacggagatctcagaattgtcactgagaatctcctgtgcttcagcagtagcttgggaacaccgggcaggtccgtgagaactttcttctactcgaggcctttttggcgtggtggcattaatgtccagtggggtaaatgcaccttgactgtaatcactggcaaagggcatgcttgggcatgctgtacctgatgagtcacaccccacggccatgctatcttgtaacggcatagggggaggggggaatcttgttggaatggggcgtatgggggctcggggctggggagatgaccatgatggtgcagaggatgagaccagtggcaccaatgaaagttgaagacgtggtgggcctgtctccgattgcagatgtgggaactgggagacctgatcctggccatgtcctgcagatccatcccactgag

SEQ ID NO: 55 - DNA, 500 bp, Epstein Barr virus
tagaatgacagcctggtccaagagtaaaagcagaacagtaaacactgccataagtcctcatggcaggagaggcggggggtatgtgctgcgttgggaactgagtaggcttgatagcagtgactggttgtaacctatgcctggaagaatcatggcctacccgagacccccaacgtcttgggtaggccatacgtctagccacatagcaggtctccagagggcagacgttagtaacatttgtattgtgaggaaaggcctttagatatagaggctctcccaacacaatagaatttttgcagctaagttttctaagggcacgtgcctttcccccaccctggaacaaacatgggctgctatagtgagccaggctttctatgcctgaaacccaagtttccttgccatctaaagctgcaactttcagtttagatctgtggttacatggtgcatttgcaggtgtgaaatgcttggccttgagttactctaaggctagtccgatccccggg

SEQ ID NO: 56 - DNA, 500 bp, Epstein Barr virus
cctttctttacttctaggcattaccatgtcataggcttgcctgactgactctccctccatttactgggaatgccttagctaatcaccttaactggcacacactcccttagccacactgtctgtctaggctgaaaagccacattcatattctatttcaaaacaaggggaaaggaggacatgcgagaattggcagacacctttacccagcccttaacacaccacacaggtagcaaggacccgggcgttgccagactccgccaccaacgcccctgcgttgaacccacccctcctacacacatcagacctctgcacaacacaactaccaggcagatgaggccccttacttccacagggtactggcataccagcgggggaccacatacatccctgtctcccacccagtaactccagcaactttgctttccatcttgtgccaatacacatttggattcagcccaagccacacctaactcatgccagcagaggcaggaacacctgtt

SEQ ID NO: 57 - DNA, 500 bp, Epstein Barr virus
aggtaagtattattaaattttagagacactatcacgtgtaacttgacgtgcaaggatggaagagaggggcagggaaacgcaaatgccggttgcccggtatgggggcccgtttattatggtaaggctcttcgggcaagatggagaggcaaacatacaggaggaaaggctatatgagctactctctgacccacgctccgcgctcggcctagacccggggcccctgattgctgagaacctgctgctagtggcgctgcgtggcaccaacaacgatcccaggcctcagcgtcaggagagggccagagaactggccctcgttggcattctactaggaaacggcgagcagggtgaacacttgggcacggagagtgccctggaggcctcaggcaacaactatgtgtatgcctacggaccagactggatggcaaggccttccacatggtccgcggaaatccagcaattcctgcgactcctgggcgccacgtacgtgcttcgcgtggaga

SEQ ID NO: 58 - DNA, 500 bp, Epstein Barr virus; identical to SEQ ID NO: 57

SEQ ID NO: 59 - DNA, 92 bp, Human cytomegalovirus FIX
actattgtatatatatcagttactgttatggatcccacgtcactattgtatactctatattatactctatgttatactctgtaatcctactc

SEQ ID NO: 60 - DNA, 92 bp, Human cytomegalovirus; identical to SEQ ID NO: 59

SEQ ID NO: 61 - DNA, 83 bp, Human cytomegalovirus FIX
gtgaaaaactggaaagagagacatggactcttgtacatagtgattccccgtgacagtattaacgtgtggtgagaaggctgttt

SEQ ID NO: 62 - DNA, 81 bp, Human cytomegalovirus
gtgaaaaactggaaagagacatggactcttgtacatagtgattccccgtgacagtattaacgtgtggtgagaatgctgttt

SEQ ID NO: 63 - DNA, 67 bp, Human cytomegalovirus FIX
acgtggtagggggatctaccagcccagggatcgcgtctttcgccgccacgctgcttcaccgatatcc

SEQ ID NO: 64 - DNA, 67 bp, Human cytomegalovirus
acggggtagggggatctaccagcccagggatcgcgtatttcgccgccacgctgcttcaccgatatcc

SEQ ID NO: 65 - DNA, 500 bp, Human cytomegalovirus FIX
caaggaaggcgagaacgtgttttgcaccatgcagacctacagcacccccctcacgcttgtcatagtcacgtcgctgtttttgttcacaactcagggaagttcatcgaacgccgtcgaaccaaccaaaaaacccctaaagctcgccaattaccgcgccacctgcgaggaccgtacacgtactctggttaccaggcttaacactagccatcacagcgtagtctggcaacgttatgatatctacagcagatacatgcgtcgtatgccgccactttgcatcattacagacgcctataaagaaaccacgcatcagggtggcgcaactttcacgtgcacgcgccaaaatctcacgctgtacaatcttacggttaaagatacgggagtctacctcctgcaggatcagtataccggtgatgtcgaggctttttacctcatcatccacccacgtagcttctgccgagctttggaaacgcgtcgatgcttttatccgggaccagggagag

SEQ ID NO: 66 - DNA, 500 bp, Human cytomegalovirus; identical to SEQ ID NO: 65

SEQ ID NO: 67 - DNA, 401 bp, Human cytomegalovirus FIX
cgacgacgcatacccgtcgttcggcaccctacccgcttcgcacgctcagtacggctttcgactactacgcggcatatttttgattacgctcgtcatctggaccgtagtgtggctcaaactgcttcgagacgctcttttataaaaacatacgcagaaaacatttatgttccgtgatctcctgtggtaacatagcaacaggaacctgcactttccttgaattatgttctcataaactgtaccgtcctggagtacgctatgtatcacgcgtcttttcatggagcgcactgtatgccgacacacggagataacgaaggaaattccactcgcagatctgccttgtctggagatggggtaggaatacaacggcgtttaaagtaaagacagatgaggcacatggtgaa

SEQ ID NO: 68 - DNA, 401 bp, Human cytomegalovirus; identical to SEQ ID NO: 67

SEQ ID NO: 69 - DNA, 500 bp, Human cytomegalovirus FIX
acggataaccgcaaaggccacgtgcaacgttcacgctgctataagaaggccatgtcccccgtggacgggtctctttgacacgagcgcggcacgccgttgccacgagcatggatcacgcgctcttcacacacttcgtcggccggccccgtcactgtcggttggaaatgttgattctggacgaacaggtgtctaagagatcctgggacaccacggtttaccacaggcgccgcagacatctacctcgacgccgcgctccgtgcggcccccagaggcccgccgagattcccaaaagaagaaaaaaggcggccgtccttctattttggcacgatttgtgctggctgtttcgacgacttttctttcctcgggaggactcggagccactgatgtcggatccggcacggtctcccgaagaggaggagtaaacaacacacggctaagaggatacatcatcaaagaagataggaggggtcaaaacgcggactgaaagtatataacgccga

SEQ ID NO: 70 - DNA, 499 bp, Human cytomegalovirus
acggataaccgcaaaggccacgtgcaacgttcacgctgctataagaaggccatgtcccccgtggacgggtctctttgacacgagcgcggcacccgttgccacgagcatggatcacgcgctcttcacacacttcgtcggccggccccgtcactgtcggttggaaatgttgattctggacgaacaggtgtctaagagatcctgggacaccacggtttaccacaggcgccgcaaacatctacctcgacgtcgcgctccgtgcggcccccagaggcccgccgagattcccaaaagaagaaaaaaggcggccgtccttctgttttggcacgatttgtgctggctgtttcgacgacttttctttcctcgggaggactcggagccactgatgtcggatccggcacggtctcccgaagaggaggagtaaacaacacacggctaagaggatacatcatcaaagaagataggaggggtcaaaacgtggactgaaagtatataacgccga

SEQ ID NO: 71 - DNA, 97 bp, Human cytomegalovirus FIX
acaacacacggctaagaggatacatcatcaaagaagataggaggggtcaaaacgcggactgaaagtatataacgccgatcatgtccgaggaactgtt

SEQ ID NO: 72 - DNA, 97 bp, Human cytomegalovirus; identical to SEQ ID NO: 71

SEQ ID NO: 73 - DNA, 128 bp, Human cytomegalovirus FIX
cggactttggactgagccccaagcggtacggactatatattttccacaagtctacactgaacttgagcacacaaatactgacaatagactggatatatagacttttatatgatccctgtacagatgta

SEQ ID NO: 74 - DNA, 130 bp, Human cytomegalovirus
cggactttggactctgagccccaagcggtacggactacatattttccataaatctatactgaacttaagcacaaaaatactgacaatggactggatatacagacttttatataatccctgtacagatgta

SEQ ID NO: 75 - DNA, 97 bp, Human cytomegalovirus FIX
caaaacaggaaggaaaaaaacacacacatgaaaaacccggagaagacagagaggacgagcgtccacacaccgctttggtcgtagacgtactttttat

SEQ ID NO: 76 - DNA, 100 bp, Human cytomegalovirus
caaaataggaaggaaaaaaaccacacgtgaaaaaaaaaacccggagaagacagagaggacgagcgtccacacaccgctttggtcgtagacgcatttttat

SEQ ID NO: 77 - DNA, 500 bp, Human cytomegalovirus FIX
gtcatcagtgtacacacgtccagaaatagggcgacggtgtttttataaccgaaagtagcgtgtttgagacacgcgcttatagtcggttttttcaccgtcgtcgctctaggtttgattttcgcgctcttgtgtctcccgacaggctcgtcgtgggctactttgactcgctatcgtcgctctatctgcgcgggcagcccaagttcagcagcatctggcgcggtctgcgtgatgcctggacccacaagcgcccgaagccgcgcgagcgtgcgagcggggttcacctgcagcgctacgtacgcgccacggcgggtcgttggctcccgctgtgctggccgccgctgcacggcatcatgctgggcgacactcagtactttggggtggtgcgcgatcacaagacctaccggcgcttctcgtgcctgcgccaggctggccgcttgtactttatcggcctcgtcagtgtgtacgaatgcgtgccggacgcaaacacggcgcccgagatc

SEQ ID NO: 78 - DNA, 500 bp, Human cytomegalovirus
gtcatcagtgtacacgcccagaaatagggcgacggtgtttttataaccgaaagtagcgtgtttgagacacgcgcttctggtcggttttttcaccgtcgtcgctctaggtttgattttcgcgctcttgtgtctcccgacaggctcgtcgtgggctactttgactcgctctcgtcgctctatctgcgcgggcagcccaagttcagcagcatctggcgcggtctgcgtgatgcctggacccacaagcgcccgaagccgcgcgagcgtgcgagcggggttcacctgcagcgctacgtgcgcgccacggcgggtcgttggctcccgctgtgctggccgccgctgcacggcatcatgctgggcgacactcagtactttggggtggtgcgcgatcacaagacctaccggcgcttctcgtgcctacgccaggctggccgcttgtactttatcggcctcgtcagtgtgtacgaatgcgtgccggacgcaaacacggcgcccgagatctg

SEQ ID NO: 79 - DNA, 500 bp, Human cytomegalovirus FIX
ccctccgtccgtcctcctttcccgacacgtcactatccgatgatttcattaaaaagtacgtctgcgtgtgtgtttcttaactattcctccgtgttcttaatcttctcgatcttttgaaggatgttctgcacggcgtccgacggcgttttggcgccccccatgccggcagaacccggttgcggccccgtaccgctcttctggggcgacgataggtcgaaagccaccgttttcatgcccgtcgtgctcttgacgggggaacctacggcggcggtccccgtcgagcggcgtgattgcaaagccgcgctcgcccccggtttcaggatggagggggaggccacaggcggcgcattcgatacgctgcttttggccgtagacgacggtgggtaaacggtggttaccgcgggatacgtcggcgtggtcgaggcggcccggctgctgccggacaggcgacccggcgcgctaccgctcacggggaccgagggcggtcgacctaccaccgc

SEQ ID NO: 80 - DNA, 500 bp, Human cytomegalovirus; identical to SEQ ID NO: 79

SEQ ID NO: 81 - DNA, 500 bp, Human cytomegalovirus FIX
ttaagaaacacacacgcagacgtactttttaatgaaatcatcggatagtgacgtgtcgggaaaggaggacggacggagggtcagggatggggagatgtgagaaagttgtccgcgggcaattgcatgtcgcccagaaagaacgtggttgctccggcggcgtgcatctgccgaaacaccgtgtggtgattgtacgagtacacgttaccgtcgccctcggtgatttgatacaacgtggcgatgggggtgccctgcgggatcacgatggaacgcgtgcgcgtccacagcgtgactttgagcggctcgccgccgcgccacacgctgagccccgtgtaaaaggcgtcctcgtgtggcaagttggccaccaagaaacaccggtctgtgatctgcacgtagcgcaagtccaactccaccgtctgccgcggttgcactccgaagtggatatcgtaaggcgcgtgcaccgtgagcgaaaacacgttgggctcgttgagaagcggacagtt

SEQ ID NO: 82 - DNA, 500 bp, Human cytomegalovirus
ttaagaaacacacacgcagacgtactttttaatgaaaccatcggatagtgacgtgtcgggaaaggaggacggacggagggtcagggatggggagacgtgagaaagttgtccgcgggcaattgcatgtcgcccagaaagaacgtggttgctccggcggcgtgcatctgccgaaacaccgtgtggtggttgtacgagtacacgttaccgtcgccctcggtgatttgatacaacgtggcgatgggggtgccctgcgggatcacgatggaacgcgtgcgcgtccacagcgtgactttgagcggctcgccaccgcgccacacgctgagccccgtgtaaaaggcgtcctcgtgtggcaagttggccaccaagaaacaccggtctgtgatctgcacgtagcgcaagtccaactccaccgtctgccgcggttgcaccccgaagtggatatcgtaaggcgcgtgcaccgtgagcgaaaacacgttgggctcattgagaagcggacagtt

SEQ ID NO: 83 - DNA, 18 bp, Human cytomegalovirus FIX
gctttcctgttactttat

SEQ ID NO: 84 - DNA, 18 bp, Human cytomegalovirus
gctttcctgttactttat

SEQ ID NO: 85 - DNA, 14 bp, Human cytomegalovirus FIX
cgtcactggagaac

SEQ ID NO: 86 - DNA, 14 bp, Human cytomegalovirus
cgtcactggagaac

SEQ ID NO: 87 - DNA, 500 bp, Human cytomegalovirus FIX
cgtcaacgctgatagtgtctataaaggccgtgccgccgcgccgtagttctccgaaggcggacggaggagtctgtcgaccgcagcggtggctggagaagcgcagcgtcggcgagcgaaggtagaggagtccgtcatggacgacctacgggacacgctgatggcctacggctgcatcgccatccgagccggggactttaacggtctcaacgactttctggagcaggaatgcggcacccggctgcacgtggcctggcctgaacgctgcttcatccagctccgttcgcgcagcgccctggggcctttcgtgggcaagatgggcaccgtctgttcgcaaggtaagccccacgtcgttgaagacacctggaaagaggacgttcgctcgggcacgttctttccaggtgttttcaacgtgcgtggattttttctctctaccaggtgcttacgtctgctgtcaggagtacctgcacccctttggcttcgtcgagggtccgggctttatg

SEQ ID NO: 88 - DNA, 500 bp, Human cytomegalovirus; identical to SEQ ID NO: 87

SEQ ID NO: 89 - DNA, 106 bp, Human cytomegalovirus FIX
aaggagaactttgctgctagatgaccatgttcagctttttttttgtagtattttttcatagttgctatacctcagttatcccccctattagccccacatgctgctt

SEQ ID NO: 90 - DNA, 105 bp, Human cytomegalovirus
aaggagaactttgctgctagatgaccatgtcagctttttttttgtagtattttttcatagttgctatacctcagttatcccccctattagccccacatgctgctt

SEQ ID NO: 91 - DNA, 51 bp, Human cytomegalovirus FIX
taatgataactgcacatcctcacgagtgcccttacctatcatcacactaag

SEQ ID NO: 92 - DNA, 50 bp, Human cytomegalovirus
taatgataactgcacatcctcacgagtgccttacctatcatcacactaag

SEQ ID NO: 93 - DNA, 500 bp, Human cytomegalovirus FIX
gccgcggacgccgtcggtaccgtctccaccacagttgccaccgtcgccgtcactgccaccgacatggagcccacgccgatgctccgcgagcgggatcacgacgacgcgccccccacctacgagcaagccatgggcctgtgcccaacgacggtttccacgccaccgccgccaccacccgattgcagcccaccgccctatcgacccccgtactgcctggttagttcgccgtcgccgcgacacacgttcgacatggatatgatggaaatgcccgccaccatgcatcccaccacgggggcgtactttgacaacggctggaaatggacttttgctctcttagtggtcgctatattagggatcattttcttggccgtggtgttcaccgtggtgattaaccgggacagtgccaatacaacaacgggggtttcctcatcatcggggtaacggggatagagcatgtgcttgactgtaccatcattgctgctacggaataataactacgc

SEQ ID NO: 94 - DNA, 499 bp, Human cytomegalovirus
gccgcggacgccgtcggtaccgtctccacccagttaccaccgtcgccgtcactgccaccgacatggagcccacgccgatgctccgcgaccgggatcacgacgacgcgccccccacctacgagcaggccatgggtctgtgcccgacgacggtttccacaccaccgccgccaccacccgactgcagcccaccgccctatcgacccccgtactgcctggttagttcgccgtcgccgcgacacacgttcgacatggatatgatggaaatgcccgccaccatgcatcccaccacgggggcgtactttgacaacggctggaaatggacttttgctctcttagtggtcgctatattagggatcattttcttggccgtggtgttcaccgtggtgattaaccgggacaattccactacaacgggtacatcatcggggtaacgggaaatagagcatgtgcttgactgtaccatcattgctgctacggaataataactacgctacgacct

SEQ ID NO: 95 - DNA, 500 bp, Human cytomegalovirus FIX
agcgcgtgcccgggaacgcggcccgcgcgcacggcgcggtcccgcgatggagaaaacgccggcggagacgacggcggtttcagctggcaacgtgccacgtgactcaatcccgtgtataactaacgtgtccgcggacacccgcggccgtacccgccccagcagaccagccaccgttcctcagcgacgtcccgcgcggatcggacactttaggcggcgcagcgccagccttagctttcttgactggccggacgacagcgtcacagagggcgttcggacgacctccgcgtcggtcgccgcctccgcggcccgtttcgacgaaatccggcgacgccgccagagcattaacgacgagatgaaggaacgcacgctggaggacgcgctggctgtcgagctggtcaacgagaccttccgctgctctgtcaccgccgacgcccgcaaggacctgcagaagctggttcgtcgcgtcagtggcacggtgctgcgtctcaactggccgaacg

SEQ ID NO: 96 - DNA, 500 bp, Human cytomegalovirus
agcgtgggccgcgtgcctgggaacgcgcgcacggcgcggtcccgtgatggagaaaacgccggcggagacgacggcggtttcagctggcaacgtgccacgtgactcaattccgtgtataactaacgtgtccgcggacacccgcggccgtacccgtcccagcagaccagccaccgtccctcagcgacgtcccgcgcggatcggacactttaggcggcgcagcgccagccttagctttcttgactggccggacgacagcgtcacagagggcgttcggacgacctccgcgtcggtcgccgcctccgcggcccgtttcgacgaaatccggcggcgccgccagagcatcaacgacgagatgaaggaacgtacgctggaggacgcgctggctgtcgagctggtcaacgagaccttccgctgctctgtcaccgccgacgcccgcaaggacctgcagaagctggttcgtcgcgtcagcggcacggtgctgcgtctcagctggccgaacg

SEQ ID NO: 97 - DNA, 480 bp, Human cytomegalovirus FIX
tcgggggcccgctggctcggcgcggctgtattattagacgccgggcgtcttcgcagcgttcccggtcgtcgtgtgtgctctctataaaactttcgctcgctcgcgcccgctccttagtcgagacttgcacgctgtccgggatggatcgcaagacgcgcctctcggagccgccgacgctggcgctgcggctgaagccgtacaagacggctatccagcagctgcgatctgtgatccgtgcgctcaaggagaacaccacggttaccttcttgcccacgccgtcgcttatcttgcaaacggtacgcagtcactgcgtgtcaaaaatcacttttaacagctcatgcctctacatcactgacaagtcgtttcagcccaagaccattaacaattccacgccgctgctgggtaatttcatgtacctgacttccagcaaggacctgaccaagttctacgtgcaggacatctcggacctgtcggccaaga

SEQ ID NO: 98 - DNA, 500 bp, Human cytomegalovirus
tcgggggcccgctggctcggcgcggctgtattattagacgccgggcgtcttcgcagcgttcccggtcgtcgtgtgtgctctctataaaactttcgctcgctcgcgcccgctccttagtcgagacttgcacgctgtccgggatggatcgcaagacgcgcctctcggagccgccgacgctggcgctgcggctgaagccgtacaagacggctatccagcagctgcgatctgtgatccgtgcgctcaaggagaacaccacggttaccttcttgcccacgccgtcgcttatcttgcaaacggtacgcagtcactgcgtgtcaaaaatcacttttaacagctcatgcctctacatcactgacaagtcgtttcagcccaagaccattaacaattccacgccgctgctgggtaatttcatgtacctgacttccagcaaggacctgaccaagttctacgtgcaggacatctcggacctgtcggccaagatctccatgtgcgcgcccgat

SEQ ID NO: 99 - DNA, 500 bp, Human cytomegalovirus FIX
cgagttccaccaggctctgtgccgtctcttcgcgcccctctgcgttcacgaggaccatttccatgtgcagctggtgatcggccgcggtgcgctgcagccggaggaagcggcggtagaaacgtcgcagccaccggcgcagtttgcggcgcagacgtcggcggtcctccagcagcagctggtgcatcacgtgccacgttcttgcgtccttcatctcttcgtgacggataagcgctttctgaatcgcgagctgggcgaccgtctctaccaacgcttcctgcgcgaatggctggtgtgtcggcaggccgagcgggaggcggtgacggcgctctttcagcgtatggttatgaccaagccctactttgtgtttctcgcttacgtctacagcatggactgtctgcacaccgtggccgtccgcacgatggcctttctgcgtttcgaacgctacaacaccgactacctgctgcgccgtctgcggctctacccgcccgagcggctgcacg

SEQ ID NO: 100 - DNA, 500 bp, Human cytomegalovirus
tgagttccaccaggctctgcgccgtctcttcgcgcccctctgcgttcacgaggaccatttccatgtgcagctggtgatcggccgcggtgcgctgcagccggaggaagcggcggtagaaacgtcgcagccaccggcgcagtttgcggcgcagacgtcggcggtcctccagcagcagctggtgcatcacgtgccacgttcttgcgtccttcatctcttcgtgacggataagcgctttctgaatcgcgagctgggcgaccgtctctaccaacgcttcctgcgcgaatggctggtgtgtcggcaagccgagcgggaggcggtgacggcgctctttcagcgtatggttatgaccaagccctactttgtgtttctcgcttacgtctacagcatggactgtctgcacaccgtggccgtccgcacgatggcctttctgcgtttcgaacgctacgacgccgactacctgctgcgccgtctgcggctctacccgcccgagcggctgcacg

SEQ ID NO: 101 - DNA, 500 bp, Human cytomegalovirus FIX
atcggcggtggcgtcggtgcgatggagatgaacaaggttctccatcaggatctggtgcaggccacgcggcgtatcctcaagttgggtcccagcgagctgcgcgtcaccgatgccggcctcatctgtaaaaaccccaattactcggtgtgcgacgccatgctcaagacagacacggtctattgtgtcgagtatctgctcagctactgggagagccgcacagaccacgtgccttgttttatctttaaaaacactggctgtgccgtctccctctgctgttttgtgcgagcgcccgtcaagctcgtttcgccggcgcgccacgtaggtgagttcaatgtgcttaaggtgaacgagtcgctcatcgtcacgctcaaggacatcgaggagatcaagccctcggcctacggagtgctgacgaagtgcgtggtgcgcaaatccaattcggcgtcggtcttcaacatcgagctcatcgccttcggacccgaaaacgagggcgagtacga

SEQ ID NO: 102 - DNA, 500 bp, Human cytomegalovirus
atcggcggtggcgtcggtgcgatggagatgaacaaggttctccatcaggatctggtgcaggccacgcggcgtatcctcaagttgggtcccagcgagctgcgcgtcaccgacgccggcctcatctgtaaaaaccccaattactcggtgtgcgacgccatgctcaagacagacacggtctattgtgtcgagtatctgctcagctactgggagagccgcacagaccacgtgccttgttttatctttaaaaacactggctgtgccgtctccctctgctgttttgtgcgagcgcccgtcaagctcgtctcgccggcgcgccacgtaggtgagttcaatgtgcttaaggtgaacgagtcgctcatcgtcacgctcaaggacatcgaggagatcaagccctcggcctacggagtgctgacgaagtgcgtggtgcgcaaatccaattcggcgtcggtcttcaacatcgagctcatcgccttcggacccgaaaacgagggcgagtacga

SEQ ID NO: 103 - DNA, 457 bp, Human cytomegalovirus FIX
cgtgagcggcgtgcgcacgccgcgcgaacgacgctcggccttgcgctccctgctccgcaagcgccgccaacgcgagctggccagcaaagtggcgtcaacggtgaacggcgctacgtcggccaacaaccacggcgaaccgccgtcgccggccgacgcgcgcccgcgcctcacgctgcacgacttgcacgacatcttccgcgagcaccccgaactagagctcaagtacctcaacatgatgaagatggccatcacgggcaaagagtccatctgcttacccttcaatttccactcgcaccggcagcacacctgcctcgacatctcgccgtacggcaacgagcaggtctcgcgcatcgcctgcacctcgtgcgaggacaaccgcatcctgcccaccgcctccgacgccatggtggccttcatcaatcagacgtccaacatcatgaaaaatagaaacttttat

SEQ ID NO: 104 - DNA, 457 bp, Human cytomegalovirus
cgtgagcggcgtgcgcacgccgcgcgaacgacgctcggccttgcgctccctgctccgcaagcgccgccaacgcgaactggccagcaaagtggcgtcgacggtgaacggcgctacgtcggccaacaaccacggcgaaccgccgtcgccggccgacgcgcgcccgcgcctcacgctgcacgacctgcacgacatcttccgcgagcaccccgaactggagctcaagtacctcaacatgatgaagatggccatcacgggcaaagagtccatctgcttacccttcaatttccactcgcaccggcagcacacctgcctcgacatctcgccgtacggcaacgagcaggtctcgcgcatcgcctgcacctcgtgcgaggacaaccgcatcctgcccaccgcctccgacgccatggtggccttcatcaatcagacgtccaacatcatgaaaaatagaaacttttat

SEQ ID NO: 105 - DNA, 500 bp, Human cytomegalovirus FIX
gaaacagcggcggcggtggtgactggggacggtgatgatgctgctgagactgagactggtggtgagagtagtggtggggctgcgtcgcctgcgacggcgggtggagatgaggcggcgtggactgggacgaggaggaggggccgcagccgttggtggaaactacgtgcaacggcgacgcggttaagggagaccgtatcgcgtaggacgacgtggcctcctcgtataggttgttgccgctggactgacacagctcctgaatgagctctttgtagcgctcaaaggactcgctcacgtcgttgggaatgtccatctcgtcaatcttgcgttgcaaaatagtcacgtcgatcttgacgctgctggccgagacggcgtgacacagcacgctgataacgacgtggtcgcgcacgatgttgagcgtgacgctgtagtcttcgcgcgccgccgtgagcatctgcgtgatgcagtcgcaggggatgtgcacgtcggggttttcgaagatg

SEQ ID NO: 106 - DNA, 500 bp, Human cytomegalovirus
gaaacagcggcggcggtggtgactggggacggtgatgatgctgctgagactgagactggtggtgagagtagtggtggggctgcgtcgcctgcgacggcgggtggagatgaggcggcgtggactgggacgaggaggaggggccgcagccgttggtggaaactacgtgcaacggcgacgcggttaagggagaccgtatcgcgtaggacgacgtggcctcctcgtataggttgctgccgctggactgacacagctcctgaatgagctctttgtagcgctcaaaggactcgctcacgtcgttgggaatgtccatctcgtcaatcttgcgttgcaaaatagtcacgtcgatcttgacgctgctggccgagacggcgtgacacagcacgctgataacgacgtggtcgcgcacgatgttgagcgtgacgctgtagtcttcgcgcgccgccgtgagcatctgcgtgatgcagtcgcaggggatgtgcacgtcggggttttcgaagatg

SEQ ID NO: 107 - DNA, 500 bp, Human cytomegalovirus FIX
ccgccagcaaacgccgcgacaacggccgccgcagccacgagcatcgcaacaacagcagcaacagtcgcagcccccgtggccgcttttcagaccgcaacaacagcagcaacagcagccaccgacacagcagcaccaggcgacaccgtatcagctaccgccgcaacagcggcgacagacggcgtcgcatcatcaacagcagcaacagccccgaaggttagcgccgcggcaccagagacagagaccgccgccgcgctggcaaactccgacattcgcgtcggcgcccgggccgcctgaggaaggggaggagtgtcagacacagccggtcatctccgagcccccgtcgcccgaggcggaggagccggcggcggcggtggtggaggaggttgcgccgcaagcggcggcaacagcttcgggagcagaacccgcgtcgtcgacgacgtcgttatatattaacgtcaacgtcagtcggcatagcgagcggcccgcgagttatttgtgca

SEQ ID NO: 108 - DNA, 500 bp, Human cytomegalovirus
ccgccagcaaacgccgcgacaacggccgccgcagccacgagcgttgcaacaacagcagcaacagtcgcagcccccgtggccgcttttcagaccgcaacaacagcagcaacagcagccaccgacacagcagcaccaggcgataccgtatcagctaccgccgcaacagcggcgacagacggcgtcgcatcatcaacagcagcaacagccccgaaggttagcgccgcggcaccagagacagagaccgccgccgcgctggcaaactccgacattcgcgtcggcgcccgggccgcctgaggaaggggaggagtgtcagacacagccggtcatctccgagcccccgtcgcccgaggcggaggagccggcggcggcggtggtggaggaggttgcgccgcaagcggcggcaacagcttcgggagcagaacccgcgtcgtcgacgacgtcgttatatattaacgtcaacgtcagtcggcatagcgagcggcccgcgagttatttgtgca

SEQ ID NO: 109 - DNA, 500 bp, Human cytomegalovirus FIX
aacggactgatgacgtagctcgcttcgctcgctacgtcatcagagatgatttccgccggaggtggcgcacgcatacgtgacgtagctcgctacgctcgctacgtcatcgtatgtccggaattccacgggatgacgtatatccggagtgggtgtggtcacgcgagtgtgacgtaggcttgtcaggggtcacgtgagaagcggcggcgttaagtttactaggccaaaacagaggaagggggcggataccctaagtaagggggcgtgcacgtagccctgtagacactcccccctagggtccagtagcttatgacgcgtatccgggagtagcgtctacgtcagcaggtgtatatttccggtaaacggagaagcctgtacgtacaccgaggacggtggaaccctaacgggttccacctatctgaaatttccgtacaaggggtggagtctagggaggggtcattgtatattcgtttctgtgattggtagataaggtagcgtaccta

SEQ ID NO: 110 - DNA, 500 bp, Human cytomegalovirus
aacggactgatgacgtagctcgcttcgctcgctacgtcatcagagatgatttccgccggaggtgacgcacgcatacgtgacgtagctcgctacgctcgctacgtcaccgtatgtccggaattccacaggatgacgtatatccggagtgggtgtggctacgcgagtgtgacgtaggcttgtcaggggtcacgtgagaagcggcggcgttaagtttactaggccaaaacagaggaagggggcggataccctaggtaagggggcgtgcacgtagccctgtagacactcccccctagggtccagtagcttatgacgcgtatccgggagtagcgtctacgtcagcaggtgtatatttccggtagacggagaagcctgtacgtacaccgaggacggtggaaccctaacgggttccacctatctgaaatttccgtacaaggggtggagtctagggaggggtcattgtatatccgtttctgtgattggtagataaggtggcgtaccta

SEQ ID NO: 111 - DNA, 500 bp, Human cytomegalovirus FIX
ggcgggaagcaggcgggagcgggcgcagcgtgcggaccgcagcacggccggaaccctgccgcggactgcgccggggggcggcgggcacgccgggttttataggttttcagatgccccgcctaggtgggcggagcggtaattttccaccgccgcggcccatgcccggcacggggctcgcgctccctaggtgcggccgcccagtggaaaaacaccggcgcatgcgcacggcgcacatccagtggaattttaccgacgcatgcgcactgaccgcctccagtggaaaaatactggcgcatgcgcacgacacacacccggtggaattttaccggcgcatgcgcagggcgaccctcccgcggtccctggctcgcgcatgcgcaccggggcccctggttcacccctccttatatataggttttccatgcggcatccccggcgcatgcgcactcgagtccccatcccataatccgcgtggcaacgccctgacaaccaaaaactcgccc

SEQ ID NO: 112 - DNA, 500 bp, Human cytomegalovirus; identical to SEQ ID NO: 111

SEQ ID NO: 113 - DNA, 213 bp, Human cytomegalovirus FIX
ggttatagcatcatctagtttgttcatttcatacctgttgagaacgtttatgttctagcaattgatttcgcgtcatagggctgtgacggtgattcttcagagaatcagaaaaaaaaaagaggctcaacgagcaccagagactaagtcggaaaactcgcgcccgcttccccggacggtttcagcttagcctctggcctgcgatggtttttttat

SEQ ID NO: 114 - DNA, 215 bp, Human cytomegalovirus
ggttatagcatcatctagtttgttcatttcatacctgttgagaacgtttatgttctagcaattgatttcgcgtcatagggctgtgacggtgattcttcagagaatcaggaaaaaaaaaaagaggctcaacgagcaccagagactaagtcggaaaactcgcgcccgcttccccggacggtttcggcttagcctctggcctgcgatggtttttttat

SEQ ID NO: 115 - DNA, 323 bp, Human cytomegalovirus FIX
aaagagagtgaggggtgttgtgcgtgattgctgtcccttatcccgttacaaagaaaaaagaaaaaatggtgttacacactccttggtactactatgactcgtggtgagatatccgatgatgataatgatgtacgcgtgcctgagcttggtgtttttttttctctctgtgagcttttttccccataagctgtgtactgttcgtgtccggaccccatacacggtttccgttaatgacggccccctccttttcccccaccgtaaaaaaaaaaaacaaagcacaatacacatgtggttttttggttcgaatcgagcttggcgtttat

SEQ ID NO: 116 - DNA, 323 bp, Human cytomegalovirus
aaagagagtgaagggtgttgtgcgtgatgattgctgtcccttatcccgttacaaagaaaagaaaaaatggtgttacacactccttggtactactatgacccgtggtgagatatccgatgatgataataatgatgtacgcgtgcctgagcttggtgttttttctctctgtgagcttttttccccataagctgtgtactgttcgtgtccggaccccatacacggtttccgttaatgacggccccctccttttcccccaccgtaaaaaaaaaaacaaagcacaatacacatgtggttttttggttcgaatcgagcttggcgtttat

SEQ ID NO: 117 - DNA, 246 bp, Human cytomegalovirus FIX
gcggcggcgctgtacggcagcggggagaaaagtggcagataaatcacgttaggttcacacgtcgttagccagcgtcggcatatgaagggcgcgggcggccagtacggcctctgggctgagacaggacgaggcagggtgagaaagaggaggatgggggggaccggggtggtggtgctgctgctgttgtgggtgcggacggtgcgggtgccgggacagcgtgccggcgaacgttctgtaatcttccat

SEQ ID NO: 118 - DNA, 246 bp, Human cytomegalovirus
gcggcggcgctgtacggcagcggggagaaaagtggcagataaatcacgtcaggttcacacgtcgttagccagcgtcggcatatgaagggcgcgggcggccagtacggcctctgggctgagacaggacgaggcagggtgagaaagaggaggatgggggggaccggggtggtggtgctgctgctgttgtgggtgcggacggtgcgggtgccgggacaacgtgccggcgaacgttctgtaatcttccat

SEQ ID NO: 119 - DNA, 500 bp, Human cytomeglaovirus FIX
acctaacgtgatttatctgccacttttctccccgctgccgtacagcgccgccgctcataatgccgtcaccgtcgcgtcggacgcgacggtgttttcgccgtcgatgcagaggacggaggaactttcggccgaaacatcgatcgtagtcccaggacacatttcggaagccatgccttccgcgtgcttcaccaacgtggctttctccgacgtggttgtcgttaccacaacggccgccgacgtcgcgtcggcgtaacaacggctggaggactttttcaccgcctcggcgacgtctcgaacggacgtagaaaagtaacacacggccagctccacgctatacatagcccgtttcaacgcctgcaccaaccgacgtacgaaatgaccgtggcagctttgctgacatctctcgaccagataatcaaaggagtcatccagatccttggtgggctcgcgggagaagaacgcaatgataaagagcggcagaatgccaagacgcatggtga

SEQ ID NO: 120 - DNA, 500 bp, Human cytomegalovirus; identical to SEQ ID NO: 119

SEQ ID NO: 121 - DNA, 57 bp, Human cytomegalovirus FIX
gagagacgctatatttagggcttccctctcttttttttttctacaccgtgataccct

SEQ ID NO: 122 - DNA, 57 bp, Human cytomegalovirus; identical to SEQ ID NO: 121

SEQ ID NO: 123 - DNA, 500 bp, Human cytomegalovirus FIX
ggccgtccggtgaggaggacggcgacgaccgcaggttagcggcgagtcacctagacgcaaacgcgggcccggacgcgccacgctcgctctgacgccgcgcccggtgcagacgttgttcgtctctgcttctcctccgtcgcggccaggatttcaccgccgctatggcggccatggaggccaacatcttctgcactttcgaccacaagctcagcatcgccgacgtaggcaaactgaccaagctagtagcggccgttgtgcccattccgcagcgtctacatctcatcaagcactaccagctgggcctacaccagttcgtagatcacacccgcggctacgtacgactgcgcggcctgctgcgcaatatgacgctgacgttgatgcggcgcgtagaaggcaaccagatcctcctacacgtaccgacgcacggactgctctacaccgtcctcaacacgggacccgtgacttgggagaagggcgacgcgctatgcgtgctgccgccg

SEQ ID NO: 124 - DNA, 500 bp, Human cytomegalovirus
ggccgtccggtgaggaggacggcgacgaccgcaggttaacggcgaatcacctagacgcaaacgcgggcccggacgcgccacgctcgctctgacgccgcgcccggtgcagacgttgttcgtctctgcttctcctccgtcgcggccaggatttcaccgccgctatggcggccatggaggccaacatcttctgcactttcgaccacaagctcagcatcgccgacgtaggcaaactgaccaagctagtagcggccgttgtgcccattccgcagcgtctacatctcatcaaacactaccagctgggcctacaccagttcgtagatcacacccgcggctacgtacgactgcgcggcctgctgcgcaatatgacgctgacgttgatgcggcgcgtagaaggcaaccagatcctcctacacgtaccgacgcacggactgctctacaccgtcctcaacacgggacccgtgacttgggagaagggcgacgcgctatgcgtgctgccgccg

SEQ ID NO: 125 - DNA, 209 bp, Human cytomegalovirus FIX
tggaagccgcggccgctgccgccgcggcgtttcgtccggaggagcgtccgacgccgggttggcacgacgcggcgttgttaatggacgacggtacggtgcgcgagcacgcgtttcgcaacggaccgctgtcgcaactgattcgccgtgtgttaccgccgccgcccgacgccgaagacgacgtggtttttgcttccgagctgtgtttttat

SEQ ID NO: 126 - DNA, 209 bp, Human cytomegalovirus; identical to SEQ ID NO: 125

SEQ ID NO: 127 - DNA, 430 bp, Human cytomegalovirus FIX
ggcacgtccagaacgcgtttaccgaggagatccagttacactcgctctacgcgtgcacgcgctgctttcgcacgcacctgtgtgatctgggcagcggctgcgcgctcgtctccacgctcgagggctccgtctgcgtcaagacgggcctggtatacgaagctctctatccggtggcgcgtagccacctgttggaacccatcgaggaggccgcactggacgacgtcaacatcatcagcgccgtgctcagcggcgtgtacagctacctcatgacgcacgccggccgttacgccgacgtgatccaagaggtggtcgagcgcgaccgcctcaaaaagcaggtggaggacagtatttacttcacctttaataaggttttccgttctatgcataacgtcaatcgtatttcggtgcccgtcatcagccaactttttat

SEQ ID NO: 128 - DNA, 430 bp, Human cytomegalovirus
ggcacgtccagaacgcgtttaccgaggagatccagttacattcgctctacgcgtgcacgcgctgctttcgcacgcacctgtgtgatctgggcagcggctgcgcgctcgtctccacgctcgagggctccgtctgcgtcaagacgggcctggtatacgaggctctctatccggtggcgcgtagccacctgttggaacccatggaggaggcctcactggacgacgtcaacatcatcagcgccgtgctcagcggcgtgtacagctacctcatgacgcacgcaggccgttacgccgacgtgatccaagaggtggtcgagcgcgaccgcctcaaaaagcaggtggaggacagtatttacttcacctttaataaggttttccgttctatgcataacgtcaaccgtatttcggtgcccgtcatcagccaactttttat

SEQ ID NO: 129 - DNA, 500 bp, Human cytomeglaovirus FIX
ggcgcggttcgctgacgatgagcaattgcctctacacctggtgctcgaccaggaggtgctgagtaacgaggaggccgagacgctgcgctacgtctactatcgtaatgtagacagcgctggccgatccgcgggccgcgttccgggcggagatgaggacgacgcaccggcctccgacgacgccgaggacgccgtgggcggcgatcgcgcttttgaccgcgagcggcggacttggcagcgggcctgttttcgtgtactaccgcgcccactggagttgctcgattacctacgtcaaagcggtctcactgtgacgttagagaaagagcagcgcgtgcgcatgttctatgccgtcttcactacgttgggtctgcgctgccccgataatcggctctcaggcgcgcagacgctacacctgagactggtctggcccgacggcagctatcgtgactgggagtttttagcgcgtgacctgttacgagaagaaatggaagcgaataagcgcg

SEQ ID NO: 130 - DNA, 500 bp, Human cytomegalovirus
ggcgcggttcgctgacgatgagcaattgcctctacacttggtgctcgaccaggaggtgctgagtaacgaggaggccgagacgctgcgctacgtctactatcgtaatgtagacagcgctggccgatccgcgggccgcgctccgggcggagatgaggacgacgcaccggcctccgacgacgccgaggacgccgtgggcggcgatcgcgcttttgaccgcgagcggcggacttggcagcgggcctgttttcgtgtactaccgcgcccactggagttgctcgattacctacgtcaaagcggtctcactgtgacgttagagaaagagcagcgcgtgcgcatgttctatgccgtcttcactacgttgggtctgcgctgccccgataatcggctctcaggcgcgcagacgctacacctgagactggtctggcccgacggcagctatcgtgactgggagtttttagcgcgtgacctgttacgagaagaaatggaagcgaataagcgcg

SEQ ID NO: 131 - DNA, 421 bp, Human cytomegalovirus FIX
cgtcggtcaacaaacagctcttaaaggacgtgatgcgcgtcgaccttgagcgacagcagcatcagtttctgcggcgtacctacggaccgcagcaccggctcaccacgcagcaggctttgacggtgatgcgtgtggccgctcgggaacagacccgatacagtcagcgaacgacgcagtgcgtggccgcacacctgttggagcaacgggcggccgtgcagcaagagttgcaacgcgcccgacagctgcaatccggtaacgtggacgacgcgctggactctttaaccgagctgaaggacacggtagacgacgtgagagccaccttggtggactcggtttcggcgacgtgcgatttggacctggaggtcgacgacgccgtctaacaggtatagcaatccccgtcacgcctctgttcagattttat

SEQ ID NO: 132 - DNA, 421 bp, Human cytomegalovirus; identical to SEQ ID NO: 131

SEQ ID NO: 133 - DNA, 500 bp, Human cytomegalovirus FIX
ccgggacgcggaacgtgacggttgctgaggggaaaggcaacagagaaggtacaaacccaccggcggggaaaataccgaggcgccgccatcatcatgtggggcgtctcgagtttggactacgacgacgatgaggagctcacccggctgctggcggtttgggacgatgagcccctcagtctctttctcatgaacacctttttgctgcaccaggagggcttccgtaatctgccctttacggtgctgcgtctgtcttacgcctaccgcatcttcgccaagatgctgcgggcccacggtacgccagtagccgaggactttatgacgcgcgtggccgcgctggctcgcgacgagggtctgcgcgacattttgggtcagcggcacgccgccgaagcctcacgcgccgagatcgccgaggccctggagcgcgtggccgagcggtgcgacgaccggcacggcggctcggacgactacgtgtggctcagccggttgctggatttggcgcc

SEQ ID NO: 134 - DNA, 500 bp, Human cytomegalovirus
ccgggacgcggaacgtgacggttgctgaggggaaaggcaacagagaaggtacaaacccaccggcggggaaaataccgaggcgccgccatcatcatgtggggcgtctcgagtttggactacgacgacgatgaggagctcacccggctgctggcggtttgggacgatgagcccctcagtctctttctcatgaacacctttttgctgcaccaggagggcttccgtaatctgccctttacggtgctgcgtttgtcttacgcctaccgcatcttcgccaagatgctgcgggcccacggtacgccagtagccgaggactttatgacgcgcgtggccgcgctggctcgcgacgagggtctgcgcgacattttgggtcagcggcacgccgccgaagcctcgcgcgccgagatcgccgaggccctggagcgcgtggccgagcggtgcgacgaccggcacggcggctcggacgactacgtgtggcttagccggttgctggatttggcgcc

SEQ ID NO: 135 - DNA, 500 bp, Human cytomegalovirus FIX
aagatgctctgggtcgccaggtgtctctacgctcctacgacaacatccctccgacttcctcctcggacgaaggggaggacgatgacgacggggaggatgacgataacgaggagcggcaacagaagctgcggctctgcggtagtggctgcgggggaaacgacagtagtagcggcagccaccgcgaggccacccacgacggctccaagaaaaacgcggtgcgctcgacgtttcgcgaggacaaggctccgaaaccgagcaagcagtcaaaaaagaaaaagaaaccctcaaaacatcaccaccatcagcaaagctccattatgcaggagacggacgacctagacgaagaggacacctcaatttacctgtccccgcccccggtcccccccgtccaggtggtggctaagcgactgccgcggcccgacacacccaggactccgcgccaaaagaagatttcacaacgtccacccacccccgggacaaaaaagcccgccgcctccttg

SEQ ID NO: 136 - DNA, 500 bp, Human cytomegalovirus; identical to SEQ ID NO: 135

SEQ ID NO: 137 - DNA, 89 bp, Human cytomegalovirus FIX
ccccgccgccacccgcaccagacttggagacatggacataaaaaagagacacgcagaccgtgggtcgggagcacatactttttttttat

SEQ ID NO: 138 - DNA, 90 bp, Human cytomegalovirus
ccccgccgccacccgcaccagacttggagacatggacataaaaaagagacacgcagaccgtgggtcgggagcacatacttttttttttat

SEQ ID NO: 139 - DNA, 500 bp, Human cytomegalovirus FIX
gaagcgaactagacacgcatatcatagaaaaaaaaaaaacacgcaacacgtagtgagctttgacgtcccttttactagtatccacgtcacacgctgagaactttgacgcactttttttttactagtatccacgtcacttacccgcgtagttcccctacgtgactcgttaagcgttgagccggaaaaacctcaggccctcggaagccacccgcttagcagcgtgttgcgcgtcaaccgccagcgaacgcacccactcgtcgcgctcctcgagccaagtcgccgacgaagaagaacaagacggaggagacaccgtcgccgtgcccgaagaggacgaagtgacggacggcaaggcggaggagagaacggaagaagaacaagcggtggtagaagcggtggaggacgacaataactctcgcgcccagacctccacgcaagccgtgagcatggcaaaagccttgtccacatagacgccgtagccgatatcggccgctaacgccgta

SEQ ID NO: 140 - DNA, 500 bp, Human cytomegalovirus
gaagcgaactagacacgcatatcatagaaaaaaaaacacgcaacacgtagtgagctttgacgtcccttttactagtatccacgtcacacgctgagaactttgacgcacttttttttactagtatccacgtcacttacccacgtagttctcctacgtgactcgttaagcgttgagccggaaaaaccgcaggccctcggaagccacccgcttagcagcgtgttgcgcgtcaaccgccagcgagcgcacccactcgtcgcgctcctcgagccaagttgccgacgaagaagaacaagacggaggagacaccgtcgccgtgcccgaagaggacgaagtgacggacggcaaggcggaggagagaacggaagaagaagaacaagtggtggtggaagcggtggaggacgacaataactctcgcgcccagacctccacgcaagccgtgagcatggcaaaggccttgtccacatagacgccgtagccgatatcggccgccaacgccgtat

SEQ ID NO: 141 - DNA, 32 bp, Human cytomegalovirus FIX
cacaacaccgtgtaaggaaaacgtgactttat

SEQ ID NO: 142 - DNA, 32 bp, Human cytomegalovirus; identical to SEQ ID NO: 141

SEQ ID NO: 143 - DNA, 443 bp, Human cytomegalovirus FIX
ggcatcctctctgccacacgcgcagtcacggataggatcagtgcgtattcattataaaaaaaacacaaacaacccatatatgtgaagcagaatgatgaccgaccgcacggagcgacgccgtcgactgacccacgcgggatgtacgccgtccgcgaacaaccaaaggacgacccgtctccccccgcatccgggtttttctcttggtcgaacccggcttgcgacgacgggttgttgctttaccggacgacggtcagccgcggggttgatacccagcgacggcgtcgctcccacccgggtttcttctcttgtaggtaccactcgtagactgtcagccttacgaggagacaccgcggaccggggaaacggataagtttacgaacagaaatctcaagagaaagatgctgacccgataagtaccgtcacggagacacggtggtttttat

SEQ ID NO: 144 - DNA, 441 bp, Human cytomegalovirus
ggcatcctctctgccacacgcgcagtcacggataggatcagtgcgtattcattataaaaaaaaacacaaacaacccatatatgtgaagcagaatgatgaccgaccgcacggagcgacgccgtcgactgacccacgcggcatgtacgccgtccgcgaacaaccaaaggacgacccgtctccccccgcacccgggttttttctcttggtcgaacccggcttgcgacgacgggttgttcctttaccggacgacggtcagccgcggggttgatacccagcgacggcgtcgctcccacccgggtttcttctcttgcaggtaccacccgtcgactgtcagcctcgcgaggagacaccgcggaccggggaaacggataagtttacgaacagaaatctcaaaagacgctgacccgataagtaccgtcacggagacacggtggtttttat

SEQ ID NO: 145 - DNA, 111 bp, Human cytomegalovirus FIX
aaaacagagccgagaccggaaaaattatgaaacaggacgcgcttggacatttgggtttccacccctttcggtgtgtgtctatatatattgtggtcactgatttttttttac

SEQ ID NO: 146 - DNA, 111 bp, Human cytomegalovirus; identical to SEQ ID NO: 145

SEQ ID NO: 147 - DNA, 500 bp, Human cytomegalovirus FIX
agcggcggcggcgatggcggggctggttgcttttcctggccctgtgcttttgcttactgtgtgaagcggtggaaaccaacgcgaccaccgttaccagtaccaccgctgccgccgccacgacaaacactaccgtcgccaccaccggtaccactactacctcccctaacgtcacttcaaccacgagtaacaccgtcatcactcccaccacggtttcctcggtcagcaatctgacatccagcgccacgtcgattcccatctcaacgtcaacggtttctggaacaagaaacacaaggaataataataccacaaccatcggtacgaacgttacttccccctccccttctgtatccatacttaccaccgtgacaccggccgcgacttctaccacctccaacaacggggatgtaacatccgactacactccaacttttgacctggaaaacattaccaccacccgcgctcccacgcgtcctcccgcccaggacctttgtagccataac

SEQ ID NO: 148 - DNA, 500 bp, Human cytomegalovirus
agcggcggcggcgatggcggggctggttgcttttcccggccctgtgcttttgcttactgtgtgaagcggtggaaaccaacgcgaccaccgttaccagtaccaccgctgccgccgccacgacaaacactaccgtcgccaccaccggtaccactactacctcccctaacgtcacttcaaccacgagtaacaccgtcaccactcccaccacggtttcctcggtcagcaatctgacgtccagcaccacgtcgattcccatctcaacgtcaacggtttctggaacaagaaacacagggaataataataccacaaccatcggtacgaacgctacttccccctccccttctgtatccatacttaccaccgtgacaccggccgcaacttctaccatctccgtcgacggtgtcgtcacggcgtcagactacactccgacttttgacgatctggaaaacattaccaccacccgcgctcccacgcgtcctcccgcccaggacctgtgtagc

SEQ ID NO: 149 - DNA, 500 bp, Human cytomegalovirus FIX
cgcggccccctgccacatatagctcgtccacacgccgtctcgtcacacagcaacatgtgtcccgtgctggcgatcgtactcgtggttgcgctcttgggcgacacgcacccgggagtggaaagtagcaccacaagcgccgtcacgtcccctagtaataccaccgccacatccactacgtcaataagtacctctaacaacgtcacttctgctgtcaccaccacggtacaaacctctacctcgtccgcctccacctccgtgatagccacgacgcagaaagaggggcgcctgtatactgtgaattgcgaagccagctacagctacgaccaagtgtctctaaacgccacctgcaaagttatcctgttgaataacaccaaaaatccagacattttatcagttacttgttatgcacggacagactgcaagggtcccttcactcaggtggggtatcttagcgctttcccccccgataatgaaggtaagtagcacctacctttctgttc

SEQ ID NO: 150 - DNA, 500 bp, Human cytomegalovirus; identical to SEQ ID NO: 149

SEQ ID NO: 151 - DNA, 500 bp, Human cytomegalovirus
tgttaccccgccagcacctccgccggcaaccgcgtcgtcgttgctatcgtcgccggtttcgggcgatgacagcgccggcggcgcgggtctcgtctcgtccaccatttccaccgtgtcgaagcgacagccgctgccgtagtacatggccccgttcaacggccggcgggccgggtcgccgagttccgggtcgggcacatccatggctcgccgtctgcttctctgccgctcgtggtgccgacggcacttctcaggataatgacagccgcaaaatagatcgtggagcatgtctcgccaactgtcctggtggtaatatcttaagtacgcgatgagcgcgccgatggccataatcataagcgtaagcaaaacggcacagataacgtgaaacaccgcggtcatccaagtcgggcggcgtcggggacgcggtgggtcggtttctcttacgccggcgtcactcagccaccacacccgtagtcgacattcccagaaccggtgaatgcgac

SEQ ID NO: 152 - DNA, 500 bp, Human cytomegalovirus
tgttaccccgccagcacctccgccggcaaccgcgtcgtcgttgctatcgtcgccggtttcgggcgatgacagcgccggcggcgcgggtctcgtctcgtccaccatttccaccgtgtcgaagcgacagccgctgccgtagtacatagctccgttcaacggccggcgggccgggtcgccgagttccgggtcgggcacatccatggcttgccgtctccttctctgccgctcgtggtgccgacggcacttctcgggataatgacagccgcaaaatagatcgtggagcatgtctcgccaactgtcctggtggtaatatcttaagtacgcgatgagcgcgccgatggccataatcataagcgtaagcaaaacggcacagataacgtgaaacaccgcggtcatccaagtcgggcggcgtcggggacgcggtgggtcggtttctcttacgccggcgtcactcagccaccacacccgtagccgacattcccagaaccggtgaatgcgac

SEQ ID NO: 153 - DNA, 500 bp, Human cytomegalovirus FIX
gctgcccgcgactcctcgaatattcttcctcttcgttccccttcgccaccgctgacattgccgaaaagatgtgggccgagaattatgagaccacgtcgccggcgccggtgttggtcgccgagggagagcaagttaccatcccctgcacggtcatgacacactcctggcccatggtctccattcgcgcacgtttctgtcgttcccacgacggcagcgacgagctcatcctggacgccgtcaaaggccatcggctgatgaacggactccagtaccgcctgccgtacgccacttggaatttctcgcaattgcatctcggccaaatattctcgctgactttcaacgtatcgacggacacggccggcatgtacgaatgcgtgctgcgcaactacagccacggcctcatcatgcaacgcttcgtaattctcacgcaactggagacgctcagccggcccgacgaaccttgctgcacgccggcgttaggtcgttactcgctgggagac

SEQ ID NO: 154 - DNA, 500 bp, Human cytomegalovirus; identical to SEQ ID NO: 153

SEQ ID NO: 155 - DNA, 500 bp, Human cytomegalovirus FIX
agaaggggaggacgacgttctcgccacaatccgcaacacgttgtccgccccaacctcacctgctgcggctaccacgcatcgactgtcgttccctggagaatcgaccttctgcctcaccgctgtttccgagtgctcacaacgtcgaacatcaacggctgcattaacgccgccgccgccagcggtagctgctgcgttctctttttcgtccacggtctccgagaccggcacttttccgcagagcacaacaggccgcacacgtgtcgacgacaccgccgtcgttaccgccggagacccgcgctctcctgtgacacacgtaactctcctccagatattccgtctgcgtagctcgctgctgacgagcaggtccggcggcgctctccgcggaggtgagcacgaggccatccccaaagtcgcgtcgctgttctggacgctgctcaaagcaacacagatagttgacatgactcacaaaacaccgagtgccgactctcaccgcaacccac

SEQ ID NO: 156 - DNA, 500 bp, Human cytomegalovirus
ctggaacgtcgtacgctgccgcggcacaggctttcgcgcacacgattccgaggacggcgtctctgtctggcgtcagcacttggtttttttactcggaggccacggccgccgtgtacagttagaacgtccatccgcgggagaagcccaagctcgaggcctattgccacgcatccggatcacccccatctccacatctccacgcccaaaaccaccccagcccaccatatccaccgcatcgcacccacatgctacgactcgcccacatcacacgctctttcctatcccttctacaccctcagccacggttcacaatccccgaaactacgccgtccaacttcacgccgaaacgacccgcacatggcgctgggcacgacgcggtgaacgtggcgcgtggatgccggccgagacatttacatgtcccaaggataaacgtccctggtagacggggtagggggatctaccagcccagggatcgcgtatttcgccgccacgctgcttca

SEQ ID NO: 157 - DNA, 500 bp, Human cytomegalovirus FIX
acgccgtgcaccacaaactctgcggcgcgatgatatcttcgtcgtgttccaccacttgcacaccgctgattatggacttgccgtcgctgtccgtggaactatctgcaggacacaagaaaaaagaaacaccaaccgagggtgggtggggcggtgaagaaggggaggacgacgttctcgccacaatccgcaacacgttgtccgccccaacctcacctgctgcggctaccacgcatcgactgtcgttccctggagaatcgaccttctgcctcaccgctgtttccgagtgctcacaacgtcgaacatcaacggctgcattaacgccgccgccgccagcggtagctgctgcgttctctttttcgtccacggtctccgagaccggcacttttccgcagagcacaacaggccgcacacgtgtcgacgacaccgccgtcgttaccgccggagacccgcgctctcctgtgacacacgtaactctcctccagatattccgtctgcgtagc

SEQ ID NO: 158 - DNA, 500 bp, Human cytomegalovirus; identical to SEQ ID NO: 157

SEQ ID NO: 159 - DNA, 161 bp, Human cytomegalovirus FIX
cattcccctgggaattcatgctgtatgggcgggtatagtggtatctgtggcacttatagccttatacatgggtagccgtcgcgtccccagaagaccgcgttatacaaaacttcccaaatacgacccagatgaattttagactaaaacctaacatgcacatc

SEQ ID NO: 160 - DNA, 161 bp, Human cytomegalovirus; identical to SEQ ID NO: 159

SEQ ID NO: 161 - DNA, 383 bp, Human cytomegalovirus FIX
taaactgttaggttcgttataagcgtggatggtcatatataaaccgtatgcacaaaaggtatgtgtgaatggaaatacatgatgaatgtcatcatcacgcaaagcagccgtgggaatggtaaagacatcgtcacacctatcataaagaatgcaacgctttcaggataggtgtggcgaaagcctcctccgttccgtattctatcgtaacaaatatatggagtttgtgtaatgcgtacttcatgccccgatgaacgctctcgtcaggcttgtcatggtctgtaaaagctgcatgaaaaacacgacgaaagcgttcagtgttggatcagactcccacgttaattaagggcggccggatccatgtttaaacaggcgcgcctagcttc

SEQ ID NO: 162 - DNA, 500 bp, Human cytomegalovirus
taaactgttaggcttgttataagcgtggatgatcatatataaaccgtatgcacaaaaggtatgtgtgaatggaaatacatgatgaatgtcatcgtcacgcaaagcagccgtgggaatggtaaagacatcgtcacacctatcataaagaatgcaacgctttcaggataggtgtggcgaaagcctcctccgttccgtattctatcgtaacaaatatatagagtttatgtaatgcgtacttcatgccccgatgaacgctctcgtcaggcttgtcatggtccgtaaaagttgcatgaaaaacacgacgaaagcgttcagtgttggatcagactcacgtcacacgttacatcatacaacgtagggcggtattgttgagaacatatataatcgccgtttcgtaagtacgtcgatatcgctccttcttcactatggacctcttgatccgtctcggttttctgttgatgtgtgcgttgccgacccccggtgagcggtcttcgcgtgac

SEQ ID NO: 163 - DNA, 500 bp, Human cytomegalovirus FIX
aatgatttgttatgatgtcattgttgtttactgaaaaggaatgtgctttcccggcatgggcccgattccgagaaatggtatgatgaatcatgtggtcaggcgctgctctcaacgtccatataaacgtgggtttcggtgaccacaaccacgtcggggctgacgcggatcggacatcatactgacgtgaggcgctccgtcacctctcgggccgaaccccgtcagcaccccgcgtcacttacaaatcacgttcgtcgtgacgggggtttcccctgacacgtaatactcgcgtcacgtcgggacgatataaagaggcacggtgtttcggctcccgcacacagacgacgcgccgggcggcttcctgcggccggccgcggtgccggcggctatgatcctgtggtccccgtccacctgttccttcttctggcactggtgtctgatcgcagtaagtgtactctcgagccgctccaaggagtcgctccggttgtcgtggtccagcgacg

SEQ ID NO: 164 - DNA, 500 bp, Human cytomegalovirus; identical to SEQ ID NO: 163

SEQ ID NO: 165 - DNA, 500 bp, Human cytomegalovirus FIX
aaaaaaaacgtttctatcacctaatctgtcgtactgtcctttgtcccccgcaccctaaaacaccgtgttctcccgacgtcactagatcaccaccctgttccccatgacgtgcaagactacatgctataagacagccttacagcttttgagtctagacaggggaacagccttcccttgtaagacagaatgaatcttgtaatgcttattctagccctctgggccccggtcgcgggtagtatgcctgaattatccttgactcttttcgatgaacctccgcccttggtggagacggagccgttaccgcctctgcccgatgtttcggagtaccgagtagagtattccgaggcgcgctgcgtgctccgatcgggcggtcgattggaggctctgtggaccctgcgcgggaacctgtccgtgcccacgccgacaccccgggtgtactaccagacgctggagggctacgcggatcgagtgccgacgccggtggaggacgtctccgaaag

SEQ ID NO: 166 - DNA, 500 bp, Human cytomegalovirus; identical to SEQ ID NO: 165

SEQ ID NO: 167 - DNA, 500 bp, Human cytomegalovirus FIX
gctccgctggtttataagaagactccaccgagacgctcacccgttcactcgggcgcatcacccgcctcatggactcgccgctaccgtcgctacattcgccgcaatgggcttccctcctgcagctgcaccacggccttatgtggctgcgccgttttgctgtcctcgtccgggtctacgccctagtggtctttcacatcgccatcagtacggctttctgcggaatgatttggctgggtatccccgattcccacaacatatgtcaacatgaatcttcccctctgctgctggtttttgccccctcccttctctggtgtttggtcttgatacagggcgaaaggcaccccgacgacgtggtattgaccatgggctacgtaggcctcctctccgttaccacggttttctacacctggtgctccgacctgcccgccatcctcatcgactacacactggtcctcacgctgtggatagcttgcaccggcgctgtcatggttggggacagc

SEQ ID NO: 168 - DNA, 500 bp, Human cytomegalovirus; identical to SEQ ID NO: 167

SEQ ID NO: 169 - DNA, 20 bp, Human cytomegalovirus FIX
gcgtcgagcggaggacgcgg

SEQ ID NO: 170 - DNA, 20 bp, Human cytomegalovirus
gcgtcgagcggaggacgcgg

SEQ ID NO: 171 - DNA, 45 bp, Human cytommegalovirus FIX
aaacaacgtcaacagtttacgagtacaaaacaggaaaggaacaca

SEQ ID NO: 172 - DNA, 45 bp, Human cytomegalovirus
aaacaacatcaacagtttacgagtacaaaacaggaaaggaataca

SEQ ID NO: 173 - DNA, 500 bp, Human cytomegalovirus FIX
ttcgatcctctctcacgcgtccgccgcacatctatttttgctaattgcacgtttcttcgtggtcacgtcggctcgaagaggttggtgtgaaaacgtcatctcgccgacgtggtgaaccgctcatatagaccaaaccggacgctgcctcagtctctcggtgcgtggaccagacggcgtccatgcaccgagggcagaactggtgctatcatgacaccgacgacgacgaccgcggaactcacgacggagtttgactacgatgaagacgcgactccttgtgttttcaccgacgtgcttaatcagtcaaagccagttacgttgtttctgtacggcgttgtctttctcttcggttccatcggcaacttcttggtgatcttcaccatcacctggcgacgtcggattcaatgctccggcgatgtttactttatcaacctcgcggccgccgatttgcttttcgtttgtacactacctctgtggatgcaatacctcctagatcacaactc

SEQ ID NO: 174 - DNA, 500 bp, Human cytomegalovirus; identical to SEQ ID NO: 173

SEQ ID NO: 175 - DNA, 500 bp, Human cytomegalovirus FIX
taaaaaagcgctacctcggccttttcatacaaaccccgtgtccgcccctcttttccccgtgcccgatatacacgatattaaacccacgaccatttccgtgcgattagcgaaccggaaaagtttatggggaaaaagacgtaggaaaggatcatgtagaaaaacatgcggtgtttccaatggtggctctacagtgggtggtggtggctcacgtttggatgtgctcggaccgtgacggtgggtttcgtcgcgcccacggtccgggcacaatcaaccgtggtccgctctgagccggctccgccgtcggaaacccgacgagacaacaatgacacgtcttacttcagcagcacctctttccattcttccgtgtcccctgccacctcagtggaccgtcaatttcgacggaccacgtacgaccgttgggacggtcgacgttggctgcgtacccgctacgggaacgccagcgcctgcgtgacgggcacccaatggagcaccaacttttt

SEQ ID NO: 176 - DNA, 500 bp, Human cytomegalovirus
taaaaaagcgctacctcggtcttttcgtacaaaccccgtgtccgcccctcttttccccgtgcccgatatacacgatattaaacccacgaccatttccgtgcgattagcgaaccggaaaagtttatggggaaaaagacgtaggaaaggatcatgtagaaaaacatgcggtgtttccgatggtggctctacagtgggtggtggtggctcacgtttggatgtgctcggaccgtgacggtgggtttcgtcgcgcccacggtccgggcacaatcaaccgtggtccgctctgagccggctccgccgtcgaaaacccgacgagacaacaatgacacgtcttacttcagcagcacctctttccattcttccgtgtcccctgccacctcagtggaccgtcaatttcgacggcccacgtacgaccgttgggacggtcgacgttggctgcgcacccgctacgggaacgccagcgcctgcgtgacgggcacccaatggagcaccaacttttt

SEQ ID NO: 177 - DNA, 500 bp, Human cytomegalovirus FIX
aaaatgataatgatgataataacgattacgaccgctaaaacccagagggcgtgtgtagccacgtgttggtgctgtgggcttggttgtaacggtgtttccgctgctgtggcttcaaaaccaacgtgatgttctacgtgactgttaggggtggtggattttttgggactggagtgtttatgatgggtagtgcttatcgtcgtcttcttggcggtggtggttgttctcgtggtggttgttttttgtgttgtggtagttgtcgttctcgtagtcgtagtgggctttttggtggtggtagtggggaatgtaccgttttcgttcactgtcagattgtaacatgtgtctaaagtccatcgaaaaccatggttatgttgttggtgacgccaatcgtctagcgatgtcatagtacgataggtagtactatactgcgcggtaacgttaatgaggaggaggctgtaattactcagacatgaaaaattaaagcgcgtgctgttaaacgttgt

SEQ ID NO: 178 - DNA, 500 bp, Human cytomegalovirus; identical to SEQ ID NO: 177

SEQ ID NO: 179 - DNA, 500 bp, Human cytomegalovirus FIX
ttttctcccccatccgacaaaaccgtgtcccttaaaattccccacctttctctgttcaaatggccccgaaactgtaaaacaccgtttgaccgcaccccaaccggcgccatcttggtgacctcgacggttctctcgctcgtcatgccgttctgagctccgacatggcggacgagagaaaatggcgtcgagagcctaggagcgttttcgctccaggcgggtaaaaaaatagcacgataacttttctgtgctttttttgagacgttttagaagagcttttttctgctcagagcgaaaaaatgatagccctgaaaatctcgacgagtctggccgagcggcgccatcttggaggaggggcgagtcgcgggcaccgcctcggtaccccctggctgaggcgagtccgcggtcgccgcctgttccgtgatgctacctagagggcgctgtcgaggcgactcttcctgttttcgccctgagggctaacggtcgctgacgtcaaaccatct

SEQ ID NO: 180 - DNA, 500 bp, Human cytomegalovirus
aacaccgtttgactgcaccccaaccggcgccatcttggtgaccttctcgacggttctctcgctcgtcatgccgttctgagctccgacatggcggacgagagaaaatggtgtcgagagccgaggagcgttttcgctccaggcgggtaaaaaaatagcacgataacttttctgtgcttttttgagacgtttttgaagagctttttttctgctcagagcgaaaaaatgatagccctgaaaatctcgacgagtctggccgagcggcgccatcttggaggaggggcgagtcgcgggcaccgcctcggtacccccctggccgaggcgagtccgcggtcgccgcctgttccgtgatgctacctagagggcgccgtcgaggcgactcttcctgttttcgccctgagggctaacggtcgctgacgtcaaaccatctcgtgctcgctgagtcacatccggttgttgacaagcgatggaggaccgcacccaaagtgcgccctctagtcatc

SEQ ID NO: 181 - DNA, 396 bp, Kaposi′s sarcoma-associated herpesvirus
ttgtgtacccgtaacgatggcaaaggaactggcggcggtctatgccgatgtgtcagccctagccatggacctctgtcttcttagttacgcagacccggcaacactggacactaaaagtctggccctcactacagggaagtttcagagccttcacggcacactactccccctcctcagacgacaaaacgcacacgaatgctcaggtctgtcactagaattggagcacttttggaaaacgtggctgatgctctggccacgttgggagtgtgcactagcagaaaactgtctccagaagagcatttttccctcctgcatttggacacaacatgcaacaagcaaccggagcgttaggtttaatttttacggaaattgggccttggagttaaagctgtcact

SEQ ID NO: 182 - DNA, 3858 bp, Kaposi′s sarcoma-associated herpesvirus
attggccaccctggggactgtcatcctgttggtctgcttttgcgcaggcgcggcgcactcgaggggtgacacctttcagacgtccagttcccccacacccccaggatcttcctctaaggcccccaccaaacctggtgaggaagcatctggtcctaagagtgtggacttttaccagttcagagtgtgtagtgcatcgatcaccggggagctttttcggttcaacctggagcagacgtgcccagacaccaaagacaagtaccaccaagaaggaattttactggtgtacaaaaaaaacatagtgcctcatatctttaaggtgcggcgctataggaaaattgccacctctgtcacggtctacaggggcttgacagagtccgccatcaccaacaagtatgaactcccgagacccgtgccactctatgagataagccacatggacagcacctatcagtgctttagttccatgaaggtaaatgtcaacggggtagaaaacacatttactgacagagacgatgttaacaccacagtattcctccaaccagtagaggggcttacggataacattcaaaggtactttagccagccggtcatctacgcggaacccggctggtttcccggcatatacagagttaggaccactgtcaattgcgagatagtggacatgatagccaggtctgctgaaccatacaattactttgtcacgtcactgggtgacacggtggaagtctcccctttttgctataacgaatcctcatgcagcacaacccccagcaacaaaaatggccttagcgtccaagtagttctcaaccacactgtggtcacgtactctgacagaggaaccagtcccactccccaaaacaggatctttgtggaaacgggagcgtacacgctttcgtgggcctccgagagcaagaccacggccgtgtgtccgctggcactgtggaaaaccttcccgcgctccatccagactacccacgaggacagcttccactttgtggccaacgagatcacggccaccttcacggctcctctaacgccagtggccaactttaccgacacgtactcttgtctgacctcggatatcaacaccacgctaaacgccagcaaggccaaactggcgagcactcacgtccctaacgggacggtccagtacttccacacaacaggcggactctatttggtctggcagcccatgtccgcgattaacctgactcacgctcagggcgacagcgggaaccccacgtcatcgccgcccccctccgcatcccccatgaccacctctgccagccgcagaaagagacggtcagccagtaccgctgctgccggcggcggggggtccacggacaacctgtcttacacgcagctgcagtttgcctacgacaaactgcgggatggcattaatcaggtgttagaagaactctccagggcatggtgtcgcgagcaggtcagggacaacctaatgtggtacgagctcagtaaaatcaaccccaccagcgttatgacagccatctacggtcgacctgtatccgccaagttcgtaggagacgccatttccgtgaccgagtgcattaacgtggaccagagctccgtaaacatccacaagagcctcagaaccaatagtaaggacgtgtgttacgcgcgccccctggtgacgtttaagtttttgaacagttccaacctattcaccggccagctgggcgcgcgcaatgagataatactgaccaacaaccaggtggaaacctgcaaagacacctgcgaacactacttcatcacccgcaacgagactctggtgtataaggactacgcgtacctgcgcactataaacaccactgacatatccaccctgaacacttttatcgccctgaatctatcctttattcaaaacatagacttcaaggccatcgagctgtacagcagtgcagagaaacgactcgcgagtagcgtgtttgacctggagacgatgttcagggagtacaactactacacacatcgtctcgcgggtttgcgcgaggatctggacaacaccatagatatgaacaaggagcgcttcgtaagggacttgtcggagatagtggcggacctgggtggcatcggaaaaacggtggtgaacgtggccagcagcgtggtcactctatgtggctcattggttaccggattcataaattttattaaacaccccctaggtggcatgctgatgatcattatcgttatagcaatcatcctgatcatttttatgctcagtcgccgcaccaataccatagcccaggcgccggtgaagatgatctaccccgacgtagatcgcagggcacctcctagcggcggagccccaacacgggaggaaatcaaaaacatcctgctgggaatgcaccagctacaacaagaggagaggcagaaggcggatgatctgaaaaaaagtacaccctcggtgtttcagcgtaccgcaaacggccttcgtcagcgtctgagaggatataaacctctgactcaatcgctagacatcagtccggaaacgggggagtgacagtggattcgaggttattgtttgatgtaaatttaggaaacacggcccgcctctgaagcaccacatacagactgcagttatcaaccctactcgttgcacacagacacaaattaccgtccgcagatcatggattttttcaatccatttatcgacccaactcgcggaggcccgagaaacactgtgaggcaacccacgccgtcacagtcgccaactgtcccctcggagacaagagtatgcaggcttataccggcctgtttccaaaccccggggcgacccggcgtggttgccgtggacaccacatttccacccacctacttccagggccccaagcggggagaagtattcgcgggagagactgggtctatctggaaaacaaggcgcggacaggcacgcaatgctcctatgtcgcacctcatattccacgtatacgacatcgtggagaccacctacacggccgaccgctgcgaggacgtgccatttagcttccagactgatatcattcccagcggcaccgtcctcaagctgctcggcagaacactagatggcgccagtgtctgcgtgaacgttttcaggcagcgctgctacttctacacactagcaccccagggggtaaacctgacccacgtcctccagcaggccctccaggctggcttcggtcgcgcatcctgcggcttctccaccgagccggtcagaaaaaaaatcttgcgcgcgtacgacacacaacaatatgctgtgcaaaaaataaccctgtcatccagtccgatgatgcgaacgcttagcgaccgcctaacaacctgtgggtgcgaggtgtttgagtccaatgtggacgccattaggcgcttcgtgctggaccacgggttctcgacattcgggtggtacgagtgcagcaatccggccccccgcacccaggccagagactcttggacggaactggagtttgactgcagctgggaggacctaaagtttatcccggagaggacggagtggcccccatactcaatcctatcctttgatatagaatgtatgggcgagaagggttttcccaacgcgactcaagacgaggacatgattatacaaatctcgtgtgttttacacacagtcggcaacgataaaccgtacacccgcatgctactgggcctggggacatgcgacccccttcctggggtggaggtctttgagtttccttcggagtacgacatgctggccgccttcctcagcatgctccgcgattacaatgtggagtttataacggggtacaacatagcaaactttgaccttccatacatcatagcccgggcaactcaggtgtacgacttcaagctgcaggacttcaccaa

SEQ ID NO: 183 - DNA, 1337 bp, Kaposi′s sarcoma-associated herpesvirus
cagtggattcgaggttattgtttgatgtaaatttaggaaacacggcccgcctctgaagcaccacatacagactgcagttatcaaccctactcgttgcacacagacacaaattaccgtccgcagatcatggattttttcaatccatttatcgacccaactcgcggaggcccgagaaacactgtgaggcaacccacgccgtcacagtcgccaactgtcccctcggagacaagagtatgcaggcttataccggcctgtttccaaaccccggggcgacccggcgtggttgccgtggacaccacatttccacccacctacttccagggccccaagcggggagaagtattcgcgggagagactgggtctatctggaaaacaaggcgcggacaggcacgcaatgctcctatgtcgcacctcatattccacgtatacgacatcgtggagaccacctacacggccgaccgctgcgaggacgtgccatttagcttccagactgatatcattcccagcggcaccgtcctcaagctgctcggcagaacactagatggcgccagtgtctgcgtgaacgttttcaggcagcgctgctacttctacacactagcaccccagggggtaaacctgacccacgtcctccagcaggccctccaggctggcttcggtcgcgcatcctgcggcttctccaccgagccggtcagaaaaaaaatcttgcgcgcgtacgacacacaacaatatgctgtgcaaaaaataaccctgtcatccagtccgatgatgcgaacgcttagcgaccgcctaacaacctgtgggtgcgaggtgtttgagtccaatgtggacgccattaggcgcttcgtgctggaccacgggttctcgacattcgggtggtacgagtgcagcaatccggccccccgcacccaggccagagactcttggacggaactggagtttgactgcagctgggaggacctaaagtttatcccggagaggacggagtggcccccatactcaatcctatcctttgatatagaatgtatgggcgagaagggttttcccaacgcgactcaagacgaggacatgattatacaaatctcgtgtgttttacacacagtcggcaacgataaaccgtacacccgcatgctactgggcctggggacatgcgacccccttcctggggtggaggtctttgagtttccttcggagtacgacatgctggccgccttcctcagcatgctccgcgattacaatgtggagtttataacggggtacaacatagcaaactttgaccttccatacatcatagcccgggcaactcaggtgtacgacttcaagctgcaggacttcaccaa

SEQ ID NO: 184 - DNA, 2653 bp, Kaposi′s sarcoma-associated herpesvirus
tgactcagacgcggaaacagcgcctagaaagtttcctcttgcgctatgtgggacaactagagtccaacctggcaagcagtggagcaagacgccagacagccgatctcgaaaaaaataatgcagacagaggcaacgttcatcctaggtgactgggagataacggtgtctaactgccggtttacttgcagcagcctaacatgtggccccctttacagatctagcggcgactacacgcggctaagaatccccttctctctggatcgactaatacgtgaccatgccatctttgggctagtgccaaatattgaggatctgttaacccatgggtcatgcgtcgccgtagtggccgacgcaaacgccacaggcggcaacgcgcgacgcatcgtcgcgcctggcgtgataaacaatttttcagaacccatcggcatttgggtacgcggccctccgccgcaaacgcgcaaggaagctattaagttctgcatattttttgtcagtcccctgcccccgcgggagatgaccacatatgtgttcaagggcggcgatttgcctcccggagcagaggaacccgaaacactacactccgccgaggcacccctaccgtcgcgcgagacgctggtaactggacagctgcgatccacctcgccgcgaacgtatacgggatactttcacagtcctgtcccgctctcttttttggacctcctgacattcgagtccattgggtgtgacaacgtggaaggtgaccccgagcaattgacacccaagtacttgacgttcacgcagacgggagaaagactttgcaaagtaaccgtttacaacacccattcgacagcatgcaagaaggcccgtgttcgtttcgtctacagaccgacgccgtccgcccgtcagcttgtcatgggtcaggcttcacccctcataacaacccctctgggagccagggtattcgcagtctatccagactgtgagaaaactatcccacctcaggaaaccaccaccctgaggattcaattgctgttcgagcagcatggtgccaacgccggagactgcgcctttgtcatcatggggctcgcccgtgaaacaaagtttgtctcatttcccgcagtactccttccgggcaagcacgaacaccttattgtattcaacccacagacacatcctctgaccattcaacgggacacaatagtgggcgtggcaatggcttgctatatccaccccggtaaggcagccagccaggcaccatacagcttctacgactgcaaggaagagagctggcacgtggggctcttccagatcaaacgcggaccgggaggggtctgtacaccaccttgccacgtagcgattagggccgaccgccacgaggaacccatgcaatcgtgactgtccgagcacatatggcgcaggagtcagagcagtgctcccgtgcgtttgcagtgtgcagtagtaaacgacagctcgggcgcggcgagcccgtgtgggattccgtcattcacccgagccacatcgtcatctctaatcgagtacccctcttactaagagaacagcacatatgtctcccttcgtgccccagcgtcggccagatcctccacagagcctaccccaactttacatttgacaacacgcaccgcaagcagcaaacggagacctacactgcattctacgcttttggggaccaaaataacaaggttaggatcttgcccactgttgtggaaagctcctcgagcgtgctgatttttagactgcgtgcatcggtctctgcgaacatcgccgtgggagggctcaaaataataatacttgctctcaccctggtgcatgcccaaggagtgtacctgcgttgcggtaaggacctttctacaccacactgcgcaccggctattgttcagcgtgaggtgctgagcagcgggtttgagccgcagtttaccgtaactggcattccagtgacatcctcgaacttaaaccaatgctactttctggtaagaaagccaaaaagccggctggcaaagccgtttgcacgcctgtccgcggagacgactgaggagtgtcgcgtcaggtctatccgccttgggaagacacacctgcggatatcggtgactgcgcctgcgcaggaaacgcccgtctgggggctcgtgaccacgagcttcagccttacccccaccgcaccgctggcctttgatcgtaacccgtacaatcacgagacatttgcctgtaatgccaagcactacatcccagtcatctacagcggaccaaaaattacgctggccccgcgcggccgccaggtagtctggcacaacaacagctacacgtcctccctgccatgcaaagtcacagccatcgtgtcaaaccactgctgtaactgtgacatatttttagaggactcggaatggcgcccaaacaagccagcacccctgaaactggtgaacacgagtgatcatcccgtcatattggagccggacacacacattggaaacgccctcttcatcatcgcacccaaggcccgaggtttacgcagactgactcgcttaaccacaaaaaccattgaacttcctggcggggtaaagatagacagcaggaaattacaaacattcagaaaaatgtatgttgccaccggacgcagttaggtgtccggttcccacccacacatttgtctttattgctttca

SEQ ID NO: 185 - DNA, 4069 bp, Kaposi′s sarcoma-associated herpesvirus
cgcgtaattcgaggtccccggaagagtagagggttgcatgttatacaaacaacataaacattaaatgaacattgttcaaaacgtatgtttattttttttcaaacaggggagtagggtaggaagggtacgtctaatacgtaactgttcgctactgcttgttcaggagctcctcgcagaacatcttgcgaattttagattttggactagagcgactgctggcttcaacgcggttcgatgtagggttcggcgtaggagcgtctttctccaccgccgcgcatggtgtatgcgtggtctccggtgcctgttgttggatgctctgcgtgctggaggcgggggtgggttcagcgggtggtgcgccaactaccgcgagtcctgtagagactggcgggtggctcacatgtggctgagcaaaaaggatgggcgccgcttgctggaactgaccgtgtggcgcctgcacgtaaatgggtgggtgtacgtaggttcctccgtgctccttcattgtcgggaattgacacgggaccgctgaattggcgtggggcctgtagtgtggatctactgcggctgctgctgcagaggaggacggcggtggccctgcgtgccaaccgttcagtttcatctctttgagttcagactgtatttccgctatgttctttgacatggacaagatatccttgtgatacgccggctcctctcctggaaagaggtgtccttcgtcgtcctctgcgccgcgcttgcgcttccccgtcctatatccaggcagctgtggcgagtaataccatggatcgtatgggttcttgtaagcgtagccgtatggtggcgctgggtttgaaacatacgaaggtaggtgatggtcggtggggaacatctggcccccacaccccattaggcctggccctgaaagtgtatgtgacatttttgccgctgtggtcttcattccatcgatgctgctttgtagcatgctcaggaaggcggatttggggatggatatgatatcctcttgaccagagctgttcatggctggtctgggtggtgtgacggcttggatgccgaccgggaattggctggcctttaaatacgccgggctcaatatgctggccacacctctgtcagttttcaataggtcgaggcggtcccgtatgaagctggcatctatagcttttgccattaaggtctccaggggactgacgaaatttggtgtggaaaggtcctccagcctgcagctacttacgtgctggaggatgtgggcgcgctccgacttagatactgatgagaatctggaaaccacccactcggcgtcgtgtccgtacacggccactgtgccgcgtcggcgccccagggcgcatagtgatacgtgttgaaacacgggaccgctgggagtctgggataactcgcggggatgtatagacgataaagacagccccgggagccacgtgtggagtatctccaacagtggttccttagggagatttttcacgggggctctggccacgtgggaggtgtccgccagcctggatgccagctctaggaaggctggcgacgtgatggctccggtgcagaaaataccgtgggacacttgaaatagacccagtgtccagcccacttctgtctctggtaggtgttcgattgttattggaaggggttctgtgactgggagataatccgtcacctgatccggatcgagatagagctcttgctccagcttggggcaggacacaacatctacaaaccctccgacgtacaggccctgtgccatgctcggaaaatacgtgtgtgagaccgagccgctgagcccggggcttaggaggctcatgtggcgctttttgcaaaataagaatttaaatacattccacgcccaagagctgcgttttattcatttggttctctgcaggatgtacaatttcggtctaaatgtgtacctgttaagggaggctactgccaatgccgggacctacgacgaggtggtcctgggacgcaaggttcctgcggaggtgtggaagctcgtgtacgatgggctcgaggagatgggcgtgtcaagtgagatgctgctgtgtgaggcataccgggacagcctctggatgcacttgaacgataaggtggggctcttgaggggcctggcgaattatctgtttcaccggctaggggtcacccacgacgttcgcatcgccccggaaaacctggtggacggaaactttttgtttaatctgggaagtgtgctcccctgcaggctgctccttgcggcgggctactgcctcgccttttggggcagcgatgaacacgaacgctgggtgcgcttcttcgcccagaagcttttcatttgctacctgatagtctccgggcgtcttatgccacagaggtctctgctagtttgggccagcgaaacgggctatcccggtccggtggaggcagtctgtcgcgacatccgctccatgtacggcatacgaacgtatgcggtctcgggttatcttccggctccgtccgaagcgcagctggcctaccttggtgcgtttaacaacaacgcggtttaaacgaccgcgaggaccaccggcaggcagccaagaaccataaagtacgctctatcgtagtcatcgccgccgccaaactgggacttgataatctcctggagaagggtgggtggggatgggtgtgaaagcaggacgtccaggccctcttctgttgccaggcggagggctgttctcgcctggagcagcgccagtggatctcggaatgtaagctgctggttcaggatttcgaatatctcattaaacctactgcctgtcagatttacaaatggtccgggttgtttgtgggacacggtcgatcgcgcctcgagggcggccagtattatgccagggaagatgaaggacacgggggcgtttggattagcctgcagtgtggggattatgtagtgctccgatatgaacgaaaatagctggccccttttcagcatgggggcgtttggatccggtagggcaccgggctgaaatttgggtcccagcagggataccaggttcaagcggcggtttgggtgccctcgcgcgacttgcccaaactccagcaatccatacgcgaggataaacacctccagcgcaacaatccccgctcgcaggttccactggtatgcggaaaatggtggtatatcggacccaaacatggcgctcgtaatggcgaataccaagtccatggcgggcgctgtccctggcgcgcccgtacccttgttgtggggaaataatccagccttagccatcattgcgtgaagcttgtggcgctggaagaaggctgtcggatagcggctctccttattgagaggcgccagcgaggcgcgctcctgggggtttgagtatgtgaagctgaagtccccaggaccgctttcctgttttagctgagtgattagcaggtctagcttttgaggcaggtctgctaacaggtcatcgggagtagcgggcagttgcctggatgtcttttgacaaaagtacgcgttgacgaggcaaagcgcggcctgggtgtccgtgagatgcctggcgtcggcgaaaaagtcagcggtggtcgaggcgaccgtcgtcagggtgtgagagatgagtttgagcgatgtggaattctgaaagttaacagtcccctttagttctttagggaagacgcgccgctgcatggcgttgtccgtgaggctgatgaaccacggcccaaaggatggcaaccactgattctggttcatgtacagggtgggcatgagctcgccgcgcaggtccctgtcaacggagaagtgagggtccccggggacgatcgccacggtgaagttacggtggctggcctgcgggggggatgtcactaagggaggctcatgggaacggctttggggcatgtctatgttgtcagaccatgtcatgttgcctatcatctgtttcaccgcgtcgatatctgcgttaatgacgcggacgcgtgagtcatggacctgaacaagccggtccagctctagggaaagcaggtgtgcctttgtctttcgttctcgatttcgcacgagttggctgcgcagtccaagggcgacccttcttgtttcttccatggtgggcttgtg

SEQ ID NO: 186 - DNA, 1544 bp, Kaposi′s sarcoma-associated herpesvirus
acgaccgcgaggaccaccggcaggcagccaagaaccataaagtacgctctatcgtagtcatcgccgccgccaaactgggacttgataatctcctggagaagggtgggtggggatgggtgtgaaagcaggacgtccaggccctcttctgttgccaggcggagggctgttctcgcctggagcagcgccagtggatctcggaatgtaagctgctggttcaggatttcgaatatctcattaaacctactgcctgtcagatttacaaatggtccgggttgtttgtgggacacggtcgatcgcgcctcgagggcggccagtattatgccagggaagatgaaggacacgggggcgtttggattagcctgcagtgtggggattatgtagtgctccgatatgaacgaaaatagctggccccttttcagcatgggggcgtttggatccggtagggcaccgggctgaaatttgggtcccagcagggataccaggttcaagcggcggtttgggtgccctcgcgcgacttgcccaaactccagcaatccatacgcgaggataaacacctccagcgcaacaatccccgctcgcaggttccactggtatgcggaaaatggtggtatatcggacccaaacatggcgctcgtaatggcgaataccaagtccatggcgggcgctgtccctggcgcgcccgtacccttgttgtggggaaataatccagccttagccatcattgcgtgaagcttgtggcgctggaagaaggctgtcggatagcggctctccttattgagaggcgccagcgaggcgcgctcctgggggtttgagtatgtgaagctgaagtccccaggaccgctttcctgttttagctgagtgattagcaggtctagcttttgaggcaggtctgctaacaggtcatcgggagtagcgggcagttgcctggatgtcttttgacaaaagtacgcgttgacgaggcaaagcgcggcctgggtgtccgtgagatgcctggcgtcggcgaaaaagtcagcggtggtcgaggcgaccgtcgtcagggtgtgagagatgagtttgagcgatgtggaattctgaaagttaacagtcccctttagttctttagggaagacgcgccgctgcatggcgttgtccgtgaggctgatgaaccacggcccaaaggatggcaaccactgattctggttcatgtacagggtgggcatgagctcgccgcgcaggtccctgtcaacggagaagtgagggtccccggggacgatcgccacggtgaagttacggtggctggcctgcgggggggatgtcactaagggaggctcatgggaacggctttggggcatgtctatgttgtcagaccatgtcatgttgcctatcatctgtttcaccgcgtcgatatctgcgttaatgacgcggacgcgtgagtcatggacctgaacaagccggtccagctctagggaaagcaggtgtgcctttgtctttcgttctcgatttcgcacgagttggctgcgcagtccaagggcgacccttcttgtttcttccatggtgggcttgtg

SEQ ID NO: 187 - DNA, 2186 bp, Kaposi′s sarcoma-associated herpesvirus
ccttcttggcggcccttgcatgctggcgatgcatatcgttgacatgtggagccactggcgcgttgccgacaacggcgacgacaataacccgctccgccacgcagctcatcaatgggagaaccaacctctccatagaactggaattcaacggcactagtttttttctaaattggcaaaatctgttgaatgtgatcacggagccggccctgacagagttgtggacctccgccgaagtcgccgaggacctcagggtaactctgaaaaagaggcaaagtctttttttccccaacaagacagttgtgatctctggagacggccatcgctatacgtgcgaggtgccgacgtcgtcgcaaacttataacatcaccaagggctttaactatagcgctctgcccgggcaccttggcggatttgggatcaacgcgcgtctggtactgggtgatatcttcgcatcaaaatggtcgctattcgcgagggacaccccagagtatcgggtgttttacccaatgattgtcatggccgtcaagttttccatatccattggcaacaacgagtccggcgtagcgctctatggagtggtgtcggaagatttcgtggtcgtcacgctccacaacaggtccaaagaggctaacgagacggcgtcccatcttctgttcggtctcccggattcactgccatctctgaagggccatgccacctatgatgaactcacgttcgcccgaaacgcaaaatatgcgctagtggcgatcctgcctaaagattcttaccagacactccttacagagaattacactcgcatatttctgaacatgacggagtcgacgcccctcgagttcacgcggacgatccagactaggatcgtatcaatcgaggccaggcgcgcctgcgcagctcaagaggcggcgccggacatattcttggtgttgtttcagatgttggtggcacactttcttgttgcgcggggcattaccgagcaccgatttgtggaggtggactgcgtgtgtcggcagtatgcggaactgtattttctccgccgcatctcgcgtctgtgcatgcccacgttcaccactgtcgggtataaccacaccacccttggcgctgtggccgccacacaaatagctcgcgtgtccgccacgaagttggccagtttgccccgctcttcccaggaaacagtgctggccatggtccagcttggcgcccgtgatggcgccgtcccttcctccattctggagggcattgctatggtcgtcgaacatatgtataccgcctacacttatgtgtacacactcggcgatactgaaagaaaattaatgttggacatacacacggtcctcaccgacagctgcccgcccaaagactccggagtatcagaaaagctactgagaacatatttgatgttcacatcaatgtgtaccaacatagagctgggcgaaatgatcgcccgcttttccaaaccggacagccttaacatctatagggcattctccccctgctttctaggactaaggtacgatttgcatccagccaagttgcgcgccgaggcgccgcagtcgtccgctctgacgcggactgccgttgccagaggaacatcgggattcgcagaattgctccacgcgctgcacctcgatagcttaaatttaattccggcgattaactgttcaaagattacagccgacaagataatagctacggtacccttgcctcacgtcacgtatatcatcagttccgaagcactctcgaacgctgttgtctacgaggtgtcggagatcttcctcaagagtgccatgtttatatctgctatcaaacccgattgctccggctttaacttttctcagattgataggcacattcccatagtctacaacatcagcacaccaagaagaggttgccccctttgtgactctgtaatcatgagctacgatgagagcgatggcctgcagtctctcatgtatgtcactaatgaaagggtgcagaccaacctctttttagataagtcacctttctttgataataacaacctacacattcattatttgtggctgagggacaacgggaccgtagtggagataaggggcatgtatagaagacgcgcagccagtgctttgtttctaattctctcttttattgggttctcgggggttatctactttctttacagactgttttccatcctttattagacggtc

SEQ ID NO: 188 - DNA, 1833 bp, Kaposi′s sarcoma-associated herpesvirus
ctaacccttctagcgttggctagtcatggcactcgacaagagtatagtggttaacttcacctccagactcttcgctgatgaactggccgcccttcagtcaaaaatagggagcgtactgccgctcggagattgccaccgtttacaaaatatacaggcattgggcctggggtgcgtatgctcacgtgagacatctccggactacatccaaattatgcagtatctatccaagtgcacactcgctgtcctggaggaggttcgcccggacagcctgcgcctaacgcggatggatccctctgacaaccttcagataaaaaacgtatatgcccccttttttcagtgggacagcaacacccagctagcagtgctacccccattttttagccgaaaggattccaccattgtgctcgaatccaacggatttgacctcgtgttccccatggtcgtgccgcagcaactggggcacgctattctgcagcagctgttggtgtaccacatctactccaaaatatcggccggggccccggatgatgtaaatatggcggaacttgatctatataccaccaatgtgtcatttatggggcgcacatatcgtctggacgtagacaacacggatccacgtactgccctgcgagtgcttgacgatctgtccatgtacctttgtatcctatcagccttggttcccagggggtgtctccgtctgctcacggcgctcgtgcggcacgacaggcatcctctgacagaggtgtttgagggggtggtgccagatgaggtgaccaggatagatctcgaccagttgagcgtcccagatgacatcaccaggatgcgcgtcatgttctcctatcttcagagtctcagttctatatttaatcttggccccagactgcacgtgtatgcctactcggcagagactttggcggcctcctgttggtattccccacgctaacgatttgaagcggggggggggtatggcgtcatctgatattctgtcggttgcaaggacggatgacggctccgtctgtgaagtctccctgcgtggaggtaggaaaaaaactaccgtctacctgccggacactgaaccctgggtggtagagaccgacgccatcaaagacgccttcctcagcgacgggatcgtggatatggctcgaaagcttcatcgtggtgccctgccctcaaattctcacaacggcttgaggatggtgcttttttgttattgttacttgcaaaattgtgtgtacctagccctgtttctgtgcccccttaatccttacttggtaactccctcaagcattgagtttgccgagcccgttgtggcacctgaggtgctcttcccacacccggctgagatgtctcgcggttgcgatgacgcgattttctgtaaactgccctataccgtgcctataatcaacaccacgtttggacgcatttacccgaactctacacgcgagccggacggcaggcctacggattactccatggcccttagaagggcttttgcagttatggttaacacgtcatgtgcaggagtgacattgtgccgcggagaaactcagaccgcatcccgtaaccacactgagtgggaaaatctgctggctatgttttctgtgattatctatgccttagatcacaactgtcacccggaagcactgtctatcgcgagcggcatctttgacgagcgtgactatggattattcatctctcagccccggagcgtgccctcgcctaccccttgcgacgtgtcgtgggaagatatctacaacgggacttacctagctcggcctggaaactgtgacccctggcccaatctatccacccctcccttgattctaaatttta

SEQ ID NO: 189 - DNA, 890 bp, Kaposi′s sarcoma-associated herpesvirus
cgatttgaagcggggggggggtatggcgtcatctgatattctgtcggttgcaaggacggatgacggctccgtctgtgaagtctccctgcgtggaggtaggaaaaaaactaccgtctacctgccggacactgaaccctgggtggtagagaccgacgccatcaaagacgccttcctcagcgacgggatcgtggatatggctcgaaagcttcatcgtggtgccctgccctcaaattctcacaacggcttgaggatggtgcttttttgttattgttacttgcaaaattgtgtgtacctagccctgtttctgtgcccccttaatccttacttggtaactccctcaagcattgagtttgccgagcccgttgtggcacctgaggtgctcttcccacacccggctgagatgtctcgcggttgcgatgacgcgattttctgtaaactgccctataccgtgcctataatcaacaccacgtttggacgcatttacccgaactctacacgcgagccggacggcaggcctacggattactccatggcccttagaagggcttttgcagttatggttaacacgtcatgtgcaggagtgacattgtgccgcggagaaactcagaccgcatcccgtaaccacactgagtgggaaaatctgctggctatgttttctgtgattatctatgccttagatcacaactgtcacccggaagcactgtctatcgcgagcggcatctttgacgagcgtgactatggattattcatctctcagccccggagcgtgccctcgcctaccccttgcgacgtgtcgtgggaagatatctacaacgggacttacctagctcggcctggaaactgtgacccctggcccaatctatccacccctcccttgattctaaatttta

SEQ ID NO: 190 - DNA, 1151 bp, Kaposi′s sarcoma-associated herpesvirus
aacggggtgtgtgctataatggatggctatgggggggctgtagataattgagcgctgtgcttttattgtggggatatgggcttgtacatgtgtctatcatcggtagccataaaatgggccatgacaactgccacaagtaagtcgtccgacatgtgcttttgcttggcgctgtatgactgccctccatccctaagcgggacgcacttgatcgcgcggacctgttctaccaggtaggtcaccgggtcaaatgatattttgatggtgttggacaccaccgtctggctggcgctcagggtgccggagttcagagcgtagatgaatgtctcaaacgcggaggatttctcgcctcccaacatgtaaattggccactgcagggcgctgctcttgtcagtatagtgtagaaaatgtatggggagcgggcatatttcgttaaggacggttgcaatggccaccccagaatcttggctgctgttgccttcgaccgccgcgttcacgcgctcaattgtggggtggagcacagcgatcgccttaatcatcgtgcatgcgcaggacgctatctcgtaagcagctgcgccagtgaggtcgcgcaggaagaaatgctccatgcccaatatgaggcttctggtgggagtctgagtactcgtgacaacggcgcccacgccagtaccggacgcctccgtgttgttcgtatacgcggggtcgatgtaaacaaacagctgttttccaaggcacttctgaacctgctgggcggtggtgtctacccgacacatgtcaaactgtgtcagcgctgcgtcacccaccacgcggtaaagcgtagcatttgacgacgctgctccctcgcccattagttcggtgtcgaatgccccctccataaagaggttggtggtggttttgatggattcgtcgatggtgatgtacgtcggaatgtgcagtctgtaacaaggacaggacactagtgcgtcttgcaggtggaaatcttcgcggtggtccgcacacacgtaactgaccacattcagcatcttttcctgggcgttcctgaggttaagcaggaaactcgtggagcggtctgacgagttcacggatgatataaatataagcttggcgtctttctgaagcatgaaacccagaatagccggcagtgcatccttttt

SEQ ID NO: 191 - DNA, 1303 bp, Kaposi′s sarcoma-associated herpesvirus
ccggaggcgcaaacttcggaatttcctaaacaaggaatgcatatggactgttaacccaatgtcaggggaccatatcaaggtctttaacgcctgcacctctatctcgccggtgtatgaccctgagctggtaaccagctacgcactgagcgtgcctgcttacaatgtgtctgtggctatcttgctgcataaagtcatgggaccgtgtgtggctgtgggaattaacggagaaatgatcatgtacgtcgtaagccagtgtgtttctgtgcggcccgtcccggggcgcgatggtatggcgctcatctactttggacagtttctggaggaagcatccggactgagatttccctacattgctccgccgccgtcgcgcgaacacgtacctgacctgaccagacaagaattagttcatacctcccaggtggtgcgccgcggcgacctgaccaattgcactatgggtctcgaattcaggaatgtgaacccttttgtttggctcgggggcggatcggtgtggctgctgttcttgggcgtggactacatggcgttctgtccgggtgtcgacggaatgccgtcgttggcaagagtggccgccctgcttaccaggtgcgaccacccagactgtgtccactgccatggactccgtggacacgttaatgtatttcgtgggtactgttctgcgcagtcgccgggtctatctaacatctgtccctgtatcaaatcatgtgggaccgggaatggagtgactagggtcactggaaacagaaattttctgggtcttctgttcgatcccattgtccagagcagggtaacagctctgaagataactagccacccaacccccacgcacgtcgagaatgtgctaacaggagtgctcgacgacggcaccttggtgccgtccgtccaaggcaccctgggtcctcttacgaatgtctgactacttcagccgcttgctgatatatgagtgtaaaaaacttaaggccctgggcttacgttcttattgaagcatgttgcgcacatcagcgagctggaccgtcctccgggtcgcgtgtagattatggttccgttctccttcttgatgtttaaatttttgggggggaaccaccgacaaagcgtctttatgatttccgcgaacacggagttggctacgtgcttttggtgggctacgtacccaatgttaatgttctctacggatgccagtagcatgctgatgatcgccaccactatccatgtctttccgtgtctccttggtattaggaatacgcttgccttttgcttaaacgtctgtaaaacactgtttggagtttca

SEQ ID NO: 192 - DNA, 858 bp, Kaposi′s sarcoma-associated herpesvirus
agcggagagggggtggtgcgagttggcagttgacgggtttgtgatagctggagtgctgaccacggcacaggacccattaactttcctatgtgtttatttttagcaatggtctccagaattcaaggatctcaaaagggcctgccagatggccgggtttactctgaagggggggacttcgggggatcttgtattctcatcgcatgcgaacttgctcttttcaacctcgatgggatatttcctccatgcaggcagtccaaggtcgacagcggggacggggggtgagcctaacccacgtcacatcaccggaccagacactgagggaaatggggaacacagaaactcccccaacctctgcggctttgttacctggctgcaaagcttaaccacatgcattgaacgagccctaaacatgcctcccgacacttcctggctgcagctgatagaggaagtgatacccctgtattttcataggcgaagacaaacatcattctggctcatccccctatcgcactgtgaagggatcccagtatgcccccctttaccatttgactgcctagcaccaaggctgtttatagtaacaaagtccggacccatgtgttaccgggcaggcttttcgcttcctgtggatgttaattacctgttctatttagagcagactctgaaagctgtccggcaagttagcccacaggaacacaacccccaagacgcaaaggaaatgactctacagctagaggcctggaccaggcttttatctttattttgaaaaaagggaaacaatggggggtttgaaaagggtgcacattttcagatattttaaaacttcattgttctccaggtgcttggtaaagatggtatcac

SEQ ID NO: 193 - DNA, 2061 bp, Kaposi′s sarcoma-associated herpesvirus
gttcaacatggacgcatggttgcaacagacggtctttaggggcaccctatccatcagtcagggggtggacgaccgggatctgttactggcacctaagtggatttcctttctgagcctctcatcatttctgaaacagaaactgctctcgctgctcagacagattcgggaacttaggctaaccaccacagtgtatcccccacaggacaagctgatgtggtggtcccactgctgcgatccagaggatattaaagtggtgatcttaggccaggacccgtaccacaagggccaagctactggcctggcgtttagtgtggatccgcaatgtcaggttccacccagtttgagaagcatctttagagagctagaggcttccgtccccaatttcagtactccttcccacgggtgcctcgacagctgggctcgccagggtgtgttgctactaaacacagttttgacggtggagaaggggagggccggctcacacgagggacttggctgggattggttcacgagtttcatcatcagtagcatatcctcaaagttagaacattgcgtttttctcctgtgggggcgcaaggccattgacagaactccgctcataaacgcacagaaacacctggtgcttacggcccagcatccatctccgctggcctctcttggtggccgacactcgcgatggcctcggttccagggctgtaatcactttaacctagccaacgactatttgactcgccaccggcgtgagactgtggactggggcctgttggagcagtaaaggcaataactcgtgtgctttgtaaatttccgcccctagcggtcaaccccgtacaaggccatggcgatgtttgtgaggacctcgtctagcacacacgatgaagagagaatgcttccaattgaaggagcgcctcgcagacgaccccccgtgaagttcatattcccacctccacctctttcatcacttccaggatttggcaggccgcgcggctatgctggacccacggtgatagatatgtctgccccagacgacgtcttcgccgaggacacgccatcgccgccagcaacccctctggatctacagatatccccggatcagtcgagcggcgaatctgaatatgacgaggatgaggaagatgaagatgaagaagaaaatgacgatgttcaggaggaagacgagccagaggggtaccctgcagacttttttcaacctttatctcacttgcgcccgaggcctctggccagacgggcccatacgcccaaaccggtagcagtggtagcgggccgcgtgcgcagttcaacggacacggcggagtccgaggcgtccatgggatgggttagtcaggatgacggattttcccctgctgggctctcaccttcagacgacgagggggttgctatcctggaaccgatggcggcatacactgggaccggggcatacggactttcacctgcttccagaaatagtgtacctggaacacaaagttcaccatacagcgaccctgatgaagggccctcgtggcgccccctgcgcgccgcacccaccgcgatcgtcgacctgacatcggactctgatagcgatgacagttccaactctccggacgtgaacaatgaggccgcgtttaccgacgcgcgccatttttcccaccagccaccctcgtccgaggaggacggagaagaccaaggggaagtattgagtcagagaatcgggctcatggacgtgggccagaagcgcaaaaggcagtctaccgcctcctctggtagcgaggatgtggtgcgctgccagagacaaccaaacttaagccgcaaagcagtggcgtccgtgataattatatcctcggggagtgacacagacgaggagccctcgtccgccgtgagcgtgatcgtgtctccgtcgagcacaaagggtcacctcccaacccaatctcccagtacttccgcccactcgatttcatcaggaagcacaactaccgcggggtccaggtgcagcgacccaacccgcatcctggcctccacgccacccctgtgtggaaacggtgcatataactggccgtggctggactgata

SEQ ID NO: 194 - DNA, 1123 bp, Kaposi′s sarcoma-associated herpesvirus
aaaggtcgatctttaccttgtcatcttgcgccatttttgtggctgcctggacagtattctcacaacagactaccccttgcggagtaaggttgactttttaaaggggacgtgtcattgccacccagctactggtttctgggcggggcttaatgagtcgccggtagctgcctggtatttagtggaggataagctgtagctgggtcctatgggggttgggtggggagaccctagcgtacatgtgactgaacatggaggtgtgtatcccaattccgggtattggagatgaaaattgtgagagctggagggcacagattgtggcattcggtaccacatcgggtttcgtcaagaccgagcgtattctcagaggtctgtttccggagcgcggacacccggggttcttagcgtccctggtggtcctgaagcatacgctggcttccccgggggggctcaacaccagactgaatctacttccagtattacagatgttaaaatatgtgggacaggaaatgtacatgcgggcaaaatgccaggcaacagcatctgacatgactttgatctgggatgactgcaaagatagatttatgctgatactggaacaggcctgtgggtgccaccaatgtatgaccgtggtagaagaaatcacccactgtagcgccatctctgcccccccaagctctttgtcccacgggagacacattctttctgcggggctcatcaactttgcaagacgccaggttctccttggtgggtcagtgtctttttctgagttttctattccagacctaatacagacaccggagcaatacccctttgtggatgtggagttccggcgggagcttagcttgatttcatcgtgtttgaacgtctgctggctctaccacatcttcatagagcacattacctcggacgtgagacggttggagtcatgcatggccagtgtcctggaagagtatggcggactgtcacccacccgcccatgggcagaggcagtgacctttttgagtcagctgccgcgccccaccaggaaaccctggaaagaactgtcggtaagccggatcaacgtggaagcccggcttttggataccctggtgatgcaattagagaaaccggttcctgtggaaat

SEQ ID NO: 195 - DNA, 2084 bp, Kaposi′s sarcoma-associated herpesvirus
agtgttcgcaagggcgtctgtgcctgcgttaacttcccaggcagtttatttttaacagtttggtgcaaagtggagttaacctacagattctacttaaaatagctcattttctcacgaatctggttgattgtgactatttgtgaaacaataatgattaaagggggtggtatttcctccgttgtcgactataacctggcgtgtaaacgtgtaaccctgccaaatgcccagaatgaaggacatacctactaagagttccccgggaacggacaattctgagaaagatgaagctgtcattgaggaagatctaagcctcaacgggcaaccattttttacggacaatactgacggtggggaaaacgaagtctcttggacaagctcgctgttgtcaacctacgtaggttgccagcccccggccataccggtctgtgaaacggtcattgaccttacagcgccttcccaaagtggcgcgcccggtgacgaacatctgccatgctcactgaatgcagaaactaaattccacatccccgatccttcctggacgctctctcacacaccaccaagaggaccacacatttcgcaacagcttccaactcgcagatccaagaggcgactacatagaaagtttgaagaggaacgcttatgcactaaggccaaacagggcgcaggtcgccccgtgcctgcgtctgtagttaaggtagggaacatcaccccccattatggggaagaactgacaaggggtgacgccgtcccagccgcccctataacacccccctccccgcgcgttcaacgcccagcacagcccacacatgtcctgttttctcctgtttttgtctctttaaaggccgaagtatgtgatcagtcacattctcccacgcgaaagcaaggcagatacggccgcgtgtcatcgaaagcatacacaagacagctgcagcaggtatagacgggaaacaggtgtctatcttggccggctggttactcaaatgggaacaatggcgccaccttgctgtctttgtaggcattagaagaaaaggatgcacaactatgtttcctagcggcgagattggaggcacataaggaacagattattttccttcgcgacatgctgatgcgaatgtgccagcagccagcgtcgccaacggacgcgccactcccaccatgttgaagcttggttgtgccgtcgtccgggagaaccatgccagactttgtgtggtaagaaggaattgttatccggcagcaatattaaagggacccaagttaatcccttaatcctctgggattaataaccatgagttccacacagattcgcacagaaatccctgtggcgctcctaatcctatgcctttgtctggtggcgtgccatgccaattgtcccacgtatcgttcgcatttgggattctggcaagagggttggagtggacaggtttatcaggactggctaggcaggatgaactgttcctacgagaatatgacggccctagaggccgtctccctaaacgggaccagactagcagctggatctccgtcgagtgagtatccaaatgtctccgtatctgttgaagatacgtctgcctctgggtctggagaagatgcaatagatgaatcggggtcgggggaggaagagcgtcccgtgacctcccacgtgacttttatgacacaaagcgtccaggccaccacagaactgaccgatgccttaatatcagccttttcaggtgtattacacgtttcaactgtaatccctcgcaattgggtaaaccgtcggtgtgtagggataaagcgtaaccttacgttctgtctcatctacaggatcatattcatctggggaaccatccaggaccacgcgaattcgcgtatcaccggtcgcagaaaacggcagaaatagtggtgctagtaaccgtgtgccattttctgccaccactacaacgactagaggaagagacgcgcactacaatgcagaaatacggacccatctttacatactatgggctgtgggtttattgctgggacttgtccttatactttacctgtgcgttccacgatgccggcgtaagaaaccctacatagtgtaacacaaaaccataaaagta

SEQ ID NO: 196 - DNA, 1640 bp, Kaposi′s sarcoma-associated herpesvirus
tcccactatataacctggctgccaggttcccaaaatagcccgcggcatacggctcacttccccccacattccccccgtgcacaatataagaaccaaaggacatggtacaagcaatgatagacatggacattatgaagggcatcctagagggtaagtcctcgtctacaacagacttttcccatttctaacgtatcgtgctatcttcgtcgcccggcggaccatccccccacccctcatttatcgcgtttgatattacagactctgtgtcctcctctgagtttgacgaatcgagggacgacgagacggacgcaccgacactggaagacgagcaattgtccgaacccgccgagcctccggcagacgagcgcatccgtggtacccagtcggcccagggaatcccaccccccctgggccgcatcccaaaaaaatctcaaggtcgttctcaactgcgcagtgagatccagttttgctccccactgtctcgacccaggtccccctcaccagtaaacaggtacggtaaaaaaatcaagtttggaaccgccggtcaaaacacacgtcctccccctgaaaagcgtcctcggcgcagaccacgcgaccgcctacaatacggcagaacaacacggggcggacagtgtcgcgctgcaccgaagcgagcgacccgccgtccgcaggtcaattgccagcggcaggatgacgacgtcagacagggtgtgtctgacgccgtaaagaaactcagactccctgcgagcatgataattgacggtgagagcccccgcttcgacgactcgatcatcccccgccaccatggcgcatgtttcaatgtcttcattcccgccccaccatcccacgtcccggaggtgtttacggacagggatatcaccgctctcataagagcagggggcaaagacgacgaactcataaacaaaaaaatcagcgcaaaaaagattgaccacctccacagacagatgctgtcttttgtgaccagccgccataatcaagcgtactgggtgagttgccgtcgagaaaccgcagccgccggaggcctgcaaacgcttggggctttcgtggaggaacaaatgacgtgggcccagacggttgtgcgccacggggggtggtttgatgagaaggacatagatataattttggacaccgcaatatttgtctgcaatgcgtttgttaccagatttagattacttcatctttcctgcgtttttgacaagcagagcgagctagcactgatcaaacaggtggcatatttggtagcgatgggaaaccgcttagtagaggcatgtaaccttcttggcgaggtcaagcttaacttcaggggagggctgctcttggcctttgtcctaactatcccaggcatgcagagtcgcagaagtatttctgcgcgcggacaggagctgtttagaacacttctggaatactacaggccaggggatgtgatggggctactaaacgtgatagtaatggaacatcacagcttgtgcagaaacagtgaatgtgcagcggcaacccgggccgcaatggggtcggccaaatttaacaagggtttattcttttatccactttcttaaggattgccaaaccccatggcagagtgtctcccgtattccatgtaactcacgtagcctttctct

SEQ ID NO: 197 - DNA, 63 bp, Kaposi′s sarcoma-associated herpesvirus
ggattgccaaaccccatggcagagtgtctcccgtattccatgtaactcacgtagcctttctct

SEQ ID NO: 198 - DNA, 2717 bp, Kaposi′s sarcoma-associated herpesvirus
ttgaataatacatgtgtttttcttggtttgttgaccatgacacccctccctcgcgtccaaaggccgcttgtattagagggtggacagtgcctgggtgctgtcccgggttatgggtgtgtgccagtagttcaactgcattggttcccttttccgtagtgagttctaaccacaagtttccgcagcccgacaaccggctggggggggcggtgttgagctgcatatattgagttttgttgttagatggcacagagtctacgtgccagtggggttggggtccagctagttgtggcgagaaagtcgcccacggaaaaggtgttttgtgtcgtggcttttgcctaaaaagatgcctcgctacacggagtcggaatggctcacggactttattatagatgctttagacagtggacgcttctggggggtagggtggttggatgaacaaaagagaatattcaccgtgccgggtcgaaaccggcgggagagaatgccagaaggcttcgatgacttctatgaggcatttttggaggagcgacgtaggcacgggctgccagaaatcccggagactgagactggcctgggctgctttggacggctattaaggaccgccaatcgagccagacaggagaggccctttaccatctataagggaaaaatgaaactcaaccgctggattatgacacctaggccatacaagggatgtgaaggatgtcttgtgtacttgacgcaggaaccagccatgaaaaacatgctaaaagcattgtttgggatctatccccatgatgacaaacacagagaaaaggcacttagaaggagccttagaaaaaaagcccagaggtaggatggttgatgtactgggcggtgggttgtgtgggcggcgggatgtacgtgcagcgggcatcacgggaaattggagatgtcactcagacttacctttgtgtaattaacttttgtttagggaggccgccaggaaacaggcggcggcagtcgccacgcccacaacatcctccgcagctgaagtttcatcacggtcacagagcgaagatacggaatcgagtgacagcgaaaacgaactttgggtgggggctcagggttttgtagggagggatatgcacagtttgttttttgaagagccagaaccgtcggggtttgggtcatctggtcagtcatcgagcttattagctccggattccccgcgtccctccacgagccaggtgcagggcccattacacgtgcacaccccgacggatctatgtttgccaacggggggtttaccttctcctgttatttttccacatgagacacaaggcttattagcgccgcctgctggacagtcgcaaaccccattttccccagaaggccccgtccccagtcatgtcagtgggctggatgattgcctaccgatggtggatcacattgaggggtgtttgttagatctcttgtcagatgttggccaggagcttcctgacttaggcgacctgggtgaacttctgtgtgaaactgcgagccctcagggcccgatgcagtcggagggaggtgaggaggggtccacggagagtgtctcagtacttcccgccacgcatccccttgagagttcggcacctggggcctctgtcatgggttcaggccaggagcttcctgacttaggcgacctgagtgaacttctgtgtgaaactgcgagccctcagggcccgatgcagtcggagggaggtgaggaggggtccacggagagtgtctcagtacttcccgccacgcatccccttgagagttcggcacctggggcctctgtcatgggttcatctttccaagcttccgacaatgtggatgattttattgattgtattccaccgttgtgtcgtgatgaccgggacgtcgaggaccaagagaaagctgaccagacattttactggtatggaagcgacatgaggcccaaggtcttaaccgccacccaatccgtggcagcatacctgagtaagaaacaggctatttacaaagtgggtgacaagcttgtgcccctagtggtggaagtgtattatttcggagaaaaggtgaagacccactttgatttaacggggggcatcgttatttgctcccaagtcccagaggcctcccctgaacacatatgtcagacggtacccccgtataaatgcttacttcccagaacggcccactgtagtgtggacgcaaaccgaactttggaacagacgctggacaggttttccatgggagttgtggccatcggtacaaacatgggcatttttctgaagggattattggaatacccagcatactttgttggaaatgcatcgcgaagaagaataggcaaatgtaggcccctgtcccaccgccacgagatccaacaagcttttgacgtggagcgacataatcgagaacctgaagggtcccggtacgcgtccctgtttctgggccgccggccgtcgcctgaatatgactcggatcactatccagtcattttgcacatttaccttgccccattttaccacagagactaaaattttgacaagtcttcttgtcactctgtccgggtacctccctttgtcttaccgccctccgttttgcactataaatatcattgccgttagaaaccaggctctatccgcaacttctatgtttcctgttatagtaggcccatgtgggcttgggagtggccaaactcactgagtgggacatcattaaaggttagcgccaccgtgtggctgcaa

SEQ ID NO: 199 - DNA, 1056 bp, Kaposi′s sarcoma-associated herpesvirus
caccatgtgccgcctggacagtgagcgcgctctgtcgctcttcagttatctgagcgggacgttggcggcgaccccctttctgtggtgttttatcttcaaggccctgtactcgttcacactctttaccacagagatcacggccgtgtttttctggtcgctgccagtcacgcacttggccctgatatgcatgtgtctgtgccctgcggcgcaaaaacagctggaccggaggctggaatggatctgcgcgtcagcagtgtttgctgctgtagtttgcgcggccttttctgggtttacattttctcgtgtgcccttcataccgggtctgtgcgtacttaactgtttactgctgttaccttatccgctagccaccgcaacggcggtgtatcaggcgccgccaatagtacacaggtactatgagctgggcttctgcggagcatttatggtgtactaccttctgttgtttaagaaggtctttgtgtccggcgttttctggctgcccttcattgtcttcttggtcgggggacttttggcatttaggcacctggaacagcatgtgtacatcagggccggaatgcaaaggaggagggccatattcatcatgcccgggaagtacatcacctattcagtgttccaggcctgggcctactgtaggcgcgaggttgtcgtgtttgtgaccttactgctggccaccctgatatcgacggcctcgatcggcctgctgactccggtcctgattggcctggataagtatatgacgctattttatgttgggttactgtcatgcgtgggcgtatccgtcgcctcccgacgagcgctatttgttctcctgcctttggcggcagtgttgctcaccttggtgcacatacttggatcaggtccggatatgctcctagttaggtcctgcctctgctgcctattcctcgtgagcatgctggccgcaatgggggtcgagattcagctaattaggcgaaaactccacagggcacttaacgctccacagatggtattggccctatgcacggttggaaatttatgtatctcatgtctcctgtcggt

SEQ ID NO: 200 - DNA, 2452 bp, Kaposi′s sarcoma-associated herpesvirus
aggccatggcagcccagcctctgtacatggagggaatggcctccacccaccaagctaactgtatattcggagaacatgctggatcccagtgcctcagcaactgcgtcatgtacctggcgtccagctattataacagcgaaacccccctcgtcgacagagccagcctggacgatgtacttgaacagggcatgaggctggacctcctcctacgaaaatctggcatgctgggatttagacaatatgcccaacttcatcacatccccggattcctccgcacagacgactgggccaccaagatcttccagtctccagagttttatgggctcatcggacaggacgcggccatccgcgagccattcatcgagtccttgaggtcggttttgagtcgaaactacgcgggcacggtacagtacctgatcattatctgccagtccaaagccggagcaatcgtcgtcaaggacaaaacgtattacatgtttgacccccactgcataccaaacatccccaacagtcctgcacacgtcataaagactaacgacgttggcgttttattaccgtacatagccacacatgacactgaatacaccgggtgcttcctttactttatcccacatgactacatcagcccagagcactacatcgcaaaccactaccgcaccattgtgttcgaagaactccacgggcccagaatggatatctcccgcggggtggaatcatgctccatcaccgaaatcacgtccccttctgtatcccccgcgcctagtgaggcaccattgcgcagggactccacccaatcacaagacgaaacgcgcccgcgcagacctcgcgtcgtcattcctccttacgatccgacagaccgcccacgaccgcctcaccaagaccgcccgccagagcaggcagcgggatacggtggaaacaaaggacgcggcggtaacaaaggacgcggcggaaagacgggacgtggcggaaatgaaggacgcggtggccaccagccaccagacgagcaccagcccccacacatcaccgcggaacacatggaccagtccgacggacaaggcgccgatggagacatggatagtacacccgcaaatggtgagacatccgttacggaaaccccgggccccgaacccaatcccccagcacggcctgacagagagccaccgcccactcccccggcgaccccaggcgccacagcgctgctctctgacctaactgccacaagagggcagaaacgcaaattttcctcgcttaaagaatcttatcccatcgacagcccaccctctgacgacgatgatgtgtcccagccctcccaacaaacggctccggatactgaagatatttggattgacgacccactcacacccttgtacccactaacggatacaccatctttcgacataacggcggacgtcacacccgacaacacccaccccgagaaagcagcggacggggactttaccaacaagaccacaagcacggatgcggacaggtatgccagcgccagtcaggaatcgctgggcaccctggtctcgccatacgattttacaaacttggatacactgctggcagagctgggccggttgggaacggcacagcctatccctgtaatcgtggacagactaacatcgcgaccttttcgagaagccagcgctctacaggctatggataggatactaacacacgtggtcctagaatacggtctggtttcgggttacagcacagctgccccatccaaatgcacccacgtcctccagtttttcattttgtggggcgaaaaactcggcataccaacggaggacgcaaagacgctcctggaaagcgcactggagatccccgcaatgtgcgagatcgtccaacagggccggttgaaggagcccacgttctcccgccacattataagcaagctaaacccctgcttggaatccctacacgccactagtcgtcaggacttcaagtccctgatacaggcattcaacgccgaagggattaggatcgcctcgcgtgagagggagacgtccatggccgaactgatagaaacgataaccgcccgccttaaaccaaattttaacattgtctgtgcccgccaggacgcacaaaccattcaagacggcgtcggtctcctcagggccgaggttaacaagagaaacgcacagatagcccaggaggctgcgtattttgagaatataatcacggccctctccacattccaaccacctccccaatcgcaacagacgttcgaagtgctgccggacctcaaactgcgcacgctcgtggagcacctgaccctggttgaggcgcaggtgacaacgcaaacggtggaaagtctacaggcatacctacagagcgctgccactgctgagcatcaccttaccaacgtgcccaacgtccacagtatactgtctaacatatccaacactctaaaagttatagattatgtaattccaaaatttat

SEQ ID NO: 201 - DNA, 26 bp, Kaposi′s sarcoma-associated herpesvirus
gcttgtgattttgtttagggcggaaa

SEQ ID NO: 202 - DNA, 1041 bp, Kaposi′s sarcoma-associated herpesvirus
aagccacacctctccccctttttcctccctagaagccaccgtcgccgctccgcacttgcatttggcgccatgggtgctggtgtgtgtggggggcagtgttctcacgacccatctacctcaactgaacacacggacaacggctagcgtactctcgcggcccagcgtcgtcgatgggagaacctgacagagcaccctgaaactccaggctctacaggtaggccacatacgctcgccactctatatggcaactgccaataacccgccctcgggacttctggatcccacgctatgtgaggatcggatcttttacaatattcttgaaattgagccgcgctttttaacttctgactctgtatttgggacctttcaacaatctcttacttcgcatatgcgtaagttactgggcacatggatgttttcagtttgccaggaatacaacctagaacctaacgtggtcgcgttggcccttaatcttttggacagactcctacttataaagcaggtgtccaaagaacactttcaaaagacagggagcgcctgcctgttagtggccagtaagctcagaagcctcacgcctatttctaccagttcactttgctatgccgcggcagactccttttcccgccaagaacttatagaccaggagaaagaactccttgagaagttggcgtggcgaacagaggcagtcttagcgacggacgtcacttccttcttgttacttaaattgctggggggctcccaacacctggacttttggcaccacgaggtcaacaccctgattacaaaagccttagttgacccaaagactggctcattgcccgcctctattatcagcgctgcaggctgtgcgctgttggttcctgccaacgtcattccgcaggatacccactcgggtggggtagttcctcagctggcaagcatattgggatgcgatgtttccgttctacaggcggcagtggaacagatcctaacatctgtttcggactttgatctgcgcattctggacagctattaagcttgtgattttgtttagggcggaaa

SEQ ID NO: 203 - DNA, 4756 bp, Kaposi′s sarcoma-associated herpesvirus
cccgcggatgtctacgtgcccttcccccttaatttaatctagcctcccgttcccatgatgcagagaggcgaatttggtttgtacacagatgtgactatgtatttgttttattatgcgattaaatgaggggtctgatcccaaaagcaatgtttagtggtggtcgttgatcttcttgacgctccataggtagattgactggaacgccatggcccacggggacatggacaggggtgttaggtctggtggaacatgctgccactgccacggatggaacatcagagatgggtctatgatcagggcagcgtgtcgcccgtcactggatgtaagtccggccaccgtggagttgcctgtggggtttctgggatagtgtctggctggcagggtctcatccgcggcatttccatggtaggtgagggttatctcgcctcgctgtctcagtatgtactcgagggcgtcctgctcgtaccggacccccaggtactctccctgggcccagctgggcagcaccgtcccccgcaacactcggaggaaaacgctcttagtgttctgagggatctgtatgtttagccagtggctgtcatacagcttggacacgttggtctccaggtttaccgcccagcgctggggtggtgtgggtccgtacgtgtatggtgaggattccgaccggcccactacacccagggccaccagcagctggaagcccacctcgccacagcagatggagaatgtgtcgggtctgtttagaaactctgtcagggtggaggcacaggtagggtcgttacacagcgccaggacccatcccctggcgctggcgtagctggcctggcagcctgttctgagacatgtaatcagaccagagaaccccgacaaggactgtcctcgtttaagctcttccacagtcaccgtggccacctcaaagcccgtgttctgcaacgcggccatgagcgcgtacggggcactgctcccaggcagcaccaacgcggccacacggcgcggggaggtggggcacgaaaacaggcgcagctgactcccaaggcacatggcccttaggctgcccaggtgatgctccagacgacccaggtccttcctgtgcatgtcctccagtgggtgcaggggaggcgtcaccaggttccacatttcgtcagaaaaggaggtccatgagacttgcaaggaagtcagggtctcttgaaacacaactgtctcgttctgcaaaaccgtgacgttgttgccttgtccctcggggccaacggtgcccagtgggtgtgccacgcagcggtagtccctggccgcccgcagcacctctgacaagtgtacctggggcacctcaaccagtgccccaggggtctctgaaaccataagttcgagcgggttagggtgggcgggtagtgagagctgcagtcccctgcagccggccagggccatctcgattgcagatgggagaagccctccgtcccctatgtcgtgcccagatacaatgagcctcttggacatcaggtacttaacaagcatgaacaggctggcgaccgtggacgggttcagagggggtattgggtgcctggatgccaggaagttgtgctcgaaggtggacccggctatgagacagctctgattcacggccaggtataccagggcgttgccttcgacctttacgtccggggtgaccctgtatctggatcccttgacctcggcccagctggtaaacaccaccgagttgaagggaaggacctccaccgtttcttgctgttgtgtgatgcgcacatggcgctccgaaagcgtcggagagctggcagccgaggagatggacagtgccactcccagctcccggcagaattccttgcaggcgaagaggcactcctgtaggaggccggcttggtggtcctctggactccacgccacggcgccagttagcactacgtcctggagcttggacacgggactgaacatgaggttggtgagagcctcggtgatggcataggtggccccggtggatacattagtagccatcttgtaggcctgctcccccatggccattgcctgacccctccacgctggcactggaagcagctcctggggcagggccttcacccaggtctcgaagtccttgtgtaggaggttggccatggacggagtgatggcctccaccgtgtcgggcactctgggcgccaccctctcggccagcatggacgagtgcagcaccaggtggtagtctgaaaccggtatgtccaggggtcccacgccagcctgttgggcgatgaggccgttggagcatcggtccatgtgtcgcgtaaagaactccttgctgccaaccgtcgagtggcgaagtaactggtggattgtggagccggtggcaaaaaggccccagtcaacatcctcggggtgccccgagacgcggacaccatcggacagcgccagccagggggacgggggggtggacgacggctggtctacagagaagaccctcgtggtctccccggtcaggtcgtctactattctgatgcctgggtgctccgaggtcctcccgaggaccgttacctggcacgcgcacaggcgcgcggcgcgctgcagtacctccaacggggtctcgcccagatccccaggcaccgcgcccgactctgccaccaccgcaaacaccagggagcaatacacgttgagaaagtgctctgccaccgccgccttcacggcatccggaccggccgcgggatccgcaggcaggtgggtgcgcacctcgtcgggtagcttggagacaaacagctccaggccggtccgcggcgccagcgcctgcaggtgcctcaccaccggggccgggtcatgcgatctgtttagtccggagaagatagggcccttggcaagccgctggaccagcttcagggtctccaagatgcgcaccgcattgtcggagctgtcgcgatagaggttagggtaggtgtccggtccatccgtgggctcaaacctgcccagacacaccactgtctgctgggggatcatccttctcagggagatgcattctttggaagtagtggtagagatggagcagactgccagggcgttgccaggagtggtggcgatggtgcgcaccgtttttaagaaaccccccagggtggggactcccgctccctgcagcatctcggcctgctgtacgcccttggcgaatatgcgacggaatcggctgtgcgcacggggtcccagggccggttcggtggcatacaggccggtgagggccccctgtgtctgtccgcctggaaacagggtgctgtgaaacagcaggttgccaaggccgcgaatacccctctgcacgctgctgtggacgtgggtgtacgctccgtggatcccgaacgcctgtctggcacagttccagggccaccgttccatggtgcatcttcccggtatcacaaagtacctggccacgttataattgtccccggttgaagcctgcaccgccagcggtagcaggtctgcccccagggatatcataacagcctgcataatgacatcatcttcaatgtgtggcctagccacgggctggggaccctcgggcacttccaacccctcgtacggtaccaggtcggtattttgtgtaaatgccctgataaactgaggtgggtgtggttctagcagggtctgtgtgattttggacaccaggtgcctgcccacttccactctagcccactcctgcaatcctagctcttgcagcagaactgcaagctctgttgacaatgttgtgggccggtggtgcatgtttggcccgtagccaaaggatacaacacgctcgctcccccgtggcacagaccgcctgatgacatggggatatccaaggagcggtgacagcacagcgagcaccgtctgtatttccacatcccgtctctctcgctcctccctcgaagtgggaggtcttcggaaagttatccatagcagatagtagcctccggtgccaccgggtacgagagtgagtgtgcccgtacggcttgtataaaagttcacaaaagcttcctcatccgcggtgagatcactctccaaccacagcccagtgacgtcgtaggccatgcctagagggcgcaccgcccccggggacaccctctgtagtcaggctgccgagaaacccgcgagatctctggggagtaggaagaaacttagaatccccaaatatgtcgcagtcacaggttgtcgggcagagtctgtttccgctttcatgggatccacagttacttgtagccatgtcactaacctcaaatactcaaaaaaagctatcgatggaaaaatgctgtggtcctaggttagtccgtgggaaacaaaacttcctcatacacttcatctgcaggctgaaatggtggcggatccagactccttacaccacagttgctcacattagagatacctgattggttaatacaagcggacgcacgcgttggtggaggcgtgttgtcgcccaagatactagcataggtgactgtgcgttcgctatgtagttgctgcatttcaagttgggtcgttacttctgtgttgcaaacccttactggagataatgccatgtctgttgtggaacttaaaatacgcgagtgtataacatttctagatggtagaggtggtaaacggcgagctaaatgattaacatcgggacatatcctgcctgcatgagcatgtggtgtgtcgtgtggtgtatatattggtaatcttgttgttacattgttgaacgacacaagtctgctctctcggtagagataacccaccagtacggcttggccagtacctaataagaaaa

SEQ ID NO: 204 - DNA, 38 bp, Kaposi′s sarcoma-associated herpesvirus
acattgcttttgggatcagacccctcatttaatcgcat

SEQ ID NO: 205 - DNA, 199 bp, Kaposi′s sarcoma-associated herpesvirus
agaatgctttgccagctgcgcatttacgcgacggatctctaacgatacccatgttgggtccacaagtctaaggccagcgagacaagagcgtttcgtgaaacgtgcctgccaaggagtgggatctcccaattacaggagaacagcgaacggcgcggggtgtcggaaggcacaactctactgcacaaaattgtcttgtaaa

SEQ ID NO: 206 - DNA, 1144 bp, Kaposi′s sarcoma-associated herpesvirus
acgggaaacaggtgtctatcttggccggctggttactcaaatgggaacaatggcgccaccttgctgtctttgtaggcattagaagaaaaggatgcacaactatgtttcctagcggcgagattggaggcacataaggaacagattattttccttcgcgacatgctgatgcgaatgtgccagcagccagcgtcgccaacggacgcgccactcccaccatgttgaagcttggttgtgccgtcgtccgggagaaccatgccagactttgtgtggtaagaaggaattgttatccggcagcaatattaaagggacccaagttaatcccttaatcctctgggattaataaccatgagttccacacagattcgcacagaaatccctgtggcgctcctaatcctatgcctttgtctggtggcgtgccatgccaattgtcccacgtatcgttcgcatttgggattctggcaagagggttggagtggacaggtttatcaggactggctaggcaggatgaactgttcctacgagaatatgacggccctagaggccgtctccctaaacgggaccagactagcagctggatctccgtcgagtgagtatccaaatgtctccgtatctgttgaagatacgtctgcctctgggtctggagaagatgcaatagatgaatcggggtcgggggaggaagagcgtcccgtgacctcccacgtgacttttatgacacaaagcgtccaggccaccacagaactgaccgatgccttaatatcagccttttcaggtgtattacacgtttcaactgtaatccctcgcaattgggtaaaccgtcggtgtgtagggataaagcgtaaccttacgttctgtctcatctacaggatcatattcatctggggaaccatccaggaccacgcgaattcgcgtatcaccggtcgcagaaaacggcagaaatagtggtgctagtaaccgtgtgccattttctgccaccactacaacgactagaggaagagacgcgcactacaatgcagaaatacggacccatctttacatactatgggctgtgggtttattgctgggacttgtccttatactttacctgtgcgttccacgatgccggcgtaagaaaccctacatagtgtaacacaaaaccataaaagta

SEQ ID NO: 207 - DNA, 50 bp, Kaposi′s sarcoma-associated herpesvirus
ataacaagctgttgctaatttttggtccgtagaatgtatgtatctgattt

SEQ ID NO: 208 - DNA, 6226 bp, Kaposi′s sarcoma-associated herpesvirus
ctagatggacaccccgtgaaccgtcgtgcttacccacccccttctgattctgacagacaacactactatgtcccaaagactgttttttacagcccgatggcccttcaggcctccttgagtgtctagctggtcccgtggtcattgtgtggtttggcagtcacttccccattttggtgtcgcgttttgggttttgccctgcccccagccaacgtggatcatattctttcccgtcaggggagtgacaagctataggacagaaaggtcacctggcccaaacggaggatcctaggtgggtgtgcatttattagacgttggtgtgttgaaggacggatcaggcggggaggagggggtgggggagacttactgcagcactaggttaggttgaaagccggggtaaaaggcgtggctaaacaacacctatactacttgttattgtaggccatggcggccgaggatttcctaaccatcttcttagatgatgatgaatcctggaatgaaactctaaatatgagcggatatgactactctggaaacttcagcctagaagtgagcgtgtgtgagatgaccaccgtggtgccttacacgtggaacgttggaatactctctctgattttcctcataaatgttcttggaaatggattggtcacctacattttttgcaagcaccgatcgcgggcaggagcgatagatatactgctcctgggtatctgcctaaactcgctgtgtcttagcatatctctattggcagaagtgttgatgtttttgtttcccaatatcatctccacaggcttgtgcagacttgaaatttttttttactatttatatgtctacttggatatcttcagtgttgtgtgcgtcagtctagtgaggtacctcctggtggcatattctacgcgttcctggcccaagaagcagtccctcggatgggtactgacatccgctgcactgttaattgcattggtgctgtcgggggatgcctgtcgacacaggagcagggtggtcgacccggtcagcaagcaggccatgtgttatgagaacgcgggaaacatgactgcagactggcgactgcatgtcagaaccgtgtcagttactgcaggtttcctgttacccctggccctccttattctgttttatgctctcacctggtgtgtggtgaggaggacaaagctgcaagccaggcggaaggtaaggggggtgattgttgctgtggtgctgctgttttttgtgttttgcttcccttaccacgtactaaatctactggacactctgctaaggcgacgctggatccgggacagctgctatacgcgggggttgataaacgtgggtctggcagtaacctcgttactgcaggcactgtacagcgccgtggttcccctgatatactcctgcctgggatccctctttaggcagaggatgtacggtctcttccaaagcctcaggcagtctttcatgtccggcgccaccacgtagcccgcggatgtctacgtgcccttcccccttaatttaatctagcctcccgttcccatgatgcagagaggcgaatttggtttgtacacagatgtgactatgtatttgttttattatgcgattaaatgaggggtctgatcccaaaagcaatgtttagtggtggtcgttgatcttcttgacgctccataggtagattgactggaacgccatggcccacggggacatggacaggggtgttaggtctggtggaacatgctgccactgccacggatggaacatcagagatgggtctatgatcagggcagcgtgtcgcccgtcactggatgtaagtccggccaccgtggagttgcctgtggggtttctgggatagtgtctggctggcagggtctcatccgcggcatttccatggtaggtgagggttatctcgcctcgctgtctcagtatgtactcgagggcgtcctgctcgtaccggacccccaggtactctccctgggcccagctgggcagcaccgtcccccgcaacactcggaggaaaacgctcttagtgttctgagggatctgtatgtttagccagtggctgtcatacagcttggacacgttggtctccaggtttaccgcccagcgctggggtggtgtgggtccgtacgtgtatggtgaggattccgaccggcccactacacccagggccaccagcagctggaagcccacctcgccacagcagatggagaatgtgtcgggtctgtttagaaactctgtcagggtggaggcacaggtagggtcgttacacagcgccaggacccatcccctggcgctggcgtagctggcctggcagcctgttctgagacatgtaatcagaccagagaaccccgacaaggactgtcctcgtttaagctcttccacagtcaccgtggccacctcaaagcccgtgttctgcaacgcggccatgagcgcgtacggggcactgctcccaggcagcaccaacgcggccacacggcgcggggaggtggggcacgaaaacaggcgcagctgactcccaaggcacatggcccttaggctgcccaggtgatgctccagacgacccaggtccttcctgtgcatgtcctccagtgggtgcaggggaggcgtcaccaggttccacatttcgtcagaaaaggaggtccatgagacttgcaaggaagtcagggtctcttgaaacacaactgtctcgttctgcaaaaccgtgacgttgttgccttgtccctcggggccaacggtgcccagtgggtgtgccacgcagcggtagtccctggccgcccgcagcacctctgacaagtgtacctggggcacctcaaccagtgccccaggggtctctgaaaccataagttcgagcgggttagggtgggcgggtagtgagagctgcagtcccctgcagccggccagggccatctcgattgcagatgggagaagccctccgtcccctatgtcgtgcccagatacaatgagcctcttggacatcaggtacttaacaagcatgaacaggctggcgaccgtggacgggttcagagggggtattgggtgcctggatgccaggaagttgtgctcgaaggtggacccggctatgagacagctctgattcacggccaggtataccagggcgttgccttcgacctttacgtccggggtgaccctgtatctggatcccttgacctcggcccagctggtaaacaccaccgagttgaagggaaggacctccaccgtttcttgctgttgtgtgatgcgcacatggcgctccgaaagcgtcggagagctggcagccgaggagatggacagtgccactcccagctcccggcagaattccttgcaggcgaagaggcactcctgtaggaggccggcttggtggtcctctggactccacgccacggcgccagttagcactacgtcctggagcttggacacgggactgaacatgaggttggtgagagcctcggtgatggcataggtggccccggtggatacattagtagccatcttgtaggcctgctcccccatggccattgcctgacccctccacgctggcactggaagcagctcctggggcagggccttcacccaggtctcgaagtccttgtgtaggaggttggccatggacggagtgatggcctccaccgtgtcgggcactctgggcgccaccctctcggccagcatggacgagtgcagcaccaggtggtagtctgaaaccggtatgtccaggggtcccacgccagcctgttgggcgatgaggccgttggagcatcggtccatgtgtcgcgtaaagaactccttgctgccaaccgtcgagtggcgaagtaactggtggattgtggagccggtggcaaaaaggccccagtcaacatcctcggggtgccccgagacgcggacaccatcggacagcgccagccagggggacgggggggtggacgacggctggtctacagagaagaccctcgtggtctccccggtcaggtcgtctactattctgatgcctgggtgctccgaggtcctcccgaggaccgttacctggcacgcgcacaggcgcgcggcgcgctgcagtacctccaacggggtctcgcccagatccccaggcaccgcgcccgactctgccaccaccgcaaacaccagggagcaatacacgttgagaaagtgctctgccaccgccgccttcacggcatccggaccggccgcgggatccgcaggcaggtgggtgcgcacctcgtcgggtagcttggagacaaacagctccaggccggtccgcggcgccagcgcctgcaggtgcctcaccaccggggccgggtcatgcgatctgtttagtccggagaagatagggcccttggcaagccgctggaccagcttcagggtctccaagatgcgcaccgcattgtcggagctgtcgcgatagaggttagggtaggtgtccggtccatccgtgggctcaaacctgcccagacacaccactgtctgctgggggatcatccttctcagggagatgcattctttggaagtagtggtagagatggagcagactgccagggcgttgccaggagtggtggcgatggtgcgcaccgtttttaagaaaccccccagggtggggactcccgctccctgcagcatctcggcctgctgtacgcccttggcgaatatgcgacggaatcggctgtgcgcacggggtcccagggccggttcggtggcatacaggccggtgagggccccctgtgtctgtccgcctggaaacagggtgctgtgaaacagcaggttgccaaggccgcgaatacccctctgcacgctgctgtggacgtgggtgtacgctccgtggatcccgaacgcctgtctggcacagttccagggccaccgttccatggtgcatcttcccggtatcacaaagtacctggccacgttataattgtccccggttgaagcctgcaccgccagcggtagcaggtctgcccccagggatatcataacagcctgcataatgacatcatcttcaatgtgtggcctagccacgggctggggaccctcgggcacttccaacccctcgtacggtaccaggtcggtattttgtgtaaatgccctgataaactgaggtgggtgtggttctagcagggtctgtgtgattttggacaccaggtgcctgcccacttccactctagcccactcctgcaatcctagctcttgcagcagaactgcaagctctgttgacaatgttgtgggccggtggtgcatgtttggcccgtagccaaaggatacaacacgctcgctcccccgtggcacagaccgcctgatgacatggggatatccaaggagcggtgacagcacagcgagcaccgtctgtatttccacatcccgtctctctcgctcctccctcgaagtgggaggtcttcggaaagttatccatagcagatagtagcctccggtgccaccgggtacgagagtgagtgtgcccgtacggcttgtataaaagttcacaaaagcttcctcatccgcggtgagatcactctccaaccacagcccagtgacgtcgtaggccatgcctagagggcgcaccgcccccggggacaccctctgtagtcaggctgccgagaaacccgcgagatctctggggagtaggaagaaacttagaatccccaaatatgtcgcagtcacaggttgtcgggcagagtctgtttccgctttcatgggatccacagttacttgtagccatgtcactaacctcaaatactcaaaaaaagctatcgatggaaaaatgctgtggtcctaggttagtccgtgggaaacaaaacttcctcatacacttcatctgcaggctgaaatggtggcggatccagactccttacaccacagttgctcacattagagatacctgattggttaatacaagcggacgcacgcgttggtggaggcgtgttgtcgcccaagatactagcataggtgactgtgcgttcgctatgtagttgctgcatttcaagttgggtcgttacttctgtgttgcaaacccttactggagataatgccatgtctgttgtggaacttaaaatacgcgagtgtataacatttctagatggtagaggtggtaaacggcgagctaaatgattaacatcgggacatatcctgcctgcatgagcatgtggtgtgtcgtgtggtgtatatattggtaatcttgttgttacattgttgaacgacacaagtctgctctctcggtagagataacccaccagtacggcttggccagtacctaataagaaaa

SEQ ID NO: 209 - DNA, 500 bp, Varicella zoster virus
gtgcaacttttgcttatattttacatacaaacttgtgtgtaccatagatgaacacatttttatttgttttgaattattaaacttaagacatggccgtgaatggtgaaagagctgtccatgatgaaaacctgggtgtgttagacagagaattaatccgcgctcaatcaatccaaggatgtgtcggaaaccctcaagaatgtaattcgtgtgcaataacctcagcatcgcggttgtttctcgtgggactacaagcaagcgttatcacgtccgggttaattttacaatatcacgtctgcgaagctgccgtcaatgcaactattatggggttgatcgtcgtttcggggttatggccaacatccgtgaaatttctacgcacattagcaaaattgggacgatgtttgcagacggtggtcgtgttgggttttgctgtgttatgggcggttggttgcccaatatcccgggatcttccatttgtagaattactgggaatttccatatcc

SEQ ID NO: 210 - DNA, 500 bp, Varicella zoster virus
gcccccagccagccaaaaaaattgcccgtgtgggaggtctacagcacccttttgtaaaaacggatattaacacgattaacgttgaacaccattttatagacacgctacagaagacatcaccgaacatggactgtcgcgggatgacagcgggtatttttattcgtttatcccacatgtataaaattctaacaactctggagtctccaaatgatgtaacctacacaacacccggttctaccaacgcactgttctttaagacgtccacacagcctcaggagccgcgtccggaagagttagcatccaaattaacccaagacgacattaaacgtattctattaacaatagaatcggagactcgtggtcagggcgacaatgccatttggacactactcagacgaaatttaatcaccgcatcaactcttaaatggagtgtatctggacccgtcattccacctcagtggttttaccaccataacactacagacacatacggtgatgcg

SEQ ID NO: 211 - DNA, 67 bp, Varicella zoster virus
caaaaaaacacgccgcaacaacccatccttaaaataaaaggtttatttactttacaacccgtggtga

SEQ ID NO: 212 - DNA, 500 bp, Varicella zoster virus
agcattgtataaaaacacgcatgcgggcttgctgttctcatttctaggttttgtcttaaatacacccgccatgagcatctctggacccccaacgacgtttattttatataggttacatggggttaggcgggttcttcactggactttaccggatcatgaacaaacactctacgcatttacgggtgggtcaagatcaatggcggtgaagacggacgctcgatgtgatacaatgagcggtggtatgatcgtccttcaacacacccatacagtgaccctgctaaccatagactgttctactgacttttcatcatacgcatttacgcaccgggatttccacttacaggacaaaccccacgcaacatttgcgatgccgtttatgtcctgggtcggttctgacccaacatctcagctgtacagtaatgtggggggggtactatccgtaataacggaagatgacctatccatgtgtatctcaattgttatatacggtttacgggtaa

SEQ ID NO: 213 - DNA, 500 bp, Varicella zoster virus
cactccaatcgaccctcttgcgtaccataatgttttcggagttgcctccttccgtaccgacggcattgcttcaatggggttggggattgcatcgtggaccgtgttcgatcccaaattttaaacaggtagccagccaacacagtgttcagaacgattttacagaaaatagcgttgatgcaaatgaaaaatttccgattgggcacgcgggctgtattgagaaaaccaaagacgactatgtaccatttgatacgttgttcatggtatcatctattgacgaacttgggcggagacaattaaccgacaccatccgccgcagcttggttatgaacgcctgtgaaataacggtcgcgtgtacgaaaaccgcagccttttctggtcgaggcgtgtcacgacaaaaacacgtgaccctatctaaaaataaattcaatccatccagtcataagagcctgcaaatgtttgtgttgtgtcaaaaaacccatgcaccccgtgtcagaaaccta

SEQ ID NO: 214 - DNA, 110 bp, Varicella zoster virus
tttgttgggagggggaaggaaatgccttaaacatccacagtctgctttattaccaactgtatgtaaattatgatcattaaacgtgcattttaaaaatacctgagtgttgc

SEQ ID NO: 215 - DNA, 88 bp, Varicella zoster virus
cggagtcccctccttttctcgtgagcgccactggcgcgcggactgtttgttgttaataaaagcggaacggtttttatgaaaaaagtgt

SEQ ID NO: 216 - RNA, 21 nt, Herpes simplex virus
uggaaggacgggaaguggaag

SEQ ID NO: 217 - RNA, 20 nt, Herpes simplex virus
uggcggcccggcccggggcc

SEQ ID NO: 218 - RNA, 23 nt, Epstein Barr virus
uagcaccgcuauccacuaugucu

SEQ ID NO: 219 - RNA, 23 nt, Epstein Barr virus
ucuuaguggaagugacgugcugu

SEQ ID NO: 220 - RNA, 22 nt, Epstein Barr virus
uauuuucugcauucgcccuugc

SEQ ID NO: 221 - RNA, 22 nt, Epstein Barr virus
cgcaccacuagucaccaggugu

SEQ ID NO: 222 - RNA, 22 nt, Epstein Barr virus
aaccuaguguuaguguugugcu

SEQ ID NO: 223 - RNA, 22 nt, Epstein Barr virus
gaccugaugcugcuggugugcu

SEQ ID NO: 224 - RNA, 24 nt, Epstein Barr virus
caaggugaauauagcugcccaucg

SEQ ID NO: 225 - RNA, 22 nt, Epstein Barr virus
cggggaucggacuagccuuaga

SEQ ID NO: 226 - RNA, 22 nt, Epstein Barr virus
gguugguccaauccauaggcuu

SEQ ID NO: 227 - RNA, 22 nt, Epstein Barr virus
caucauaguccaguguccaggg

SEQ ID NO: 228 - RNA, 22 nt, Epstein Barr virus
gucacaaucuauggggucguag

SEQ ID NO: 229 - RNA, 22 nt, Epstein Barr virus
uacgguuuccuagauuguacag

SEQ ID NO: 230 - RNA, 22 nt, Epstein Barr virus
uaacacuucaugggucccguag

SEQ ID NO: 231 - RNA, 22 nt, Epstein Barr virus
acauaaccauggaguuggcugu

SEQ ID NO: 232 - RNA, 21 nt, Epstein Barr virus
acgcacaccaggcugacugcc

SEQ ID NO: 233 - RNA, 22 nt, Epstein Barr virus
gacaguuuggugcgcuaguugu

SEQ ID NO: 234 - RNA, 23 nt, Epstein Barr virus
uccugugguguuuggugugguuu

SEQ ID NO: 235 - RNA, 23 nt, Epstein Barr virus
uguaacuugccagggacggcuga

SEQ ID NO: 236 - RNA, 22 nt, Epstein Barr virus
uaaaugcugcaguaguagggau

SEQ ID NO: 237 - RNA, 22 nt, Epstein Barr virus
uacccuacgcugccgauuuaca

SEQ ID NO: 238 - RNA, 22 nt, Epstein Barr virus
agugguuuuguuuccuugauag

SEQ ID NO: 239 - RNA, 22 nt, Epstein Barr virus
auagagugggugugugcucuug

SEQ ID NO: 240 - RNA, 22 nt, Epstein Barr virus
uuguaugccugguguccccuua

SEQ ID NO: 241 - RNA, 22 nt, Epstein Barr virus
aagaggacgcaggcauacaagg

SEQ ID NO: 242 - RNA, 22 nt, Epstein Barr virus
caaguucgcacuuccuauacag

SEQ ID NO: 243 - RNA, 22 nt, Epstein Barr virus
uguuuuguuugcuugggaaugc

SEQ ID NO: 244 - RNA, 22 nt, Epstein Barr virus
caugaaggcacagccuguuacc

SEQ ID NO: 245 - RNA, 22 nt, Epstein Barr virus
guagcaggcaugucuucauucc

SEQ ID NO: 246 - RNA, 22 nt, Epstein Barr virus
uaaccugaucagccccggaguu

SEQ ID NO: 247 - RNA, 22 nt, Epstein Barr virus
aaauucuguugcagcagauagc

SEQ ID NO: 248 - RNA, 23 nt, Epstein Barr virus
uaacgggaaguguguaagcacac

SEQ ID NO: 249 - RNA, 21 nt, Human cytomegalovirus
ucacgggaaggcuaguuagac

SEQ ID NO: 250 - RNA, 20 nt, Human cytomegalovirus
uaacuagccuucccgugaga

SEQ ID NO: 251 - RNA, 21 nt, Human cytomegalovirus
cggcauguugcgcgccgugau

SEQ ID NO: 252 - RNA, 22 nt, Human cytomegalovirus
ucguugaagacaccuggaaaga

SEQ ID NO: 253 - RNA, 21 nt, Human cytomegalovirus
agacaccuggaaagaggacgu

SEQ ID NO: 254 - RNA, 21 nt, Human cytomegalovirus
ugcgcgagaccugcucguugc

SEQ ID NO: 255 - RNA, 21 nt, Human cytomegalovirus
ugcgcgucucggugcucucgg

SEQ ID NO: 256 - RNA, 20 nt, Human cytomegalovirus
ggggaugggcuggcgcgcgg

SEQ ID NO: 257 - RNA, 21 nt, Human cytomegalovirus
ugcgucucggccucguccaga

SEQ ID NO: 258 - RNA, 21 nt, Human cytomegalovirus
uggccaugucguuucgcgucg

SEQ ID NO: 259 - RNA, 21 nt, Human  cytomegalovirus
uggcgucgucgcucggcgggu

SEQ ID NO: 260 - RNA, 21 nt, Human cytomegalovirus
ugacguuguuuguggguguug

SEQ ID NO: 261 - RNA, 22 nt, Human cytomegalovirus
aagugacggugagauccaggcu

SEQ ID NO: 262 - RNA, 21 nt, Human cytomegalovirus
ucguccuccccuucuucaccg

SEQ ID NO: 263 - RNA, 22 nt, Human cytomegalovirus
cgacauggacgugcagggggau

SEQ ID NO: 264 - RNA, 21 nt, Human cytomegalovirus
ugacaagccugacgagagcgu

SEQ ID NO: 265 - RNA, 22 nt, Human cytomegalovirus
uuaugauaggugugacgauguc

SEQ ID NO: 266 - RNA, 21 nt, Human cytomegalovirus
ugauaggugugacgaugucuu

SEQ ID NO: 267 - RNA, 21 nt, Human cytomegalovirus
aaccgcucaguggcucggacc

SEQ ID NO: 268 - RNA, 22 nt, Human cytomegalovirus
agcggucuguucagguggauga

SEQ ID NO: 269 - RNA, 23 nt, Human cytomegalovirus
auccacuuggagagcucccgcgg

SEQ ID NO: 270 - RNA, 21 nt, Human cytomegalovirus
uuggaugugcucggaccguga

SEQ ID NO: 271 - RNA, 22 nt, Human cytomegalovirus
gauugugcccggaccgugggcg

SEQ ID NO: 272 - RNA, 23 nt, Kaposi′s sarcoma-associated herpesvirus
auuacaggaaacuggguguaagc

SEQ ID NO: 273 - RNA, 22 nt, Kaposi′s sarcoma-associated herpesvirus
aacuguaguccgggucgaucug

SEQ ID NO: 274 - RNA, 22 nt, Kaposi′s sarcoma-associated herpesvirus
ucacauucugaggacggcagcg

SEQ ID NO: 275 - RNA, 21 nt, Kaposi′s sarcoma-associated herpesvirus
ucgcggucacagaaugugaca

SEQ ID NO: 276 - RNA, 22 nt, Kaposi′s sarcoma-associated herpesvirus
agcuaaaccgcaguacucuagg

SEQ ID NO: 277 - RNA, 22 nt, Kaposi′s sarcoma-associated herpesvirus
uagaauacugaggccuagcuga

SEQ ID NO: 278 - RNA, 22 nt, Kaposi′s sarcoma-associated herpesvirus
uaggaugccuggaacuugccgg

SEQ ID NO: 279 - RNA, 22 nt, Kaposi′s sarcoma-associated herpesvirus
ccagcagcaccuaauccaucgg

SEQ ID NO: 280 - RNA, 22 nt, Kaposi′s sarcoma-associated herpesvirus
ugaugguuuucgggcuguugag

SEQ ID NO: 281 - RNA, 21 nt, Kaposi′s sarcoma-associated herpesvirus
ugaucccauguugcuggcgcu

SEQ ID NO: 282 - RNA, 22 nt, Kaposi′s sarcoma-associated herpesvirus
uaggcgcgacugagagagcacg

SEQ ID NO: 283 - RNA, 22 nt, Kaposi′s sarcoma-associated herpesvirus
acccagcugcguaaaccccgcu

SEQ ID NO: 284 - RNA, 22 nt, Kaposi′s sarcoma-associated herpesvirus
cuggguauacgcagcugcguaa

SEQ ID NO: 285 - RNA, 22 nt, Kaposi′s sarcoma-associated herpesvirus
uaguguuguccccccgaguggc

SEQ ID NO: 286 - RNA, 22 nt, Kaposi′s sarcoma-associated herpesvirus
ugguguuguccccccgaguggc

SEQ ID NO: 287 - RNA, 22 nt, Kaposi′s sarcoma-associated herpesvirus
uuaaugcuuagccuguguccga

SEQ ID NO: 288 - RNA, 22 nt, Kaposi′s sarcoma-associated herpesvirus
accaggccaccauuccucuccg

SEQ ID NO: 289 - RNA, 22 nt, Homo sapiens
ugagguaguagguuguauaguu

SEQ ID NO: 290 - RNA, 22 nt, Homo sapiens
cuauacaaccuacugccuuccc

SEQ ID NO: 291 - RNA, 22 nt, Homo sapiens
ugagguaguagguuguaugguu

SEQ ID NO: 292 - RNA, 22 nt, Homo sapiens
agagguaguagguugcauaguu

SEQ ID NO: 293 - RNA, 22 nt, Homo sapiens
ugagguaggagguuguauaguu

SEQ ID NO: 294 - RNA, 22 nt, Homo sapiens
ugagguaguagauuguauaguu

SEQ ID NO: 295 - RNA, 22 nt, Homo sapiens
ugagguaguaguuuguacaguu

SEQ ID NO: 296 - RNA, 22 nt, Homo sapiens
ugagguaguaguuugugcuguu

SEQ ID NO: 297 - RNA, 22 nt, Homo sapiens
uggaauguaaagaaguauguau

SEQ ID NO: 298 - RNA, 23 nt, Homo sapiens
ucuuugguuaucuagcuguauga

SEQ ID NO: 299 - RNA, 22 nt, Homo sapiens
caggccauauugugcugccuca

SEQ ID NO: 300 - RNA, 22 nt, Homo sapiens
cgaaucauuauuugcugcucua

SEQ ID NO: 301 - RNA, 22 nt, Homo sapiens
uagcagcacguaaauauuggcg

SEQ ID NO: 302 - RNA, 23 nt, Homo sapiens
caaagugcuuacagugcagguag

SEQ ID NO: 303 - RNA, 24 nt, Homo sapiens
caaagugcuuacagugcagguagu

SEQ ID NO: 304 - RNA, 23 nt, Homo sapiens
uaaggugcaucuagugcagauag

SEQ ID NO: 305 - RNA, 23 nt, Homo sapiens
uaaggugcaucuagugcagauag

SEQ ID NO: 306 - RNA, 22 nt, Homo sapiens
acugcauuaugagcacuuaaag

SEQ ID NO: 307 - RNA, 23 nt, Homo sapiens
caaagugcucauagugcagguag

SEQ ID NO: 308 - RNA, 21 nt, Homo sapiens
aucacauugccagggauuucc

SEQ ID NO: 309 - RNA, 21 nt, Homo sapiens
aucacauugccagggauuacc

SEQ ID NO: 310 - RNA, 22 nt, Homo sapiens
uggcucaguucagcaggaacag

SEQ ID NO: 311 - RNA, 22 nt, Homo sapiens
uguaaacauccucgacuggaag

SEQ ID NO: 312 - RNA, 22 nt, Homo sapiens
cuuucagucggauguuugcagc

SEQ ID NO: 313 - RNA, 22 nt, Homo sapiens
cugggagguggauguuuacuuc

SEQ ID NO: 314 - RNA, 23 nt, Homo sapiens
uguaaacauccuacacucucagc

SEQ ID NO: 315 - RNA, 20 nt, Homo sapiens
uguaaacauccuugacugga

SEQ ID NO: 316 - RNA, 22 nt, Homo sapiens
cuuucagucggauguuuacagc

SEQ ID NO: 317 - RNA, 23 nt, Homo sapiens
caaagugcuguucgugcagguag

SEQ ID NO: 318 - RNA, 22 nt, Homo sapiens
ugagguaguaaguuguauuguu

SEQ ID NO: 319 - RNA, 22 nt, Homo sapiens
aacccguagauccgaucuugug

SEQ ID NO: 320 - RNA, 22 nt, Homo sapiens
cacccguagaaccgaccuugcg

SEQ ID NO: 321 - RNA, 22 nt, Homo sapiens
aacccguagauccgaacuugug

SEQ ID NO: 322 - RNA, 23 nt, Homo sapiens
agcagcauuguacagggcuauga

SEQ ID NO: 323 - RNA, 23 nt, Homo sapiens
ucaaaugcucagacuccuguggu

SEQ ID NO: 324 - RNA, 23 nt, Homo sapiens
aaaagugcuuacagugcagguag

SEQ ID NO: 325 - RNA, 21 nt, Homo sapiens
uaaagugcugacagugcagau

SEQ ID NO: 326 - RNA, 23 nt, Homo sapiens
agcagcauuguacagggcuauca

SEQ ID NO: 327 - RNA, 22 nt, Homo sapiens
uuaaggcacgcggugaaugcca

SEQ ID NO: 328 - RNA, 22 nt, Homo sapiens
acaggugagguucuugggagcc

SEQ ID NO: 329 - RNA, 22 nt, Homo sapiens
ucccugagacccuaacuuguga

SEQ ID NO: 330 - RNA, 22 nt, Homo sapiens
ucguaccgugaguaauaaugcg

SEQ ID NO: 331 - RNA, 21 nt, Homo sapiens
cuuuuugcggucugggcuugc

SEQ ID NO: 332 - RNA, 22 nt, Homo sapiens
uaacagucuacagccauggucg

SEQ ID NO: 333 - RNA, 22 nt, Homo sapiens
ugugacugguugaccagagggg

SEQ ID NO: 334 - RNA, 23 nt, Homo sapiens
uuauugcuuaagaauacgcguag

SEQ ID NO: 335 - RNA, 23 nt, Homo sapiens
agcugguguugugaaucaggccg

SEQ ID NO: 336 - RNA, 22 nt, Homo sapiens
uaacacugucugguaaagaugg

SEQ ID NO: 337 - RNA, 23 nt, Homo sapiens
uguaguguuuccuacuuuaugga

SEQ ID NO: 338 - RNA, 21 nt, Homo sapiens
cauaaaguagaaagcacuacu

SEQ ID NO: 339 - RNA, 23 nt, Homo sapiens
guccaguuuucccaggaaucccu

SEQ ID NO: 340 - RNA, 22 nt, Homo sapiens
ucucccaacccuuguaccagug

SEQ ID NO: 341 - RNA, 22 nt, Homo sapiens
uagguuauccguguugccuucg

SEQ ID NO: 342 - RNA, 23 nt, Homo sapiens
aacauucaacgcugucggugagu

SEQ ID NO: 343 - RNA, 23 nt, Homo sapiens
aacauucauugcugucggugggu

SEQ ID NO: 344 - RNA, 22 nt, Homo sapiens
aacauucaaccugucggugagu

SEQ ID NO: 345 - RNA, 23 nt, Homo sapiens
aacauucauuguugucggugggu

SEQ ID NO: 346 - RNA, 21 nt, Homo sapiens
ugguucuagacuugccaacua

SEQ ID NO: 347 - RNA, 22 nt, Homo sapiens
uggacggagaacugauaagggu

SEQ ID NO: 348 - RNA, 22 nt, Homo sapiens
uguaacagcaacuccaugugga

SEQ ID NO: 349 - RNA, 21 nt, Homo sapiens
uagcagcacagaaauauuggc

SEQ ID NO: 350 - RNA, 22 nt, Homo sapiens
uagguaguuucauguuguuggg

SEQ ID NO: 351 - RNA, 22 nt, Homo sapiens
uagguaguuuccuguuguuggg

SEQ ID NO: 352 - RNA, 22 nt, Homo sapiens
uucaccaccuucuccacccagc

SEQ ID NO: 353 - RNA, 23 nt, Homo sapiens
cccaguguucagacuaccuguuc

SEQ ID NO: 354 - RNA, 23 nt, Homo sapiens
cccaguguuuagacuaucuguuc

SEQ ID NO: 355 - RNA, 22 nt, Homo sapiens
uaacacugucugguaacgaugu

SEQ ID NO: 356 - RNA, 22 nt, Homo sapiens
uaauacugccugguaaugauga

SEQ ID NO: 357 - RNA, 23 nt, Homo sapiens
uaauacugccggguaaugaugga

SEQ ID NO: 358 - RNA, 21 nt, Homo sapiens
gugccagcugcagugggggag

SEQ ID NO: 359 - RNA, 22 nt, Homo sapiens
uccuucauuccaccggagucug

SEQ ID NO: 360 - RNA, 22 nt, Homo sapiens
uggaauguaaggaagugugugg

SEQ ID NO: 361 - RNA, 22 nt, Homo sapiens
cugugcgugugacagcggcuga

SEQ ID NO: 362 - RNA, 21 nt, Homo sapiens
uaacagucuccagucacggcc

SEQ ID NO: 363 - RNA, 22 nt, Homo sapiens
accaucgaccguugauuguacc

SEQ ID NO: 364 - RNA, 22 nt, Homo sapiens
acagcaggcacagacaggcagu

SEQ ID NO: 365 - RNA, 21 nt, Homo sapiens
aggguaagcugaaccucugau

SEQ ID NO: 366 - RNA, 21 nt, Homo sapiens
agggcccccccucaauccugu

SEQ ID NO: 367 - RNA, 22 nt, Homo sapiens
uaugugggaugguaaaccgcuu

SEQ ID NO: 368 - RNA, 23 nt, Homo sapiens
uaagugcuuccauguuuugguga

SEQ ID NO: 369 - RNA, 23 nt, Homo sapiens
uaagugcuuccauguuuuaguag

SEQ ID NO: 370 - RNA, 23 nt, Homo sapiens
uaagugcuuccauguuucagugg

SEQ ID NO: 371 - RNA, 23 nt, Homo sapiens
uaagugcuuccauguuugagugu

SEQ ID NO: 372 - RNA, 20 nt, Homo sapiens
acugccccaggugcugcugg

SEQ ID NO: 373 - RNA, 20 nt, Homo sapiens
ccucugggcccuuccuccag

SEQ ID NO: 374 - RNA, 22 nt, Homo sapiens
cuggcccucucugcccuuccgu

SEQ ID NO: 375 - RNA, 22 nt, Homo sapiens
aacacaccugguuaaccucuuu

SEQ ID NO: 376 - RNA, 22 nt, Homo sapiens
ucucugggccugugucuuaggc

SEQ ID NO: 377 - RNA, 23 nt, Homo sapiens
gcaaagcacacggccugcagaga

SEQ ID NO: 378 - RNA, 23 nt, Homo sapiens
uccagcuccuauaugaugccuuu

SEQ ID NO: 379 - RNA, 23 nt, Homo sapiens
uccagcaucagugauuuuguuga

SEQ ID NO: 380 - RNA, 21 nt, Homo sapiens
ucccuguccuccaggagcuca

SEQ ID NO: 381 - RNA, 22 nt, Homo sapiens
uuauaaagcaaugagacugauu

SEQ ID NO: 382 - RNA, 23 nt, Homo sapiens
ugucugcccgcaugccugccucu

SEQ ID NO: 383 - RNA, 22 nt, Homo sapiens
aauugcacuuuagcaaugguga

SEQ ID NO: 384 - RNA, 21 nt, Homo sapiens
gugccgccaucuuuugagugu

SEQ ID NO: 385 - RNA, 23 nt, Homo sapiens
aaagugcugcgacauuugagcgu

SEQ ID NO: 386 - RNA, 23 nt, Homo sapiens
gaagugcuucgauuuuggggugu

SEQ ID NO: 387 - RNA, 22 nt, Homo sapiens
uuauaauacaaccugauaagug

SEQ ID NO: 388 - RNA, 22 nt, Homo sapiens
uauacaagggcaagcucucugu

SEQ ID NO: 389 - RNA, 22 nt, Homo sapiens
cagcagcaauucauguuuugaa

SEQ ID NO: 390 - RNA, 23 nt, Homo sapiens
aaugacacgaucacucccguuga

SEQ ID NO: 391 - RNA, 22 nt, Homo sapiens
uaauacugucugguaaaaccgu

SEQ ID NO: 392 - RNA, 22 nt, Homo sapiens
uugcauauguaggaugucccau

SEQ ID NO: 393 - RNA, 22 nt, Homo sapiens
uuuugcaauauguuccugaaua

SEQ ID NO: 394 - RNA, 22 nt, Homo sapiens
uugggaucauuuugcauccaua

SEQ ID NO: 395 - RNA, 22 nt, Homo sapiens
aaaccguuaccauuacugaguu

SEQ ID NO: 396 - RNA, 23 nt, Homo sapiens
agguuguccguggugaguucgca

SEQ ID NO: 397 - RNA, 22 nt, Homo sapiens
uaugugccuuuggacuacaucg

SEQ ID NO: 398 - RNA, 22 nt, Homo sapiens
caaccuggaggacuccaugcug

SEQ ID NO: 399 - RNA, 23 nt, Homo sapiens
aguggggaacccuuccaugagga

SEQ ID NO: 400 - RNA, 23 nt, Homo sapiens
aggaccugcgggacaagauucuu

SEQ ID NO: 401 - RNA, 22 nt, Homo sapiens
aaacaaacauggugcacuucuu

SEQ ID NO: 402 - RNA, 21 nt, Homo sapiens
cagcagcacacugugguuugu

SEQ ID NO: 403 - RNA, 21 nt, Homo sapiens
auccuugcuaucugggugcua

SEQ ID NO: 404 - RNA, 23 nt, Homo sapiens
uagcagcgggaacaguucugcag

SEQ ID NO: 405 - RNA, 22 nt, Homo sapiens
uacucaggagaguggcaaucac

SEQ ID NO: 406 - RNA, 22 nt, Homo sapiens
caaagcgcuccccuuuagaggu

SEQ ID NO: 407 - RNA, 23 nt, Homo sapiens
caaagcgcuucucuuuagagugu

SEQ ID NO: 408 - RNA, 21 nt, Homo sapiens
caaagcgcuucccuuuggagc

SEQ ID NO: 409 - RNA, 22 nt, Homo sapiens
caaagugccucccuuuagagug

SEQ ID NO: 410 - RNA, 21 nt, Homo sapiens
cuccagagggaaguacuuucu

SEQ ID NO: 411 - RNA, 21 nt, Homo sapiens
aaagugcuuccuuuuagaggg

SEQ ID NO: 412 - RNA, 22 nt, Homo sapiens
aaagugcuuccuuuuagagggu

SEQ ID NO: 413 - RNA, 23 nt, Homo sapiens
aaagugcuucucuuugguggguu

SEQ ID NO: 414 - RNA, 24 nt, Homo sapiens
acaaagugcuucccuuuagagugu

SEQ ID NO: 415 - RNA, 22 nt, Homo sapiens
acaaagugcuucccuuuagagu

SEQ ID NO: 416 - RNA, 22 nt, Homo sapiens
aaaaugguucccuuuagagugu

SEQ ID NO: 417 - RNA, 21 nt, Homo sapiens
cuccagagggaugcacuuucu

SEQ ID NO: 418 - RNA, 23 nt, Homo sapiens
cucuugagggaagcacuuucugu

SEQ ID NO: 419 - RNA, 22 nt, Homo sapiens
caaaaaccacaguuucuuuugc

SEQ ID NO: 420 - RNA, 22 nt, Homo sapiens
aaaaguacuugcggauuuugcu

SEQ ID NO: 421 - RNA, 21 nt, Homo sapiens
gcgacccacucuugguuucca

SEQ ID NO: 422 - RNA, 21 nt, Homo sapiens
gcgacccauacuugguuucag

SEQ ID NO: 423 - RNA, 21 nt, Homo sapiens
aacaggugacugguuagacaa

SEQ ID NO: 424 - RNA, 22 nt, Homo sapiens
uugugucaauaugcgaugaugu

SEQ ID NO: 425 - RNA, 22 nt, Homo sapiens
uacgucaucguugucaucguca

SEQ ID NO: 426 - RNA, 21 nt, Homo sapiens
aauggcgccacuaggguugug

SEQ ID NO: 427 - RNA, 23 nt, Homo sapiens
cugggaucuccggggucuugguu

SEQ ID NO: 428 - RNA, 22 nt, Homo sapiens
ucaccagcccuguguucccuag

SEQ ID NO: 429 - DNA, 50 bp, Artificial Sequence
  Chemically synthesized
gtcgtatccagtgcagggtccgaggtattcgcactggatacgacagcctg